(19) United States
(12) Patent Application Publication
(10) Pub. No.: US 2008/0058356 A1
(43) Pub. Date: Mar. 6, 2008

(54) 2,6 Bisheteroaryl-4-Aminopyrimidines as Adenosine Receptor Antagonists

(75) Inventors: Maria Isabel Crespo Crespo, Barcelona (ES); Maria Prat Quinones, Barcelona (ES); Siliva Gual Roig, Cabrera de Mar (ES); Julio Cesar Castro Palomino Laria, Premia de Mar (ES); Deborah H. Slee, Cardiff by the Sea, CA (US)

Correspondence Address: SEED INTELLECTUAL PROPERTY LAW GROUP PLLC, 701 FIFTH AVE, SUITE 5400, SEATTLE, WA, 98104, US

(73) Assignee: Neurocrine Biosciences, Inc., San Diego, CA (US)

(21) Appl. No.: 10/582,704

(22) PCT Filed: Dec. 14, 2004
(86) PCT No.: PCT/US04/41970

§ 371 Date: Jun. 1, 2007

(30) Foreign Application Priority Data
Dec. 15, 2003 (ES) P2000302951
Oct. 5, 2004 (EP) PCT/EP04/11086

(51) Int. Cl. A61K 31/506; A61P 25/16; C07D 401/04; C07D 403/04; C07D 409/14

(52) U.S. Cl. 514/275; 514/256; 544/324; 544/327; 544/328

(57) ABSTRACT

4-Aminopyrimidine derivatives of formula (I) FORMULA heteroaryl groups, including pharmaceutically acceptable salts thereof, wherein R¹ and R² are adenosine A_{2A} receptor antagonists useful in the treatment of movement disorders such as Parkinson's disease.

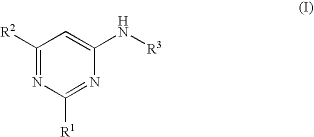

### BACKGROUND OF THE INVENTION

1. Field of the Invention

The present invention relates to new antagonists of adenosine receptors, in particular antagonist of the A_{2A} adenosine receptor subtype, the use of said compounds in the treatment of diseases, and disorders susceptible of being ameliorated by antagonism of adenosine receptors, in particular in the treatment of disorders of the central nervous which are known to be improved by the use of antagonists of the A_{2A} adenosine receptors, more specifically movement disorders such as Parkinson's disease, restless leg syndrome and dyskinesia and to pharmaceutical compositions comprising said compounds.

2. Description of the Related Art

The effects of adenosine are mediated through at least four specific cell membrane receptors so far identified and classified as receptors A₁, A_{2A}, A_{2B} and A₃ belonging to the G protein-coupled receptor family. The A₁ and A₃ receptors down-regulate cellular cAMP levels through their coupling to G proteins, which inhibit adenylate cyclase. In contrast, A_{2A} and A_{2B} receptors couple to G proteins that activate adenylate cyclase and increase intracellular levels of CAMP. Through these receptors, adenosine regulates a wide range of physiological functions.

Thus, in the cardiovascular system the activation of the A₁ receptor protects-cardiac tissue from the effects of ischemia and hypoxia. A similar protective effect is also produced by antagonism of the A_{2A} receptor, which enhances A₁-receptor-induced antiadrenergic responses and may also be useful in the treatment of acute myocardial ischemia and supraventricular arrhythmias (Norton G R et al. Am J. Physiol. 1999; 276(2 Pt 2):H341-9; Auchampach J A, Bolli R. Am J. Physiol. 1999; 276(3 Pt 2):H113-6). In addition, the A_{2B} adenosine receptor subtype (Feoktistov, I. et al., Phammacol. Rev. 1997, 49, 381402) appears to be involved in the control of vascular tone and the regulation of vascular smooth muscle growth.

In the kidney, adenosine exerts a biphasic action, inducing vasodilation at high concentrations and vasoconstriction at low concentrations. Thus, adenosine plays a role in the pathogenesis of some forms of acute renal failure that may be ameliorated by A₁ receptor antagonists (Costello-Boerrigter L C, et al. Med Clin North Am. 2003 March; 87(2): 475-91; Gottlieb S S., Drugs. 2001; 61(10): 1387-93).

Adenosine is also involved in the physiopathology of the immune system. It can induce degranulation of activated human mast cells through the A_{2B} and/or A₃ receptor. Thus A_{2B} and for A₃ antagonists prevent mast cell degranulation and are, therefore, useful in the treatment, prevention or suppression of disease states induced by activation of the A_{2B} and/or A₃ receptor and mast cell degranulation. These disease states include but are not limited to asthma, myocardial reperfusion injury, allergic reactions including but not limited to rhinitis, urticaria, scleroderm arthritis, other autoimmune diseases and inflammatory bowel diseases.

Furthermore, in the respiratory system adenosine induces bronchoconstriction, modulates airway inflammation and promotes neutrophil chemotaxis. Therefore, an adenosine antagonist would be particularly useful in the treatment of asthma.

In the gastrointestinal and metabolic system, the A_{2B} adenosine receptor subtype (Feoktistov, I. et al., Pharmacol. Rev. 1997, 49, 381-402) seems to be involved in the regulation of hepatic glucose production, the modulation of intestinal tone, as well as intestinal secretion. Thus, A_{2B} antagonists may also be useful in the treatment of diabetes mellitus and obesity.

In the central nervous system adenosine is a potent endogenous neuromodulator, which controls the presynaptic release of many neurotransmitters and is thus involved in motor function, sleep, anxiety, pain and psychomotor activity. All adenosine receptor subtypes are present in the brain, with A₁ and A_{2A} subtypes being differentially distributed. The former are found predominantly in the hippocampus and cortex, whilst the latter are found mainly in the striatum. Adenosine A_{2A} receptors modulate the release of GABA in the striatum, which possibly regulates the activity of medium spiny neurons.

Thus, A_{2A} receptor antagonists may be a useful treatment for neurodegenerative movement disorders such as Parkinson and Huntington's disease (Tuite P, et al., J. Expert Opin Investig Drugs. 2003; 12: 1335-52; Popoli P. et al. J. Neurosci. 2002; 22:1967-75), dystonias such as restless leg syndrome (Happe S, et al., Neuropsychobiology. 2003; 48: 82-6), and dyskinesias such as those caused by prolonged use of neuroleptic and dopaminergic drugs (Jenner P. J Neurol. 2000; 247 Suppl 2: 1143-50).

In the treatment of Parkinson's disease an A_{2A} antagonist may be useful not only as monotherapy, but also when administered in combination with L-DOPA and/or one or more of the following drugs: dopamine agonists, inhibitors of dopamine decarboxylase, catechol-O-methyltransferase inhibitors and inhibitors of monoamine oxidase.

In addition, A_{2A} antagonists may have therapeutic potential as neuroprotectants (Stone T W. et al., Drug. Dev. Res. 2001; 52: 323-330), and in the treatment of sleep disorders (Dunwiddie T V et al., Ann. Rev. Neurosci. 2001; 24: 31-55).

It has now been found that certain 4-aminopyrimidine derivatives are novel potent antagonists the A_{2A} adenosine receptor and can therefore be used in the treatment or prevention of diseases susceptible to amelioration by antagonism of the adenosine receptor

Further objectives of the present invention are to provide a method for preparing said compounds; pharmaceutical compositions comprising an effective amount of said compounds; the use of the compounds in the manufacture of a medicament for the treatment of pathological conditions or diseases susceptible of being improved by antagonism of an adenosine receptor, in particular by antagonism of the A_{2A} adenosine receptor; methods of treatment of pathological conditions or diseases susceptible to amelioration by antagonism of an adenosine receptor, in particular by antagonism of the A_{2A} adenosine receptor comprising the administration of the compounds of the invention to a subject in need of treatment and combinations of said compounds with one or more of the following drugs: L-DOPA, dopamine agonists, inhibitors of dopamine decarboxylase, catechol-O-methyltransferase inhibitors and inhibitors of monoamine oxidase.

### BRIEF SUMMARY OF THE INVENTION

Thus, the present invention is directed to 4-aminopyrimidine derivatives of formula (I)

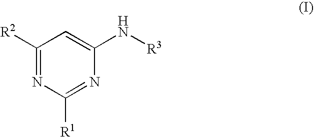

wherein
R¹ and R² independently represent a monocyclic or polycyclic heteroaryl group optionally substituted by one or more substituents selected from the group consisting of halogen atoms, straight or branched, optionally substituted lower alkyl, cycloalkyl, hydroxy, straight or branched, optionally substituted lower alkoxy, —SH, straight or branched optionally substituted lower alkylthio, cyano, —NR′R″, —CO₂R′, wherein R′ and R″ each independently represents a hydrogen atom or a straight or branched, optionally substituted lower alkyl group or R′ and R″ together with the nitrogen atom to which they are attached form a cyclic group;

### R³ represents a group selected from —COR⁴, —CON(R⁴)R⁵, —COOR⁴ and —R⁶

wherein R⁴ represents a group selected from:
hydrogen atoms,
a straight or branched lower alkyl group which is optionally substituted by one or more halogen atoms or by one or more cycloalkyl, hydroxy, lower alkoxy, lower alkylthio, amino, mono- or dialkylamino, alkoxyalkyl, hydroxycarbonyl, alkoxycarbonyl and nitrile groups;
a group of formula:

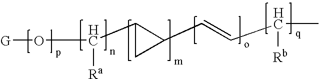

wherein:
m, o and p are independently 0 or 1;
n and q are independently selected from integers from 0 to 6;
R^{a} and R^{b} are independently a hydrogen atom or a lower alkyl group;
G is a group selected from cycloalkyl, aryl or heteroaryl groups which are optionally substituted by one or more halogen atoms or by one or more lower alkyl, cycloalkyl, lower haloalkyl, hydroxy, lower alkoxy, lower alkylthio, amino, mono- or dialkylamino, hydroxyalkyl, alkoxyalkyl, hydroxycarbonyl, alkoxycarbonyl and nitrile groups;

### R⁵ represents a hydrogen atom or a lower alkyl, cycloalkyl or benzyl group; or

### R⁴ and R⁵ together with the nitrogen atom to which they are attached form a saturated or unsaturated ring which is optionally substituted by one or more lower alkyl, cycloalkyl or benzyl groups;

and R⁶ represents a group selected from:
hydrogen atoms,
a straight or branched lower alkyl group which is optionally substituted by one or more halogen atoms or by one or more cycloalkyl, hydroxy, lower alkoxy, lower alkylthio, amino, mono- or dialkylamino, alkoxyalkyl, hydroxycarbonyl, alkoxycarbonyl and nitrile groups;
a group of formula:

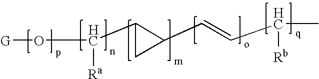

wherein:
m, o and p are independently 0 or 1;
n and q are independently selected from integers from 0 to 6;
R^{a} and R^{b} are independently a hydrogen atom or a lower alkyl group;
G is a group selected from cycloalkyl, aryl or heteroaryl groups which are optionally substituted by one or more halogen atoms or by one or more lower alkyl, cycloalkyl, lower haloalkyl, hydroxy, lower alkoxy, lower alkylthio, amino, mono- or dialkylamino, hydroxyalkyl, alkoxyalkyl, hydroxycarbonyl, alkoxycarbonyl and nitrile groups;
   or pharmaceutically acceptable salts thereof;
   with the proviso that the compound is not one of 2,6-dipyridin-4-ylpyrimidin-4-amine, 4-(3-methoxyanilino)-2,6-di(2-pyridinyl)pyrimidine, 4-(2,5-dimethoxyanilino)-2,6-di(2-pyridinyl)pyrimidine, 4-(5-methoxy-2-methylanilino)-2,6-di(2-pyridinyl)pyrimidine, 4-(2-methoxy-5-methylanilino)-2,6-di(2-pyridinyl)pyrimidine, 4-(2-chloro-5-methoxyanilino)-2,6-di(2-pyridinyl)pyrimidine, 4-(2,5-dimethylanilino)-2,6-di(2-pyridinyl)pyrimidine.

As mentioned above, the present invention is generally directed to 4-aminopyridine derivatives of formula (I):

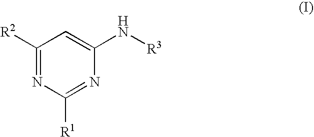

wherein
R¹ and R² independently represent a monocyclic or polycyclic heteroaryl group optionally substituted by one or more substituents selected from the group consisting of halogen atoms, straight or branched, optionally substituted lower alkyl, cycloalkyl, hydroxy, straight or branched, optionally substituted lower alkoxy, —SH, straight or branched optionally substituted lower alkylthio, cyano, —NR′R″, —CO₂R′, wherein R′ and R″ each independently represents a hydrogen atom or a straight or branched, optionally substituted lower alkyl group or R′ and R″ together with the nitrogen atom to which they are attached form a cyclic group;

### R³ represents a group selected from —COR⁴, —CON(R⁴)R⁵, —COOR⁴ and —R⁶

wherein R⁴ represents a group selected from:
hydrogen atoms,
a straight or branched lower alkyl group which is optionally substituted by one or more halogen atoms or by one or more cycloalkyl, hydroxy, lower alkoxy, lower alkylthio, amino, mono- or dialkylamino, alkoxyalkyl, hydroxycarbonyl, alkoxycarbonyl and nitrile groups;
a group of formula:

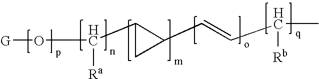

wherein:
m, o and p are independently 0 or 1;
n and q are independently selected from integers from 0 to 6;
R^{a} and R^{b} are independently a hydrogen atom or a lower alkyl group;
G is a group selected from cycloalkyl, aryl or heteroaryl groups which are optionally substituted by one or more halogen atoms or by one or more lower alkyl, cycloalkyl, lower haloalkyl, hydroxy, lower alkoxy, lower alkylthio, amino, mono- or dialkylamino, hydroxyalkyl, alkoxyalkyl, hydroxycarbonyl, alkoxycarbonyl and nitrile groups;

### R⁵ represents a hydrogen atom or a lower alkyl, cycloalkyl or benzyl group; or

### R⁴ and R⁵ together with the nitrogen atom to which they are attached form a saturated or unsaturated ring which is optionally substituted by one or more lower alkyl, cycloalkyl or benzyl groups;

and R⁶ represents a group selected from:
hydrogen atoms,
a straight or branched lower alkyl group which is optionally substituted by one or more halogen atoms or by one or more cycloalkyl, hydroxy, lower alkoxy, lower alkylthio, amino, mono- or dialkylamino, alkoxyalkyl, hydroxycarbonyl, alkoxycarbonyl and nitrile groups;
a group of formula:

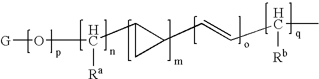

wherein:
m, o and p are independently 0 or 1;
n and q are independently selected from integers from 0 to 6;
R^{a} and R^{b} are independently a hydrogen atom or a lower alkyl group;
G is a group selected from cycloalkyl, aryl or heteroaryl groups which are optionally substituted by one or more halogen atoms or by one or more lower alkyl, cycloalkyl, lower haloalkyl, hydroxy, lower alkoxy, lower alkylthio, amino, mono- or dialkylamino, hydroxyalkyl, alkoxyalkyl, hydroxycarbonyl, alkoxycarbonyl and nitrile groups;
   or pharmaceutically acceptable salts thereof;
   with the proviso that the compound is not one of 2,6-dipyridin-4-ylpyrimidin-4-amine, 4-(3-methoxyanilino)-2,6-di(2-pyridinyl)pyrimidine, 4-(2,5-dimethoxyanilino)-2,6-di(2-pyridinyl)pyrimidine, 4-(5-methoxy-2-methylanilino-2,6-di(2-pyridinyl)pyrimidine, 4-(2-methoxy-5-methylanilino)-2,6-di(2-pyridinyl)pyrimidine, 4-(2-chloro-5-methoxyanilino)-2,6-di(2-pyridinyl)pyrimidine, and 4-(2,5-dimethylanilino)-2,6-di(2-pyridinyl)pyrimidine.

Other aspects of the present invention are: a) pharmaceutical compositions comprising an effective amount of said compounds, b) the use of said compounds in the manufacture of a medicament for the treatment of diseases susceptible of being improved by antagonism of an adenosine receptor, in particular by antagonism of the A_{2A} adenosine receptor, c) methods of treatment of diseases susceptible to amelioration by antagonism of an adenosine receptor, in particular by antagonism of the A_{2A} adenosine receptor, which methods comprise the administration of the compounds of the invention to a subject in need of treatment and combinations of said compounds with one or more of the following drugs: L-DOPA, dopamine agonists, inhibitors of dopamine decarboxylase, catechol-O-methyltransferase inhibitors and inhibitors of monoamine oxidase.

As used herein the term lower alkyl embraces optionally substituted, linear or branched radicals having 1 to 8, preferably 1 to 6 and more preferably 1 to 4 carbon atoms. The preferred substituents in said alkyl groups are selected from halogen atoms, hydroxy groups and amino groups.

Examples include methyl, ethyl, n-propyl, i-propyl, n-butyl, sec-butyl and tert-butyl, n-pentyl, 1-methylbutyl, 2-methylbutyl, isopentyl, 1-ethylpropyl, 1,1-dimethylpropyl, 1,2-dimethylpropyl, n-hexyl, 1-ethylbutyl, 2-ethylbutyl, 1,1-dimethylbutyl, 1,2-dimethylbutyl, 1,3-dimethylbutyl, 2,2-dimethylbutyl, 2,3-dimethylbutyl, 2-methylpentyl, 3-methylpentyl and iso-hexyl radicals.

As used herein, the term lower alkoxy embraces optionally substituted, linear or branched oxy-containing radicals each having alkyl portions of 1 to 8, preferably 1 to 6 and more preferably 1 to 4 carbon atoms. The preferred substituents in said alkoxy groups are selected from halogen atoms, hydroxy groups and amino groups.

Preferred alkoxy radicals include methoxy, ethoxy, n-propoxy, i-propoxy, n-butoxy, sec-butoxy, t-butoxy, trifluoromethoxy, difluoromethoxy, hydroxymethoxy, 2-hydroxyethoxy or 2-hydroxypropoxy.

As used herein, the term lower alkylthio embraces radicals containing an optionally substituted, linear or branched alkyl radicals of 1 to 8, preferably 1 to 6 and more preferably 1 to 4 carbon atoms. The preferred substituents in said alkylthio groups are selected from halogen atoms, hydroxy groups and amino groups

Preferred optionally substituted alkylthio radicals include methylthio, ethylthio, n-propylthio, i-propylthio, n-butylthio, sec-butylthio, t-butylthio, trifluoromethylthio, difluoromethylthio, hydroxymethylthio, 2-hydroxyethylthio or 2-hydroxypropylthio.

As used herein, the term cyclic group embraces, unless otherwise specified, carbocyclic and heterocyclic radicals. The cyclic radicals can contain one or more rings. Carbocyclic radicals may be aromatic or alicyclic, for example cycloalkyl radicals. Heterocyclic radicals also include heteroaryl radicals.

As used herein, the term aromatic group embraces typically a 5- to 14-membered aromatic ring system, such as a 5- or 6-membered ring which may contain one or more heteroatoms selected from O, S and N. When no heteroatoms are present the radical is named aryl radical and when at least one heteroatom is present it is named heteroaryl radical. The aromatic radical can be monocyclic or polycyclic, such as phenyl or naphthyl. When an aromatic radical or moiety carries 2 or more substituents, the substituents may be the same or different.

As used herein, the term aryl radical embraces typically a C₅-C₁₄ monocyclic or polycyclic aryl radical such as phenyl or naphthyl, anthranyl or phenanthryl. Phenyl is preferred.

When an aryl radical carries 2 or more substituents, the substituents may be the same or different.

As used herein, the term heteroaryl radical embraces typically a 5- to 14-membered ring system comprising at least one heteroaromatic ring and containing at least one heteroatom selected from O, S and N. A heteroaryl radical may be a single ring or two or more fused rings wherein at least one ring contains a heteroatom.

Examples include pyridyl, pyrazinyl, pyrimidinyl, pyridazinyl, furyl, oxadiazolyl, oxazolyl, imidazolyl, thiazolyl, thiadiazolyl, thienyl, pyrrolyl, benzothiazolyl, indolyl, indazolyl, purinyl, quinolyl, isoquinolyl, phthalazinyl, naphthyridinyl, quinoxalinyl, quinazolinyl, quinolizinyl, cinnolinyl, triazolyl, indolizinyl, indolinyl, isoindolinyl, isoindolyl, imidazolidinyl, pteridinyl and pyrazolyl radicals. Pyridyl, thienyl, furyl, pyrazolyl, pyridazinyl, pyrimidinyl thiazolyl and quinolyl radicals are preferred. Still more preferred are pyridyl, thienyl, furyl, pyrazolyl, pyridazinyl, pyrimidinyl and quinolyl radicals are preferred

When a heteroaryl radical carries 2 or more substituents, the substituents may be the same or different.

As used herein, the term cycloalkyl embraces saturated optionally substituted carbocyclic radicals and, unless otherwise specified, a cycloalkyl radical typically has from 3 to 7 carbon atoms. The preferred substituents in said cycloalkyl groups are selected from halogen atoms, hydroxy groups, alkyl groups and amino groups.

Examples include cyclopropyl, cyclobutyl, cyclopentyl, cyclohexyl and cycloheptyl. It is preferably cyclopropyl, cyclopentyl or cyclohexyl. When a cycloalkyl radical carries 2 or more substituents, the substituents may be the same or different.

As used herein, some of the atoms, radicals, moieties, chains or cycles present in the general structures of the invention are “optionally substituted”. This means that these atoms, radicals, moieties, chains or cycles can be either unsubstituted or substituted in any position by one or more, for example 1, 2, 3 or 4, substituents, whereby the hydrogen atoms bound to the unsubstituted atoms, radicals, moieties, chains or cycles are replaced by chemically acceptable atoms, radicals, moieties, chains or cycles. When two or more substituents are present, each substituent may be the same or different.

As used herein, the term halogen atom embraces chlorine, fluorine, bromine or iodine atoms typically a fluorine, chlorine or bromine atom, most preferably chlorine or fluorine. The term halo when used as a prefix has the same meaning.

As used herein, the term pharmaceutically acceptable salt embraces salts with a pharmaceutically acceptable acid or base. Pharmaceutically acceptable acids include both inorganic acids, for example hydrochloric, sulphuric, phosphoric, diphosphoric, hydrobromic, hydroiodic and nitric acid and organic acids, for example citric, fumaric, maleic, malic, mandelic, ascorbic, oxalic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases, for example alkyl amines, arylalkyl amines and heterocyclic amines.

Other preferred salts according to the invention are quaternary ammonium compounds wherein an equivalent of an anion (X−) is associated with the positive charge of the N atom. X− may be an anion of various mineral acids such as, for example, chloride, bromide, iodide, sulphate, nitrate, phosphate, or an anion of an organic acid such as, for example, acetate, maleate, fumarate, citrate, oxalate, succinate, tartrate, malate, mandelate, trifluoroacetate, methanesulphonate and p-toluenesulphonate. X− is preferably an anion selected from chloride, bromide, iodide, sulphate, nitrate, acetate, maleate, oxalate, succinate or trifluoroacetate. More preferably X− is chloride, bromide, trifluoroacetate or methanesulphonate.

As used herein, an N-oxide is formed from the tertiary basic amines or imines present in the molecule, using a convenient oxidising agent.

According to one embodiment of the present invention in the compounds of formula (I), R⁶ represents a group selected from:
hydrogen atoms,
a straight or branched lower alkyl group which is optionally substituted by one or more halogen atoms or by one or more cycloalkyl, hydroxy, lower alkoxy, lower alkylthio, amino, mono- or dialkylamino, alkoxyalkyl, hydroxycarbonyl, alkoxycarbonyl and nitrile groups;
a group of formula:

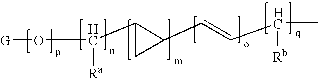

wherein:
m, o and p are independently 0 or 1;
n and q are independently selected from integers from 0 to 6;
R^{a} and R^{b} are independently a hydrogen atom or a lower alkyl group;
G is a group selected from cycloalkyl, aryl or heteroaryl groups which are optionally substituted by one or more halogen atoms or by one or more lower alkyl, cycloalkyl, lower haloalkyl, hydroxy, lower alkoxy, lower alkylthio, amino, mono- or dialkylamino, hydroxyalkyl, alkoxyalkyl, hydroxycarbonyl, alkoxycarbonyl and nitrile groups
   with the proviso that when p, m and o are simultaneously zero then G is not an optionally substituted aryl group and with the further proviso that the compound is not one of 2,6-dipyridin-4-ylpyrimidin-4-amine, 4-(3-methoxyanilino)-2,6-di(2-pyridinyl)pyrimidine, 4-(2,5-dimethoxyanilino)-2,6-di(2-pyridinyl)pyrimidine, 4-(5-methoxy-2-methylanilino)-2,6-di(2-pyridinyl)pyrimidine, 4-(2-methoxy-5-methylanilino)-2,6-di(2-pyridinyl)pyrimidine, 4-(2-chloro-5-methoxyanilino)-2,6-di(2-pyridinyl)pyrimidine, and 4-(2,5-dimethylanilino)-2,6-di(2-pyridinyl)pyrimidine.

According to one embodiment of the present invention in the compounds of formula (I), R¹ represents a monocyclic heteroaryl group selected from the group consisting of furyl, thienyl, thiazolyl, oxazolyl, pyrazinyl, pyrazolyl, pyridazinyl, imidazolyl, triazolyl, pyrimidinyl and pyridyl groups which are optionally substituted by one or more substituents selected from the group consisting of halogen atoms, straight or branched, optionally substituted lower alkoxy and straight or branched, optionally substituted lower alkyl, more preferably selected from the group consisting of halogen atoms and straight or branched, optionally substituted lower alkyl.

According to a preferred embodiment of the present invention in the compounds of formula (I), R¹ represents a monocyclic heteroaryl group selected from the group consisting of furyl, thienyl, pyrazolyl, triazolyl, thiazolyl and pyridyl groups which are optionally substituted by one or more substituents selected from the group consisting of halogen atoms and straight or branched, optionally substituted lower alkyl.

According to a further preferred embodiment of the present invention in the compounds of formula (I), R¹ represents a monocyclic heteroaryl group selected from the group consisting of furyl, thienyl and pyrazolyl groups which groups are optionally substituted by one or more substituents selected from the group consisting of halogen atoms and straight or branched, optionally substituted lower alkyl.

In a still more preferred embodiment in the compounds of formula (I) R¹ represents an unsubstituted furyl group.

According to another embodiment of the present invention in the compounds of formula (I), R² represents a monocyclic heteroaryl group selected from the group consisting of pyrazolyl, furyl, thiazolyl, oxazolyl, pyridyl, pyrimidinyl, pyrazinyl, pyridazinyl, thienyl, imidazolyl and triazolyl groups which are optionally substituted by one or more substituents selected from the group consisting of halogen atoms and straight or branched, optionally substituted lower alkyl.

According to another preferred embodiment of the present invention in the compounds of formula (I), R² represents a monocyclic heteroaryl group selected from the group consisting of pyrazolyl, furyl, thiazolyl, pyridyl, thienyl and triazolyl groups which groups are optionally substituted by one or more substituents selected from the group consisting of halogen atoms, straight or branched, optionally substituted lower alkoxy and straight or branched, optionally substituted lower alkyl, more preferably selected from the group consisting of halogen atoms and straight or branched, optionally substituted lower alkyl.

According to still another embodiment of the present invention in the compounds of formula (I), R⁴ and R⁶ independently represent a group selected from:
hydrogen atoms,
a straight or branched lower alkyl group which is optionally substituted by one or more halogen atoms;
a group of formula:

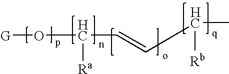

wherein:
o and p are independently 0 or 1;
n and q are independently selected from integers from 0 to 6;
R^{a} and R^{b} are independently a hydrogen atom or a lower alkyl group;
G is a group selected from cycloalkyl, aryl or heteroaryl groups which are optionally substituted by one or more halogen atoms or by one or more lower alkoxy groups;
   and R⁵ represents a hydrogen atom.

According to still another preferred embodiment of the present invention in the compounds of formula (I), R⁴ and R⁶ independently represent a group selected from:
hydrogen atoms,
a straight or branched lower alkyl group which is optionally substituted by one or more halogen atoms;
a group selected from cycloalkylalkyl, phenylalkyl, heteroarylalkyl, phenoxyalkyl and heteroaryloxyalkyl groups which groups are optionally substituted by one or more halogen atoms, by one or more lower alkoxy groups or by one or more lower alkyl groups;
   and R⁵ represents a hydrogen atom.

According to still another preferred embodiment of the present invention in the compounds of formula (I), R³ represents a hydrogen atom or a group selected from the groups of formula —COR⁴; wherein R⁴ represents a group of formula:

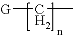

wherein:
   n is an integer selected from 0 or 1;

### G is a group selected from phenyl or heteroaryl groups which phenyl and heteroaryl groups are optionally substituted by one or more halogen atoms, by one or more lower alkoxy groups or by one or more lower alkyl groups.

According to still another preferred embodiment of the present invention in the compounds of formula (I), R¹ is a 2-furyl group and R² is a pyrazolyl group which is optionally substituted by one or more lower alkyl groups.

In a further embodiment of the present invention R¹ represents a 2-furyl group, R² represents a pyrazolyl group which is optionally substituted by one or more lower alkyl groups and R³ represents a hydrogen atom or a group selected from the groups of formula —COR⁴; wherein R⁴ represents a group of formula:

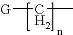

wherein:
n is an integer selected from 0 or 1;

### G is a group selected from phenyl or heteroaryl groups which phenyl and heteroaryl groups are optionally substituted by one or more halogen atoms or by one or more lower alkoxy groups.

Particular individual compounds of the invention include:
2-(2-Furyl)-6-(1H-pyrazol-1-yl)pyrimidin-4-amine (Compound 1);
N-[2-(2-Furyl)-6-(1H-pyrazol-1-yl)pyrimidin-4-yl]acetamide (Compound 2);
N-[2-(2-Furyl)-6-(1H-pyrazol-1-yl)pyrimidin-4-yl]propanamide (Compound 3);
N-[2-(2-Furyl)-6-(1H-pyrazol-1-yl)pyrimidin-4-yl]-2-methylpropanamide (Compound 4);
N-[2-(2-Furyl)-6-(1H-pyrazol-1-yl)pyrimidin-4-yl]-2,2-dimethyl-propanamide (Compound 5);
N-[2-(2-Furyl)-6-(1H-pyrazol-1-yl)pyrimidin-4-yl]cyclopropane-carboxamide (Compound 6);
N-[2-(2-Furyl)-6-(1H-pyrazol-1-yl)pyrimidin-4-yl]cyclobutane-carboxamide (Compound 7);
N-[2-(2-Furyl-6-(1H-pyrazol-1-yl)pyrimidinyl]cyclohexane-carboxamide (Compound 8);
3-Cyclopentyl-N-[2-(2-furyl)-6-(1H-pyrazol-1-yl)pyrimidin-4-yl]propanamide (Compound 9);
N-[2-(2-Furyl)-6-(1H-pyrazol-1-yl)pyrimidin-4-yl]-2-(4-methoxyphenyl)acetamide (Compound 10);
2-(3,4-Dimethoxyphenyl)-N-[2-(2-furyl)-6-(1H-pyrazol-1-yl)pyrimidin-yl]acetamide (Compound 11);
N-[2-(2-Furyl)-6-(1H-pyrazol-1-yl)pyrimidin-4-yl]-3-phenyl-propanamide (Compound 12);
(2S)—N-[2-(2-Furyl)-6-(1H-pyrazol-1-yl)pyrimidinyl]-2-phenyl-cyclopropanecarboxamide (Compound 13);
3,3,3-Trifluoro-N-[2-(2-furyl)-6-(1H-pyrazol-1-yl)pyrimidin-4-yl]propanamide (Compound 14);
3-(3,4-Dimethoxyphenyl)-N-[2-(2-furyl)-6-(1H-pyrazol-1-yl)pyrimidin-4-yl]propanamide (Compound 15);
N-[2-(2-Furyl)-6-(1H-pyrazol-1-yl)pyrimidin-4-yl]-2-methyl-3-phenyl-propanamide (Compound 16);
N-[2-(2-Furyl)-6-(1H-pyrazol-1-yl)pyrimidin-4-yl]-3-phenoxy-propanamide (Compound 17);
N-[2-(2-Furyl)-6-(1H-pyrazol-1-yl)pyrimidin-4-yl]-3-pyridin-3-ylpropanamide (Compound 18);
N-[2-(2-Furyl)-6-(1H-pyrazol-1-yl)pyrimidin-4-yl]-2-pyridin-3-yl-acetamide (Compound 19);
(2E)-3,4-Dimethoxyphenyl)-N-[2-(2-furyl)-6-(H-pyrazol-1-yl)pyrimidin-4-yl]acrylamide (Compound 20);
6-(3,5-Dimethyl-1H-pyrazol-1-yl)-2-(2-furyl)pyrimidin-4-amine (Compound 21);
N-[6-(3,5-Dimethyl-1H-pyrazol-1-yl)-2-(2-furyl)pyrimidin-4-yl]acetamide (Compound 22);
N-[2-(3,5-Dimethyl-1H-pyrazol-1-yl-6-(2-furyl)pyrimidin-4-yl]propanamide (Compound 23);
N-[6-(3,5-Dimethyl-1H-pyrazol-1-yl)-2-(2-furyl)pyrimidin-4-yl]-2-methyl-propanamide (Compound 24);
N-[6-(3,5-Dimethyl-1H-pyrazol-1-yl)-2-(2-furyl)pyrimidin-4-yl]-2,2-dimethylpropanamide (Compound 25);
N-[6-(3,5-Dimethyl-1H-pyrazol-1-yl)-2-(2-furyl)pyrimidin-4-yl]-cyclopropanecarboxamide (Compound 26);
3-Cyclopentyl-N-[6-(3,5-dimethyl-1H-pyrazol-1-yl)-2-(2-furyl)pyrimidin-4-yl]propanamide (Compound 27);
N-[6-(3,5-dimethyl-1H-pyrazol-1-yl)-2-(2-furyl)pyrimidin-4-yl]-2-(4-methoxyphenyl)acetamide (Compound 28);
2-(3,4-Dimethoxyphenyl)-N-[6-(3,5-dimethyl-1H-pyrazol-1-yl)-2-(2-furyl)pyrimidin-4-yl]acetamide (Compound 29);
N-[6-(3,5-Dimethyl-1H-pyrazol-1-yl)-2-(2-furyl)pyrimidin-4-yl]-3-phenylpropanamide (Compound 30);
N-[6-(3,5-Dimethyl-1H-pyrazol-1-yl)-2-(2-furyl)pyrimidin-4-yl]-3,3,3-trifluoropropanamide (Compound 31);
3-(3,4-Dimethoxyphenyl)-N-[6-(3,5-dimethyl-1H-pyrazol-1-yl)-2-(2-furyl)pyrimidin-4-yl]propanamide (Compound 32);
N-[6-(3,5-Dimethyl-1H-pyrazol-1-yl)²-(2-furyl)pyrimidin-4-yl]-3-phenoxypropanamide (Compound 33);
N-[6-(3,5-Dimethyl-1H-pyrazol-1-yl)-2-(2-furyl)pyrimidin-4-yl]-2-pyridin-3-ylacetamide (Compound 34);
N-[6-(3,5-Dimethyl-1H-pyrazol-1-yl)-2-(2-furyl)pyrimidin-4-yl]-3-pyridin-3-ylpropanamide (Compound 35);
2-(2-Furyl)-6-(4-methyl-1H-pyrazol-1-yl)pyrimidin-4-amine (Compound 36);
N-[2-(2-Furyl)-6-(4-methyl-1H-pyrazol-1-yl)pyrimidin-4-yl]-propanamide (Compound 37);
2-(2-Furyl)-6-(3-methyl-1H-pyrazol-1-yl)pyrimidin-4-amine (Compound 38);
N-[2-(2-Furyl)-6-(3-methyl-1H-pyrazol-1-yl)pyrimidin-4-yl]propanamide (Compound 39);
2-(2-Furyl)-6-[3-(trifluoromethyl)-1H-pyrazol-1-yl]pyrimidin-4-amine (Compound 40);
N-{2-(2-Furyl)-6-[3-(trifluoromethyl)-1H-pyrazol-1-yl]pyrimidin-4-yl}propanamide (Compound 41);
2-(2-Furyl)-6-[5-methyl-3-(trifluoromethyl)-1H-pyrazol-1-yl]pyrimidin-4-amine (Compound 42);
N-{2-(2-furyl)-6-[5-methyl-3-(trifluoromethyl)-1H-pyrazol-1-yl]-pyrimidin-4-yl}propanamide (Compound 43);
2-(2-Furyl)-6-(1H-1,2,4-triazol-1-yl)pyrimidin-4-amine (Compound 44);
N-[2-(2-furyl)-6-(1H-1,2,4-triazol-1-yl)pyrimidin-4-yl]acetamide (Compound 45);
N-[2-(2-Furyl)-6-(1H-1,2,4-triazol-1-yl)pyrimidin-4-yl]propanamide (Compound 46);
3,3,3-Trifluoro-N-[2-(2-furyl)-6-(1H-1,2,4-triazol-1-yl)pyrimidin-4-yl]-propanamide (Compound 47);
2-(5-Bromo-2-furyl)-6-(1H-pyrazol-1-yl)pyrimidin-4-amine (Compound 48);
N-[2-(5-bromo-2-furyl)-6-(1H-pyrazol-1-yl)pyrimidin-4-yl]propanamide (Compound 49);
2-(5-Chloro-2-furyl)-6-(1H-pyrazol-1-yl)pyrimidin-4-amine (Compound 50);
N-[2-(5-Chloro-2-furyl)-6-(1H-pyrazol-1-yl)pyrimidin-4-yl]propanamide (Compound 51);
2-(5-Methyl-2-furyl)-6-(1H-pyrazol-1-yl)pyrimidin-4-amine (Compound 52);
N-[2-(5-methyl-2-furyl)-6-(1H-pyrazol-1-yl)pyrimidin-4-yl]propanamide (Compound 53);
N-[2-(2-Furyl)-6-pyridin-3-ylpyrimidin-4-yl]propanamide (Compound 54);
2-(2-Furyl)-6-pyridin-3-ylpyrimidin-4-amine (Compound 55);
6-(1H-Pyrazol-1-yl)-2-(2-thienyl)pyrimidin-4-amine (Compound 56);
N-[6-(1H-Pyrazol-1-yl)₂₋₂-thienyl)pyrimidin-4-yl]acetamide (Compound 57);
N-[6-(1H-Pyrazol-1-yl)-2-(2-thienyl)pyrimidin-4-yl]propanamide (Compound 58);
3-Cyclopentyl-N-[6-(1H-pyrazol-1-yl)₂₂-thienyl)pyrimidin-4-yl]propanamide (Compound 59);
3-Phenyl-N-[6-(1H-pyrazol-1-yl)-2-(2-thienyl)pyrimidin-4-yl]-propanamide (Compound 60);
3,3,3-Trifluoro-N-[6-(1H-pyrazol-1-yl)-2-(2-thienyl)pyrimidin-4-yl]-propanamide (Compound 61);
3-(3,4-Dimethoxyphenyl)-N-[6-(1H-pyrazol-1-yl)-2-(2-thienyl)pyrimidin-4-yl]propanamide (Compound 62);
3-Phenoxy-N-[6-(1H-pyrazol-1-yl)-2-(2-thienyl)pyrimidin-4-yl]-propanamide (Compound 63);
N-[6-(1H-Pyrazol-1-yl)-2-(2-thienyl)pyrimidinyl]-2-pyridin-3-yl-acetamide (Compound 64);
N-[6-(1H-Pyrazol-1-yl)-2-(2-thienyl)pyrimidin-4-yl]-3-pyridin-3-ylpropanamide (Compound 65);
(2E)-3-(3,4-Dimethoxyphenyl)-N-[6-(1H-pyrazol-1-yl)-2-(2-thienyl)-pyrimidin-4-yl]acrylamide (Compound 66);
6-(3,5-Dimethyl-1H-pyrazol-1-yl)-2-(2-thienyl)pyrimidin-4-amine (Compound 67);
N-[6-(3,5-Dimethyl-1H-pyrazol-1-yl)-2-(2-thienyl)pyrimidin-4-yl]-acetamide (Compound 68);
N-[6-(3,5-Dimethyl-1H-pyrazol-1-yl)-2-(2-thienyl)pyrimidin-4-yl]-propanamide (Compound 69);
N-[6-(3,5-Dimethyl-1H-pyrazol-1-yl)-2-(2-thienyl)pyrimidin-4-yl]-3,3,3-trifluoropropanamide (Compound 70);
2-(2-Thienyl)-6-(1H-1,2,4-triazol-1-yl)pyrimidin-4-amine (Compound 71);
N-[2-(2-Thienyl)-6-(1H-1,2,4-triazol-1-yl)pyrimidin-4-yl]acetamide (Compound 72);
N-[2-(2-Thienyl)-6-(1H-1,2,4-triazol-1-yl)pyrimidin-4-yl]propanamide (Compound 73);
3,3,3-Trifluoro-N-[2-(2-thienyl)-6-(1H-1,2,4-triazol-1-yl)pyrimidin-4-yl]propanamide (Compound 74);
N-[2-(3-Methyl-2-thienyl)-6-(1H-pyrazol-1-yl)pyrimidin-4-yl]-propanamide (Compound 75);
6-(2-Furyl)-2-(1H-pyrazol-1-yl)pyrimidin-4-amine (Compound 76);
N-[6-(2-Furyl)-2-(1H-pyrazol-1-yl)pyrimidin-4-yl]acetamide (Compound 77);
N-[6-(2-Furyl)-2-(1H-pyrazol-1-yl)pyrimidin-4-yl]propanamide (Compound 78);
3,3,3-Trifluoro-N-[6-(2-furyl)-2-(1H-pyrazol-1-yl)pyrimidin-4-yl]-propanamide (Compound 79);
2-(3,5-Dimethyl-1H-pyrazol-1-yl)-6-(2-furyl)pyrimidin-4-amine (Compound 80);
N-[2-(3,5-Dimethyl-1H-pyrazol-1-yl)₆₋₂-furyl)pyrimidin-4-yl]-propanamide (Compound 81);
N-[2-(3,5-Dimethyl-1H-pyrazol-1-yl)-6-(2-furyl)pyrimidin-4-yl]-2-(4-methoxyphenyl)acetamide (Compound 82);
6-(2-Furyl)-2-(1H-1,2,4-triazol-1-yl)pyrimidin-4-amine (Compound 83);
N-[6-(2-Furyl)-2-(1H-1,2,4-triazol-1-yl)pyrimidin-yl]propanamide (Compound 84);
2-(1H-Pyrazol-1-yl)-6-pyridin-2-ylpyrimidin-4-amine (Compound 85);
N-[2-(1H-Pyrazol-1-yl)-6-pyridin-2-ylpyrimidin-4-yl]propanamide (Compound 86);
2-(3,5-Dimethyl-1H-pyrazol-1-yl)-6-pyridin-2-ylpyrimidin-4-amine (Compound 87);
N-[2-(3,5-Dimethyl-1H-pyrazol-1-yl)-6-pyridin-2-ylpyrimidin-4-yl]propanamide (Compound 88);
N-[2-(3,5-Dimethyl-1H-pyrazol-1-yl)-6-pyridin-2-ylpyrimidin-4-yl]-2-(4-methoxyphenyl)acetamide (Compound 89);
2-(1H-Pyrazol-1-yl)-6-pyridin-3-ylpyrimidin-4-amine (Compound 90);
N-[2-(1H-Pyrazol-1-yl)-6-pyridin-3-ylpyrimidin-4-yl]propanamide (Compound 91);
2-(3,5-Dimethyl-1H-pyrazol-1-yl)-6-pyridin-3-ylpyrimidin-4-amine (Compound 92);
N-[2-(3,5-Dimethyl-1H-pyrazol-1-yl)-6-pyridin-3-ylpyrimidin-yl]-propanamide (Compound 93);
N-[2-(3,5-Dimethyl-1H-pyrazol-1-yl)-6-pyridin-3-ylpyrimidin-4-yl]-2-(4-methoxyphenyl)acetamide (Compound 94);
2-(1H-Pyrazol-1-yl)-6-pyridin-4-ylpyrimidin-4-amine (Compound 95);
N-[2-(1H-Pyrazol-1-yl)-6-pyridin-4-ylpyrimidin-4-amine (Compound 96);
6-(2-Furyl)-2-pyridin-2-ylpyrimidin-4-amine (Compound 97);
N-[6-(2-Furyl)-2-pyridin-2-ylpyrimidin-4-yl]propanamide (Compound 98);
2-(3-Methylpyridin-2-yl)-6-(1H-pyrazol-1-yl)pyrimidin-4-amine (Compound 99);
N-[2-(3-methylpyridin-2-yl)-6-(1H-pyrazol-1-yl)pyrimidin-4-yl]propanamide (Compound 100);
6-(1H-Pyrazol-1-yl)-2-pyridin-3-ylpyrimidin-4-amine (Compound 101);
N-[6-(1H-Pyrazol-1-yl)-2-pyridin-3-ylpyrimidin-4-yl]acetamide (Compound 102);
N-[6-(1H-Pyrazol-1-yl)-2-pyridin-3-ylpyrimidin-4-yl]propanamide (Compound 103);
6-(3,5-Dimethyl-1H-pyrazol-1-yl)-2-pyridin-3-ylpyrimidin-4-amine (Compound 104);
N-[6-(3,5-Dimethyl-1H-pyrazol-1-yl)-2-pyridin-3-ylpyrimidin-4-yl]-acetamide (Compound 105);
N-[6-(3,5-Dimethyl-1H-pyrazol-1-yl)-2-pyridin-3-ylpyrimidin-4-yl]-propanamide (Compound 106);
N-[6-(3,5-Dimethyl-1H-pyrazol-1-yl)-2-pyridin-3-ylpyrimidin-4-yl]-3,3,3-trifluoropropanamide (Compound 107);
2-Pyridin-3-yl-6-(1H-1,2,4-triazol-1-yl)pyrimidin-4-amine (Compound 108);
3,3,3-Trifluoro-N-[2-pyridin-3-yl-6-(1H-1,2,4-triazol-1-yl)pyrimidin-4-yl]propanamide (Compound 109);
6-(2-Furyl)-2-pyridin-3-ylpyrimidin-4-ylamine (Compound 110);
N-[6-(2-Furyl)-2-pyridin-3-ylpyrimidin-4-yl]propanamide (Compound 111);
N-[6-(3,5-Dimethyl-1H-pyrazol-1-yl)-2-pyridin-4-ylpyrimidin-4-yl]propanamide (Compound 112);
6-(3,5-dimethyl-1H-pyrazol-1-yl)-2-pyridin-4-ylpyrimidin-4-amine (Compound 113);
6-(2-Furyl)-2-pyridin-4-ylpyrimidin-4-ylamine (Compound 114);
N-[6-(2-Furyl)-2-pyridin-4-ylpyrimidin-4-yl]propanamide (Compound 115);
6-(2-Furyl)-2-(1,3-thiazol-2-yl)pyrimidin-4-amine (Compound 116);
N-[6-(2-Furyl)-2-(1,3-thiazol-2-yl)pyrimidin-4-yl]propanamide (Compound 117);
2-(4-Fluorophenyl)-N-[6-(2-furyl)-2-(1,3-thiazol-2-yl)pyrimidinyl]acetamide (Compound 118);
N-(Cyclopropylmethyl)-2-(2-furyl)-6-(1H-pyrazol-1-yl)pyrimidin-4-amine (Compound 119);
(2R)-2-{[2-(2-Furyl)-6-(1H-pyrazol-1-yl)pyrimidin-4-yl]amino}propan-1-ol (Compound 120);
3-{[2-(2-Furyl)-6-(1H-pyrazol-1-yl)pyrimidin-4-yl]amino}propan-1-ol (Compound 121);
N-[2-(2-Furyl)-6-(1H-pyrazol-1-yl)pyrimidin-4-yl]ethane-1,2-diamine (Compound 122);
2-(2-Furyl)-N-[2-(4-methoxyphenyl)ethyl]-6-(1H-pyrazol-1-yl)-pyrimidin-4-amine (Compound 123);
N-[2-(3,4-Dimethoxyphenyl)ethyl]-2-(2-furyl)-6-(1H-pyrazol-1-yl)pyrimidin-4-amine (Compound 124);
2-(2-Furyl)-6-(1H-pyrazol-1-yl)-N-[2-(pyridin-2-yl)ethyl]pyrimidin-4-amine (Compound 125);
2-(2-Furyl)-6-(1H-pyrazol-1-yl)-N-[2-pyridin-3-yl)ethyl]pyrimidin-4-amine (Compound 126);
2-(2-Furyl)-N-3-phenylpropyl)-6-(1H-pyrazol-1-yl)pyrimidin-4-amine (Compound 127);
2-(2-Furyl)-N-[3-(1H-imidazol-1-yl)propyl]-6-(1H-pyrazol-1-yl)-pyrimidin-4-amine (Compound 128);
N-(Cyclopropylmethyl)-6-(1H-pyrazol-1-yl)-2-(2-thienyl)pyrimidin-4-amine (Compound 129);
(2R)-2-{[6-(1H-Pyrazol-1-yl)-2-(2-thienyl)pyrimidin-4-yl]amino}-propan-1-ol (Compound 130);
3-{[6-(1H-Pyrazol-1-yl)-2-(2-thienyl)pyrimidin-4-yl]amino}propan-1-ol (Compound 131);
N-(2-Aminoethyl)-N-[6-(1H-pyrazol-1-yl)-2-(2-thienyl)pyrimidin-4-yl]amine (Compound 132);
N-[2-(4-Methoxyphenyl)ethyl]-6-(1H-pyrazol-1-yl)-2-(2-thienyl)pyrimidin-4-amine (Compound 133);
N-[2-(3,4-Dimethoxyphenyl)ethyl]-6-(1H-pyrazol-1-yl)-2-(2-thienyl)pyrimidin-4-amine (Compound 134);
6-(1H-Pyrazol-1-yl)-N-(2-pyridin-3-ylethyl)-2-(2-thienyl)pyrimidin-4-amine (Compound 135);
6-(1H-Pyrazol-1-yl)-N-(2-pyridin-2-ylethyl)-2-(2-thienyl)pyrimidin-4-amine (Compound 136);
N-(3-Phenylpropyl)-6-(1H-pyrazol-1-yl)-2-(2-thienyl)pyrimidin-4-amine (Compound 137);
N-[3-(1H-imidazol-1-yl)propyl]-6-(1H-pyrazol-1-yl)-2-(2-thienyl)-pyrimidin-4-amine (Compound 138);
Ethyl 6-(1H-pyrazol-1-yl)-2-(2-thienyl)pyrimidin-4-yl]carbamate (Compound 139);
N-[2-(2-Furyl)-6-(1H-pyrazol-1-yl)pyrimidinyl]-N′-[(1S*,2R*)-2-phenylcyclopropyl]urea (*relative trans configuration) (Compound 140);
N-[2-(2-Furyl)-6-(1H-pyrazol-1-yl)pyrimidin-4-yl]-N′-propylurea (Compound 141);
N-[2-(2-Furyl)-6-(1H-pyrazol-1-yl)pyrimidin-4-yl]-N′-isopropylurea (Compound 142);
N-Cyclopentyl-N′-[2-(2-furyl)-6-(1H-pyrazol-1-yl)pyrimidin-4-yl]urea (Compound 143);
N-[2-(2-Furyl)-6-(1H-pyrazol-1-yl)pyrimidin-4-yl]-N′-(4-methoxy-phenyl)urea (Compound 144);
N-[2-(2-Furyl)-6-(1H-pyrazol-1-yl)pyrimidin-4-yl]-N′-(2-phenylethyl)urea (Compound 145);
N-Benzyl-N′-[2-(2-furyl)-6-(1H-pyrazol-1-yl)pyrimidin-4-yl]urea (Compound 146);
N-[2-(2-Furyl)-6-(1H-pyrazol-1-yl)pyrimidin-4-yl]-3-methylbutanamide (Compound 147);
N-[2-(2-Furyl)-6-(1H-pyrazol-1-yl)pyrimidinyl]-3,3-dimethyl-butanamide (Compound 148);
N-[2-(2-Furyl)-6-(1H-pyrazol-1-yl)pyrimidin-4-yl]cyclopentane-carboxamide (Compound 149);
2-Chloro-N-[2-(2-furyl)-6-(1H-pyrazol-1-yl)pyrimidin-4-yl]-2-phenyl-acetamide (Compound 150);
N-[2-(2-Furyl)-6-(1H-pyrazol-1-yl)pyrimidin-yl]-2-phenylacetamide (Compound 151);
2-(4-Fluorophenyl)-N-[2-(2-furyl-6-(1H-pyrazol-1-yl)pyrimidin-4-yl]acetamide (Compound 152);
N-[2-(2-Furyl)-6-(1H-pyrazol-1-yl)pyrimidin-4-yl]-2-(3-methoxy-phenyl)acetamide (Compound 153);
N-[2-(2-Furyl)-6-(1H-pyrazol-1-yl)pyrimidinyl]-2-(2-methoxy-phenyl)acetamide (Compound 154);
2-(3,4-Dichlorophenyl)-N-[2-(2-furyl)-6-(1H-pyrazol-1-yl)pyrimidinyl]acetamide (Compound 155);
2-(1,3-Dibenzodioxol-5-yl)-N-[2-(2-furyl)-6-(1H-pyrazol-1-yl)pyrimidin-4-yl]acetamide (Compound 156);
2-(3,4-Dihydroxyphenyl)-N-[2-(2-furyl)-6-(1H-pyrazol-1-yl)pyrimidin-4-yl]acetamide (Compound 157);
2-(2,5-Dimethoxyphenyl)-N-[2-(2-furyl)-6-(1H-pyrazol-1-yl)pyrimidin-4-yl]acetamide (Compound 158);
2-(4-Chloro-3-methylphenyl)-N-[2-(2-furyl)-6-(1H-pyrazol-1-yl)pyrimidin-4-yl]acetamide (Compound 159);
2-(3,5-Dimethoxyphenyl)-N-[2-(2-furyl)-6-(1H-pyrazol-1-yl)pyrimidin-4-yl]acetamide (Compound 160);
2-[3-(Benzyloxy)-4-methoxyphenyl]-N-[2-(2-furyl)-6-(1H-pyrazol-1-yl)pyrimidin-4-yl]acetamide (Compound 161);
2-[4-(Cyclobutyloxy)-3-methoxyphenyl]-N-[2-(2-furyl)-6-(1H-pyrazol-1-yl)pyrimidin-4-yl]acetamide (Compound 162);
N-[2-(2-Furyl)-6-(1H-pyrazol-1-yl)pyrimidin-4-yl]-2-(4-difluoromethoxy-3-methoxyphenyl)acetamide (Compound 163);
N-[2-(2-Furyl)-6-(1H-pyrazol-1-yl)pyrimidin-4-yl]-2-(3,4,5-trimethoxy-phenyl)acetamide (Compound 164);
2-(3,4-Dimethoxyphenyl)-N-[2-(2-furyl)-6-(1H-pyrazol-1-yl)pyrimidin-4-yl]propanamide (Compound 165);
N-[2-(2-Furyl)-6-(1H-pyrazol-1-yl)pyrimidin-4-yl]benzamide (Compound 166);
N-[2-(2-Furyl)-6-(1H-pyrazol-1-yl)pyrimidin-4-yl]-3,4-dimethoxy-benzamide (Compound 167);
2,6-Difluoro-N-[2-(2-furyl)-6-(1H-pyrazol-1-yl)pyrimidin-4-yl]-benzamide (Compound 168);
N-[2-(2-Furyl)-6-(1H-pyrazol-1-yl)pyrimidin-4-yl]-2-furamide (Compound 169);
N-[2-(2-Furyl)-61(H-pyrazol-1-yl)pyrimidin-4-yl]thiophene-2-carboxamide (Compound 170);
N-[2-(2-Furyl)-6-(1H-pyrazol-1-yl)pyrimidin-4-yl]nicotinamide (Compound 171);
N-[2-(2-Furyl)-6-(1H-pyrazol-1-yl)pyrimidin-4-yl]isonicotinamide (Compound 172);
N-[2-(2-Furyl)-6-(1H-pyrazol-1-yl)pyrimidin-4-yl]-1-naphthamide (Compound 173);
N-[2-(2-Furyl)-6-(1H-pyrazol-1-yl)pyrimidinyl]quinoline-2-carboxamide (Compound 174);
(2E)-3-(3,4-Dimethoxyphenyl)-N-[6-(3,5-dimethyl-1H-pyrazol-1-yl)-2-(2-furyl)pyrimidin-4-yl]acrylamide (Compound 175);
2-(2-Furyl)-6-(2H-1,2,3-triazol-2-yl)pyrimidin-4-amine (Compound 176);
2-(2-furyl)-(H-1,2,3-triazol-1-yl)pyrimidin-4-amine (Compound 177);
N-[2-(2-Furyl)-6-(2H-1,2,3-triazol-2-yl)pyrimidin-4-yl]propanamide (Compound 178);
2-(3,4-Dimethoxyphenyl)-N-[2-(2-furyl)-6-(2H-1,2,3-triazol-2-yl)pyrimidin-4-yl]acetamide (Compound 179);
N-[2-(2-Furyl)-6-(1H-1,2,3-triazol-1-yl)pyrimidin-4-yl]propanamide (Compound 180);
2-(2-Furyl)-6-(1,3-thiazol-2-yl)pyrimidin-4-amine (Compound 181);
N-[2-(2-Furyl)-6-(1,3-thiazol-2-yl)pyrimidin-4-yl]propanamide (Compound 182);
3-(3,4-Dimethoxyphenyl)-N-[2-(2-furyl)-6-(1,3-thiazol-2-yl)pyrimidin-4-yl]propanamide (Compound 183);
2,6-Di-2-furylpyrimidin-4-amine (Compound 184);
N-(2,6-Di-2-furylpyrimidin-4-yl)-2-(3,4-dimethoxyphenyl)acetamide (Compound 185);
6-(1,3-Benzothiazol-2-yl)-2-(2-furyl)pyrimidin-4-amine (Compound 186);
2-(5-Methyl-2-furyl)-6-(1,3-thiazol-2-yl)pyrimidin-4-amine (Compound 187);
6-(1,3-Thiazol-2-yl)-2-(2-thienyl)pyrimidin-4-amine (Compound 188);
2-(3,4-Dimethoxyphenyl)-N-[6-(2-furyl)-2-(1,3-thiazol-2-yl)pyrimidin-4-yl]acetamide (Compound 189);
6-(1H-Pyrazol-1-yl)-2-(1,3-thiazol-2-yl)pyrimidin-4-amine (Compound 190);
2-(3,4-Dimethoxyphenyl)-N-[6-(1H-pyrazol-1-yl)-2-(1,3-thiazol-2-yl)pyrimidin-4-yl]acetamide (Compound 191);
2-(2-Furyl)-N-methyl-6-(1,3-thiazol-2-yl)pyrimidin-4-amine (Compound 192);
N-(Cyclopropylmethyl)-2-(2-furyl)-6-(1,3-thiazol-2-yl)pyrimidin-4-amine (Compound 193);
N-[2-(3,4-Dimethoxyphenyl)ethyl]-2-(2-furyl)-6-(1,3-thiazol-2-yl)-pyrimidin-4-amine (Compound 194);
N-Cyclopropylmethyl)-6-(2-furyl)-2-(1,3-thiazol-2-yl)pyrimidin-4-amine (Compound 195);
N-[2-(3,4-Dimethoxyphenyl)ethyl]-6-(2-furyl)-2-(1,3-thiazol-2-yl)-pyrimidin-4-amine (Compound 196);
6-(2-Furyl)-N-(2-pyridin-3-ylethyl)-2-(1,3-thiazol-2-yl)pyrimidin-4-amine (Compound 197);
6-(2-Furyl)-N-[(1S*,2R*)-2-phenylcyclopropyl]-2-(1,3-thiazol-2-yl)-pyrimidin-4-amine (*relative trans configuration) (Compound 198);
Ethyl [2-(2-furyl)-6-(1H-pyrazol-1-yl)pyrimidin-4-yl]carbamate (Compound 199);
Cyclopentylmethyl [2-(2-furyl)-6-(1H-pyrazol-1-yl)pyrimidin-4-yl]-carbamate (Compound 200);
Benzyl [2-(2-furyl)-6-(1H-pyrazol-1-yl)pyrimidin-4-yl]carbamate (Compound 201);
3,4-Dimethoxybenzyl [2-(2-furyl)-6-(1H-pyrazol-1-yl)pyrimidin-4-yl]carbamate (Compound 202);
Pyridin-3-ylmethyl [2-(2-furyl)-6-(1H-pyrazol-1-yl)pyrimidin-4-yl]-carbamate (Compound 203);
4-Methoxyphenyl [2-(2-furyl)-6-(1H-pyrazol-1-yl)pyrimidin-4-yl]-carbamate (Compound 204); and
3,4-Dimethoxyphenyl [2-(2-furyl)-6-(1H-pyrazol-1-yl)pyrimidin-4-yl]carbamate (Compound 205).

Of outstanding interest are:
2-(2-Furyl)-6-(1H-pyrazol-1-yl)pyrimidin-4-amine
N-[2-(2-Furyl)-6-(1H-pyrazol-1-yl)pyrimidin-4-yl]acetamide
N-[2-(2-Furyl)-6-(1H-pyrazol-1-yl)pyrimidin-4-yl]propanamide
N-[2-(2-Furyl)-6-(1H-pyrazol-1-yl)pyrimidin-4-yl]-2-methylpropanamide
N-[2-(2-Furyl)-6-(1H-pyrazol-1-yl)pyrimidin-4-yl]-2,2-dimethyl-propanamide
N-[2-(2-Furyl)-6-(1H-pyrazol-1-yl)pyrimidin-4-yl]cyclopropane-carboxamide
N-[2-(2-Furyl)-6-(1H-pyrazol-1-yl)pyrimidin-4-yl]cyclobutane-carboxamide
N-[2-(2-Furyl)-6-(1H-pyrazol-1-yl)pyrimidinyl]cyclohexane-carboxamide
3-Cyclopentyl-N-[2-(2-furyl)-6-(1H-pyrazol-1-yl)pyrimidin-4-yl]propanamide
N-[2-(2-Furyl)-6-(1H-pyrazol-1-yl)pyrimidin-4-yl]-2-(4-methoxyphenyl)acetamide
2-(3,4-Dimethoxyphenyl)-N-[2-(2-furyl)-6-(1H-pyrazol-1-yl)pyrimidin-4-yl]acetamide
N-[2-(2-Furyl)-6-(1H-pyrazol-1-yl)pyrimidin-4-yl]-3-phenyl-propanamide
3,3,3-Trifluoro-N-[2-(2-furyl)-6-(1H-pyrazol-1-yl)pyrimidin-4-yl]propanamide
3-(3,4-Dimethoxyphenyl)-N-[2-(2-furyl)-6-(1H-pyrazol-1-yl)pyrimidin-4-yl]propanamide
N-[2-(2-Furyl)-6-(1H-pyrazol-1-yl)pyrimidin-4-yl]-2-methyl-3-phenyl-propanamide
N-[2-(2-Furyl)-6-(1H-pyrazol-1-yl)pyrimidin-4-yl]-3-phenoxy-propanamide
N-[2-(2-Furyl)-6-(1H-pyrazol-1-yl)pyrimidin-yl]-3-pyridin-3-ylpropanamide
N-[2-(2-Furyl)-6-(1H-pyrazol-1-yl)pyrimidin-4-yl]-2-pyridin-3-yl-acetamide
(2E)-3-(3,4-Dimethoxyphenyl)-N-[2-(2-furyl)-6-(1H-pyrazol-1-yl)pyrimidin-4-yl]acrylamide
N-[6-(3,5-Dimethyl-1H-pyrazol-1-yl)-2-(2-furyl)pyrimidinyl]acetamide
N-[2-(3,5-Dimethyl-1H-pyrazol-1-yl)-6-(2-furyl)pyrimidinyl]propanamide
N-[6-(3,5-Dimethyl-1H-pyrazol-1-yl)-2-(2-furyl)pyrimidin-4-yl]-2-methyl-propanamide
N-[6-(3,5-Dimethyl-1H-pyrazol-1-yl)-2-(2-furyl)pyrimidin-4-yl]-cyclopropanecarboxamide
3-Cyclopentyl-N-[6-(3,5-dimethyl-1H-pyrazol-1-yl)-2-(2-furyl)pyrimidin-4-yl]propanamide
N-[6-(3,5-dimethyl-1H-pyrazol-1-yl)-2-(2-furyl)pyrimidinyl]-2-(4-methoxyphenyl)acetamide
2-(3,4-Dimethoxyphenyl)-N-[6-(3,5-dimethyl-1H-pyrazol-1-yl)₂₋₂-furyl)pyrimidin-4-yl]acetamide
N-[6-(3,5-Dimethyl-1H-pyrazol-1-yl)-2-(2-furyl)pyrimidin-4-yl]-3-phenylpropanamide
N-[6-(3,5-Dimethyl-1H-pyrazol-1-yl)-2-(2-furyl)pyrimidin-4-yl]-3,3,3-trifluoropropanamide
3-(3,4-Dimethoxyphenyl)-N-[6-(3,5-dimethyl-1H-pyrazol-1-yl)-2-(2-furyl)pyrimidin-4-yl]propanamide
N-[6-(3,5-Dimethyl-1H-pyrazol-1-yl)-2-(2-furyl)pyrimidin-4-yl]-3-phenoxypropanamide
N-[6-(3,5-Dimethyl-1H-pyrazol-1-yl)-2-(2-furyl)pyrimidinyl]-2-pyridin-3-ylacetamide
N-[6-(3,5-Dimethyl-1H-pyrazol-1-yl)-2-(2-furyl)pyrimidin-4-yl]-3-pyridin-3-ylpropanamide
N-[2-(2-Furyl)-6-(4-methyl-1H-pyrazol-1-yl)pyrimidin-4-yl]-propanamide
2-(2-Furyl)-6-(3-methyl-1H-pyrazol-1-yl)pyrimidin-4-amine
N-[2-(2-Furyl)-6-(3-methyl-1H-pyrazol-1-yl)pyrimidin-4-yl]propanamide
N-{2-2′-Furyl)-6-[3-(trifluoromethyl)-1H-pyrazol-1-yl]pyrimidin-4-yl}-propanamide
N-{2-(2-furyl)-6-[5-methyl-3-(trifluoromethyl)-1H-pyrazol-1-yl]-pyrimidin-4-yl}propanamide
N-[2-(2-Furyl)-6-(1H-1,2,4-triazol-1-yl)pyrimidin-4-yl]propanamide
N-[2-(5-bromo-2-furyl)-6-(1H-pyrazol-1-yl)pyrimidin-4-yl]propanamide
N-[2-(5-methyl-2-furyl)-6-(1H-pyrazol-1-yl)pyrimidin-4-yl]propanamide
N-[2-(2-Furyl)-6-pyridin-3-ylpyrimidin-4-yl]propanamide
N-[6-(1H-Pyrazol-1-yl)-2-(2-thienyl)pyrimidin-4-yl]acetamide
N-[6-(1H-Pyrazol-1-yl)-2-(2-thienyl)pyrimidin-4-yl]propanamide
N-[6-(1H-Pyrazol-1-yl)-2-(2-thienyl)pyrimidinyl]-2-pyridin-3-yl-acetamide
N-[6-(3,5-Dimethyl-1H-pyrazol-1-yl)-2-(2-thienyl)pyrimidin-yl]-acetamide
N-[2-(3,5-Dimethyl-1H-pyrazol-1-yl)-6-pyridin-3-ylpyrimidin-4-yl]-2-(4-methoxyphenyl)acetamide
N-[2-(3-methylpyridin-2-yl)-6-(1H-pyrazol-1-yl)pyrimidin-4-yl]propanamide
N-[6-(2-Furyl)-2-(1,3-thiazol-2-yl)pyrimidin-4-yl]propanamide
N-[2-(2-Furyl)-6-(1H-pyrazol-1-yl)pyrimidinyl]-3-methylbutanamide
N-[2-(2-Furyl)-6-(1H-pyrazol-1-yl)pyrimidin-4-yl]cyclopentane-carboxamide
N-[2-(2-Furyl)-6-(1H-pyrazol-1-yl)pyrimidinyl]-2-(3-methoxy-phenyl)acetamide
N-[2-(2-Furyl)-6-(1H-pyrazol-1-yl)pyrimidin-4-yl]-2-(3,4,5-trimethoxy-phenyl)acetamide
N-[2-(2-Furyl)-6-(1H-pyrazol-1-yl)pyrimidinyl]-2-furamide
N-[2-(2-Furyl)-6-(1,3-thiazol-2-yl)pyrimidinyl]propanamide
N-(2,6-Di-2-furylpyrimidin-4-yl)-2-(3,4-dimethoxyphenyl)acetamide
2-(3,4-Dimethoxyphenyl)-N-[6-(2-furyl)-2-(1,3-thiazol-2-yl)pyrimidin-4-yl]acetamide

Additional compounds of the present invention include the following:
N-(2-furan-2-yl-6-pyrazol-1-yl-pyrimidin-4-yl)-2-methylaminoacetamide (Compound 206);
2-Dimethylamino-N-(2-furan-2-yl-6-pyrazol-1-yl-pyrimidin-4-yl)acetamide (Compound 207);
2-Methylamino-N-[2-(5-methylfuran-2-yl)-6-thiazol-2-yl-pyrimidin-4-yl]acetamide (Compound 208);
2-Diethylamino-N-[2-(5-methylfuran-2-yl)-6-thiazol-2-yl-pyrimidin-4-yl]acetamide (Compound 209);
N-[2-(5-Methylfuran-2-yl)-6-thiazol-2-yl-pyrimidine-4-yl]-2-pyridin-3-yl-propionamide (Compound 210);
N-[2-(5-Methylfuran-2-yl)-6-thiazol-2-yl-pyrimidin-4-yl]-3-pyridin-3-yl-propionamide (Compound 211);
6-(2-Dimethylamino-N-[2-(5-methylfuran-2-yl)-6-pyrazol-1-yl-pyrimidin-4-yl]-acetamide (Compound 212);
2-Dimethylamino-N-[2-(5-methylfuran-2-yl)-6-thiazol-2-yl-pyrimidin-4-yl]-acetamide (Compound 213);
2-Dimethylamino-N-[6-(3,5-dimethylpyrazol-1-yl)-2-(5-methylfuran-2-yl)-pyrimidinyl]-acetamide (Compound 214);
2-Dimethylamino-N-[2-(5-methylfuran-2-yl)-6-pyridin-2-yl-pyrimidin-4-yl]-acetamide (Compound 215);
2-Dimethylamino-N-(2-furan-2-yl-6-pyrazol-1-yl-pyrimidin-4-yl)-acetamide (Compound 216);
2-Dimethylamino-N-(2-furan-2-yl-6-thiazol-2-yl-pyrimidin-4-yl)-acetamide (Compound 217);
2-Dimethylamino-N-[6-(3,5-dimethylpyrazol-1-yl)-2-furan-2-yl-pyrimidinyl]-acetamide (Compound 218);
2-Dimethylamino-N-(2-furan-2-yl-6-pyridin-2-yl-pyrimidin-4-yl)-acetamide (Compound 219);
2-Dimethylamino-N-(6-pyrazol-1-yl-2-thiophen-2-yl-pyrimidin-4-yl)acetamide (Compound 220);
2-Dimethylamino-N-(6-thiazol-2-yl-2-thiophen-2-yl-pyrimidin-4-yl)-acetamide (Compound 221);
2-Dimethylamino-N-[6-(3,5-dimethylpyrazol-1-yl)-2-thiophen-2-yl-pyrimidinyl]-acetamide (Compound 222);
2-Dimethylamino-N-(6-pyridin-2-yl-2-thiophen-2-yl-pyrimidin-4-yl)-acetamide (Compound 223);
2-Dimethylamino-N-(6-pyrazol-1-yl-2-thiazol-2-yl-pyrimidin-4-yl)acetamide (Compound 224);
2-Dimethylamino-N-(6-thiazol-2-yl-2-thiazol-2-yl-pyrimidin-4-yl)acetamide (Compound 225);
2-Dimethylamino-N-[6-(3,5-dimethylpyrazol-1-yl)-2-thiazol-2-yl-pyrimidin-4-yl]-acetamide (Compound 226);
2-Dimethylamino-N-(6-pyridin-2-yl-2-thiazol-2-yl-pyrimidin-4-yl)-acetamide (Compound 227);
2-Dimethylamino-N-[2-(5-methylfuran-2-yl)-6-thiophen-2-yl-pyrimidin-4-yl]-acetamide (Compound 228);
2-Dimethylamino-N-(2-furan-2-yl-6-thiophen-2-yl-pyrimidin-4-yl)acetamide (Compound 229);
2-Dimethylamino-N-(2-thiophen-2-yl-6-thiophen-2-yl-pyrimidin-4-yl)-acetamide (Compound 230);
2-Dimethylamino-N-(2-thiazol-2-yl-6-thiophen-2-yl-pyrimidin-4-yl)-acetamide (Compound 231);
2-Dimethylamino-N-(2-pyridin-2-yl-6-thiophen-2-yl-pyrimidin-4-yl)-acetamide (Compound 232);
2-Dimethylamino-N-(6-pyrazol-1-yl-2-pyridin-2-yl-pyrimidin-4-yl)acetamide (Compound 233);
2-Dimethylamino-N-(6-thiazol-2-yl-2-pyridin-2-yl-pyrimidin-4-yl)acetamide (Compound 234);
2-Dimethylamino-N-[6-(3,5-dimethylpyrazol-1-yl)-2-pyridin-2-yl-pyrimidin-4-yl)acetamide (Compound 235);
2-Dimethylamino-N-(6-pyridin-2-yl-2-pyridin-2-yl-pyrimidin-4-yl)-acetamide (Compound 236);
2-Dimethylamino-N-(2-oxazol-2-yl-6-pyrazol-1-yl-pyrimidin-4-yl)acetamide (Compound 237);
2-Dimethylamino-N-(2-oxazol-2-yl-6-thiazol-2-yl-pyrimidin-4-yl)acetamide (Compound 238);
2-Dimethylamino-N-[2-oxazol-2-yl-6-(3,5-dimethylpyrazol-1-yl)-pyrimidin-4-yl]-acetamide (Compound 239);
2-Dimethylamino-N-(2-oxazol-2-yl-6-pyridin-2-yl-pyrimidin-4-yl)-acetamide (Compound 240);
2-Dimethylamino-N-[6-(3-methyl-[1,2,4]oxadiazol-5-yl)-2-thiophen-2-yl-pyrimidin-4-yl]-acetamide (Compound 241);
2-Methylamino-N-[2-(5-methylfuran-2-yl)-6-pyrazol-1-yl-pyrimidin-4-yl]-acetamide (Compound 242);
2-Methylamino-N-[2-(5-methylfuran-2-yl)-6-thiazol-2-yl-pyrimidin-4-yl]-acetamide (Compound 243);
2-Methylamino-N-[6-(3,5-dimethylpyrazol-1-yl)-2-(5-methylfuran-2-yl)-pyrimidin-4-yl]-acetamide (Compound 244);
2-Methylamino-N-[2-(5-methylfuran-2-yl)-6-pyridin-2-yl-pyrimidin-4-yl]-acetamide (Compound 245);
2-Methylamino-N-(2-furan-2-yl-6-pyrazol-1-yl-pyrimidin-4-yl)-acetamide (Compound 246);
2-Methylamino-N-(2-furan-2-yl-6-thiazol-2-yl-pyrimidin-4-yl)acetamide (Compound 247);
2-Methylamino-N-[6-(3,5-dimethylpyrazol-1-yl)-2-furan-2-yl-pyrimidin-4-yl]-acetamide (Compound 248);
2-Methylamino-N-(2-furan-2-yl-6-pyridin-2-yl-pyrimidin-4-yl)acetamide (Compound 249);
2-Methylamino-N-(6-pyrazol-1-yl-2-thiophen-2-yl-pyrimidin-yl)-acetamide (Compound 250);
2-Methylamino-N-(6-thiazol-2-yl-2-thiophen-2-yl-pyrimidin-4-yl)acetamide (Compound 251);
2-Methylamino-N-[6-(3,5-dimethylpyrazol-1-yl)-2-thiophen-2-yl-pyrimidin-4-yl]-acetamide (Compound 252);
2-Methylamino-N-(6-pyridin-2-yl-2-thiophen-2-yl-pyrimidin-4-yl)-acetamide (Compound 253);
2-Methylamino-N-(6-pyrazol-1-yl-2-thiazol-2-yl-pyrimidin-yl)-acetamide (Compound 254);
2-Methylamino-N-(6-thiazol-2-yl-2-thiazol-2-yl-pyrimidin-4-yl)-acetamide (Compound 255);
2-Methylamino-N-[6-(3,5-dimethylpyrazol-1-yl)-2-thiazol-2-yl-pyrimidin-4-yl]-acetamide (Compound 256);
2-Methylamino-N-(6-pyridin-2-yl-2-thiazol-2-yl-pyrimidin-4-yl)-acetamide (Compound 257);
N-[2-(5-methylfuran-2-yl)-6-pyrazol-1-yl-pyrimidin-4-yl]-2-pyridin-3-yl-acetamide (Compound 258);
N-[6-(3,5-dimethyl-pyrazol-1-yl)-2-(5-methylfuran-2-yl)-pyrimidin-4-yl]-2-pyridin-3-yl-acetamide (Compound 259);
N-[2-(5-methylfuran-2-yl)-6-pyridin-2-yl-pyrimidin-4-yl]-2-pyridin-3-yl-acetamide (Compound 260);
N-(2-furan-2-yl-6-thiazol-2-yl-pyrimidinyl)-2-pyridin-3-yl-acetamide (Compound 261);
N-[6-(3,5-dimethyl-pyrazol-1-yl)-2-furan-2-yl-pyrimidin-4-yl]-2-pyridin-3-yl-acetamide (Compound 262);
N-(2-furan-2-yl-6-pyridin-2-yl-pyrimidin-4-yl)-2-pyridin-3-yl-acetamide Compound 263);
N-(6-pyrazol-1-yl-2-thiophen-2-yl-pyrimidin-4-yl)-2-pyridin-3-yl-acetamide (Compound 264);
2-pyridin-3-yl-N-(6-thiazol-2-yl-2-thiophen-2-yl-pyrimidin-4-yl)-acetamide (Compound 265);
N-[6-(3,5-dimethyl-pyrazol-1-yl)-2-thiophen-2-yl-pyrimidin-4-yl]-2-pyridin-3-yl-acetamide (Compound 266);
2-pyridin-3-yl-N-(6-pyridin-2-yl-2-thiophen-2-yl-pyrimidin-4-yl)acetamide (Compound 267);
N-(6-pyrazol-1-yl-2-thiazol-2-yl-pyrimidin-4-yl)-2-pyridin-3-yl-acetamide (Compound 268);
2-pyridin-3-yl-N-(6-thiazol-2-yl-2-thiazol-2-yl-pyrimidin-4-yl)acetamide (Compound 269);
N-[6-(3,5-dimethyl-pyrazol-1-yl)-2-thiazol-2-yl-pyrimidin-4-yl]-2-pyridin-3-yl-acetamide (Compound 270);
2-pyridin-3-yl-N-(6-pyridin-2-yl-2-thiazol-2-yl-pyrimidin-4-yl)-acetamide (Compound 271);
N-[2-(5-methyl-furan-2-yl)-6-thiophen-2-yl-pyrimidin-4-yl]-2-pyridin-3-yl-acetamide (Compound 272);
N-[2-furan-2-yl)-6-thiophen-2-yl-pyrimidin-4-yl]-2-pyridin-3-yl-acetamide (Compound 273);
N-[2-thiophen-2-yl)-6-thiophen-2-yl-pyrimidin-4-yl]-2-pyridin-3-yl-acetamide (Compound 274);
N-[2-thiazol-2-yl)-6-thiophen-2-yl-pyrimidin-4-yl]-2-pyridin-3-yl-acetamide (Compound 275);
N-[2-(pyridin-2-yl)-6-thiophen-2-yl-pyrimidin-4-yl]-2-pyridin-3-yl-acetamide (Compound 276);
N-(6-pyrazol-1-yl-2-pyridin-2-yl-pyrimidin-4-yl)-2-pyridin-3-yl-acetamide (Compound 277);
N-(6-thiazol-2-yl-2-pyridin-2-yl-pyrimidinyl)-2-pyridin-3-yl-acetamide (Compound 278);
N-(6-[3,5-dimethylpyrazol-1-yl]-2-pyridin-2-yl-pyrimidin-4-yl)-2-pyridin-3-yl-acetamide (Compound 279);
N-(6-pyridin-2-yl-2-pyridin-2-yl-pyrimidin-4-yl)-2-pyridin-3-yl-acetamide (Compound 280);
N-(2-oxazol-2-yl-6-pyrazol-1-yl-pyrimidin-yl)-2-pyridin-3-yl-acetamide (Compound 281);
N-(2-oxazol-2-yl-6-thiazol-2-yl-pyrimidin-4-yl)-2-pyridin-3-yl-acetamide (Compound 282);
N-(6-[3,5-dimethylpyrazol-1-yl]-2-oxazol-2-yl-pyrimidin-4-yl)-2-pyridin-3-yl-acetamide (Compound 283);
N-(2-oxazol-2-yl-6-pyridin-2-yl-pyrimidin-4-yl)-2-pyridin-3-yl-acetamide (Compound 284);
N-[6-(3-methyl-[1,2,4]oxadiazol-5-yl)-2-thiophen-2-yl-pyrimidinyl]-2-pyridin-3-yl-acetamide (Compound 285);
N-[2-(5-methyl-furan-2-yl)-6-pyrazol-1-yl-pyrimidin-4-yl]-3-pyridin-3-yl-propionamide (Compound 286);
N-[6-(3,5-dimethyl-pyrazol-1-yl)-2-(5-methyl-furan-2-yl)pyrimidin-4-yl]-2-pyridin-3-yl-propionamide (Compound 287);
N-[2-(5-methyl-furan-2-yl)-6-pyridin-2-yl-pyrimidin-4-yl]-2-pyridin-3-yl-propionamide (Compound 288);
N-(2-furan-2-yl-6-pyrazol-1-yl-pyrimidin-4-yl)-3-pyridin-3-yl-propionamide (Compound 289);
N-(2-furan-2-yl-6-thiazol-2-yl-pyrimidin-4-yl)-2-pyridin-3-yl-propionamide (Compound 290);
N-[6-(3,5-dimethyl-pyrazol-1-yl)-2-furan-2-yl-pyrimidin-4-yl]-2-pyridin-3-yl-propionamide (Compound 291);
N-(2-furan-2-yl-6-pyridin-2-yl-pyrimidin-4-yl)-2-pyridin-3-yl-propionamide (Compound 292);
N-(6-pyrazol-1-yl-2-thiophen-2-yl-pyrimidin-4-yl)-2-pyridin-3-yl-propionamide (Compound 293);
2-pyridin-3-yl-N-(6-thiazol-2-yl-2-thiophen-2-yl-pyrimidin-4-yl)propionamide (Compound 294);
N-[6-(3,5-dimethyl-pyrazol-1-yl)-2-thiophen-2-yl-pyrimidin-4-yl]-2-pyridin-3-yl-propionamide (Compound 295);
2-pyridin-3-yl-N-(6-pyridin-2-yl-2-thiophen-2-yl-pyrimidin-4-yl)-propionamide (Compound 296);
N-(6-pyrazol-1-yl-2-thiazol-2-yl-pyrimidin-4-yl)-2-pyridin-3-yl-propionamide (Compound 297);
2-pyridin-3-yl-N-(6-thiazol-2-yl-2-thiazol-2-yl-pyrimidin-4-yl)-propionamide (Compound 298);
N-[6-(3,5-dimethyl-pyrazol-1-yl)-2-thiazol-2-yl-pyrimidin-4-yl]-2-pyridin-3-yl-propionamide (Compound 29.9); and
2-pyridin-3-yl-N-(6-pyridin-2-yl-2-thiazol-2-yl-pyrimidin-4-yl)-propionamide (Compound 300).

The compounds of the present invention may be prepared by one of the processes described below.

Compounds of formula (I) and in particular those of formulas (VIIIa) or (IXa) where R¹ is a monocyclic or polycyclic heteroaryl group linked to the pyrimidine ring through a carbon atom and R² is a monocyclic or polycyclic heteroaryl group linked to the pyrimidine ring through a nitrogen atom can be obtained as shown is Scheme 1.

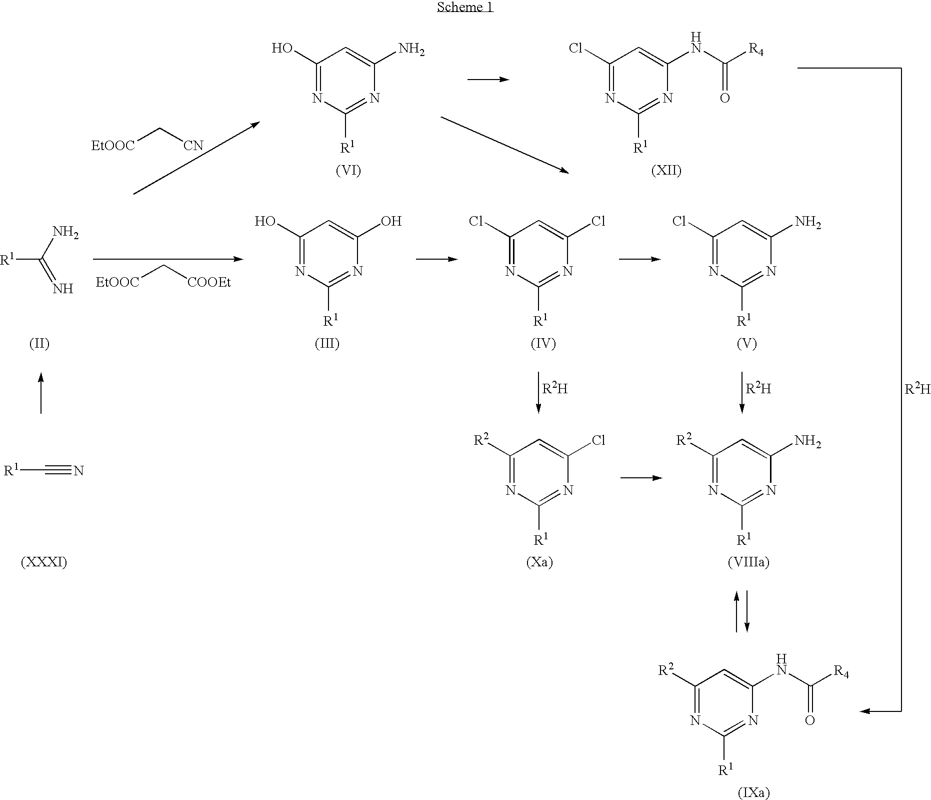

The carboxyamidines of formula (II), wherein R¹ is a monocyclic or polycyclic heteroaryl group linked to the carboxyamidine group through a carbon atom can be obtained by reacting a nitrite of formula (XXXI) with trimethylaluminum and ammonium chloride, in a solvent such as benzene, toluene or xylene, at a temperature from 80° to 120°. It also can be obtained by reaction of a nitrile of formula (XXXI) with sodium methoxide in methanol at room temperature, followed by reaction with ammonium chloride at the same temperature.

The carboxyamidines of formula (II) can be reacted with diethyl malonate in a solvent such as methanol, ethanol, isopropyl alcohol, butyl alcohol or tetrahydrofuran, in the presence of a base, such as sodium methoxide, sodium ethoxide or potassium tertbutoxide and at a temperature from room temperature to the boiling point of the solvent to yield the pyrimidine-4,6-diols of formula (III).

The resulting pyrimidine-4,6-diols of formula (III) can be reacted with a chlorinated agent such a phosphorus oxychloride, phosphorus pentachloride or a mixture of them, in a solvent such as phosphorus oxychloride, benzene or toluene, at a temperature from room temperature to the boiling point of the solvent to yield the 4,6-dichloropyrimidine compounds of formula (IV). Optionally, the presence of a base such as dimethylaminoaniline, triethylamine or diisopropyl-ethylamine may be needed in this reaction step.

The reaction of the 4,6-dichloropyrimidine compounds of formula (IV) with ammonium hydroxide in a solvent such as methanol, ethanol, isopropyl alcohol or tetrahydrofuran, at a temperature from 80° to 140° produces the 6-chloropyrimidin-4-amines of formula (V).

The resulting the 6-chloropyrimidin-amines of formula (V) are reacted with a compound of formula R²—H wherein R² is a monocyclic or polycyclic heteroaryl group linked to the pyrimidine ring through a nitrogen atom to yield the compounds of formula (VIIIa) which is a particular case of the compounds of formula (I) according to the invention. The reaction is carried out in a solvent such as dimethylformamide, dimethylacetamide or dimethylsulfoxide, in the presence of a base, such as sodium hydride, potassium carbonate or cesium carbonate, at a temperature from 60° to 140° C.

The compounds of formula (VIIIa) can be acylated by an acid chloride and a base, such as pyridine, triethylamine or diisopropylethylamine, in a solvent such as tetrahydrofuran, methylene chloride, chloroform or pyridine, at a temperature from room temperature to the boiling point of the solvent to yield the compounds of formula (IXa) which is a particular case of the compounds of formula (I) according to the invention. Compounds of formula (IXa) can also be prepared by reaction of amine (VIIIa) with an anhydride, at a temperature from 80° to 160° C.

The 4,6-dichloropyrimidine compounds of formula (IV) can also be converted into the 4-chloropyrimidines of formula (Xa) by reaction with a compound of formula R²—H wherein R² is a monocyclic or polycyclic heteroaryl group linked to the pyrimidine ring through a nitrogen atom. The reaction is carried out in a solvent such as dimethylformamide, dimethylacetamide or dimethylsulfoxide, in the presence of a base, such as sodium hydride, potassium carbonate or cesium carbonate, at a temperature from 60° to 140° C.

The resulting 4-chloropyrimidines of formula (Xa) can then be converted to the compounds of formula (VIIIa) according to the invention by reaction with ammonium hydroxide in a solvent such as methanol, ethanol, isopropyl alcohol or tetrahydrofuran, at a temperature from 80° C. to 140° C.

Alternatively, the compounds of formula (VIIIa) according to the invention can also be obtained from the compounds of formula (IXa) by reaction with a mineral acid, such as hydrochloric acid or sulphuric acid, in a solvent such as water, methanol, ethanol or isopropyl alcohol, at a temperature from room temperature to the boiling point of the solvent.

The compounds of formula (IXa) according to the invention can be obtained by reaction of the compounds of formula (XII) with compounds of formula R²H wherein R² is as hereinabove-defined. The reaction is carried out in a solvent such as dimethylformamide, dimethylacetamide or dimethylsulfoxide, in the presence of a base, such as sodium hydride, potassium carbonate or cesium carbonate, at a temperature from 60° to 140° C.

The compounds of formula (XII) can be obtained from the 6-aminopyrimidin-4-ol compounds of formula (VI) by reaction with a carboxylic acid of formula R⁴COOH, wherein R⁴ is as hereinabove-defined in the presence of a chlorinated agent such as phosphorus oxychloride, phosphorus pentachloride or thionyl chloride, at a temperature from 60° to 120° C.

The 6-aminopyrimidin-4-ol compounds of formula (VI) are in turn obtained by reaction of the carboxyamidines of formula (II) with ethylcyanoacetate. The reaction is carried out in a solvent such as methanol, ethanol, isopropyl alcohol, butyl alcohol or tetrahydrofuran, in the presence of a base, such as sodium methoxide, sodium ethoxide or potassium tertbutoxide and at a temperature from room temperature to the boiling point of the solvent.

Compounds of formula (I) and in particular those of formulas (VIIIb) or (IXb) where R¹ is a monocyclic or polycyclic heteroaryl group linked to the pyrimidine ring through a nitrogen atom and R² is a monocyclic or polycyclic heteroaryl group linked to the pyrimidine ring through a carbon atom can be obtained as shown is Scheme 2.

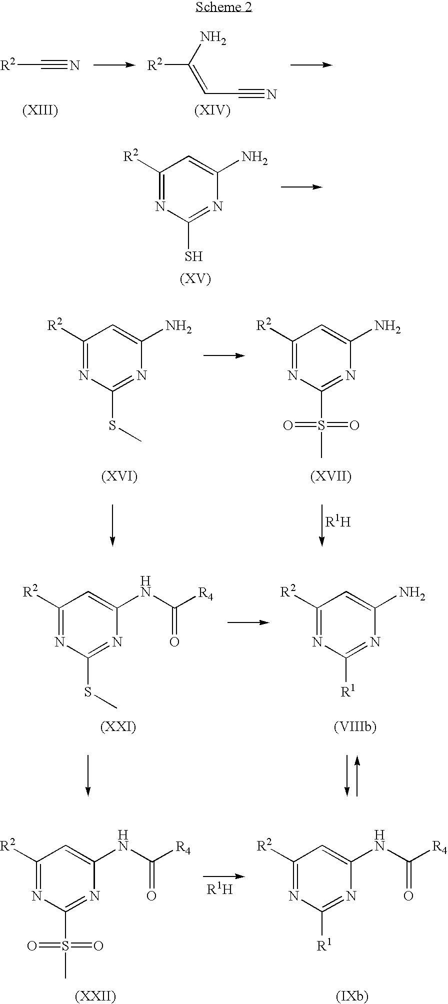

The aminonitriles of formula (XIV) can be obtained by reacting the nitriles of formula (XIII) wherein R² is as hereinabove-defined and acetonitrile, in the presence of a base, preferably as lithium diisopropylamide or potassium tertbutoxide, in a solvent such as benzene, toluene or xylene, at a temperature from room temperature to the boiling point of the solvent.

The resulting aminonitriles (XIV) are reacted with thiourea, in a solvent such as methanol, ethanol, isopropyl alcohol, butyl alcohol or tetrahydrofuran, in the presence of a base such as sodium methoxide, sodium ethoxide or potassium tertbutoxide, at a temperature from 60° to 140° C. to yield 4-aminopyrimidine-2-thiols of formula (XV).

The 4-aminopyrimidine-2-thiols of formula (XV) can be reacted in a solvent such as water, methanol, ethanol, dimethylformamide or dimethylsulfoxide, with methyl iodide or dimethylsulfate, in the presence of a base such as sodium hydroxide, sodium carbonate, potassium carbonate or sodium hydride, and a temperature from room temperature to 80° C. to yield the 2-methylthio)pyrimidin-4-amines of formula (XVI).

The 2-(methylthio)pyrimidin-4-amines of formula (XVI) can either be reacted with an oxidizing agent, preferably m-chloroperbenzoic acid, oxone or magnesium monoperoxyphthalate, in a solvent such as methanol, ethanol, acetone, methylene chloride or chloroform, and at a temperature from 0° to 70° C. to yield 2-(methylsulfonyl)pyrimidin-4-amines of formula (XVII) or in the alternative they can be acylated by an acid chloride and a base, such as pyridine, triethylamine or diisopropylethylamine, in a solvent such as tetrahydrofuran, methylene chloride, chloroform or pyridine, at a temperature from room temperature to the boiling point of the solvent to yield the 2-(methylthio)pyrimidin-4-amides of formula (XXI).

The 2-(methylsulfonyl)pyrimidin-4-amines of formula (XVII) can be converted to the compounds (VIIIb) according to the present invention by reaction with compounds of formula R¹—H, wherein R¹ is a monocyclic or polycyclic heteroaryl group linked to the pyrimidine ring through a nitrogen atom. The reaction is carried out in a solvent such as dimethylformamide, dimethylacetamide or dimethylsulfoxide, in the presence of a base, preferably sodium hydride, potassium carbonate or cesium carbonate, and at a temperature from 60° to 160° C. Similarly the 2-(methylsulfonyl)pyrimidin-4-amides of formula (XXII) can be converted to the compounds (IXb) according to the present invention following the same procedure.

The 2-(methylthio)pyrimidin-4-amides of formula (XXI) can be reacted with an oxidizing agent, preferably m-chloroperbenzoic acid, oxone or magnesium monoperoxyphthalate, in a solvent such as methanol, ethanol, acetone, methylene chloride or chloroform, and at a temperature from 0° to 70° C. to yield the 2-methylsulfonyl)pyrimidin-4-amides of formula (XXII).

Finally the compounds (VIIIb) according to the invention can be converted to the compounds (IXb) also according to the invention by reaction with an acid chloride and a base, such as pyridine, triethylamine or diisopropylethylamine, in a solvent such as tetrahydrofuran, methylene chloride, chloroform or pyridine, at a temperature from room temperature to the boiling point of the solvent. Compounds of formula (IXb) can also be prepared by reaction of amine (VIIIb) with an anhydride, at a temperature from 80° to 160° C.

The reverse operation through which compounds of formula (IXb) are converted into compounds of formula (VIIIb) is also possible and can be carried out by reaction with a mineral acid, such as hydrochloric acid or sulphuric acid, in a solvent such as water, methanol, ethanol or isopropyl alcohol, at a temperature from room temperature to the boiling point of the solvent.

Compounds of formula (I) and in particular those of formulas (VIIIc) or (IXc) where R¹ is a monocyclic or polycyclic heteroaryl group linked to the pyrimidine ring through a carbon atom and R² is a monocyclic or polycyclic heteroaryl group linked to the pyrimidine ring through a carbon atom can be obtained as shown is Scheme 3.

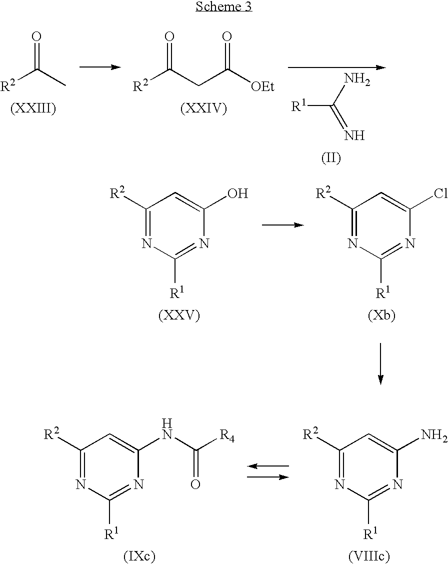

The reaction between methyl ketones of formula (XXIII), wherein R² is a monocyclic or polycyclic heteroaryl group linked to the pyrimidine ring through a carbon atom and diethyl carbonate can be carried out in the presence of a base, preferably sodium hydride, in a solvent such as benzene, toluene, ethyl ether, tetrahydrofuran or dioxane, and at a temperature from 40° to 120° C. to yield the substituted ethyl 3-oxo-propanoates of formula (XXIV).

The pyrimidin-4-ol compounds of formula (XXV) can be obtained from the substituted ethyl 3-oxo-propanoates of formula (XXIV) by reaction with carboxyamidines of formula (II) in a solvent such as methanol, ethanol, isopropyl alcohol, butyl alcohol or tetrahydrofuran, in the presence of a base, such as sodium methoxide, sodium ethoxide or potassium tertbutoxide and at a temperature from room temperature to the boiling point of the solvent.

The pyrimidin-4-ol compounds of formula (XXV) can be reacted with a chlorinated agent such a phosphorus oxychloride, phosphorus pentachloride or a mixture of them, in a solvent such as phosphorus oxychloride, benzene or toluene, at a temperature from room temperature to the boiling point of the solvent to yield the 4-chloropyrimidines of formula (Xb). Optionally, the presence of a base such as dimethylaminoaniline, triethylamine or diisopropyl-ethylamine may be needed in this reaction step.

The compounds of formula (VIIIc) according to the present invention can be prepared from 4-chloropyrimidines of formula (Xb) by reaction with ammonium hydroxide in a solvent such as methanol, ethanol, isopropyl alcohol or tetrahydrofuran, at a temperature from 80° C. to 140° C.

Finally the compounds of formula (IXc) according to the present invention can be prepared from the compounds of formula (VIIIc) by acylation with an acid chloride and a base, such as pyridine, triethylamine or diisopropylethylamine, in a solvent such as tetrahydrofuran, methylene chloride, chloroform or pyridine, at a temperature from room temperature to the boiling point of the solvent. Compounds of formula (IXc) can also be prepared by reaction of amine (VIIIc) with an anhydride, at a temperature from 80° to 160° C.

Compounds of formula (VIIIc) can also be obtained from compounds of formula (IXc) by reaction with a mineral acid, such as hydrochloric acid or sulphuric acid, in a solvent such as water, methanol, ethanol or isopropyl alcohol, at a temperature from room temperature to the boiling point of the solvent.

Compounds of formulae (VIIIc) and (IXc) where R¹ is a monocyclic or polycyclic heteroaryl group linked to the pyrimidine ring through a carbon atom and R² is a monocyclic or polycyclic heteroaryl group linked to the pyrimidine ring through a carbon atom can also be obtained as shown is Scheme 4.

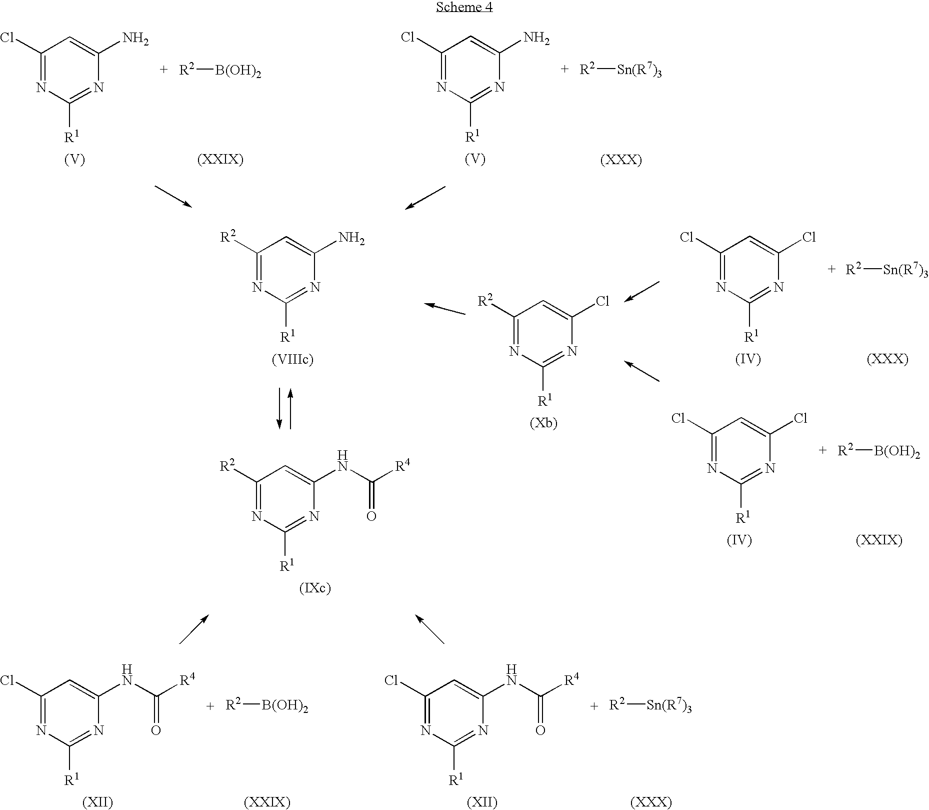

The Suzuki reaction between the 4-aminopirimidines of formulae (IV), (V) or (XII) and the boronic acid of formula (XXIX), wherein R² is a monocyclic or polycyclic heteroaryl group linked to the pyrimidine ring through a carbon atom, is preferably carried out in an organic solvent such as methanol, ethanol, acetonitrile, dioxane, tetrahydrofuran, dimethoxyethane, benzene or toluene, optionally in the presence of water, at a temperature between 60° and 120° C., with a base such as sodium or potassium carbonate and a palladium(0) catalyst such as tetrakis(triphenylphosphine)palladium(0).

The Stille reaction between the 4-aminopirimidines of formulae (IV), (V) or (XII) and the organotin derivative of formula (XXX), wherein R² is a monocyclic or polycyclic heteroaryl group linked to the pyrimidine ring through a carbon atom, is preferably carried out in an organic solvent such as methanol, ethanol, acetonitrile, dioxane, tetrahydrofuran, dimethoxyethane, benzene or toluene, optionally in the presence of water, at a temperature between 60° and 120° C., with a base such as sodium or potassium carbonate and a catalyst such as tetrakis(triphenylphosphine)palladium(0) or bis(triphenylphosphine)palladium(II) chloride.

The 4-chloropyrimidine compounds of formula (Xb) can be converted to the compounds of formula (VIIIc) by reaction with ammonium hydroxide in a solvent such as methanol, ethanol, isopropyl alcohol or tetrahydrofuran, at a temperature from 80° to 140° C.

Finally the compounds of formula (IXc) according to the present invention can be prepared from the compounds of formula (VIIIc) by acylation with an acid chloride and a base, such as pyridine, triethylamine or diisopropylethylamine, in a solvent such as tetrahydrofuran, methylene chloride, chloroform or pyridine, at a temperature from room temperature to the boiling point of the solvent. Compounds of formula (IXc) can also be prepared by reaction of amine (VIIIc) with an anhydride, at a temperature from 80° to 160° C.

Compounds of formula (VIIIc) can also be obtained from compounds of formula (IXc) by reaction with a mineral acid, such as hydrochloric acid or sulphuric acid, in a solvent such as water, methanol, ethanol or isopropyl alcohol, at a temperature from room temperature to the boiling point of the solvent.

Compounds of formulae (VIIId) and (IXd) where R¹ is a monocyclic or polycyclic heteroaryl group linked to the pyrimidine ring through a carbon atom and R² is a substituted heterocyclic group can be obtained as shown is Scheme 5.

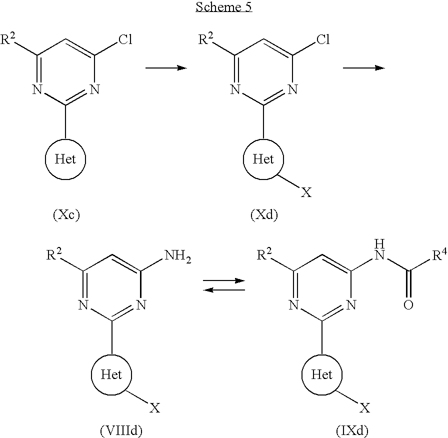

The substituted 4-chloro-2-(2-heteroaryl)pyrimidines of formula (Xd) can be obtained by reaction of the corresponding unsubstituted 4-chloro-2-(2-heteroaryl)pyrimidines of formula (Xc). When the heteroaryl group is a furyl group the reaction is preferably carried out with N-chlorosuccinimide (X=chloro) or N-bromosuccinimide (X=bromo), with a solvent such as dimethylformamide or dimethylsulfoxide, at a temperature from 40° to 100° C. Alternatively halogenating agent can be selected from the group consisting of Cl₂, Br₂, SOCl₂ and SOBr₂.

The 4-chloropyrimidine compounds of formula (Xd) can then be converted to the compounds of formula (VIIId) by reaction with ammonium hydroxide in a solvent such as methanol, ethanol, isopropyl alcohol or tetrahydrofuran, at a temperature from 80° to 140° C.

Finally the compounds of formula (IXd) according to the present invention can be prepared from the compounds of formula (VIIId) by acylation with an acid chloride and a base, such as pyridine, triethylamine or diisopropylethylamine, in a solvent such as tetrahydrofuran, methylene chloride, chloroform or pyridine, at a temperature from room temperature to the boiling point of the solvent. Compounds of formula (IXd) can also be prepared by reaction of amine (VIIId) with an anhydride, at a temperature from 80° to 160° C.

Compounds of formula (VIIId) can also be obtained from compounds of formula (IXd) by reaction with a mineral acid, such as hydrochloric acid or sulphuric acid, in a solvent such as water, methanol, ethanol or isopropyl alcohol, at a temperature from room temperature to the boiling point of the solvent.

Carbamates of formula (XXVI), and ureas of formula (XX) can be synthesised as it is summarised on Scheme 6

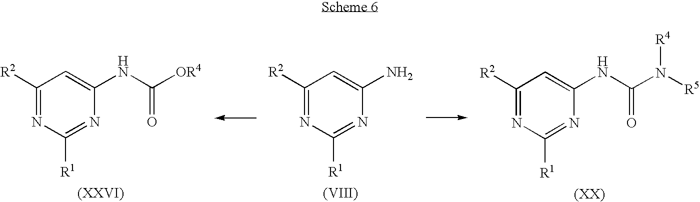

The carbamates of formula (XXVI) are obtained by reaction of a compound of formula (VIII) with a compound of formula Z-COOR⁴, wherein Z represents a leaving group such as halogen atom, preferably chlorine or a group selected from ethoxy, methoxy, p-nitrophenoxy and imidazolyl. The reaction is carried out in a solvent, such as tetrahydrofuran, chloroform, methylene chloride or dimethylformamide, in the presence of a base, preferably triethylamine, diisopropylethylamine, potassium carbonate or sodium hydroxide, at a temperature from −70° to 100° C.

The compounds of formula (VIII) can also be converted to the ureas of formula (XX) wherein R⁵ is a hydrogen atom by reaction with an isocyanate of formula R⁴—N═C═O in a solvent such as benzene, toluene or xylene, at a temperature from room temperature to 140° C.

The synthesis of amines of formula (XXVII) can be prepared following Scheme 7

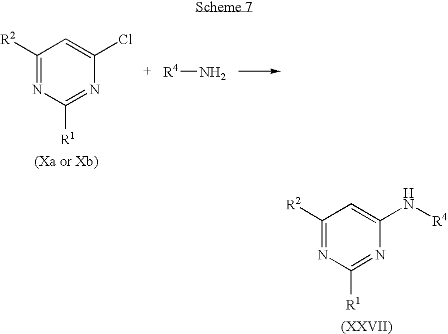

When R¹ represents a monocyclic or polycyclic heteroaryl group linked to the pyrimidine ring through a carbon atom, the compounds of formula (XXVII) can be obtained from the compounds of formulae (Xa and Xb) by reaction with an amine of formula R⁴NH₂. The reaction is carried out in a solvent such as methanol, ethanol, isopropanol, butanol, pentanol, tetrahydrofuran or dimethylformamide, in the presence of a base such as an excess of the reacting amine R⁴NH₂ or potassium carbonate, sodium carbonate, triethylamine or diisopropylethylamine, and at a temperature between room temperature and the boiling point of the solvent.

When the defined groups R¹ to R⁵ are susceptible to chemical reaction under the conditions of the hereinbefore described processes or are incompatible with said processes, conventional protecting groups may be used in accordance with standard practice, for example see T. W. Greene and P. G. M. Wuts in ‘Protective Groups in Organic Chemistry’, 3rd Edition, John Wiley & Sons (1999). It may be that deprotection will form the last step in the synthesis of compounds of formula (I).

The compounds of formulae (XIII), (XXIII), (XXIX), (XXX) and (XXXI), are known compounds or can be prepared by analogy with known methods.

In particular compounds of formulae (XXIX) and (XXX) can be prepared by the methods described in Tyrrell, E.; Brookes, P; Synthesis, 2003, 4, 469-483; Condret, C. Synthetic Communications 1996, 26(19), 3543-3547 and Handbook of Organopalladium Chemistry for Organic Synthesis, Two Volume Set Edited by Ei-ichi Negishi. John Wiley and Sons, 2002.

### Pharmacological Activity

### Adenosine 2A Receptor Subtype Competition Radioligand Binding Assay

Human membranes from recombinant A2a receptors were purchased from Receptor Biology, Inc. (USA)

Competition assays were carried out by incubation of membranes from hA2a receptors transfected to HEK293 cells, [³H]ZM241385 as radioligand, buffer (50 mM Tris-HCl (pH=7.4), 10 mM MgCl₂, 1 mM EDTA, 2 units/ml adenosine deaminase), and unlabelled ligand in a total volume of 0.2 ml for 90 min at 25° C. NECA was used to determinate non-specific binding. Filter over Schleicher&Schuell GF/52 filters (pre-soaked 0.5% polyethylenimine) in a Brandel cell harvester. Unbound radioligand was removed with 3×3 ml ice-cold 50 mM Tris-HCl (pH=7.4), 0.9% NaCl.

With reference to A2a receptor binding affinities, A2a receptor antagonists of this invention may have a IC₅₀ of less than 10 μM. In one embodiment of this invention, a A2a receptor antagonist has a IC₅₀ of less than 1 μM. In another embodiment the IC₅₀ is less than 0.25 μM (i.e., 250 nM).

The pyrimidin-4-amine derivatives of the invention are useful in the treatment or prevention of diseases known to be susceptible to improvement by treatment with an antagonist of an adenosine receptor, in particular those susceptible to improvement by treatment with and antagonist of the A_{2A} adenosine receptor. Such diseases are, for example ischemia, supraventricular arrhythmias, acute renal failure, myocardial reperfusion injury, allergic reactions including but not limited to rhinitis, urticaria, scleroderm arthritis, other autoimmune diseases, inflammatory bowel diseases, asthma, diabetes mellitus, obesity, Parkinson disease, Huntington's disease, dystonias such as restless leg syndrome, dyskinesias such as those caused by prolonged use of neuroleptic and dopaminergic drugs or sleep disorders.

Accordingly, the pyrimidin-4-amine derivatives of the invention and pharmaceutically acceptable salts thereof, and pharmaceutical compositions comprising such compound and/or salts thereof, may be used in a method of treatment of disorders of the human body which comprises administering to a subject requiring such treatment an effective amount of pyrimidin-4-amine derivative of the invention or a pharmaceutically acceptable salt thereof.

The present invention also provides pharmaceutical compositions which comprise, as an active ingredient, at least a pyrimidin-4-amine derivative of formula (I) or a pharmaceutically acceptable salt thereof in association with a pharmaceutically acceptable excipient such as a carrier or diluent. The active ingredient may comprise 0.001% to 99% by weight, preferably 0.01% to 90% by weight of the composition depending upon the nature of the formulation and whether further dilution is to be made prior to application. Preferably the compositions are made up in a form suitable for oral, topical, nasal, rectal, percutaneous or injectable administration.

The pharmaceutically acceptable excipients which are admixed with the active compound, or salts of such compound, to form the compositions of this invention are well-known per se and the actual excipients used depend inter alia on the intended method of administering the compositions.

Compositions of this invention are preferably adapted for injectable and per os administration. In this case, the compositions for oral administration may take the form of tablets, retard tablets, sublingual tablets, capsules, inhalation aerosols, inhalation solutions, dry powder inhalation, or liquid preparations, such as mixtures, elixirs, syrups or suspensions, all containing the compound of the invention; such preparations may be made by methods well-known in the art.

The diluents which may be used in the preparation of the compositions include those liquid and solid diluents which are compatible with the active ingredient, together with colouring or flavouring agents, if desired. Tablets or capsules may conveniently contain between 2 and 500 mg of active ingredient or the equivalent amount of a salt thereof.

The liquid composition adapted for oral use may be in the form of solutions or suspensions. The solutions may be aqueous solutions of a soluble salt or other derivative of the active compound in association with, for example, sucrose to form a syrup. The suspensions may comprise an insoluble active compound of the invention or a pharmaceutically acceptable salt thereof in association with water, together with a suspending agent or flavouring agent.

Compositions for parenteral injection may be prepared from soluble salts, which may or may not be freeze-dried and which may be dissolved in pyrogen free aqueous media or other appropriate parenteral injection fluid.

Effective doses are normally in the range of 2-2000 mg of active ingredient per day. Daily dosage may be administered in one or more treatments, preferably from 1 to 4 treatments, per day.

The present invention will be further illustrated by the following examples. The examples are given by way of illustration only and are not to be construed as a limiting.

The syntheses of the compounds of the invention and of the intermediates for use therein are illustrated by the following Examples (1 to 205) including the preparation of intermediates 1 to 83 which do not limit the scope of the invention in any way.

General. Reagents, starting materials, and solvents were purchased from commercial suppliers and used as received. Concentration refers to evaporation under vacuum using a Büchi rotatory evaporator. Reaction products were purified, when necessary, by flash chromatography on silica gel (40-63 μm) with the solvent system indicated. Spectroscopic data were recorded on a Varian Gemini 200 spectrometer, Varian Gemini 300 spectrometer, Varian Inova 400 spectrometer and Brucker DPX-250 spectrometer. Melting points were recorded on a Büchi 535 apparatus. HPLC-MS were performed on a Gilson instrument equipped with a Gilson piston pump 321, a Gilson 864 vacuum degasser, a Gilson liquid handier 215, a Gilson 189 injection module, a Gilson Valvemate 7000, a 1/1000 splitter, a Gilson 307 make-up pump, a Gilson 170 diode array detector, and a Thermoquest Finnigan aQa detector. Semi-preparative purifications were carried out using a Symmetry C18 reverse phase column (100 Δ, 5 μm, 19×100 mm, purchased from WATERS), and water/ammonium formiate (0.1%, pH=3) and acetonitrile/ammonium formiate (0.1%, pH=3) as mobile phase.

### Intermediate 1. 2-Furancarboxamidine (HCl)

To a solution of sodium methoxide (5.55 mmol) in methanol (50 mL) was added 2-furonitrile (5.0 g, 53.2 mmol). The mixture was stirred at room temperature for 3 hours. To the resulting solution was slowly added ammonium chloride (3.14 g, 58.7 mmol) and the mixture was stirred at room temperature for 68 hours. The resulting suspension was filtered and the solvent removed under reduced pressure. The solid obtained was washed with ethyl ether (3×25 mL) to give 7.5 g (96%) of 2-furancarboxamidine (HCl).

δ (200 MHz, DMSO-d₆): 6.88-6.86 (m, 1H); 7.89 (d, J=3.8 Hz, 1H); 8.19 (s, 1H); 9.22 (s, 3H).

### Intermediate 2. 2-(2-Furyl)pyrimidine-4,6-diol

To a solution of sodium ethoxide (0.191 mol) in ethanol (90 mL) was slowly added Intermediate 1 (5.6 g, 38.2 mmol). The mixture was stirred at room temperature for 30 minutes and then, diethyl malonate (4.8.7 g, 30.4 mmol) was added. The suspension was refluxed for 32 hours. The solvent was removed under reduced pressure, the residue was suspended in water (100 mL) and acidified to pH=6 with 5N hydrochloric acid. The resulting solid was filtered and washed with water (50 mL), ethanol/ethyl ether (4:1, 25 mL), ethyl ether (2×25 mL). 2-(2-Furyl)pyrimidine-4,6-diol was obtained (4.2 g, 78%) as a pale yellow solid.

δ (300 MHz, DMSO-d₆): 5.00 (s, 1H); 6.60-6.70 (m, 1H); 7.40 (d, J=3.4 Hz, 1H); 7.80 (s, 1H).

### Intermediate 3. 4,6-Dichloro-2-(2-furyl)pyrimidine

A suspension of Intermediate 2 (3.0 g, 16.8 mmol) and N,N-diisopropylethylamine (3.85 g, 29.8 mmol) in phosphorous oxychloride (17 mL) was refluxed for 3 hours. The solvent was removed under pressure and methylene chloride (50 mL) and ice were slowly added. The organic layer was washed with water (2×25 mL), saturated solution of sodium bicarbonate (2×25 mL), brine, and dried (Na₂SO₄). The solvent was removed under reduced pressure to give 4,6-dichloro-2-(2-furyl)pyrimidine (3.15 g, 87%) as a grey solid.

δ (300 MHz, CDCl₃): 6.63-6.61 (m, 1H); 7.22 (s, 1H); 7.46 (d, J=3.4 Hz, 1H); 7.68 (s, 1H).

### Intermediate 4. 6-Chloro-2-(2-furyl)pyrimidin-amine

A suspension of Intermediate 3 (2.0 g, 9.3 mmol) in methanol (14 mL) and 30% ammonium hydroxide (27 mL) was heated in a pressure reactor for 20 hours. The solvent was partially removed under reduced pressure. The resulting solid was filtered, washed with water (25 mL), ethyl ether (25 mL), and dried. 6-Chloro-2-(2-furyl)pyrimidin-4-amine was obtained (1.48 g, 76%) as an off-white solid.

δ (400 MHz, CDCl₃): 5.21 (bs, 2H); 6.31 (s, 1H); 6.54 (m, 1H); 7.28 (d, J1=3.7 Hz, 1H); 7.58 (s, 1H).

### EXAMPLE 1

### 2-(2-Furyl)-6-(1H-pyrazol-1-yl)pyrimidin-4-amine

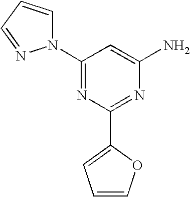

To a solution of Intermediate 4 (1.0 g, 5.1 mmol) in anhydrous DMF (20 mL) was added pyrazol (0.7 g, 10.2 mmol) and cesium carbonate (3.34 g, 10.2 mmol). The mixture was heated at 85° C. for 21 hours. The solution was poured into water (50 mL) and extracted with ethyl acetate (2×25 mL). The organic layer was washed with water (2×25 mL) and brine (25 mL), dried (Na₂SO₄), and the solvent removed under reduced pressure. The resulting solid was purified by column chromatography with silica gel, eluting with methylene chloride/methanol (3%), to give (2-furyl)-6-(1H-pyrazol-1-yl)pyrimidin-4-amine (0.64 g, 55%) as an off-white solid.

δ (250 MHz, CDCl₃): 5.12 (bs, 2H); 6.48-6.46 (m, 1H); 6.57-6.55 (m, 1H); 6.90 (s, 1H); 7.31 (d, J=3.6 Hz, 1H); 7.61 (s, 1H); 7.75 (d, J=1.2 Hz, 1H); 8.63 (d, J=3.0 Hz, 1H).

### EXAMPLE 2

### N-[2-(2-Furyl)-6-(1H-pyrazol-1-yl)pyrimidin-4-yl]acetamide

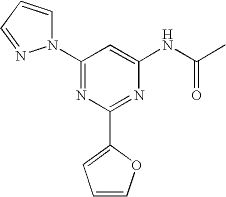

To a solution of the title compound of Example 1 (0.30 g, 1.32 mmol) in methylene chloride (7 mL) was added pyridine (0.21 g, 2.64 mmol) and acetyl chloride (0.21 g, 2.64 mmol). The mixture was stirred at room temperature for 5 hours and more pyridine (52 mg, 0.66 mmol) and acetyl chloride (52 mg, 0.66 mmol) was added. The reaction was allowed to stand for 1.5 further hours at room temperature. The solution was diluted with methylene chloride (20 mL), washed with 10% sodium hydroxide (2×10 mL), brine (10 mL), and dried (Na₂SO₄). The solvent was removed under reduced pressure. Purification by column chromatography with silica gel, eluting with ethyl acetate/n-hexane (1:3), gave N-[2-(2-furyl)-6-(1H-pyrazol-1-yl)pyrimidin-4-yl]acetamide (0.33 g, 92%) as an off-white solid.

δ (250 MHz, CDCl₃): 2.25 (s, 3H); 6.51-6.49 (m, 1H); 6.61-6.58 (m, 1H); 7.36-7.34 (m, 1H); 7.62 (s, 1H); 7.81 (s, 1H); 8.21 (bs, 1H); 8.54 (s, 1H); 8.65-8.63 (m, 1H).

### EXAMPLE 3

### N-[2-(2-Furyl)-6-(1H-pyrazol-1-yl)pyrimidin-4-yl]propanamide

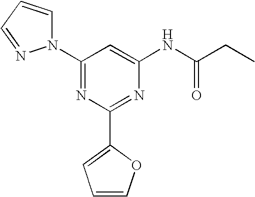

Obtained from the title compound of Example 1 (0.34 g) by the procedure described in Example 2. Purification by column chromatography with silica gel and ethyl acetate/n-hexane (1:1) as eluent gave N-[2-(2-furyl)-6-(1H-pyrazol-1-yl)pyrimidin-4-yl]propanamide (0.35 g, 83%) as an off-white solid.

δ (250 MHz, CDCl₃): 1.28 (t, J=7.3 Hz, 3H); 2.48 (q, J=7.3 Hz, 2H); 6.50-6.48 (m, 1H); 6.60-6.58 (m, 1H); 7.34 (d, J=3.6 Hz, 1H); 7.62 (s, 1H); 7.79 (s, 1H); 8.13 (bs, 1H); 8.58 (s, 1H); 8.62 (d, J=2.4 Hz, 1H).

### EXAMPLE 4

### N-[2-(2-Furyl)-6-(1H-pyrazol-1-yl)pyrimidin-4-yl]-2-methylpropanamide

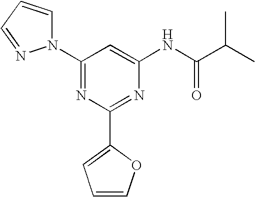

Obtained from the title compound of Example 1 (0.10 g) by the procedure described in Example 2. Purification by column chromatography with silica gel and methylene chloride/methanol (2%) as eluent gave N-[2-(2-furyl)-6-(1H-pyrazol-1-yl)pyrimidin-4-yl]-2-methylpropanamide (90 mg, 72%) as an off-white solid.

δ (250 MHz, CDCl₃): 1.28 (d, J=7.0 Hz, 6H); 2.58 (h, J=7.0 Hz, 1H); 6.49 (dd, J1=2.7 Hz, J2=1.5 Hz, 1H); 6.60 (dd, J1=3.3 Hz, J2=1.5 Hz, 1H); 7.36 (dd, J1=3.6 Hz, J2=0.9 Hz, 1H); 7.65-7.63 (m, 1H); 7.80-7.78 (m, 1H); 8.08 (bs, 1H); 8.64-8.61 (m, 2H).

### EXAMPLE 5

### N-[2-(2-Furyl)-6-(1H-pyrazol-1-yl)pyrimidin-yl]-2,2-dimethylpropanamide

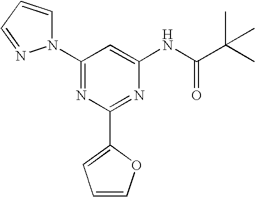

Obtained from the title compound of Example 1 (0.30 g) by the procedure described in Example 2. Purification by column chromatography with silica gel and ethyl acetate/n-hexane (from 10:90 to 15:85) as eluent gave N-[2-(2-furyl)-6-(1H-pyrazol-1-yl)pyrimidin-4-yl]-2,2-dimethylpropanamide (25 mg, 6%) as an off-white solid.

δ (250 MHz, CDCl₃): 1.35 (s, 9H); 6.49-6.47 (m, 1H); 6.59 (dd, J1=3.4 Hz, J2=1.8 Hz, 1H); 7.36-7.35 (m, 1H); 7.64-7.63 (m, 1H); 7.78-7.77 (m, 1H); 8.19 (bs, 1H); 8.62 (d, J=2.7 Hz, 1H); 8.66 (s, 1H).

### EXAMPLE 6

### N-[2-(2-Furyl)-6-(1H-pyrazol-1-yl)pyrimidin-4-yl]cyclopropanecarboxamide

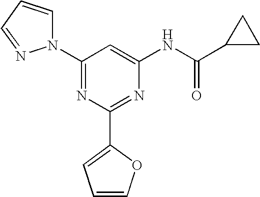

Obtained from the title compound of Example 1 (0.20 g) by the procedure described in Example 2. Purification by column chromatography with silica gel and methylene chloride/methanol (2%) as eluent gave N-[2-(2-furyl)-6-(1H-pyrazol-1-yl)pyrimidin-4-yl]cyclopropanecarboxamide (0.10 g, 39%) as an off-white solid.

δ (250 MHz, CDCl₃): 0.98-0.91 (m, 2H); 1.20-1.13 (m, 2H); 1.59-1.51 (m, 1H); 6.49 (dd, J1=2.7 Hz, J2=1.5 Hz, 1H); 6.59 (dd, J1=3.6 Hz, J2=1.8 Hz, 1H); 7.35 (d, J=3.6 Hz, 1H); 7.64-7.63 (m, 1H); 7.77 (d, J=1.5 Hz, 1H); 8.42 (bs, 1H); 8.56 (s, 1H); 8.62 (d, J=2.7 Hz, 1H).

### EXAMPLE 7

### N-[2-(2-Furyl)-6-(1H-pyrazol-1-yl)pyrimidin-4-yl]cyclobutanecarboxamide

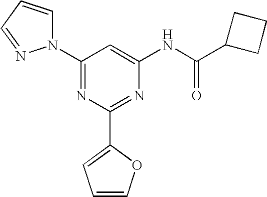

Obtained from the title compound of Example 1 (0.15 g) by the procedure described in Example 2. Purification by column chromatography with silica gel and methylene chloride/methanol (0.5%) as eluent gave N-[2-(2-furyl)-6-(1H-pyrazol-1-yl)pyrimidin-4-yl]cyclobutanecarboxamide (0.14 g, 67%) as an off-white solid.

δ (250 MHz, CDCl₃): 2.50-1.93 (m, 6H); 3.22 (q, J=8.5 Hz, 1H); 6.49 (dd, J1=2.7 Hz, J2=1.8 Hz, 1H); 6.59 (dd, J1=3.3 Hz, J2=1.8 Hz, 1H); 7.35 (d, J=3.3 Hz, 1H); 7.63 (m, 1H); 7.79 (m, 1H); 7.97 (bs, 1H); 8.62 (s, 1H); 8.63 (s, 1H).

### EXAMPLE 8

### N-[2-(2-Furyl)-6-(1H-pyrazol-1-yl)pyrimidin-4-yl]cyclohexanecarboxamide

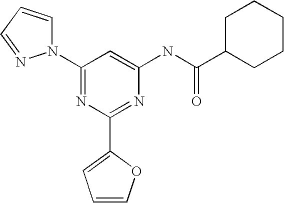

Obtained from the title compound of Example 1 (0.15 g) by the procedure described in Example 2. Purification by column chromatography with silica gel and methylene chloride/methanol (0.5%) as eluent gave N-[2-(2-furyl)-6-(1H-pyrazol-1-yl)pyrimidin-4-yl]cyclohexanecarboxamide (0.20 g, 91%) as an off-white solid.

δ (250 MHz, CDCl₃): 2.00-1.26 (m, 10H); 2.35-2.23 (m, 1H); 6.49 (dd, J1=2.7 Hz, J2=1.5 Hz, 1H); 6.59 (dd, J1=3.3 Hz, J2=1.8 Hz, 1H); 7.34 (dd, J1=3.3 Hz, J2=0.9 Hz, 1H); 7.63-7.62 (m, 1H); 7.78 (m, 1H); 8.14 (bs, 1H); 8.63-8.59 (m, 2H).

### EXAMPLE 9

### 3-Cyclopentyl-N-[2-(2-furyl)-6-(1H-pyrazol-1-yl)pyrimidin-4-yl]propanamide

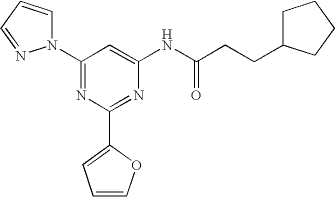

Obtained from the title compound of Example 1 (0.20 g) by the procedure described in Example 2. Purification by column chromatography with silica gel and ethyl acetate/n-hexane (from 1:9 to 2:8) as eluent gave 3-cyclopentyl-N-[2-(2-furyl)-6-(1H-pyrazol-1-yl)pyrimidin-4-yl]propanamide (0.29 g, 94%) as an off-white solid.

δ (250 MHz, CDCl₃): 1.18-1.07 (m, 2H); 1.86-1.51 (m, 9H); 2.43 (t, J=7.4 Hz, 2H); 6.49 (dd, J1=2.7 Hz, J2=1.5 Hz, 1H); 6.59 (dd, J1=3.3 Hz, J2=1.5 Hz, 1H); 7.34 (dd, J1=3.3 Hz, J2=0.6 Hz, 1H); 7.63-7.62 (m, 1H); 7.80-7.79 (m, 1H); 8.16 (bs, 1H); 8.58 (s, 1H); 8.63 (dd, J1=2.7 Hz, J2=0.6 Hz, 1H).

### EXAMPLE 10

### N-[2-(2-Furyl)-6-(1H-pyrazol-1-yl)pyrimidin-4-yl]-2-(4-methoxyphenyl)acetamide

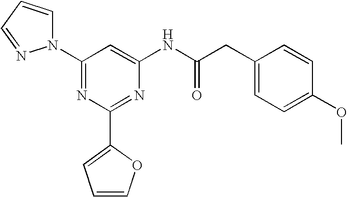

Obtained from the title compound of Example 1 (0.15 g) by the procedure described in Example 2. Purification by column chromatography with silica gel and ethyl acetate/n-hexane (1:4) as eluent gave N-[2-(2-furyl)-6-(1H-pyrazol-1-yl)pyrimidin-4-yl]-2-(4-methoxyphenyl)acetamide (63 mg, 27%) as an off-white solid.

δ (250 MHz, CDCl₃): 3.73 (s, 2H); 3.82 (s, 3H); 6.50-6.48 (m, 1H); 6.58-6.56 (m, 1H); 6.91 (s, 1H); 6.94 (s, 1H); 7.32-7.23 (m, 3H); 7.61-7.60 (m, 1H); 7.80-7.79 (m, 1H); 8.06 (bs, 1H); 8.59 (s, 1H); 8.62 (d, J=2.7 Hz, 1H).

### EXAMPLE 11

### 2-(3,4-Dimethoxyphenyl)-N-[2-(2-furyl)-6-(1H-pyrazol-1-yl)pyrimidin-4-yl]acetamide

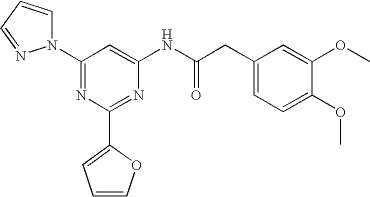

Obtained from the title compound of Example 1 (80 mg) by the procedure described in Example 2. Purification by column chromatography with silica gel and methylene chloride/methanol (0.5%) as eluent gave 2-(3,4-dimethoxyphenyl)-N-[2-(2-furyl)-6-(1H-pyrazol-1-yl)pyrimidin-4-yl]acetamide (87 mg, 61%) as an off-white solid.

δ (250 MHz, CDCl₃): 3.73 (s, 2H); 3.90 (s, 6H); 6.51-6.48 (m, 1H); 6.59-6.56 (m, 1H); 6.84 (s, 1H); 6.88 (s, 2H); 7.33 (d, J=3.3 Hz, 1H); 7.61 (s, 1H); 7.80 (s, 1H); 8.10 (bs, 1H); 8.59 (s, 1H); 8.63 (d, J=2.7 Hz, 1H).

### EXAMPLE 12

### N-[2-(2-Furyl)-6-(1H-pyrazol-1-yl)pyrimidinyl]-3-phenylpropanamide

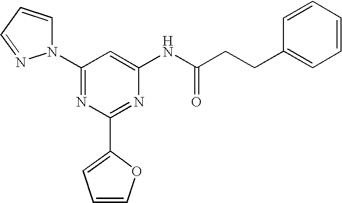

Obtained from the title compound of Example 1 (0.20 g) by the procedure described in Example 2. Purification by column chromatography with silica gel and ethyl acetate/n-hexane (1:4) as eluent gave N-[2-(2-furyl)-6-(1H-pyrazol-1-yl)pyrimidin-4-yl]-3-phenylpropanamide (0.27 g, 85%) as an off-white solid.

δ (250 MHz, CDCl₃): 2.74 (t, J=7.8 Hz, 2H); 3.08 (t, J=7.8 Hz, 2H); 6.51-6.49 (m, 1H); 6.60-6.57 (m, 1H); 7.35-7.22 (m, 6H); 7.62 (s, 1H); 7.81 (s, 1H); 8.11 (bs, 1H); 8.58 (s, 1H); 8.64 (m, 1H).

### EXAMPLE 13

### (2S)—N-[2-(2-Furyl)-6-(1H-pyrazol-1-yl)pyrimidin-4-yl]-2-phenylcyclopropanecarboxamide

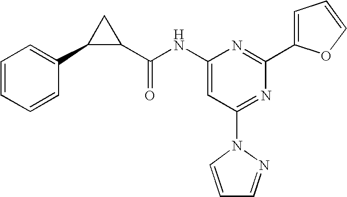

Obtained from the title compound of Example 1 (0.15 g) by the procedure described in Example 2. Purification by column chromatography with silica gel and methylene chloride/methanol (0.5%) as eluent gave (2S)—N-[2-(2-furyl)-6-(1H-pyrazol-1-yl)pyrimidin-4-yl]-2-phenylcyclopropanecarboxamide (0.23 g, 95%) as an off-white solid.

δ (250 MHz, CDCl₃): 1.49-1.40 (m, 1H); 1.86-1.75 (m, 2H); 2.71-2.63 (m, 1H); 6.50-6.49 (m, 1H); 6.57 (dd, J1=3.3 Hz, J2=1.8 Hz, 1H); 7.14-7.11 (m, 2H); 7.34-7.19 (m, 4H); 7.61 (m, 1H); 7.79 (m, 1H); 8.59-8.58 (m, 2H); 8.63 (d, J=2.7 Hz, 1H).

### EXAMPLE 14

### 3,3,3-Trifluoro-N-[2-(2-furyl)-6-(1H-pyrazol-1-yl)pyrimidin-4-yl]propanamide

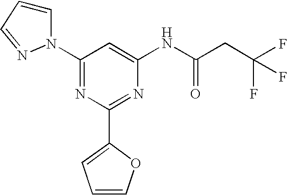

To a solution of 3,3,3-trifluoropropionic acid (0.21 g, 1.65 mmol) in methylene chloride (4 mL) was added oxalyl chloride (0.21 g, 1.65 mmol) and a catalytic amount of DMF. The mixture was stirred at room temperature for 1 hour. This solution was cooled at 0° C. and added at the same temperature to a solution of the title compound of Example 1 (125 mg, 0.55 mmol) and pyridine (123 mg, 1.65 mmol) in methylene chloride (4 mL). The mixture was stirred at room temperature for 22 hours and diluted with methylene chloride (8 mL). The organic layer was washed with water (2×8 mL) and brine (8 mL), dried (Na₂SO₄), and the solvent removed under reduced pressure. Purification by column chromatography with silica gel, eluting with ethyl acetate/n-hexane (1:4), gave 3,3,3-trifluoro-N-[2-(2-furyl)-6-(1H-pyrazol-1-yl)pyrimidin-4-yl]propanamide (0.16 g, 87%) as an off-white solid.

δ (250 MHz, CDCl₃): 3.33 (q, J=10.0 Hz, 2H); 6.52-6.50 (m, 1H); 6.61-6.59 (m, 1H); 7.36 (d, J=3.6 Hz, 1H); 7.64-7.63 (m, 1H); 7.82-7.81 (m, 1H); 8.40 (bs, 1H); 8.54 (s, 1H); 8.65-8.63 (m, 1H).

### EXAMPLE 15

### 3-(3,4-Dimethoxyphenyl)-N-[2-(2-furyl)-6-(1H-pyrazol-1-yl)pyrimidin-4-yl]propanamide

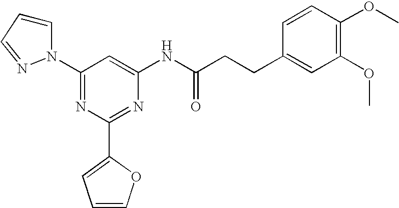

Obtained from the title compound of Example 1 (0.20 g) by the procedure described in Example 14. Purification by column chromatography with silica gel and ethyl acetate/n-hexane (from 3:7 to 2:3) as eluent gave 3-(3,4-dimethoxyphenyl)-N-[2-(2-furyl)-61(H-pyrazol-1-yl)pyrimidin-4-yl]propanamide (0.27 g, 72%) as an off-white solid.

δ (250 MHz, CDCl₃): 2.71 (t, J=7.6 Hz, 2H); 3.02 (t, J=7.6 Hz, 2H); 3.85 (s, 3H); 3.86 (s, 3H); 6.50 (dd, J1=2.7 Hz, J2=1.5 Hz, 1H); 6.59 (dd, J1=3.3 Hz, J2=1.8 Hz, 1H); 6.80-6.76 (m, 3H); 7.34 (dd, J1=3.3 Hz, J2=0.9 Hz, 1H); 7.62 (dd, J1=1.8 Hz, J2=0.9 Hz, 1H); 7.81 (dd, J1=1.1 Hz, J2=0.6 Hz, 1H); 8.07 (bs, 1H); 8.58 (s, 1H); 8.64 (dd, J1=2.7 Hz, J2=0.6 Hz, 1H).

### EXAMPLE 16

### N-[2-(2-Furyl)-6-(1H-pyrazol-1-yl)pyrimidin-4-yl]-2-methyl-3-phenylpropanamide

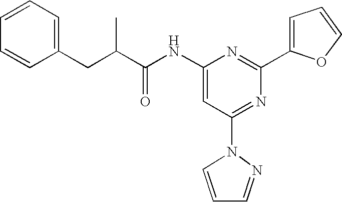

Obtained from the title compound of Example 1 (0.15 g) by the procedure described in Example 14. Purification by column chromatography with silica gel and methylene chloride/methanol (1%) as eluent gave N-[2-(2-furyl)-6-(1H-pyrazol-1-yl)pyrimidin-4-yl]-2-methyl-3-phenylpropanamide (0.14 g, 51%) as an off-white solid.

δ (250 MHz, CDCl₃): 1.29 (d, J=6.4 Hz, 3H); 2.79-2.62 (m, 2H); 3.16-3.08 (m, 1H); 6.51-6.49 (m, 1H); 6.59-6.57 (m, 1H); 7.34-7.16 (m, 6H); 7.62 (m, 1H); 7.80 (m, 1H); 7.97 (bs, 1H); 8.63-8.61 (m, 2H)

### EXAMPLE 17

### N-[2-(2-Furyl)-6-(1H-pyrazol-1-yl)pyrimidin-4-yl]-3-phenoxypropanamide

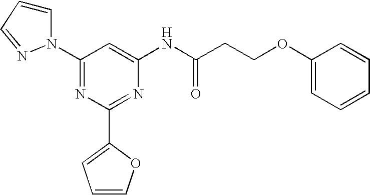

Obtained from the title compound of Example 1 (0.20 g) by the procedure described in Example 14. Purification by column chromatography with silica gel and ethyl acetate/n-hexane (30:70) as eluent gave N-[2-(2-furyl)-6-(1H-pyrazol-1-yl)pyrimidin-4-yl]-3-phenoxypropanamide (0.23 g, 70%) as an off-white solid.

δ (250 MHz, CDCl₃): 2.91 (t, 2H); 4.37 (t, 2H); 6.50-6.49 (m, 1H); 6.61-6.58 (m, 1H); 7.01-6.94 (m, 3H); 7.35-7.27 (m, 3H); 7.64 (m, 1H); 7.80 (m, 1H); 8.58 (s, 1H); 8.64 (d, J=2.4 Hz, 1H).

### EXAMPLE 18

### N-[2-(2-Furyl)-6-(1H-pyrazol-1-yl)pyrimidin-4-yl]-3-pyridin-3-ylpropanamide

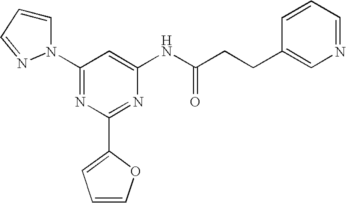

Obtained from the title compound of Example 1 (0.20 g) by the procedure described in Example 14. Purification by column chromatography with silica gel and methylene chloride/methanol (2%) as eluent gave N-[2-(2-furyl)-6-(1H-pyrazol-1-yl)pyrimidinyl]-3-pyridin-3-ylpropanamide (0.19 g, 60%) as an off-white solid.

δ (250 MHz, CDCl₃): 2.76 (t, J=7.3 Hz, 2H); 3.09 (t, J=7.3 Hz, 2H); 6.51-6.50 (m, 1H); 6.60-6.58 (m, 1H); 7.28-7.21 (m, 1H); 7.34 (d, J=3.6 Hz, 1H); 7.63-7.57 (m, 2H); 7.81 (s, 1H); 8.13 (s, 1H); 8.54-8.47 (m, 2H); 8.56 (s, 1H); 8.64 (d, J=2.4 Hz, 1H).

### EXAMPLE 19

### N-[2-(2-Furyl)-6-(1H-pyrazol-1-yl)pyrimidin-4-yl]-2-pyridin-3-ylacetamide

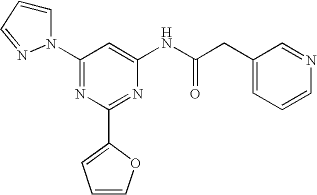

Obtained from the title compound of Example 1 (0.20 g) by the procedure described in Example 14. Purification by column chromatography with silica gel and methylene chloride/methanol (2%) as eluent gave N-[2-(2-furyl)-6-(1H-pyrazol-1-yl)pyrimidin-4-yl]-2-pyridin-3-ylacetamide (0.70 g, 23%) as an off-white solid.

δ (250 MHz, DMSO-d₆): 3.89 (s, 2H); 6.65 (dd, J1=2.6 Hz, J2=1.3 Hz, 1H); 6.75 (dd, J1=3.5 Hz, J2=1.7 Hz, 1H); 7.37 (dd, J1=7.9 Hz, J2=4.8 Hz, 1H); 7.49 (dd, J1=3.5 Hz, J2=0.9 Hz, 1H); 7.77 (dt, J1=7.9 Hz, J2=1.7 Hz, 1H); 7.91 (m, 1H); 7.97 (m, 1H); 8.40 (s, 1H); 8.48 (dd, J1=4.8 Hz, J2=1.7 Hz, 1H); 8.55 (d, J1=1.7 Hz, 1H); 8.77 (d, J1=2.6 Hz, 1H); 11.50 (s, 1H).

### EXAMPLE 20

### (2E)-3-(3,4-Dimethoxyphenyl)-N-[2-(2-furyl)-6-(1H-pyrazol-1-yl)pyrimidin-4-yl]acrylamide

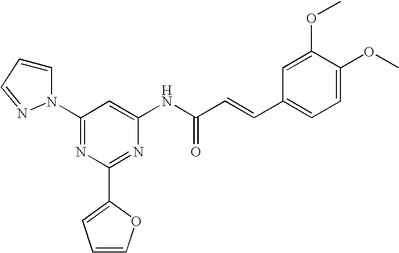

A solution of 3,4-dimethoxyphenylacrylic acid (0.55 g, 2.64 mmol) in thionyl chloride (4 mL) was stirred at 55° C. for 1 hour. The solvent was removed under reduced pressure. The resulting oil was dissolved in methylene chloride (2 mL) and the solution was cooled at 0° C. and added to a solution of the title compound of Example 1 (0.20 mg, 0.88 mmol) and pyridine (0.20 mg, 2.64 mmol) in methylene chloride (6 mL). The mixture was stirred at room temperature for 44 hours and diluted with methylene chloride (8 mL). The organic layer was washed with water (2×8 mL) and brine (8 mL), dried (Na₂SO₄), and the solvent removed under reduced pressure. Purification by column chromatography with silica gel, eluting with methylene chloride/ethanol (0.5%), gave (2E)-3-(3,4-dimethoxyphenyl)-N-[2-(2-furyl)-6-(1H-pyrazol-1-yl)pyrimidin-4-yl]acrylamide (0.70 g, 19%) as an off-white solid.

δ (250 MHz, CDCl₃): 3.93 (s, 3H); 3.94 (s, 3H); 6.40 (d, J=15.5 Hz, 1H); 6.52-6.50 (m, 1H); 6.61-6.59 (m, 1H); 6.90 (d, J=8.2 Hz, 1H); 7.06 (d, J=1.8 Hz, 1H); 7.16 (dd, J1=8.2 Hz, J2=1.8 Hz, 1H); 7.36 (dd, J1=3.3 Hz, J2=0.6 Hz, 1H); 7.64-7.63 (m, 1H); 7.76 (d, J=15.5 Hz, 1H); 7.82 (m, 1H); 8.33 (bs, 1H); 8.66-8.65 (m, 1H); 8.69 (s, 1H).

### EXAMPLE 21

### 6-(3,5-Dimethyl-1H-pyrazol-1-yl)-2-(2-furyl)pyrimidin-4-amine

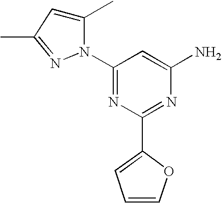

To a solution of Intermediate 4 (2.0 g, 10.2 mmol) in anhydrous DMSO (50 mL) was added 3,5-dimethylpyrazol (1.97 g, 20.5 mmol) and cesium carbonate (6.70 g, 20.6 mmol). The mixture was heated at 150° C. for 9 hours. The solution was poured into water (150 mL) and extracted with ethyl acetate (3×100 mL). The organic layer was washed with water (3×100 mL), brine (100 mL), dried (Na₂SO₄), and the solvent removed under reduced pressure. The resulting solid was purified by column chromatography with silica gel, eluting with ethyl acetate/n-hexane (from 3:7 to 1:1), to give 6-(3,5-dimethyl-1H-pyrazol-1-yl)-2-(2-furyl)pyrimidin-4-amine (1.86 g, 71%) as an off-white solid.

δ (250 MHz, CDCl₃): 2.29 (s, 3H); 2.78 (s, 3H); 5.10 (bs, 2H); 6.00 (s, 1H); 6.55-6.52 (m, 1H); 6.84 (s, 1H); 7.19 (d, J=2.4 Hz, 1H); 7.58 (s, 1H).

### EXAMPLE 22

### N-[6-(3,5-Dimethyl-1H-pyrazol-1-yl)-2-(2-furyl)pyrimidin-4-yl]acetamide

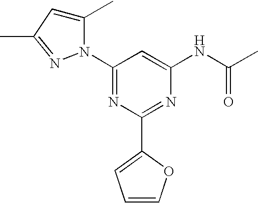

Obtained from the title compound of Example 21 (0.30 g) by the procedure described in Example 2. Purification by column chromatography with silica gel and ethyl acetate/n-hexane (1:1) as eluent gave N-[6-(3,5-dimethyl-1H-pyrazol-1-yl)-2-(2-furyl)pyrimidin-4-yl]acetamide (0.25 g, 72%) as an off-white solid.

δ (250 MHz, CDCl₃): 2.23 (s, 3H); 2.29 (s, 3H); 2.77 (s, 3H); 6.02 (s, 1H); 6.58-6.55 (m, 1H); 7.24 (d, J=3.3 Hz, 1H); 7.61-7.60 (m, 1H); 8.17 (bs, 1H); 8.48 (s, 1H).

### EXAMPLE 23

### N-[2-(3,5-Dimethyl-1H-pyrazol-1-yl)-6-(2-furyl)pyrimidin-4-yl]propanamide

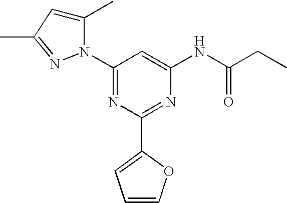

Obtained from the title compound of Example 21 (0.30 g) by the procedure described in Example 2. Purification by column chromatography with silica gel and ethyl acetate/n-hexane (1:4) as eluent gave N-[2-(3,5-dimethyl-1H-pyrazol-1-yl)-6-(2-furyl)pyrimidin-4-yl]propanamide (0.26 g, 71%) as an off-white solid.

δ (250 MHz, CDCl₃): 1.27 (t, J=7.6 Hz, 3H); 2.29 (s, 3H); 2.46 (q, J=7.6 Hz, 2H); 2.78 (s, 3H); 6.03 (s, 1H); 6.59-6.57 (m, 1H); 7.25 (d, J=2.7 Hz, 1H); 7.62-7.61 (m, 1H); 8.12 (bs, 1H); 8.55 (s, 1H).

### EXAMPLE 24

### N-[6-(3,5-Dimethyl-1H-pyrazol-1-yl)-2-(2-furyl)pyrimidin-4-yl]-2-methylpropanamide

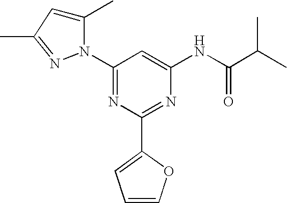

Obtained from the title compound of Example 21 (0.15 g) by the procedure described in Example 2. Purification by column chromatography with silica gel and methylene chloride/methanol (1%) as eluent gave N-[6-(3,5-dimethyl-1H-pyrazol-1-yl)-2-(2-furyl)pyrimidin-4-yl]-2-methylpropanamide (0.11 g, 60%) as an off-white solid.

δ (250 MHz, CDCl₃): 1.28 (d, J=7.0 Hz, 6H); 2.27 (s, 3H); 2.56 (h, J=7.0 Hz, 1H); 2.77 (s, 3H); 6.02 (s, 1H); 6.58-6.56 (m, 1H); 7.26 (s, 1H); 7.62-7.61 (m, 1H); 8.03 (bs, 1H); 8.58 (s, 1H).

### EXAMPLE 25

### N-[6-(3,5-Dimethyl-1H-pyrazol-1-yl)-2-(2-furyl)pyrimidin-4-yl]-2,2-dimethylpropanamide

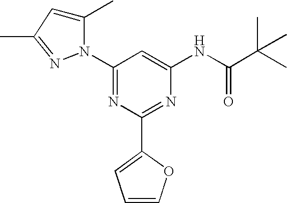

Obtained from the title compound of Example 21 (0.15 g) by the procedure described in Example 2. Purification by column chromatography with silica gel and ethyl acetate/n-hexane (15:85) as eluent gave N-[6-(3,5-dimethyl-1H-pyrazol-1-yl)₂₋₂-furyl)pyrimidin-4-yl]-2,2-dimethylpropanamide (95 mg, 48%) as an off-white solid.

δ (250 MHz, CDCl₃): 1.34 (s, 9H); 2.27 (s, 3H); 2.77 (s, 3H); 6.02 (s, 1H); 6.57 (dd, J1=3.3 Hz, J2=1.8 Hz, 1H); 7.25 (d, J=0.9 Hz, 1H); 7.62 (dd, J1=1.8 Hz, J2=0.9 Hz, 1H); 8.14 (bs, 1H); 8.62 (s, 1H).

### EXAMPLE 26

### N-[6-(3,5-Dimethyl-1H-pyrazol-1-yl)-2-(2-furyl)pyrimidin-4-yl]-cyclopropanecarboxamide

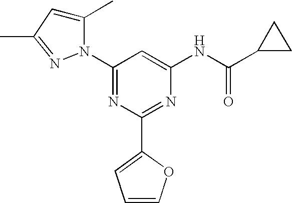

Obtained from the title compound of Example 21 (0.15 g) by the procedure described in Example 2. Purification by column chromatography with silica gel and methylene chloride/methanol (1%) as eluent gave N-[6-(3,5-dimethyl-1H-pyrazol-1-yl)-2-(2-furyl)pyrimidin-4-yl]cyclopropanecarboxamide (70 mg, 37%) as an off-white solid.

δ (250 MHz, CDCl₃): 0.97-0.89 (m, 2H); 1.21-1.13 (m, 2H); 1.59-1.49 (m, 1H); 2.26 (s, 3H); 2.77 (s, 3H); 6.01 (s, 1H); 6.57 (dd, J1=3.3 Hz, J2=1.8 Hz, 1H); 7.24 (dd, J1=3.3 Hz, J2=0.9 Hz, 1H); 7.61 (dd, J1=1.8 Hz, J2=0.9 Hz, 1H); 8.39 (bs, 1H); 8.52 (s, 1H).

### EXAMPLE 27

### 3-Cyclopentyl-N-[6-(3,5-dimethyl-1H-pyrazol-1-yl)-2-(2-furyl)pyrimidin-4-yl]propanamide

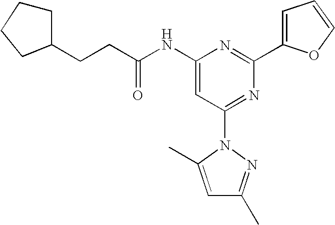

Obtained from the title compound of Example 21 (0.15 g) by the procedure described in Example 2. Purification by column chromatography with silica gel and methylene chloride/n-hexane (from 90% to pure methylene chloride) as eluent gave 3-cyclopentyl-N-[6-(3,5-dimethyl-1H-pyrazol-1-yl)-2-(2-furyl)pyrimidin-4-yl]propanamide (0.22 g, 99%) as an off-white solid.

δ (250 MHz, CDCl₃): 1.16-1.07 (m, 2H); 1.83-1.51 (m, 9H); 2.28 (s, 3H); 2.42 (t, J=7.3 Hz, 2H); 2.77 (s, 3H); 6.02 (s, 1H); 6.57 (dd, J1=3.3 Hz, J2=1.8 Hz, 1H); 7.24 (d, J=3.6 Hz, 1H); 7.61 (s, 1H); 8.16 (bs, 1H); 8.54 (s, 1H).

### EXAMPLE 28

### N-[6-(3,5-dimethyl-1H-pyrazol-1-yl)-2-(2-furyl)pyrimidin-4-yl]-2-(4-methoxyphenyl)acetamide

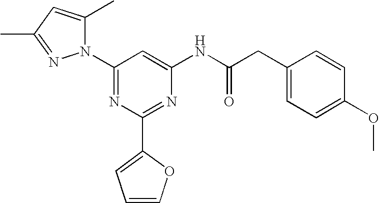

Obtained from the title compound of Example 21 (0.15 g) by the procedure described in Example 2. Purification by column chromatography with silica gel and methylene chloride/methanol (2%) as eluent gave N-[6-(3,5-dimethyl-1H-pyrazol-1-yl)-2-(2-furyl)pyrimidin-4-yl]-2-(4-methoxyphenyl)acetamide (0.11 g, 46%) as an off-white solid.

δ (250 MHz, CDCl₃): 2.28 (s, 3H); 2.76 (s, 3H); 3.71 (s, 2H); 3.82 (s, 3H); 6.01 (s, 1H); 6.55 (dd, J1=3.3 Hz, J2=1.8 Hz, 1H); 6.89 (s, 1H); 6.93 (s, 1H); 7.26-7.20 (m, 3H); 7.59-7.58 (m, 1H); 8.04 (s, 1H); 8.54 (s, 1H).

### EXAMPLE 29

### 2-(3,4-Dimethoxyphenyl)-N-[6-(3,5-dimethyl-1H-pyrazol-1-yl)-2-(2-furyl)pyrimidin-4-yl]acetamide

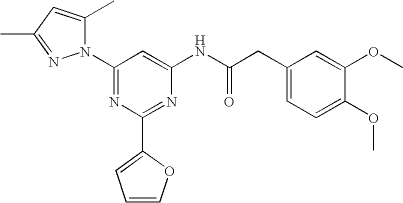

Obtained from the title compound of Example 21 (0.15 g) by the procedure described in Example 2. Purification by column chromatography with silica gel and methylene chloride/methanol (2%) as eluent gave 2-(3,4-dimethoxyphenyl)-N-[6-(3,4-dimethyl-1H-pyrazol-1-yl)-2-(2-furyl)pyrimidin-4-yl]acetamide (0.12%, 47%) as a n off-white solid.

δ (250 MHz, CDCl₃): 2.28 (s, 3H); 2.77 (s, 3H); 3.71 (s, 2H); 3.89 (s, 3H); 3.90 (s, 3H); 6.02 (s, 1H); 6.55 (dd, J1=3.3 Hz, J2=1.8 Hz, 1H); 6.82 (s, 1H); 6.87 (s, 1H); 6.88 (s, 1H); 7.22 (dd, J1=3.3 Hz, J2=0.9 Hz, 1H); 7.59 (dd, J1=1.8 Hz, J2=0.9 Hz, 1H); 8.02 (bs, 1H); 8.54 (s, 1H).

### EXAMPLE 30

### N-[6-(3,5-Dimethyl-1H-pyrazol-1-yl)-2-(2-furyl)pyrimidin-4-yl]-3-phenylpropanamide

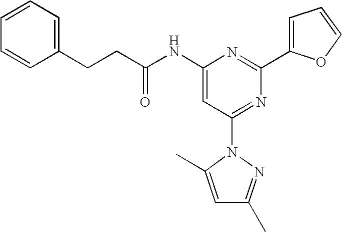

Obtained from the title compound of Example 21 (0.15 g) by the procedure described in Example 2. Purification by column chromatography with silica gel and methylene chloride/n-hexane (from 90% to pure methylene chloride) as eluent gave N-[6-(3,5-dimethyl-1H-pyrazol-1-yl)₂₋₂-furyl)pyrimidinyl]-3-phenylpropanamide (0.23 g, 99%) as an off-white solid.

δ (250 MHz, CDCl₃): 2.29 (s, 3H); 2.72 (t, J=7.6 Hz, 2H); 2.77 (5, 3H); 3.07 (t, J=7.6 Hz, 2H); 6.02 (s, 1H); 6.57-6.55 (m, 1H); 7.34-7.18 (m, 6H); 7.60 (m, 1H); 8.15 (bs, 1H); 8.54 (s, 1H).

### EXAMPLE 31

### N-[6-(3,5-Dimethyl-1H-pyrazol-1-yl)-2-(2-furyl)pyrimidin-4-yl]-3,3,3-trifluoropropanamide

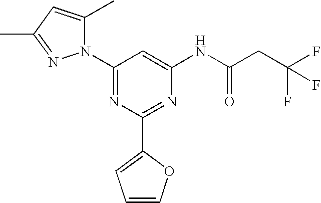

Obtained from the title compound of Example 21 (0.30 g) by the procedure described in Example 14. Purification by column chromatography with silica gel and ethyl acetate/n-hexane (1:4) as eluent gave N-[6-(3,5-dimethyl-1H-pyrazol-1-yl)-2-(2-furyl)pyrimidin-4-yl]-3,3,3-trifluoropropanamide (0.21 g, 49%) as an off-white solid.

δ (250 MHz, CDCl₃): 2.29 (s, 3H); 2.78 (s, 3H); 3.30 (c, J=10.0 Hz, 2H); 6.04-6.02 (m, 1H); 6.59-6.57 (m, 1H); 7.28-7.24 (m, 1H); 7.62-7.61 (m, 1H); 8.30 (bs, 1H); 8.50 (s, 1H).

### EXAMPLE 32

### 3-(3,4-Dimethoxyphenyl)-N-[6-(3,5-dimethyl-1H-pyrazol-1-yl)-2-(2-furyl)pyrimidin-yl]propanamide

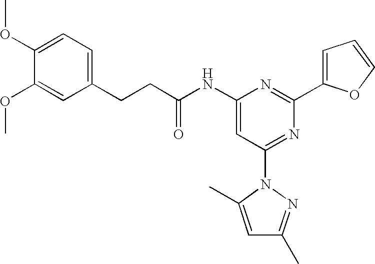

Obtained from the title compound of Example 21 (0.15 g) by the procedure described in Example 14. Purification by column chromatography with silica gel and methylene chloride/methanol (1%) as eluent gave 3-(3,4-dimethoxyphenyl)-N-[6-(3,5-dimethyl-1H-pyrazol-1-yl)-2-(2-furyl)pyrimidin-4-yl]propanamide (0.18 g, 67%) as an off-white solid.

δ (250 MHz, CDCl₃): 2.70 (t, J=7.6 Hz, 2H); 2.77 (s, 6H); 3.02 (t, J=7.6 Hz, 2H); 3.85 (s, 3H); 3.87 (s, 3H); 6.03 (s, 1H); 6.58-6.55 (m, 1H); 6.82-6.75 (m, 3H); 7.23 (dd, J1=3.3 Hz, J2=0.9 Hz, 1H); 7.60 (dd, J1=1.8 Hz, J2=0.9 Hz, 1H); 8.09 (bs, 1H); 8.54 (s, 1H).

### EXAMPLE 33

### N-[6-(3,5-Dimethyl-1H-pyrazol-1-yl)-2-(2-furyl)pyrimidin-4-yl]-3-phenoxypropanamide

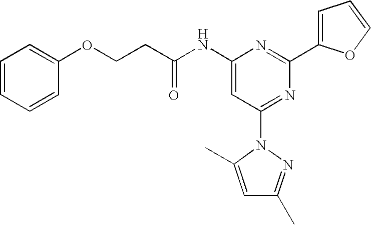

Obtained from the title compound of Example 21 (0.15 g) by the procedure described in Example 14. Purification by column chromatography with silica gel and methylene chloride/methanol (1%) as eluent gave N-[6-(3,5-dimethyl-1H-pyrazol-1-yl)-2-(2-furyl)pyrimidin-4-yl]-3-phenoxypropanamide (0.21 g, 88%) as an off-white solid.

δ (250 MHz, CDCl₃): 2.28 (s, 3H); 2.78 (s, 3H); 2.89 (t, J=6.1 Hz, 2H); 4.36 (t, J=6.1 Hz, 2H); 6.02 (s, 1H); 6.58-6.56 (m, 1H); 7.00-6.93 (m, 3H); 7.33-7.24 (m, 3H); 7.62 (m, 1H); 8.47 (bs, 1H); 8.54 (s, 1H).

### EXAMPLE 34

### N-[6-(3,5-Dimethyl-1H-pyrazol-1-yl)-2-(2-furyl)pyrimidin-4-yl]-2-pyridin-3-ylacetamide

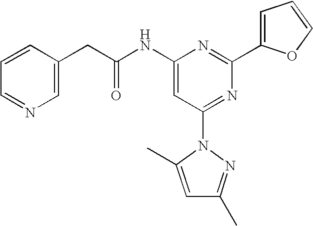

Obtained from the title compound of Example 21 (0.15 g) by the procedure described in Example 14. Purification by column chromatography with silica gel and methylene chloride/methanol (2.5%) as eluent gave N-[6-(3,5-dimethyl-1H-pyrazol-1-yl)-2-(2-furyl)pyrimidin-4-yl]-2-pyridin-3-ylacetamide (55 mg, 25%) as an off-white solid.

δ (250 MHz, DMSO-d₆): 2.19 (s, 3H); 2.74 (s, 3H); 3.87 (s, 2H); 6.20 (s, 1H); 6.73 (dd, J1=3.4 Hz, J2=1.7 Hz, 1H); 7.30 (d, J=3.4 Hz, 1H); 7.37 (dd, J1=7.7 Hz, J2=4.7 Hz, 1H); 7.79-7.74 (m, 1H); 7.96 (s, 1H); 8.35 (s, 1H); 8.50-8.46 (m, 1H); 11.41 (s, 1H)

### EXAMPLE 35

### N-[6-(3,5-Dimethyl-1H-pyrazol-1-yl)-2-(2-furyl)pyrimidin-yl]-3-pyridin-3-ylpropanamide

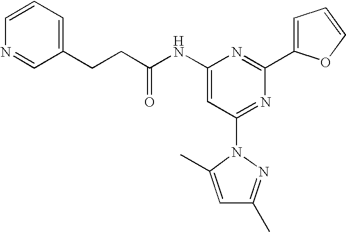

Obtained from the title compound of Example 21 (0.20 g) by the procedure described in Example 14. Purification by column chromatography with silica gel and methylene chloride/methanol (2%) as eluent gave N-[6-(3,5-dimethyl-1H-pyrazol-1-yl)-2-(2-furyl)pyrimidin-4-yl]-3-pyridin-3-ylpropanamide (97 mg, 31%) as an off-white solid.

δ (250 MHz, DMSO-d₆): 2.23 (s, 3H); 2.74 (s, 3H); 2.97-2.81 (m, 4H); 6.22 (s, 1H); 6.73-6.71 (m, 1H); 7.34-7.27 (m, 2H); 7.71-7.66 (m, 1H); 7.95 (m, 1H); 8.37 (s, 1H); 8.42-8.39 (m, 1H); 8.49 (m, 1H); 11.13 (s, 1H).

### EXAMPLE 36

### 2-(2-Furyl)-6-(4-methyl-1H-pyrazol-1-yl)pyrimidin-4-amine

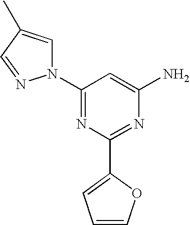

Obtained from Intermediate 4 (0.50 g) by the procedure described in Example 21. Purification by column chromatography with silica gel and ethyl acetate/n-hexane (1:1) as eluent gave 2-(2-furyl)-6-(4-methyl-1H-pyrazol-1-yl)pyrimidin-4-amine (0.29 g, 47%) as an off-white solid.

δ (250 MHz, CDCl₃): 2.16 (s, 3H); 5.10 (bs, 2H); 6.57-6.55 (m, 1H); 6.83 (s, 1H); 7.29 (dd, J1=3.3 Hz, J2=0.6 Hz, 1H); 7.56 (s, 1H); 7.61 (m, 1H); 8.39 (s, 1H).

### EXAMPLE 37

### N-[2-(2-Furyl)-6-(4-methyl-1H-pyrazol-1-yl)pyrimidin 4-yl]-propanamide

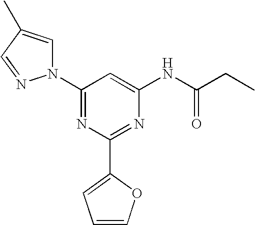

Obtained from the title compound of Example 36 (0.19 g) by the procedure described in Example 2. Purification by column chromatography with silica gel and methylene chloride as eluent gave N-[2-(2-furyl)-6-(4-methyl-1H-pyrazol-1-yl)pyrimidin-4-yl]propanamide (0.20 g, 82%) as an off-white solid.

δ (250 MHz, CDCl₃): 1.25 (t, J=7.3 Hz, 2H); 2.16 (s, 3H); 2.45 (q, J=7.3 Hz, 2H); 6.59-6.57 (m, 1H); 7.33 (d, J=3.3 Hz, 1H); 7.62-7.60 (m, 2H); 8.12 (bs, 1H); 8.37 (s, 1H); 8.51 (s, 1H).

### EXAMPLE 38

### 2-(2-Furyl)-6-(3-methyl-1H-pyrazol-1-yl)pyrimidin-4-amine

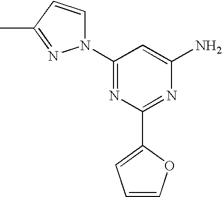

Obtained from Intermediate 4 (0.50 g) by the procedure described in Example 21. Purification by column chromatography with silica gel and ethyl acetate/n-hexane (1:1) as eluent gave 2-(2-furyl)-6-(3-methyl-1H-pyrazol-1-yl)pyrimidin-4-amine (0.47 g, 76%) as an off-white solid.

δ (250 MHz, CDCl₃): 2.37 (s, 3H); 5.10 (bs, 2H); 6.26 (d, J=2.7 Hz, 1H); 6.55 (dd, J1=3.3 Hz, J2=1.8 Hz, 1H); 6.82 (s, 1H); 7.29 (dd, J1=3.3 Hz, J2=0.9 Hz, 1H); 7.60 (dd, J1=1.8 Hz, J2=0.9 Hz, 1H); 8.52-8.51 (m, 1H).

### EXAMPLE 39

### N-[2-(2-Furyl)-6-(3-methyl-1H-pyrazol-1-yl)pyrimidin-4-yl]propanamide

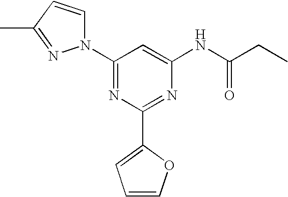

Obtained from the title compound of Example 38 (0.20 g) by the procedure described in Example 2. Purification by column chromatography with silica gel and methylene chloride/methanol (2%) as eluent gave N-[2-(2-furyl)-6-(3-methyl-1H-pyrazol-1-yl)pyrimidin-4-yl]propanamide (0.17 g, 70%) as an off-white solid.

δ (250 MHz, CDCl₃): 1.26 (t, J=7.6 Hz, 3H); 2.36 (s, 3H); 2.46 (q, J=7.6 Hz, 2H); 6.28 (d, J=2.4 Hz, 1H); 6.58 (dd, J1=3.6 Hz, J2=1.8 Hz, 1H); 7.33-7.31 (m, 1H); 7.61 (s, 1H); 8.11 (bs, 1H); 8.51-8.49 (m, 2H).

### EXAMPLE 40

### 2-(2-Furyl)-6-[3-(trifluoromethyl)-1H-pyrazol-1-yl]pyrimidin-4-amine

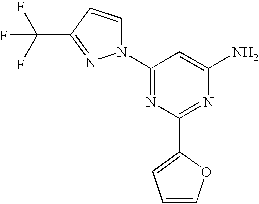

Obtained from Intermediate 4 (0.50 g) by the procedure described in Example 21. Purification by column chromatography with silica gel and methylene chloride/methanol (5%) as eluent gave 2-(2-furyl)-6-[3-(trifluoromethyl)-1H-pyrazol-1-yl]pyrimidin-4-amine (0.49 g, 65%) as an off-white solid.

δ (250 MHz, CDCl₃): 5.22 (bs, 2H); 6.58 (dd, J1=3.3 Hz, J2=1.8 Hz, 1H); 6.72 (d, J=2.7 Hz, 1H); 6.95 (s, 1H); 7.32 (dd, J1=3.3 Hz, J2=0.9 Hz, 1H); 7.62 (dd, J1=1.8 Hz, J2=0.9 Hz, 1H); 8.70-8.69 (m, 1H).

### EXAMPLE 41

### N-{2-(2-Furyl)-6-[3-(trifluoromethyl)-1H-pyrazol-1-yl]pyrimidin-4-yl}-propanamide

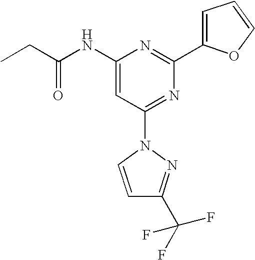

Obtained from the title compound of Example 40 (0.15 g) by the procedure described in Example 2. Purification by column chromatography with silica gel and methylene chloride/n-hexane (from 90% to pure methylene chloride) as eluent gave N-{2-(2-furyl)-6-[3]-(trifluoromethyl)-1H-pyrazol-1-yl]pyrimidin-4-yl}propanamide (0.18 g, 99%) as an off-white solid.

δ (250 MHz, CDCl₃): 1.27 (t, J1=7.6 Hz, 3H); 2.49 (q, J1=7.6 Hz, 2H); 6.60 (dd, J1=3.3 Hz, J2=1.8 Hz, 1H); 6.73 (d, J1=2.7 Hz, 1H); 7.35 (d, J1=3.3 Hz, 1H); 7.64 (s, 1H); 8.18 (bs, 1H); 8.62 (s, 1H); 8.96 (m, 1H).

### EXAMPLE 42

### 2-(2-Furyl)-6-[5-methyl-3-(trifluoromethyl)-1H-pyrazol-1-yl]pyrimidin-4-amine

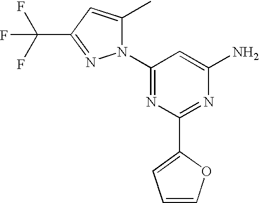

Obtained from Intermediate 4 (0.50 g) by the procedure described in Example 21. Purification by column chromatography with silica gel and ethyl acetate/n-hexane (from 1:9 to 3:7) as eluent gave 2-(2-furyl)-6-[5-methyl-3-(trifluoromethyl)-1H-pyrazol-1-yl]pyrimidin-4-amine (0.13 g, 16%) as an off-white solid.

δ (250 MHz, CDCl₃): 2.84 (s, 3H); 5.26 (bs, 2H); 6.45 (s, 1H); 6.57-6.55 (m, 1H); 6.91 (s, 1H); 7.22 (dd, J1=3.3 Hz, J2=0.9 Hz, 1H); 7.61-7.60 (m, 1H).

### EXAMPLE 43

### N-{2-(2-furyl)-6-[5-methyl-3-(trifluoromethyl)-1H-pyrazol-1-yl]-pyrimidin-4-yl}propanamide

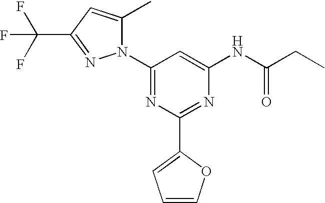

Obtained from the title compound of Example 42 (0.25 g) by the procedure described in Example 2. Purification by column chromatography with silica gel and ethyl acetate/n-hexane (1:4) as eluent gave N-{2-(2-furyl)-6-[5-methyl-3-trifluoromethyl)-1H-pyrazol-1-yl]pyrimidin-4-yl}propanamide (0.23 g, 77%) as an off-white solid.

δ (250 MHz, CDCl₃): 1.26 (t, J=7.6 Hz, 3H); 2.48 (q, J=7.6 Hz, 2H); 2.84 (s, 3H); 6.47 (s, 1H); 6.59 (dd, J1=3.3 Hz, J2=1.8 Hz, 1H); 7.28-7.26 (m, 1H); 7.63 (dd, J1=1.8 Hz, J2=0.9 Hz, 1H); 8.16 (bs, 1H); 8.58 (s, 1H).

### EXAMPLE 44

### 2-(2-Furyl)-6-(1H-1,2,4-triazol-1-yl)pyrimidin-4-amine

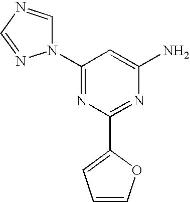

Obtained from Intermediate 4 (1.90 g) by the procedure described in Example 21. Purification by column chromatography with silica gel and methylene chloride/methanol (3%) as eluent gave 2-(2-furyl)-6-(1H-1,2,4-triazol-1-yl)pyrimidin-4-amine (1.64 g, 74%) as an off-white solid.

δ (250 MHz, CDCl₃): 6.51-6.49 (m, 1H); 6.70 (s, 1H); 7.22 (d, J=3.0 Hz, 1H); 8.01 (s, 1H); 8.54 (s, 1H); 9.19 (s, 1H).

### EXAMPLE 45

### N-[2-(2-furyl)-6-(1H-1,2,4-triazol-1-yl)pyrimidin-4-yl]acetamide

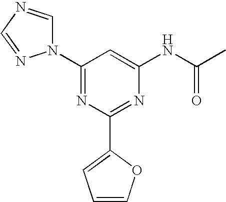

Obtained from the title compound of Example 44 (0.30 g) by the procedure described in Example 2. Purification by column chromatography with silica gel and ethyl acetate/n-hexane (1:1) as eluent gave N-[2-(2-furyl)-6-(1H-1,2,4-triazol-1-yl)pyrimidin-4-yl]acetamide (79 mg, 22%) as an off-white solid.

δ (250 MHz, DMSO-d₆): 2.20 (s, 3H); 6.78-6.76 (m, 1H); 7.54 (d, J=3.8 Hz, 1H); 7.98 (bs, 1H); 8.36 (s, 1H); 8.40 (s, 1H); 9.60 (s, 1H); 11.35 (s, 1H).

### EXAMPLE 46

### N-[2-(2-Furyl)-6-(1H-1,2,4-triazol-1-yl)pyrimidin-4-yl]propanamide

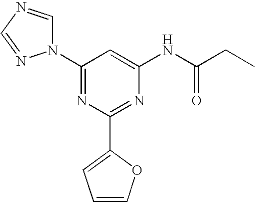

Obtained from the title compound of Example 44 (0.30 g) by the procedure described in Example 2. Purification by column chromatography with silica gel and ethyl acetate/n-hexane (1:1) as eluent gave N-[2-(2-furyl)-6-(1H-1,2,4-triazol-1-yl)pyrimidin-4-yl]propanamide (90 mg, 24%) as an off-white solid.

δ (250 MHz, DMSO-d₆): 1.09 (t, J=7.5 Hz, 3H); 2.51 (q, J=7.5 Hz, 3H); 6.79-6.77 (m, 1H); 7.57-7.54 (m, 1H); 7.99-7.98 (m, 1H); 8.41-8.39 (m, 2H); 9.61 (s, 1H); 11.30 (s, 1H).

### EXAMPLE 47

### 3,3,3-Trifluoro-N-[2-(2-furyl)-6-(1H-1,2,4-triazol-1-yl)pyrimidin-4-yl]-propanamide

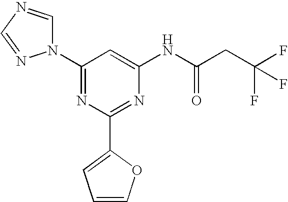

Obtained from the title compound of Example 44 (0.30 g) by the procedure described in Example 14. Purification by column chromatography with silica gel and ethyl acetate/n-hexane (2:3) as eluent gave 3,3,3-trifluoro-N-[2-(2-furyl)-6-(1H-1,2,4-triazol-1-yl)pyrimidin-4-yl]propanamide (0.18 g, 40%) as an off-white solid.

δ (250 MHz, DMSO-d₆): 3.76 (q, J=10.9 Hz, 2H); 6.78-6.76 (m, 1H); 7.57-7.55 (m, 1H); 7.99-7.98 (m, 1H); 8.31 (s, 1H); 8.41 (s, 1H); 9.61 (s, 1H); 11.71 (s, 1H).

### Intermediate 5. 4-Chloro-2-(2-furyl)-6-(1H-pyrazol-1-yl)pyrimidine

To a solution of Intermediate 3 (0.34 g, 1.57 mmol) in anhydrous DMF (8 mL) was added pyrazol (97 mg, 1.43 mmol) and cesium carbonate (0.51 g, 1.57 mmol). The mixture was heated at 65° C. for 7 hours. The solvent was removed under reduced pressure. The resulting solid was washed with water (2×25 mL) and ethyl ether to give 4-chloro-2-(2-furyl)-6-(1H-pyrazol-1-yl)pyrimidine (0.21 g, 54%) as an off-white solid.

δ (300 MHz, CDCl₃): 6.58 (dd, J1=2.7 Hz, J2=1.6 Hz, 1H); 6.65 (dd, J1=3.4 Hz, J2=1.8 Hz, 1H); 7.45 (d, J=3.4 Hz, 1H); 7.60 (s, 1H); 7.86 (d, J=1.6 Hz, 1H); 7.90 (s, 1H); 8.67 (d, J=2.7 Hz, 1H).

### Intermediate 6. 2-(5-Bromo-2-furyl)-4-chloro-6-(1H-pyrazol-1-yl)pyrimidine

To a solution of Intermediate 5 (1.0 g, 4.0 mmol) in anhydrous DMF (20 mL) was added N-bromosuccinimide (0.78 g, 4.4 mmol). The mixture was heated at 50° C. for 2 hours. The mixture was poured into water (75 mL) and extracted with ethyl acetate (2×25 mL). The organic layer was washed with water (2×25 mL), brine (25 mL), dried (Na₂SO₄), and the solvent removed under reduced pressure. Purification by column chromatography with silica gel and methylene chloride as eluent gave the title compound (0.67 g, 51%) as an off-white solid.

δ (300 MHz, CDCl₃): 6.54-6.55 (m, 2H); 7.37-7.38 (m, 1H); 7.78 (s, 1H); 7.81-7.82 (m, 1H); 8.66-8.67 (m, 1H).

### EXAMPLE 48

### 2-(5-Bromo-2-furyl)-6-(1H-pyrazol-1-yl)pyrimidin-4-amine

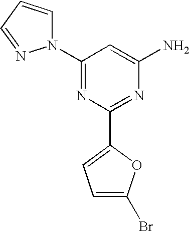

A suspension of Intermediate 6 (0.70 g, 2.13 mmol) in ethanol (22 mL) and 30% ammonium hydroxide (22 mL) was heated at 120° C. in a pressure reactor for 2.30 hours. The solvent was removed under reduced pressure and the residue was dissolved in ethyl acetate (50 mL). The resulting solution was washed with water (2×25 mL), brine (25 mL), dried (Na₂SO₄), and the solvent removed under reduced pressure. Purification by column chromatography with silica gel and methylene chloride/ethyl acetate (1:1) as eluent gave the title compound (0.23 g, 36%) as an off-white solid.

m.p.: 221.0-221.7° C.

δ (300 MHz, DMSO-d₆): 6.58 (dd, J1=2.6 Hz, J2=1.8 Hz, 1H); 6.78 (s, 1H); 6.81 (d, J=3.3 Hz, 1H); 7.34 (d, J=3.3 Hz, 1H); 7.37 (bs, 2H); 7.85 (d, J=1.8 Hz, 1H); 8.66 (d, J=2.6 Hz, 1H).

### EXAMPLE 49

### N-[2-(5-bromo-2-furyl)-6-(1H-pyrazol-1-yl)pyrimidin-4-yl]propanamide

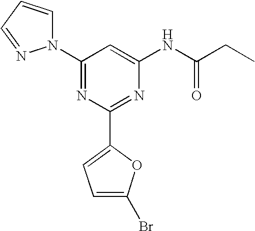

A solution of the title compound of Example 48 (0.10 g, 0.33 mmol) in propanoic anhydride (1.5 mL) was heated at 140° C. for 2 hours. The mixture was poured into ice and extracted with methylene chloride (30 mL). The organic layer was washed with saturated solution of sodium bicarbonate (2×15 mL), water (15 mL), brine (15 mL), and dried (Na₂SO₄). The solvent was removed under reduced pressure. Purification by column chromatography with silica gel and methylene chloride/ethyl acetate (1:1) as eluent gave the title compound (0.10 g, 84%) as an off-white solid.

m.p.: 199.5-200.3° C.

δ (300 MHz, DMSO-d₆): 1.08 (t, J=7.6 Hz, 3H); 2.50 (q, J=7.6 Hz, 2H); 6.67 (dd, J1=2.6 Hz, J2=1.7 Hz, 1H); 6.90 (d, J=3.3 Hz, 1H); 7.53 (d, J=3.3 Hz, 1H); 7.94 (d, J=1.7 Hz, 1H); 8.48 (s, 1H) 8.81 (d, J=2.6 Hz, 1H); 11.19 (bs, 1H).

### Intermediate 7. 4-Chloro-2-(5-chloro-2-furyl)-6-(1H-pyrazol-1-yl)pyrimidine

To a solution of Intermediate 5 (1.0 g, 4.0 mmol) in anhydrous DMF (20 mL) was added N-chlorosuccinimide (0.59 g, 4.4 mmol). The mixture was heated at 50° C. for 2 hours. The mixture was poured into water (75 mL) and extracted with ethyl acetate (2×25 mL). The organic layer was washed with water (2×25 mL), brine (25 mL), dried (Na₂SO₄), and the solvent removed under reduced pressure. 4-Chloro-2-(5-chloro-2-furyl)-6-(1H-pyrazol-1-yl)pyrimidine (1.12 g, 99%) was obtained as an off-white solid.

δ (300 MHz, CDCl₃): 6.41 (d, J=3.6 Hz, 1H), 6.55 (dd, J1=2.7 Hz, J2=1.6 Hz, 1H); 7.41 (d, J=3.6 Hz, 1H); 7.78 (s, 1H); 7.82 (d, J=1.6 Hz, 1H); 8.66 (d, J=2.7 Hz, 1H).

### EXAMPLE 50

### 2-(5-Chloro-2-furyl)-6-(1H-pyrazol-1-yl)pyrimidin-4-amine

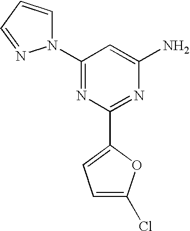

Obtained from Intermediate 7 (1.17 g) by the procedure described in Example 48. Purification by column chromatography with silica gel and methylene chloride/ethyl acetate (1:1) as eluent gave 2-(5-chloro-2-furyl)-6-(1H-pyrazol-1-yl)pyrimidin-4-amine (0.48 g, 44%) as an off-white solid.

m.p.: 209.9-211.0° C.

δ (300 MHz, DMSO-d₆): 6.58 (dd, J1=2.6 Hz, J2=1.7 Hz, 1H); 6.72 (d, J=3.6 Hz, 1H); 6.78 (s, 1H); 7.37-7.36 (m, 3H); 7.85 (s, 1H); 8.66 (d, J=2.6 Hz, 1H).

### EXAMPLE 51

### N-[2-(5-Chloro-2-furyl)-6-(1H-pyrazol-1-yl)pyrimidin-4-yl]propanamide

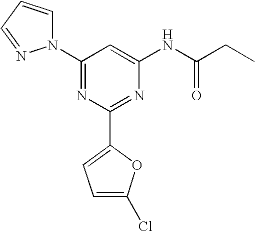

Obtained from the title compound of Example 50 (0.28 g) by the procedure described in Example 49. Purification by column chromatography with silica gel and methylene chloride/ethyl acetate (3:1) as eluent gave N-[2-(5-chloro-2-furyl)-6-(1H-pyrazol-1-yl)pyrimidin-4-yl]propanamide (0.23 g, 72%) as an off-white solid.

m.p.: 189.3-190.1° C.

δ (300 MHz, DMSO-d₆): 1.05 (t, J=7.6 Hz, 3H); 2.46 (q, J=7.6 Hz; 2H); 6.64 (dd, J1=2.8 Hz, J2=1.7 Hz, 1H); 6.78 (d, J=3.6 Hz, 1H); 7.54 (d, J=3.6 Hz, 1H); 7.92 (d, J1=1.7 Hz, J2=0.6 Hz, 1H); 8.45 (s, 1H); 8.78 (d, J1=2.8 Hz, J2=0.6 Hz, 1H); 11.16 (bs, 1H).

### Intermediate 8. 5-Methyl-2-furancarboxamidine (HCl)

The title compound (3.71 g, 87%) was obtained as a pale yellow solid starting from 5-methyl-2-furonitrile (2.85 g) by the procedure described in Intermediate 1.

δ (300 MHz, DMSO-d₆): 2.27 (s, 3H); 6.36 (d, J=3.6 Hz, 1H); 7.64 (d, J=3.6 Hz, 1H); 8.49 (bs, 4H).

### Intermediate 9. 6-Amino-2-(5-methyl-2-furyl)pyrimidin-4 of

To a solution of Intermediate 8 (3.71 g, 23 mmol) and cyanoacetic acid ethyl ester (2.60 g, 23 mmol) in butanol (25 mL) was added potassium tertbutoxide (5.45 g, 46 mmol). The mixture was stirred at 135° C. for 18 hours. The solvent was removed under reduced pressure. Purification by column chromatography with silica gel and methylene chloride/methanol (from 2% to 10%) as eluent gave 6-amino-2-(5-methyl-2-furyl)pyrimidin-4-ol (1.96 g, 44%) as an off-white solid.

δ (300 MHz, DMSO-d₆): 2.19 (s, 3H) 4.82 (s, 1H) 6.16 (d, J=3.3 Hz, 1H) 6.41 (s, 2H) 7.23 (d, J=3.3 Hz, 1H).

### Intermediate 10. 6-Chloro-2-(5-methyl-2-furyl)pyrimidin-4-amine

A suspension of Intermediate 9 (2.45 g, 10.2 mmol) and phosphorous pentachloride (2.12 g, 10.2 mmol) in phosphorous oxychloride (7 mL) was stirred at 90° C. for 2 hours. The reaction mixture was diluted with methylene chloride (50 mL) and ice was added slowly. The organic layer was decanted and washed with saturated solution of sodium bicarbonate (2×25 mL), water (2×25 mL), brine (25 mL), and dried (Na₂SO₄). The solvent was removed under reduced pressure. Purification by column chromatography with silica gel and methylene chloride/ethanol (8%) as eluent gave 6-chloro-2-(5-methyl-2-furyl)pyrimidin-4-amine (0.28 g, 13%) as an off-white solid.

δ (300 MHz, DMSO-d₆): 2.35 (s, 3H); 6.27 (s, 1H); 6.28 (d, J=3.3 Hz, 1H); 7.05 (d, J=3.3 Hz, 1H); 7.32 (bs, 2H).

### EXAMPLE 52

### 2-(5-Methyl-2-furyl)-6-(1H-pyrazol-1-yl)pyrimidin-4-amine

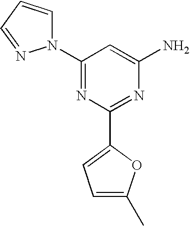

Obtained from Intermediate 10 (0.10 g) by the procedure described in Example 21. Purification by column chromatography with silica gel and methylene chloride/ethyl acetate (9:1) as eluent gave 2-(5-methyl-2-furyl)-6-(1H-pyrazol-1-yl)pyrimidin-4-amine (44 mg, 36%) as an off-white solid.

δ (300 MHz, DMSO-d): 2.23 (s, 3H); 6.15-6.16 (m, 1H); 6.42-6.43 (m, 1H); 6.58 (s, 1H); 7.05 (d, J=3.0 Hz, 1H); 7.11 (s, 2H); 7.69 (s, 1H); 8.50 (d, J=2.5 Hz, 1H).

### EXAMPLE 53

### N-[2-(5-methyl-2-furyl)-6-(1H-pyrazol-1-yl)pyrimidin-4-yl]propanamide

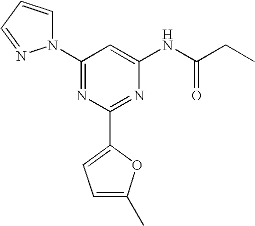

Obtained from the title compound of Example 52 (40 mg) by the procedure described in Example 49. Purification by column chromatography with silica gel and methylene chloride/ethyl acetate (4:1) as eluent gave N-[2-(5-methyl-2-furyl)-6-(1H-pyrazol-1-yl)pyrimidin-4-yl]propanamide (11 mg, 19%) as an off-white solid.

δ (300 MHz, DMSO-d₆): 0.94 (t, J=7.6 Hz, 3H); 2.26 (s, 3H); 2.36 (q, J=7.6 Hz, 2H); 6.24 (dd, J1=3.3 Hz, J2=0.6 Hz, 1H); 6.51 (dd, J1=2.8 Hz, J2=1.7 Hz, 1H); 7.25 (d, J=3.3 Hz, 1H); 7.80-7.78 (m, 1H); 8.28 (s, 1H); 8.63 (dd, J=2.8 Hz, J2=0.6 Hz, 1H); 11.00 (bs, 1H).

### Intermediate 11. 6-Amino-2-(2-furyl)pyrimidin-4-ol

To a solution of sodium methoxide (44 mmol) in methanol (10 mL) was slowly added Intermediate 1 (1.60 g, 11 mmol). The mixture was stirred at room temperature for 30 minutes and then, cyanoacetic acid ethyl ester (1.00 g, 8.8 mmol) was added. The suspension was refluxed for 18 hours. The solvent was removed under reduced pressure. The residue was suspended in water (20 mL) and acidified to pH=6 with 5N hydrochloric acid. The resulting solid was filtered and washed with water (20 mL). 6-Amino-2-(2-furyl)pyrimidin-4-ol was obtained (0.79 g, 50%) as a pale yellow solid.

δ (200 MHz, DMSO-d₆): 5.01 (s, 1H); 6.57 (s, 2H); 6.69 (dd, J1=3.4 Hz, J2=1.7 Hz, 1H); 7.43 (d, J=3.4 Hz, 1H); 7.91 (d, J=1.7 Hz, 1H).

### Intermediate 12. N-[6-Chloro-2-(2-furyl)pyrimidin-4-yl]propanamide

A solution of Intermediate 11 (1.20 g, 6.78 mmol) and propionic anhydride (1.5 mL) in phosphorous oxychloride (12 mL) was stirred at 90° C. for 18 hours. The solvent was removed under reduced pressure. The resulting oil was solved in methylene chloride (50 mL), washed with water (2×25 mL), and brine (25 mL). The organic layer was dried (Na₂SO₄) and the solvent removed under reduced pressure. The resulting solid was filtered and washed with n-pentane (20 mL) to give the title compound (1.40 g, 80%) as a brown solid.

δ (200 MHz, CDCl₃): 1.26 (t, J=7.4 Hz, 3H); 2.49 (q, J=7.2 Hz, 2H); 6.59 (dd, J1=3.4 Hz, J2=1.7 Hz, 1H); 7.39 (d, J=3.4 Hz, 1H); 7.64 (s, 1H); 8.10 (d, J=1.71 Hz, 1H); 8.38 (bs, 1H).

### EXAMPLE 54

### N-[2-(2-Furyl)-6-pyridin-3-ylpyrimidin-4-yl]propanamide

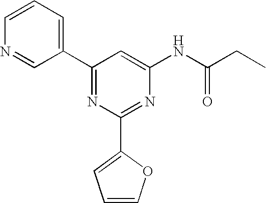

To a solution of Intermediate 12 (1.20 g, 4.77 mmol) in 1,2-dimethoxyethane (120 mL) were added pyridin-3-ylboronic acid (0.88 g, 7.15 mmol), potassium carbonate (1.31 g, 9.54 mmol), water (8 mL) and tetrakis(triphenylphosphine)palladium (0) (2.65 g, 2.38 mmol). The mixture was stirred at 80° C. overnight. The crude reaction was filtered through Celite® and the organic layer was washed with saturated solution of sodium bicarbonate (2×50 mL), water (2×50 mL), brine (50 mL), dried (Na₂SO₄), and the solvent removed under reduced pressure. Purification by column chromatography, eluting with ethyl acetate/n-hexane (from 1:6 to pure ethyl acetate), followed by digestion in hot acetonitrile gave N-[2-(2-furyl)-6-pyridin-3-ylpyrimidin-4-yl]propanamide (0.30 g, 21%) as an off-white solid.

m.p.: 251.8-253.2° C.

δ (200 MHz, DMSO-d₆): 1.10 (t, J=7.5 Hz, 3H); 2.50 (q, J=7.5 Hz, 2H) 6.75 (dd, J1=3.0 Hz, J2=1.7 Hz, 1H); 7.43 (d, J=3.5 Hz, 1H); 7.97 (s, 1H); 8.06 (d, J=5.6 Hz, 2H); 8.54 (s, 1H); 8.81 (d, J=5.6 Hz, 2H); 11.21 (bs, 1H).

### EXAMPLE 55

### 2-(2-Furyl)-6-pyridin-3-ylpyrimidin-4-amine

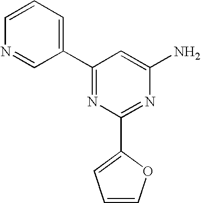

To a solution of the title compound of Example 54 (0.20 g, 0.687 mmol) in ethanol (2 mL) was added 2N hydrochloric acid (2 mL). The mixture was stirred at 80° C. for 1 hour. The solution was diluted with water (10 mL) and 2N sodium hydroxide was added until pH=10. The mixture was extracted with methylene chloride (2×10 mL). The combined organic extracts were washed with water (2×10 mL), dried (Na₂SO₄), and the solvent removed under reduced pressure. The resulting solid was washed with ethyl ether, to give 2-(2-furyl)-6-pyridin-3-ylpyrimidin-4-amine (80 mg, 49%) as an off-white solid.

m.p.: 232.7-233.1° C.

δ (200 MHz, CDCl₃) 5.13 (bs, 2H); 6.58 (dd, J1=3.5 Hz, J2=1.7 Hz, 1H); 6.75 (s, 1H); 7.37 (d, J=3.5 Hz, 1H); 7.64-7.63 (m, 1H); 7.93 (d, J=6.0 Hz, 2H); 8.77 (d, J=6.0 Hz, 2H).

### Intermediate 13. Thiophene-2-carboxamidine (HCl)

The title compound (12.7 g, 85%) was obtained as a solid starting from thiophene-2-carbonitrile (10.0 g) by the procedure described in Intermediate 1.

δ (250 MHz, DMSO-d₆): 7.32 (m, 1H); 8.13 (m, 1H); 8.17 (m, 1H); 8.94-8.33 (bs, 3H).

### Intermediate 14. 6-Amino-2-(2-thienyl)pyrimidin-4-ol

The title compound (6.13 g, 76%) was obtained as a brown solid starting from Intermediate 13 (7.00 g) by the procedure described in Intermediate 11 (reaction time: 4 days).

δ (250 MHz, DMSO-d₆): 5.04 (bs, 1H); 6.52 (bs, 2H); 7.18 (bs, 1H); 7.78 (bs, 1H); 8.09 (bs, 1H).

### Intermediate 15. 6-Chloro-2-(2-thienyl)pyrimidin-4-amine

A suspension of Intermediate 14 (6.30 g, 32.6 mmol) in phosphorous oxychloride (20 mL) was refluxed for 24 hours. The solvent was removed under pressure and ice and water were slowly added. The resulting solid was filtered, washed with 2N sodium hydroxide, and dried. 6-Chloro-2-(2-thienyl)pyrimidin-4-amine was obtained (4.40 g, 64%) as a brown solid.

MS (M⁺): 211

### EXAMPLE 56

### 6-(1H-Pyrazol-1-yl)-2-(2-thienyl)pyrimidin-4-amine

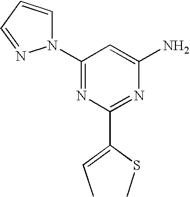

Obtained from Intermediate 15 (3.00 g) by the procedure described in Example 21. Purification by trituration with ethyl ether gave 6-(1H-pyrazol-1-yl)-2-(2-thienyl)pyrimidin-4-amine (1.00 g, 27%) as an off-white solid.

δ (250 MHz, DMSO-d₆): 6.59-6.57 (m, 1H); 6.77 (s, 1H); 7.20-7.17 (m, 1H); 7.24 (bs, 2H); 7.72-7.70 (m, 1H); 7.85-7.84 (m, 1H); 7.97-7.95 (m, 1H); 8.67 (d, J=2.5 Hz, 1H).

### EXAMPLE 57

### N-[6-(1H-Pyrazol-1-yl)-2-(2-thienyl)pyrimidin-4-yl]acetamide

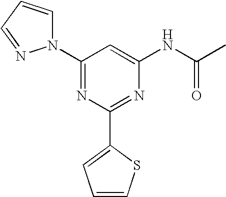

Obtained from the title compound of Example 56 (0.30 g) by the procedure described in Example 2. Purification by column chromatography with silica gel and ethyl acetate/n-hexane (1:4) as eluent gave N-[6-(1H-pyrazol-1-yl)-2-(2-thienyl)pyrimidin-4-yl]acetamide (0.19 g, 55%) as an off-white solid.

δ (250 MHz, CDCl₃): 2.20 (s, 3H); 6.44-6.43 (m, 1H); 7.08 (dd, J1=4.8 Hz, J2=3.6 Hz, 1H); 7.43 (dd, J1=4.8 Hz, J2=1.2 Hz, 1H); 7.74-7.73 (m, 1H); 7.91 (dd, J1=3.6 Hz, J2=1.2 Hz, 1H); 8.39 (m, 1H); 8.59-8.57 (m, 1H).

### EXAMPLE 58

### N-[6-(1H-Pyrazol-1-yl)-2-(2-thienyl)pyrimidin-4-yl]propanamide

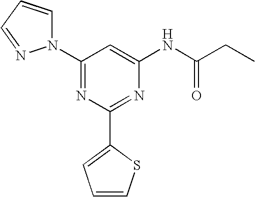

Obtained from the title compound of Example 56 (0.30 g) by the procedure described in Example 2. Purification by column chromatography with silica gel and ethyl acetate/n-hexane (1:4) as eluent gave N-[6-(1H-pyrazol-1-yl)-2-(2-thienyl)pyrimidin-4-yl]propanamide (0.2 g, 54%) as an off-white solid.

δ (250 MHz, CDCl₃): 1.28 (t, J=7.6 Hz, 3H); 2.51 (q, J=7.6 Hz, 2H); 6.50-6.49 (m, 1H); 7.15 (dd, J1=5.2 Hz, J2=3.9 Hz, 1H); 7.49 (dd, J1=5.2 Hz, J2=1.2 Hz, 1H); 7.80-7.79 (m, 1H); 8.00-7.98 (m, 2H); 8.54 (s, 1H); 8.66-8.65 (m, 1H).

### EXAMPLE 59

### 3-Cyclopentyl-N-[6-(1H-pyrazol-1-yl)-2-(2-thienyl)pyrimidin-4-yl]propanamide

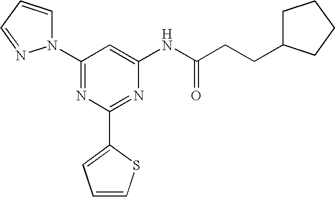

Obtained from the title compound of Example 56 (0.20 g) by the procedure described in Example 2. Purification by column chromatography with silica gel and ethyl acetate/n-hexane (1:9) as eluent gave 3-cyclopentyl-N-[6-(1H-pyrazol-1-yl)-2-(2-thienyl)pyrimidin-4-yl]propanamide (0.17 g, 57%) as an off-white solid.

δ (250 MHz, CDCl₃): 1.18-1.10 (m, 2H); 1.63-1.51 (m, H); 1.85-1.75 (m, 5H); 2.48 (t, J=7.3 Hz, 2H); 6.50-6.49 (m, 1H); 7.15 (dd, J1=4.9 Hz, J2=3.6 Hz, 1H); 7.50 (dd, J1=4.9 Hz, J2=1.2 Hz, 1H); 7.99 (dd, J1=3.9 Hz, J2=1.2 Hz, 2H); 8.00 (d, J=1.2 Hz, 1H); 8.54 (s, 1H); 8.65 (d, J=2.7 Hz, 1H).

### EXAMPLE 60

### 3-Phenyl-N-[6-(1H-pyrazol-1-yl)-2-(2-thienyl)pyrimidin-4-yl]-propanamide

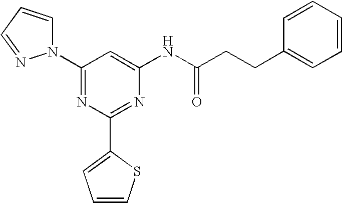

Obtained from the title compound of Example 56 (0.20 g) by the procedure described in Example 2. Purification by column chromatography with silica gel and ethyl acetate/n-hexane (1:4) as eluent gave 3-phenyl-N-[6-(H-pyrazol-1-yl)-2-(2-thienyl)pyrimidin-4-yl]propanamide (0.29 g, 94%) as an off-white solid.

δ (250 MHz, CDCl₃): 2.78 (t, J=7.6 Hz, 2H); 3.09 (t, J=7.6 Hz, 2H); 6.50 (dd, J1=2.7 Hz, J2=1.8 Hz, 1H); 7.15 (dd, J1=5.2 Hz, J2=3.6 Hz, 1H); 7.35-7.22 (m, 5H); 7.49 (dd, J1=5.2 Hz, J2=1.2 Hz, 1H); 7.81 (m, 1H); 7.94 (bs, 1H); 7.97 (dd, J1=3.6 Hz, J2=1.2 Hz, 1H); 8.54 (s, 1H); 8.65 (d, J=2.6 Hz, 1H).

### EXAMPLE 61

### 3,3,3-Trifluoro-N-[6-(1H-pyrazol-1-yl)-2-(2-thienyl)pyrimidin-4-yl]-propanamide

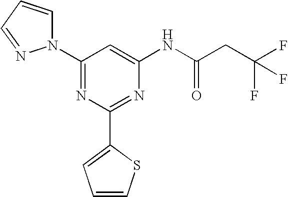

Obtained from the title compound of Example 56 (0.30 g) by the procedure described in Example 14. Purification by column chromatography with silica gel and ethyl acetate/n-hexane (1:4) as eluent gave 3,3,3-trifluoro-N-[6-(1H-pyrazol-1-yl)-2-(2-thienyl)pyrimidin-4-yl]propanamide (0.33 g, 76%) as an off-white solid.

δ (250 MHz, CDCl₃): 3.37 (q, J=10.3 Hz, 2H); 6.52-6.50 (m, 1H); 7.15 (dd, J1=4.8 Hz, J2=3.6 Hz, 1H); 7.51 (dd, J1=4.8 Hz, J2=1.2 Hz, 1H); 7.81-7.80 (m, 1H); 7.99 (dd, J1=3.6 Hz, J2=1.2 Hz, 1H); 8.23 (bs, 1H); 8.48 (bs, 1H); 8.65-8.64 (m, 1H).

### EXAMPLE 62

### 3-(3,4-Dimethoxyphenyl)-N-[6-(1H-pyrazol-1-yl)-2-(2-thienyl)pyrimidin-4-yl]propanamide

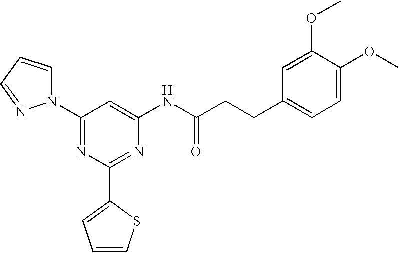

Obtained from the title compound of Example 56 (0.20 g) by the procedure described in Example 14. Purification by column chromatography with silica gel eluting with methylene chloride/methanol (0.2%), followed by a second purification by column chromatography using ethyl acetate/n-hexane (1:1) as eluent gave 3-(3,4-dimethoxyphenyl)-N-[6-(1H-pyrazol-1-yl)-2-(2-thienyl)pyrimidin-4-yl]propanamide (0.18 g, 51%) as an off-white solid.

δ (250 MHz, CDCl₃): 2.76 (t, J=7.6 Hz, 2H); 3.04 (t, J=7.6 Hz, 2H); 3.85 (s, 3H); 3.87 (s, 3H); 6.50 (dd, J1=2.7 Hz, J2=1.8 Hz, 1H); 6.80-6.77 (m, 3H); 7.15 (dd, J1=4.8 Hz, J2=3.6 Hz, 1H); 7.49 (dd, J1=4.8 Hz, J2=1.2 Hz, 1H); 7.81-7.80 (m, 1H); 7.92 (bs, 1H); 7.97 (dd, J1=3.6 Hz, J2=1.2 Hz, 1H); 8.53 (s, 1H); 8.65 (dd, J1=2.7 Hz, J2=0.6 Hz, 1H).

### EXAMPLE 63

### 3-Phenoxy-N-[6-(1H-pyrazol-1-yl)-2-(2-thienyl)pyrimidin-4-yl]-propanamide

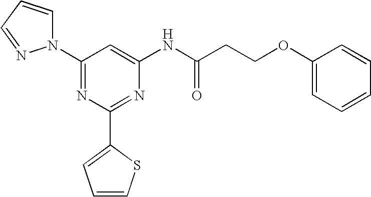

Obtained from the title compound of Example 56 (0.20 g) by the procedure described in Example 14. Purification by column chromatography with silica gel and methylene chloride/methanol (2%) as eluent gave 3-phenoxy-N-[6-(1H-pyrazol-1-yl)-2-(2-thienyl)pyrimidin-4-yl]propanamide (0.28 g, 85%) as an off-white solid.

δ (250 MHz, CDCl₃): 2.95 (t, J=6.0 Hz, 2H); 4.38 (t, J=6.0 Hz, 2H); 6.50-6.49 (m, 1H); 7.03-6.98 (m, 3H); 7.16 (dd, J1=4.8 Hz, J2=3.6 Hz, 1H); 7.37-7.29 (m, 2H); 7.50 (dd, J1=5.2 Hz, J2=1.2 Hz, 1H); 7.80-7.79 (m, 1H); 8.00 (dd, J1=3.9 Hz, J2=1.2 Hz, 1H); 8.53 (s, 1H); 8.63 (bs, 1H); 8.66 (dd, J1=2.7 Hz, J2=0.6 Hz, 1H).

### EXAMPLE 64

### N-[6-(1H-Pyrazol-1-yl)-2-(2-thienyl)pyrimidin-4-yl]-2-pyridin-3-ylacetamide

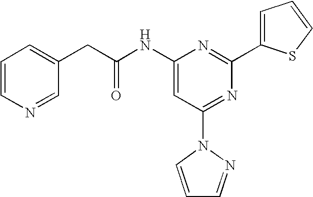

Obtained from the title compound of Example 56 (0.20 g) by the procedure described in Example 14. Purification by column chromatography with silica gel and methylene chloride/methanol (2.5%) as eluent gave N-[6-(1H-pyrazol-1-yl)-2-(2-thienyl)pyrimidin-4-yl]-2-pyridin-3-ylacetamide (0.17 g, 56%) as an off-white solid.

δ (250 MHz, CDCl₃): 3.82 (s, 2H); 6.49 (dd, J1=2.7 Hz, J2=1.5 Hz, 1H); 7.16-7.13 (m, 1H); 7.37-7.32 (m, 1H); 7.50 (dd, J1=4.9 Hz, J2=1.2 Hz, 1H); 7.77-7.73 (m, 1H); 7.78 (dd, J1=1.5 Hz, J2=0.6 Hz, 1H); 7.98 (dd, J1=3.6 Hz, J2=1.2 Hz, 1H); 8.04 (bs, 1H); 8.51 (s, 1H); 8.62-8.59 (m, 2H); 8.64 (dd, J1=2.7 Hz, J2=0.6 Hz, 1H).

### EXAMPLE 65

### N-[6-(1H-Pyrazol-1-yl)-2-(2-thienyl)pyrimidin-4-yl]-3-pyridin-3-ylpropanamide

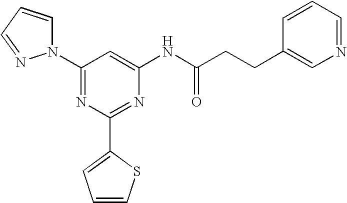

Obtained from the title compound of Example 56 (0.20 g) by the procedure described in Example 14. Purification by column chromatography with silica gel and methylene chloride/methanol (2%) as eluent gave N-[6-(1H-pyrazol-1-yl)₂₋₂-thienyl)pyrimidin-4-yl]-3-pyridin-3-ylpropanamide (0.13 g, 43%) as an off-white solid.

δ (250 MHz, CDCl₃): 2.81 (t, J=7.3 Hz, 2H); 3.10 (t, J=7.3 Hz, 2H); 6.51 (dd, J1=2.7 Hz, J2=1.5 Hz, 1H); 7.15 (dd, J1=5.2 Hz, J2=3.6 Hz, 1H); 7.25-7.21 (m, 1H); 7.50-7.48 (m, 1H); 7.61 (dt, J1=7.9 Hz, J2=2.1 Hz, 1H); 7.82-7.81 (m, 1H); 7.97 (dd, J1=3.6 Hz, J2=1.2 Hz, 1H); 8.15 (bs, 1H); 8.5 (dd, J1=4.8 Hz, J2=1.5 Hz, 1H); 8.54-8.52 (m, 2H); 8.65 (d, J=2.7 Hz, 1H).

### EXAMPLE 66

### (2E)-3-(3,4-Dimethoxyphenyl)-N-[6-(1H-pyrazol-1-yl)-2-(2-thienyl)pyrimidin-4-yl]acrylamide

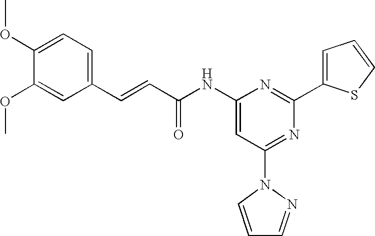

Obtained from the title compound of Example 56 (0.30 g) by the procedure described in Example 20. Purification by column chromatography with silica gel and methylene chloride/methanol (0.5%) as eluent gave (2E)-3-(3,4-dimethoxyphenyl)-N-[6-(1H-pyrazol-1-yl)-2-(2-thienyl)pyrimidinyl]acrylamide (0.20 g, 36%) as an off-white solid.

δ (250 MHz, CDCl₃): 3.82 (s, 3H); 3.84 (s, 3H); 6.68 (dd, J1=2.7 Hz, J2=1.7 Hz, 1H); 7.09-7.02 (m, 2H); 7.28-7.23 (m, 3H); 7.66 (d, J1=15.5 Hz, 1H); 7.84 (dd, J1=5.0 Hz, J2=1.3 Hz, 1H); 7.97 (d, J1=1.3 Hz, 1H); 8.12 (dd, J1=3.7 Hz, J2=1.0 Hz, 1H); 8.57 (s, 1H); 8.82 (d, J1=2.7 Hz, 1H); 11.08 (s, 1H).

### EXAMPLE 67

### 6-(3,5-Dimethyl-1H-pyrazol-1-yl)-2-(2-thienyl)pyrimidin-4-amine

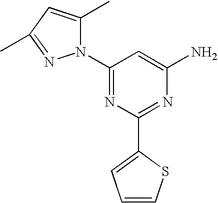

Obtained from Intermediate 15 (3.0 g) by the procedure described in Example 21. Purification by column chromatography with silica gel and ethyl acetate/n-hexane (3:10) as eluent gave 6-(3,5-dimethyl-1H-pyrazol-1-yl)₂₋₂-thienyl)pyrimidin-4-amine (1.75 g, 45%) as an off-white solid.

δ (250 MHz, DMSO-d₆): 2.19 (s, 3H); 2.72 (s, 3H); 6.13 (s, 1H); 6.73 (s, 1H); 7.08 (bs, 2H); 7.19-7.15 (m, 1H); 7.70-7.67 (m, 1H); 7.82-7.79 (m, 1H).

### EXAMPLE 68

### N-[6-(3,5-Dimethyl-1H-pyrazol-1-yl)-2-(2-thienyl)pyrimidin-4-yl]-acetamide

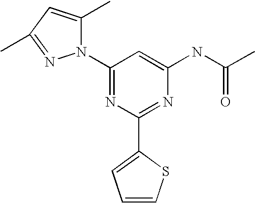

Obtained from the title compound of Example 67 (0.30 g) by the procedure described in Example 2. Purification by column chromatography with silica gel and ethyl acetate/n-hexane (3:10) as eluent gave N-[6-(3,5-dimethyl-1H-pyrazol-1-yl)-2-(2-thienyl)pyrimidin-4-yl]acetamide (80 mg, 23%) as an off-white solid.

δ (250 MHz, CDCl₃): 2.20 (s, 3H); 2.23 (s, 3H); 2.74 (s, 3H); 5.96 (s, 1H); 7.07 (dd, J1=4.8 Hz, J2=3.6 Hz, 1H); 7.40 (dd, J1=5.2 Hz, J2=1.2 Hz, 1H); 7.84 (dd, J1=3.6 Hz, J2=1.2 Hz, 1H); 7.94 (bs, 1H); 8.39 (s, 1H).

### EXAMPLE 69

### N-[6-(3,5-Dimethyl-1H-pyrazol-1-yl)-2-(2-thienyl)pyrimidin-4-yl]-propanamide

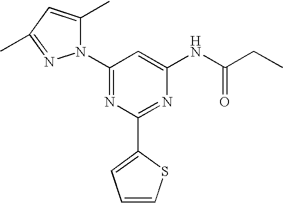

Obtained from the title compound of Example 67 (0.30 g) by the procedure described in Example 2. Purification by column chromatography with silica gel and ethyl acetate/n-hexane (1:4) as eluent gave N-[6-(3,5-dimethyl-1H-pyrazol-1-yl)-2-(2-thienyl)pyrimidin-4-yl]propanamide (0.17 g, 47%) as an off-white solid.

δ (250 MHz, DMSO-d₆): 1.27 (t, J=7.3 Hz, 3H); 2.28 (s, 3H); 2.49 (q, J=7.3 Hz, 2H); 2.80 (s, 3H); 6.02 (s, 1H); 7.13 (dd, J1=4.9 Hz, J2=3.6 Hz, 1H); 7.46 (dd, J1=4.9 Hz, J2=1.2 Hz, 1H); 7.92-7.89 (m, 2H); 8.50 (s, 1H).

### EXAMPLE 70

### N-[6-(3,5-Dimethyl-1H-pyrazol-1-yl)-2-(2-thienyl)pyrimidin-4-yl]-3,3,3-trifluoropropanamide

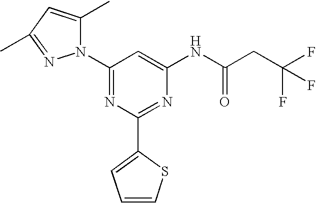

Obtained from the title compound of Example 67 (0.30 g) by the procedure described in Example 14. Purification by column chromatography with silica gel and ethyl acetate/n-hexane (15:85) as eluent gave N-[6-(3,5-dimethyl-1H-pyrazol-1-yl)₂₋₂-thienyl)pyrimidin-4-yl]-3,3,3-trifluoropropanamide (55 mg, 13%) as an off-white solid.

δ (250 MHz, CDCl₃): 2.32 (s, 3H); 2.84 (s, 3H); 3.38 (q, J=10.0 Hz, 2H); 6.06 (s, 1H); 7.18-7.16 (m, 1H); 7.52-7.50 (m, 1H); 7.96-7.94 (m, 1H); 8.12 (s, 1H); 8.48 (bs, 1H).

### EXAMPLE 71

### 2-(2-Thienyl)-6-(1H-1,2,4-triazol-1-yl)pyrimidin-amine

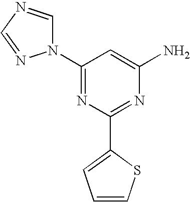

Obtained from Intermediate 15 (1.86 g) by the procedure described in Example 21. Purification by trituration with ethyl ether gave 2-(2-thienyl)-6-(1H-1,2,4-triazol-1-yl)pyrimidin-4-amine (1.81 g, 84%) as an off-white solid.

δ (250 MHz, MeOD): 6.67 (s, 1H); 7.06 (dd, J1=5.0 Hz, J2=3.6 Hz, 1H); 7.40 (dd, J1=5.0 Hz, J2=1.3 Hz, 1H); 7.88-7.86 (m, 1H); 8.01 (s, 1H); 9.19 (s, 1H).

### EXAMPLE 72

### N-[2-(2-Thienyl)-6-(1H-1,2,4-triazol-1-yl)pyrimidin-4-yl]acetamide

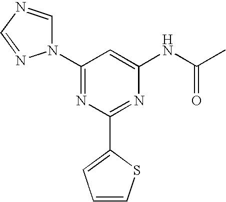

Obtained from the title compound of Example 71 (0.30 g) by the procedure described in Example 2. Purification by column chromatography with silica gel and ethyl acetate/n-hexane (from 1:1 to 4:1) as eluent gave N-[2-(2-thienyl)-6-(1H-1,2,4-triazol-1-yl)pyrimidin-4-yl]acetamide (0.21 g, 63%) as an off-white solid.

δ (250 MHz, DMSO-d₈): 2.15 (s, 3H); 7.20 (dd, J1=4.9 Hz, J2=3.8 Hz, 1H); 7.79 (dd, J1=4.9 Hz, J2=1.4 Hz, 1H); 8.11 (dd, J1=3.8 Hz, J2=1.4 Hz, 1H); 8.29 (s, 1H); 8.34 (s, 1H); 9.57 (s, 1H); 11.00 (s, 1H).

### EXAMPLE 73

### N-[2-(2-Thienyl)-6-(1H-1,2,4-triazol-1-yl)pyrimidin-4-yl]propanamide

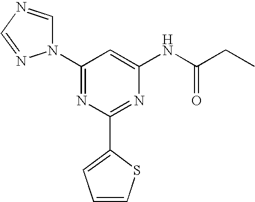

Obtained from the title compound of Example 71 (0.14 g) by the procedure described in Example 2. Purification by trituration with ethyl ether gave N-[2-(2-thienyl-6-(1H-1,2,4-triazol-1-yl)pyrimidinyl]propanamide (0.13 g, 75%) as an off-white solid.

δ (250 MHz, DMSO-d₆): 1.05 (t, J=7.4 Hz, 3H); 2.49 (q, J=7.4 Hz, 2H); 7.25-7.20 (m, 1H); 7.83-7.80 (m, 1H); 8.14-8.12 (m, 1H); 8.34 (s, 1H); 8.36 (s, 1H); 9.58 (s, 1H); 11.09 (s, 1H).

### EXAMPLE 74

### 3,3,3-Trifluoro-N-[2-(2-thienyl)-6-(1H-1,2,4-triazol-1-yl)pyrimidin-4-yl]propanamide

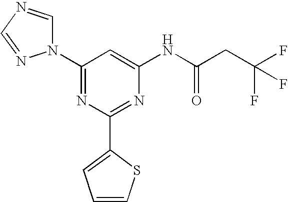

Obtained from the title compound of Example 71 (0.30 g) by the procedure described in Example 14. Purification by column chromatography with silica gel and methylene chloride/methanol (10%) as eluent gave 3,3,3-trifluoro-N-[2-(2-thienyl)-6-(1H-1,2,4-triazol-1-yl)pyrimidin-4-yl]propanamide (0.2 mg, 47%) as an off-white solid.

δ (250 MHz, DMSO-d₆): 3.57 (q, J=11.0 Hz, 2H); 7.09-7.04 (m, 1H); 7.68-7.65 (m, 1H); 8.00-7.97 (m, 1H); 8.10 (s, 1H); 8.21 (s, 1H); 9.45 (s, 1H); 11.31 (s, 1H).

### Intermediate 16. 3-Methylthiophene-2-carboxamidine (HCl)

The title compound (3.25 g, 43%) was obtained as an off-white solid starting from 3-methylthiophene-2-carbonitrile (5.26 g) by the procedure described in Intermediate 1.

δ (300 MHz, DMSO-d₆): 2.36 (s, 3H); 7.42 (bs, 4H); 8.24 (s, 1H).

### Intermediate 17. 6-Amino-2-(3-methyl-2-thienyl)pyrimidin-4-ol

Obtained from Intermediate 16 (3.20 g) by the procedure described in Intermediate 11. Purification by column chromatography with silica gel and methylene chloride/methanol (2%) as eluent, followed by a preparative HPLC-MS purification gave 6-amino-2-(3-methyl-2-thienyl)pyrimidin-4-ol (80 mg, 2%) as an off-white solid.

δ (300 MHz, DMSO-d₆): 2.36 (s, 3H); 5.07 (s, 1H); 6.36 (bs, 2H); 6.82 (d, J=4.9 Hz, 1H); 7.40 (d, J=4.9 Hz, 1H).

### Intermediate 18. N-[6-Chloro-2-(3-methyl-2-thienyl)pyrimidin-4-yl]propanamide

Obtained from Intermediate 17 (80 mg) by the procedure described in Intermediate 12. Purification by column chromatography with silica gel and methylene chloride as eluent gave N-[6-chloro-2-(3-methylthiophen-2-yl)pyrimidinyl]propionamide (40 mg, 37%) as an off-white solid.

δ (300 MHz, DMSO-d₆): 0.93 (t, J=7.4 Hz, 3H); 2.36-2.37 (m, 5H); 6.92 (d, J=5.0 Hz, 1H); 7.54 (d, J=5.0 Hz, 1H); 7.78 (s, 1H).

### EXAMPLE 75

### N-[2-(3-Methyl-2-thienyl)-6-(1H-pyrazol-1-yl)pyrimidin-4-yl]-propanamide

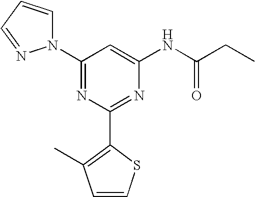

Obtained from Intermediate 18 (40 mg) by the procedure described in Example 21. Purification by column chromatography with silica gel and ethyl acetate/methylene chloride (1:5) as eluent gave N-[2-(3-methyl-2-thienyl)-6-(1H-pyrazol-1-yl)pyrimidin-4-yl]propanamide (5 mg, 11%) as an off-white solid.

δ (300 MHz, DMSO-d₆): 1.10 (t, J=7.6 Hz, 3H); 2.54 (q, J=7.6 Hz, 2H); 2.74 (s, 3H); 6.68-6.67 (m, 1H); 7.08 (d, J=5.1 Hz, 1H); 7.95-7.94 (m, 1H); 8.42 (s, 1H); 8.63 (d, J=5.1 Hz, 1H); 10.89 (bs, 1H).

### Intermediate 19. 3-Amino-3-(2-furyl)acrylonitrile

To a solution of diisopropilamine (0.92 g, 9.13 mmol) in anhydrous tetrahydrofuran (17 mL), cooled at −78° C., was slowly added 1.6M solution of n-butyllithium in hexanes (5.85 mL). The mixture was stirred at −78° C. for 30 minutes and then, a solution of acetonitrile (0.33 g, 8.06 mmol) in anhydrous tetrahydrofuran (3.5 mL) was slowly added. After 30 minutes at the same temperature, a solution of 2-furonitrile (0.50 g, 5.37 mmol) was added. The mixture was allowed to stand at −78° C. for 20 minutes and at room temperature overnight. Water (6 mL) was added and the solvent removed under reduced pressure. The resulting solid was suspended in water (50 mL) and extracted with methylene chloride (3×25 mL). The organic layer was washed with brine (2×20 mL), dried (Na₂SO₄), and the solvent removed under reduced pressure. The title compound was obtained (0.70 g, 97%) as a brown solid, which was used in the next step without further characterisation.

### Intermediate 20. 4-Amino-6-(2-furyl)pyrimidin-2-thiol

To a solution of Intermediate 19 (1.38 g, 10.3 mmol) in ethanol (17 mL) were added sodium ethoxide (1.54 g, 22.6 mmol) and thiourea (1.56 g, 22.6 mmol). The mixture was refluxed for 45 hours. The resulting suspension was cooled and water was added (12 mL). The solution was acidified with 1N hydrochloric acid until pH=5. The resulting solid was filtered, washed with water (2×20 mL), ethyl ether (10 mL) and dried (Na₂SO₄). 4-Amino-6-(2-furyl)pyrimidin-2-thiol was obtained (1.20 g, 60%) as a solid.

δ (250 MHz, DMSO-d₆): 6.27 (s, 1H); 6.71 (dd, J1=3.4 Hz, J2=1.7 Hz, 1H); 7.76-7.53 (m, 2H); 7.95 (dd, J1=1.7 Hz, J2=0.8 Hz, 1H); 12.14 (bs, 1H).

### Intermediate 21. 6-(2-Furyl)-2-methylsulfanylpyrimidin-4-amine

To a solution of Intermediate 20 (1.87 g, 9.68 mmol) in 10% sodium hydroxide (15 mL) was added methyl iodide (1.51 g, 10.6 mmol). The mixture was stirred at room temperature for 2 hours. The solvent was partially removed under reduced pressure and 2N hydrochloric acid was added until pH=10. The resulting solid was filtered, washed with water (2×20 mL) and dried. 6-(2-furyl)-2-methylsulfanylpyrimidin-4-amine was obtained (1.90 g, 95%) as an off-white solid.

δ (400 MHz, MeOD): 3.46 (s, 3H); 7.48 (s, 1H); 7.52 (dd, J1=3.4 Hz, J2=1.7 Hz, 1H); 8.08 (dd, J1=3.4 Hz, J2=0.8 Hz, 1H); 8.59 (dd, J1=1.7 Hz, J2=0.8 Hz, 1H).

### Intermediate 22. 6-(2-Furyl)-2-methanesulfonylpyrimidin-4-amine

To a suspension of Intermediate 21 (1.90 g, 9.20 mmol) in chloroform (70 mL), cooled at 0° C., was added 70% m-chloroperbenzoic acid (4.53 g, 18.4 mmol). The mixture was stirred at 0° C. for 45 minutes. The solvent was partially removed under reduced pressure and the resulting solid was filtered, washed with ethyl ether, and dried. 1.36 g of the title compound were obtained. The organic solution was washed with 2N sodium hydroxide (2×25 mL), dried (Na₂SO₄), and the solvent removed under reduced pressure. 0.47 g of the title compound were obtained (overall yield: 83%).

δ (400 MHz, MeOD): 4.27 (s, 3H); 7.6 (dd, J1=3.4 Hz, J2=1.7 Hz, 1H); 7.86 (s, 1H); 8.27 (dd, J1=3.4 Hz, J2=0.8 Hz, 1H); 8.68 (dd, J1=1.7 Hz, J2=0.8 Hz, 1H).

### EXAMPLE 76

### 6-(2-Furyl)-2-(1H-pyrazol-1-yl)pyrimidin-4-amine

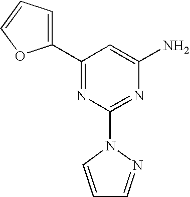

To a solution of Intermediate 22 (1.16 g, 4.85 mmol) in anhydrous DMSO (20 mL) was added pyrazol (0.36 g, 5.33 mmol) and cesium carbonate (1.71 g, 5.33 mmol). The mixture was heated at 110° C. for 2.5 hours and at room temperature overnight. The solution was poured into water (60 mL) and extracted with ethyl acetate (3×40 mL). The organic layer was washed with water (2×50 mL), brine (50 mL), dried (Na₂SO₄), and the solvent removed under reduced pressure. Purification by column chromatography with silica gel and methylene chloride/methanol (5%) as eluent, followed by trituration with ethyl ether gave 6-(2-furyl)-2-(1H-pyrazol-1-yl)pyrimidin-4-amine (0.56 g, 51%) as an off-white solid.

δ (250 MHz, CDCl₃): 5.33 (bs, 2H); 6.47-6.46 (m, 1H); 6.58-6.56 (m, 1H); 6.68 (s, 1H); 7.27 (s, 1H); 7.56 (s, 1H); 7.79 (s, 1H); 8.63 (d, J=2.4 Hz, 1H).

### EXAMPLE 77

### N-[6-(2-Furyl)-2-(1H-pyrazol-1-yl)pyrimidin-4-yl]acetamide

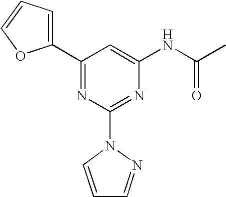

Obtained from the title compound of Example 76 (0.20 g) by the procedure described in Example 2. Purification by column chromatography with silica gel and methylene chloride/methanol (2%) as eluent gave N-[6-(2-furyl)-2-(1H-pyrazol-1-yl)pyrimidin-4-yl]acetamide (0.19 g, 80%) as an off-white solid.

δ (250 MHz, CDCl₃): 2.21 (s, 3H); 6.46 (bs, 1H); 6.56-6.55 (m, 1H); 7.31 (d, J=3.6 Hz, 1H); 7.60 (s, 1H); 7.77 (s, 1H); 8.29 (s, 1H); 8.55 (bs, 1H); 8.60 (d, J=2.4 Hz, 1H).

### EXAMPLE 78

### N-[6-(2-Furyl)-2-(1H-pyrazol-1-yl)pyrimidin-4-yl]propanamide

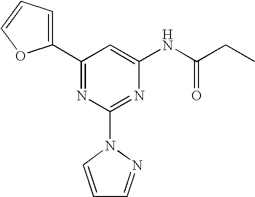

Obtained from the title compound of Example 76 (0.28 g) by the procedure described in Example 2. Purification by column chromatography with silica gel and methylene chloride/methanol (2%) as eluent gave N-[6-(2-furyl)-2-(1H-pyrazol-1-yl)pyrimidin-4-yl]propanamide (84 mg, 24%) as a pale yellow solid.

δ (250 MHz, CDCl₃): 1.27 (t, J=7.3 Hz, 3H); 2.47 (q, J=7.3 Hz, 2H); 6.49-6.48 (m, 1H); 6.58-6.57 (m, 1H); 7.33 (d, J=3.6 Hz, 1H); 7.61 (s, 1H); 7.79 (s, 1H); 8.34 (bs, 1H); 8.36 (d, J=1.2 Hz, 1H); 8.64 (d, J=2.4 Hz, 1H).

### EXAMPLE 79

### 3,3,3-Trifluoro-N-[6-(2-furyl)-2-(1H-pyrazol-1-yl)pyrimidin-4-yl]-propanamide

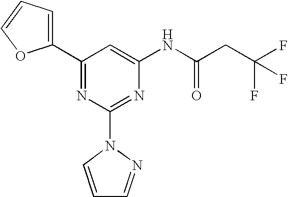

Obtained from the title compound of Example 76 (0.25 g) by the procedure described in Example 14. Purification by column chromatography with silica gel and methylene chloride/methanol (4%) as eluent gave 3,3,3-trifluoro-N-[2-(2-furyl)-2-(1H-pyrazol-1-yl)pyrimidin-yl]propanamide (0.19 mg, 49%) as an off-white solid.

δ (250 MHz, CDCl₃): 3.32 (q, J=9.8 Hz, 2H); 6.51-6.49 (m, 1H); 6.61-6.59 (m, 1H); 7.37 (d, J=3.6 Hz, 1H); 7.63 (s, 1H); 7.80 (s, 1H); 8.32 (s, 1H); 8.63 (d, J=2.4 Hz, 1H); 8.68 (bs, 1H).

### EXAMPLE 80

### 2-(3,5-Dimethyl-1H-pyrazol-1-yl)-6-(2-furyl)pyrimidin-4-amine

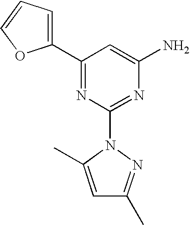

Obtained from Intermediate 22 (3.00 g) by the procedure described in Example 76. Purification by column chromatography with silica gel and chloroform/isopropanol (1:1) as eluent, followed by a second column chromatography with silica gel and methylene chloride/methanol (5%) as eluent gave 2-(3,5-dimethyl-1H-pyrazol-1-yl)-6-(2-furyl)pyrimidin-4-amine (0.15 g, 5%) as an off-white solid.

δ (250 MHz, CDCl₃): 2.33 (s, 3H); 2.72 (s, 3H); 5.18 (bs, 2H); 6.01 (s, 1H); 6.54 (dd, J1=6.3 Hz, J2=1.8 Hz, 1H); 6.63 (s, 1H); 7.15-7.13 (m, 1H); 7.55-7.54 (m, 1H).

### EXAMPLE 81

### N-[2-(3,5-Dimethyl-1H-pyrazol-1-yl)-6-(2-furyl)pyrimidin-yl]-propanamide

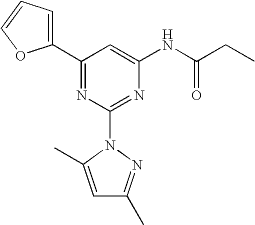

Obtained from the title compound of Example 80 (95 mg) by the procedure described in Example 2. Purification by column chromatography with silica gel and methylene chloride/methanol (10%) as eluent gave N-[2-(3,5-dimethyl-1H-pyrazol-1-yl)-6-(2-furyl)pyrimidin-4-yl]propanamide (50 mg, 43%) as an off-white solid.

δ (250 MHz, CDCl₃): 1.25 (t, J=7.6 Hz, 3H); 2.35 (s, 3H); 2.45 (q, J=7.6 Hz, 2H); 2.75 (s, 3H); 6.05 (s, 1H); 6.58-6.56 (m, 1H); 7.24-7.23 (m, 1H); 7.61 (s, 1H); 8.27 (bs, 1H); 8.33 (s, 1H).

### EXAMPLE 82

### N-[2-(3,5-Dimethyl-1H-pyrazol-1-yl)-6-(2-furyl)pyrimidin-4-yl]-2-(4-methoxyphenyl)acetamide

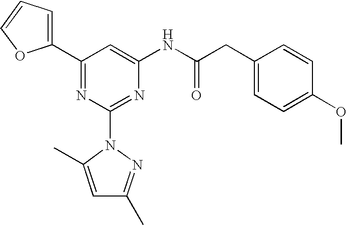

Obtained from the title compound of Example 80 (100 mg) by the procedure described in Example 2. Purification by column chromatography with silica gel and methylene chloride/methanol (10%) as eluent gave N-[2-(3,5-dimethyl-1H-pyrazol-1-yl)-6-(2-furyl)pyrimidin-4-yl]-2-(4-methoxyphenyl)acetamide (50 mg, 30%) as an off-white solid.

δ (250 MHz, CDCl₃): 2.32 (s, 3H); 2.71 (s, 3H); 3.69 (s, 2H); 3.81 (s, 3H); 6.03 (s, 1H); 6.57-6.55 (m, 1H); 6.90 (d, J=8.8 Hz, 2H); 7.24-7.20 (m, 3H); 7.60 (d, J=1.5 Hz, 1H); 8.35 (s, 2H).

### EXAMPLE 83

### 6-(2-Furyl)-2-(1H-1,2,4-triazol-1-yl)pyrimidin-4-amine

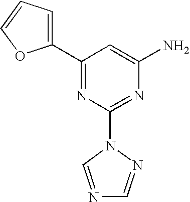

To a solution of Intermediate 22 (0.21 g, 0.88 mmol) in anhydrous DMF (3 mL) was added [1,2,4]-triazol (60 mg, 0.88 mmol) and potassium carbonate (0.12 g, 0.88 mmol). The mixture was heated at 80° C. for 2 hours. The solution was poured into water (10 mL) and extracted with ethyl acetate (2×10 mL). The organic layer was washed with water (2×10 mL), brine (10 mL), dried (Na₂SO₄), and the solvent removed under reduced pressure. Purification by column chromatography with silica gel and methylene chloride as eluent gave 6-(2-furyl)-2-(1H-1,2,4-triazol-1-yl)pyrimidin-4-amine (70 mg, 35%) as an off-white solid.

δ (200 MHz, DMSO-d₆): 6.70 (s, 1H); 6.73-6.72 (m, 1H); 7.30 (d, J=3.4 Hz, 1H); 7.57 (bs, 2H); 7.93-7.92 (m, 1H); 8.22 (s, 1H); 9.35 (s, 1H).

### EXAMPLE 84

### N-[6-(2-Furyl)-2-(1H-1,2,4-triazol-1-yl)pyrimidin-4-yl]propanamide

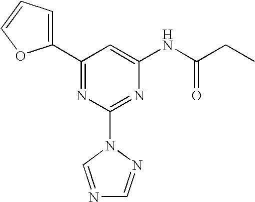

Obtained from the title compound of Example 83 (45 mg) by the procedure described in Example 49. Purification by column chromatography with silica gel and methylene chloride/methanol (1%) as eluent gave N-[6-(2-furyl)-2-(1H-1,2,4-triazol-1-yl)pyrimidin-yl]propanamide (16 mg, 28%) as an off-white solid.

δ (300 MHz, DMSO-d₆): 1.16 (t, J=7.4 Hz, 3H); 2.53 (q, J=7.4 Hz, 2H); 6.66 (dd, J1=3.4 Hz, J2=1.8 Hz, 1H); 7.43 (dd, J1=3.4, J2=0.8 Hz, 1H); 7.79 (dd, J1=1.8 Hz, J2=0.8 Hz, 1H); 8.12 (s, 1H); 8.44 (s, 1H); 9.34 (s, 1H); 11.16 (bs, 1H).

### Intermediate 23. 3-Amino-3-pyridin-2-ylacrylonitrile

To a solution of pyridine-2-carbonitrile (5.0 g, 48.0 mmol) in toluene (175 mL) was added potassium tertbutoxide (16.2 g, 0.144 mol) and acetonitrile (3.94 g, 96.0 mmol). The mixture was stirred at room temperature for 3 hours. To the reaction mixture was added saturated solution of potassium bicarbonate (200 mL) and the mixture was extracted with ethyl ether (2×200 mL). The organic layer was dried (Na₂SO₄) and the solvent removed under reduced pressure. The title compound was obtained (5.44 g, 78%) as a light brown solid, which was used in the next step without further characterisation.

### Intermediate 24. 4-Amino-6-pyridin-2-ylpyrimidin-2-thiol

Obtained from Intermediate 23 (1.14 g) by the procedure described in Intermediate 20. Purification by trituration with ethyl ether gave 4-amino-6-pyridin-2-ylpyrimidin-2-thiol (1.28 g, 80%) as an off-white solid.

δ (250 MHz, DMSO-d₆): 6.70 (bs, 1H); 7.60 (m, 1H); 7.79 (bs, 1H); 8.15-7.98 (m, 2H); 8.74 (m, 1H); 11.21 (bs, 1H).

### Intermediate 25. 2-Methylsulfanyl-6-pyridin-2-ylpyrimidin-4-amine

Obtained from Intermediate 24 (4.0 g) by the procedure described in Intermediate 21. Purification by trituration with ethyl ether gave the title compound (4.16 g, 97%) as an orange solid.

MS (M⁺): 218.

### Intermediate 26. 2-Methanesulfonyl-6-pyridin-2-ylpyrimidin-4-amine

Obtained from Intermediate 25 (4.16 g) by the procedure described in Intermediate 22. Purification by trituration with ethyl ether gave 2-methanesulfonyl-6-pyridin-2-ylpyrimidin-4-amine (3.89 g, 82%) as an off-white solid.

δ (250 MHz, DMSO-d₆): 3.38 (s, 3H); 7.59-7.53 (m, 2H); 7.86 (bs, 1H); 8.05-7.97 (m, 1H); 8.34 (m, 1H); 8.73 (m, 1H).

### EXAMPLE 85

### 2-(1H-Pyrazol-1-yl)-6-pyridin-2-ylpyrimidin-amine

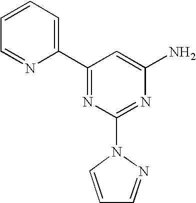

Obtained from Intermediate 26 (0.43 g) by the procedure described in Example 76. Purification by column chromatography with silica gel and methylene chloride/methanol (2%) as eluent gave 2-(1H-pyrazol-1-yl)-6-pyridin-2-ylpyrimidin-4-amine (0.25 g, 47%) as an off-white solid.

δ (250 MHz, DMSO-d₆): 6.54 (t, J=1.7 Hz, 1H); 7.38 (s, 1H); 7.46 (bs, 2H); 7.56-7.50 (m, 1H); 7.78-7.77 (m, 1H); 7.99 (dt, J1=7.7 Hz, J2=1.7 Hz, 1H); 8.45 (d, J=7.7 Hz, 1H); 8.71 (d, J=2.7 Hz, 2H).

### EXAMPLE 86

### N-[2-(1H-Pyrazol-1-yl)-6-pyridin-2-ylpyrimidin-yl]propanamide

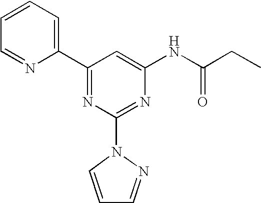

Obtained from the title compound of Example 85 (0.19 g) by the procedure described in Example 2. Purification by column chromatography with silica gel and methylene chloride/methanol (2%) as eluent gave N-[2-(1H-pyrazol-1-yl)-6-pyridin-2-ylpyrimidin-4-yl]propanamide (0.15 g, 65%) as an off-white solid.

δ (250 MHz, CDCl₃): 1.28 (t, J=7.6 Hz, 2H); 2.49 (q, J=7.6 Hz, 2H); 6.53-6.51 (m, 1H); 7.45-7.39 (m, 1H); 7.90-7.83 (m, 2H); 8.30 (bs, 1H), 8.47 (dd, J1=7.9 Hz, J2=0.9 Hz, 1H); 8.77-8.72 (m, 2H); 9.08 (s, 1H).

### EXAMPLE 87

### 2-(3,5-Dimethyl-1H-pyrazol-1-yl)-6-pyridin-2-ylpyrimidin-4-amine

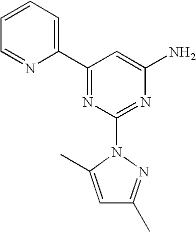

Obtained from Intermediate 26 (3.90 g) by the procedure described in Example 76. Purification by column chromatography with silica gel and methylene chloride/acetonitrile (from 4:1 to 1:4) as eluent, followed by a second column chromatography with silica gel and methylene chloride/acetonitrile/methanol (1:4:0.25) as eluent gave 2-(3,5-dimethyl-1H-pyrazol-1-yl)-6-pyridin-2-ylpyrimidin-4-amine (0.42 g, 10%) as an off-white solid.

δ (250 MHz, CDCl₃): 2.35 (s, 3H); 2.79 (s, 3H); 5.26 (bs, 2H); 6.05 (s, 1H); 7.41-7.36 (m, 2H); 7.85 (dt, J1=7.6 Hz, J2=1.8 Hz, 1H); 8.38-8.34 (m, 1H); 8.70-8.67 (m, 1H).

### EXAMPLE 88

### N-[2-(3,5-Dimethyl-1H-pyrazol-1-yl)-6-pyridin-2-ylpyrimidin-4-yl]propanamide

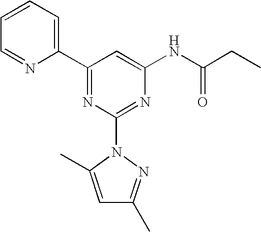

Obtained from the title compound of Example 87 (0.17 g) by the procedure described in Example 2. Purification by column chromatography with silica gel and methylene chloride/methanol (from pure methylene chloride to 5% methanol) as eluent gave N-[2-(3,5-dimethyl-1H-pyrazol-1-yl)-6-pyridin-2-ylpyrimidin-4-yl]propanamide (0.10 g, 64%) as an off-white solid.

δ (250 MHz, CDCl₃): 1.26 (t, J=7.6 Hz, 3H); 2.36 (s, 3H); 2.46 (q, J=7.6 Hz, 2H); 2.81 (s, 3H); 6.08 (s, 1H); 7.40 (ddd, J1=7.6 Hz, J2=1.8 Hz, 1H); 7.85 (dt, J1=7.6 Hz, J2=1.8 Hz, 1H); 8.36-8.31 (m, 2H); 8.76-8.73 (m, 1H); 9.05 (s, 1H).

### EXAMPLE 89

### N-[2-(3,5-Dimethyl-1H-pyrazol-1-yl)-6-pyridin-2-ylpyrimidin-4-yl]-2-(4-methoxyphenyl)acetamide

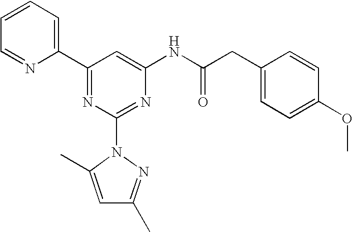

Obtained from the title compound of Example 87 (0.17 g) by the procedure described in Example 2. Purification by column chromatography with silica gel and methylene chloride/methanol (from 1% to 5%) as eluent, followed by a second column chromatography with silica gel and methylene chloride/methanol (from pure methylene chloride to 10% methanol) as eluent gave N-[2-(3,5-dimethyl-1H-pyrazol-1-yl)-6-pyridin-2-ylpyrimidin-4-yl]-2-(4-methoxyphenyl)acetamide (94 mg, 31%) as an off-white solid.

δ (250 MHz, CDCl₃): 2.34 (s, 3H); 2.78 (s, 3H); 3.71 (s, 2H); 3.81 (s, 3H); 6.07 (s, 1H); 6.89-6.87 (m, 1H); 6.93-6.91 (m, 1H); 7.26-7.21 (m, 2H); 7.40 (ddd, J1=7.6 Hz, J2=4.8 Hz, 1H); 7.84 (dt, J1=7.6 Hz, J2=1.8 Hz, 1H); 8.25 (bs, 1H); 8.33 (dt, J1=7.6 Hz, J2=1.2 Hz, 1H); 8.74 (dt, J1=4.8 Hz, J2=1.8 Hz, 1H); 9.06 (s, 1H).

### Intermediate 27. 3-Amino-3-pyridin-3-ylacrylonitrile

Obtained from pyridine-3-carbonitrile (5.00 g) by the procedure described in Intermediate 23 (reaction time: 3 days). Purification by trituration with ethyl ether gave 3-amino-3-pyridin-3-ylacrylonitrile (2.81 g, 40%) as an off-white solid.

δ (250 MHz, DMSO-d₆): 4.58 (s, 1H); 6.96 (s, 2H); 7.46 (dd, J1=7.9 Hz, J2=4.6 Hz, 1H); 7.98-7.93 (m, 1H); 8.64 (dd, J1=4.7 Hz, J2=1.6 Hz, 1H); 8.77 (dd, J1=2.5 Hz, J2=0.8 Hz, 1H).

### Intermediate 28. 4-Amino-6-pyridin-3-ylpyrimidin-2-thiol

Obtained from Intermediate 27 (2.81 g) by the procedure described in Intermediate 20. Purification by trituration with ethyl ether gave 4-amino-6-pyridin-3-ylpyrimidin-2-thiol (3.28 g, 83%) as an off-white solid.

δ (200 MHz, DMSO-d₆): 7.53 (dd, J1=8.1 Hz, J2=4.7 Hz, 2H); 7.67 (s, 2H); 8.12-8.07 (m, 1H); 8.71 (d, J=4.7 Hz, 1H); 8.85 (s, 1H); 12.40 (bs, 1H).

### Intermediate 29. 2-Methylsulfanyl-6-pyridin-3-ylpyrimidin-4-mine

Obtained from Intermediate 28 (3.00 g) by the procedure described in Intermediate 21. Purification by trituration with ethyl ether gave the title compound (2.96 g, 92%) as a solid, which was used in the next step without further characterisation.

### Intermediate 30. 2-Methanesulfonyl-6-pyridin-3-ylpyrimidin-4-amine

Obtained from Intermediate 29 (2.00 g) by the procedure described in Intermediate 22. Purification by trituration with ethyl ether gave 2-methanesulfonyl-6-pyridin-3-ylpyrimidin-4-amine (1.90 g, 83%) as an off-white solid.

MS (M⁺): 250

### EXAMPLE 90

### 2-(1H-Pyrazol-1-yl)-6-pyridin-3-ylpyrimidin-4-amine

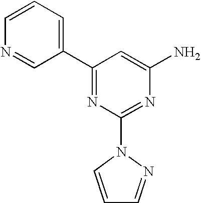

Obtained from Intermediate 30 (1.00 g) by the procedure described in Example 76. Purification by column chromatography with silica gel and methylene chloride/methanol (from 2% to 3%) as eluent gave 2-(1H-pyrazol-1-yl)-6-pyridin-3-ylpyrimidin-4-amine (0.24 g, 25%) as an off-white solid.

δ (250 MHz, DMSO-d₆): 6.55-6.53 (m, 1H); 6.91 (s, 1H); 7.45 (bs, 2H); 7.56 (dd, J1=7.9 Hz, J2=4.9 Hz, 1H); 7.78 (s, 1H); 8.46-8.41 (m, 1H); 8.72-8.69 (m, 2H); 9.26-9.24 (m, 1H).

### EXAMPLE 91

### N-[2-(1H-Pyrazol-1-yl)-6-pyridin-3-ylpyrimidin-4-yl]propanamide

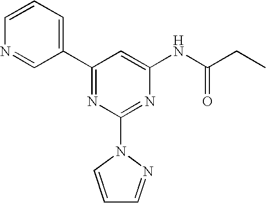

Obtained from the title compound of Example 90 (0.14 g) by the procedure described in Example 2. Purification by column chromatography with silica gel and methylene chloride/methanol (2%) as eluent, followed by a second column chromatography with silica gel and methylene chloride/methanol (5%) as eluent gave N-[2-1H-pyrazol-1-yl)-6-pyridin-3-ylpyrimidin-4-yl]propanamide (54 mg, 31%) as an off-white solid.

δ (250 MHz, DMSO-d₆): 1.10 (t, J=7.4 Hz, 3H); 2.53 (q, J=7.4 Hz, 2H); 6.63 (m, J1=2.6 Hz, J2=1.4 Hz, 1H); 7.65-7.60 (m, 1H); 7.88-7.87 (m, 1H); 8.51 (t, J=1.4 Hz, 1H); 8.55 (s, 1H); 8.78 (dd, J1=4.6 Hz, J2=1.4 Hz, 1H); 8.84 (dd, J1=2.6 Hz, J2=0.5 Hz, 1H); 9.35 (d, J=1.9 Hz, 1H); 11.36 (s, 1H).

### EXAMPLE 92

### 2-(3,5-Dimethyl-1H-pyrazol-1-yl)-6-pyridin-3-ylpyrimidin-4-amine

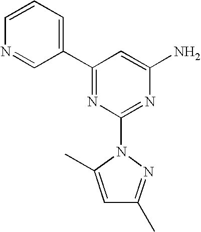

Obtained from Intermediate 30 (1.77 g) by the procedure described in Example 76. Purification by column chromatography with silica gel and methylene chloride/methanol (4%) as eluent gave 2-(3,5-dimethyl-1H-pyrazol-1-yl)-6-pyridin-3-ylpyrimidin-4-amine (0.35 g, 8%) as an off-white solid.

δ (250 MHz, CDCl₃): 2.18 (s, 3H); 2.63 (s, 3H); 6.09 (s, 1H); 6.86 (s, 1H); 7.37 (bs, 2H); 7.56 (dd, J1=8.0 Hz, J2=4.7 Hz, 1H); 8.34 (dt, J1=8.0 Hz, J2=1.6 Hz, 1H); 8.69 (dd, J1=4.7 Hz, J2=1.6 Hz, 1H); 9.17 (d, J=2.2 Hz, 1H).

### EXAMPLE 93

### N-[2-(3,5-Dimethyl-1H-pyrazol-1-yl)-6-pyridin-3-ylpyrimidin-4-yl]-propanamide

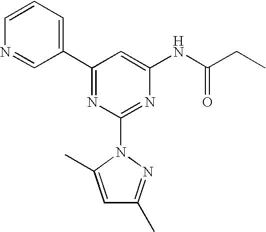

Obtained from the title compound of Example 92 (0.15 g) by the procedure described in Example 2. Purification by column chromatography with silica gel and methylene chloride/methanol (from pure methylene chloride to 2% methanol) as eluent gave N-[2-(3,5-dimethyl-1H-pyrazol-1-yl)-6-pyridin-3-ylpyrimidin-4-yl]propanamide (74 mg, 41%) as an off-white solid.

δ (250 MHz, CDCl₃): 1.27 (t, J=7.6 Hz, 3H); 2.48 (q, J=7.6 Hz, 2H); 2.80 (s, 3H); 6.09 (s, 1H); 7.46 (dd, J1=8.2 Hz, J2=5.2 Hz, 1H); 8.40-8.35 (m, 2H); 8.54 (s, 1H); 8.60 (d, J=2.4 Hz, 1H); 8.77-8.74 (m, 1H); 9.39-9.38 (m, 1H).

### EXAMPLE 94

### N-[2-(3,5-Dimethyl-1H-pyrazol-1-yl)-6-pyridin-3-ylpyrimidin-yl]-2-(4-methoxyphenyl)acetamide

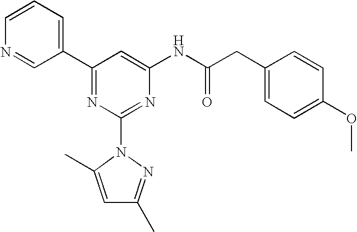

Obtained from the title compound of Example 92 (0.15 g) by the procedure described in Example 2. Purification by column chromatography with silica gel and methylene chloride/methanol (from pure methylene chloride to 5% methanol) as eluent gave N-[2-(3,5-dimethyl-1H-pyrazol-1-yl)-6-pyridin-3-ylpyrimidin-4-yl]-2-(4-methoxyphenyl)acetamide (0.20 g, 86%) as an off-white solid.

δ (250 MHz, CDCl₃): 2.34 (s, 3H); 2.77 (s, 3H); 3.72 (s, 2H); 3.81 (s, 3H); 6.07 (s, 1H); 6.91 (d, J=8.8 Hz, 2H); 7.23 (d, J=8.8 Hz, 2H); 7.47-7.42 (m, 1H); 8.40-8.33 (m, 2H); 8.56 (s, 1H); 8.76-8.73 (m, 1H); 9.38-9.36 (m, 1H).

### Intermediate 31. 3-Amino-3-pyridin-ylacrylonitrile

Obtained from pyridine-4-carbonitrile (5.00 g) by the procedure described in Intermediate 23 (reaction time: 12 hours). Purification by trituration with ethyl ether gave 3-amino-3-pyridin-4-ylacrylonitrile, which was used in the next step without further purification.

### Intermediate 32. 4-Amino-6-pyridin-4-ylpyrimidin-2-thiol

Obtained from Intermediate 31 by the procedure described in Intermediate 20. Purification by trituration with ethyl ether gave 4-amino-6-pyridin-4-ylpyrimidin-2-thiol (7.43 g, global yield: 76%) as a solid, which was used in the next step without further characterisation.

### Intermediate 33. 2-Methylsulfanyl-6-pyridin-4-ylpyrimidin-4-amine

Obtained from Intermediate 32 (7.00 g) by the procedure described in Intermediate 21. Purification by trituration with ethyl ether gave the title compound (6.12 g, 82%) as a solid, which was used in the next step without further characterisation.

### Intermediate 34. 2-Methanesulfonyl-6-pyridin-ylpyrimidin-4-amine

Obtained from Intermediate 33 (2.00 g) by the procedure described in Intermediate 22. Purification by trituration with ethyl ether gave 2-methanesulfonyl-6-pyridin-4-ylpyrimidin-4-amine (2.29 g, 99%) as a solid, which was used in the next step without further characterisation.

### EXAMPLE 95

### 2-(1H-Pyrazol-1-yl)-6-pyridin-4-ylpyrimidin-4-amine

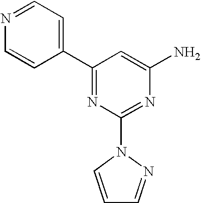

Obtained from Intermediate 34 (2.00 g) by the procedure described in Example 76. Purification by column chromatography with silica gel and methylene chloride/methanol (3%) as eluent gave 2-(1H-pyrazol-1-yl)-6-pyridin-4-ylpyrimidin-4-amine (0.32 g, 17%) as an off-white solid.

δ (250 MHz, DMSO-d₆): 6.56-6.54 (m, 1H); 6.95 (s, 1H); 7.53 (bs, 2H); 7.79-7.78 (m, 1H); 8.02-8.00 (m, 2H); 8.70-8.68 (m, 1H); 8.76-8.74 (m, 2H).

### EXAMPLE 96

### N-[2-(1H-Pyrazol-1-yl)-6-pyridin-4-ylpyrimidin-4-amine

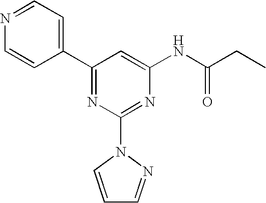

Obtained from the title compound of Example 95 (0.15 g) by the procedure described in Example 2. Purification by column chromatography with silica gel and methylene chloride/methanol (2%) as eluent gave N-[2-(1H-pyrazol-1-yl)-6-pyridin-4-ylpyrimidin-4-amine (64 mg, 22%) as an off-white solid.

δ (250 MHz, DMSO-d₆): 1.10 (t, J=7.7 Hz, 3H); 2.53 (q, J=7.7 Hz, 2H); 6.65-6.63 (m, 1H); 7.89-7.88 (m, 1H); 8.12-8.10 (m, 2H); 8.59 (s, 1H); 8.83-8.80 (m, 3H); 11.4 (bs, 1H).

### Intermediate 35. 3-(2-Furyl)-3-oxopropionic acid ethyl ester

To a solution of 60% sodium hydride (95.4 mmol) in diethyl carbonate (90 ml) was slowly added 2-acetylfurane (5.50 g, 45.4 mmol). The resulting solution was stirred at room temperature for 1 hour and at 90° C. for 2 hours. The reaction mixture was poured into ice/water and acetic acid (5 mL) was added. The mixture was extracted with ethyl acetate (2×75 mL). The organic layer was washed with water (2×50 mL), brine (50 mL), dried (Na₂SO₄), and the solvent removed under reduced pressure. Purification by flash chromatography with silica gel and ethyl acetate/n-hexane (4:1) as eluent gave the title compound (5.90 g, 71%) as a red oil.

δ (200 MHz, CDCl₃): 1.26 (t, J=7.2 Hz, 3H); 3.86 (s, 2H); 4.21 (q, J=7.2 Hz, 2H); 6.58 (dd, J1=3.4 Hz, J2=1.7 Hz, 1H); 7.28 (d, J=3.4 Hz, 1H); 7.62 (d, J=1.7 Hz, 1H).

### Intermediate 36. 6-(2-Furyl)-2-pyridin-2-ylpyrimidin-4-ol

To a solution of potassium tertbutoxide (0.87 g, 7.79 mmol) in butanol (3 ml) were added Intermediate 35 (1.00 g, 5.49 mmol) and pyridine-2-carboxamidine (HCl) (1.08 g, 6.86 mmol). The mixture was heated at 135° C. for 5 hours. The resulting solid was filtered and washed with n-pentane. Purification by flash chromatography with silica gel and methylene chloride/methanol (from 1% to 3%) as eluent gave 642-furyl)-2-pyridin-2-ylpyrimidin-4-ol (0.33 g, 25%) as an off-white solid.

δ (200 MHz, CDCl₃): 6.58 (s, 1H); 6.75 (dd, J1=3.4 Hz, J2=1.7 Hz, 1H); 7.39 (d, J=3.4 Hz, 1H); 7.66-7.72 (m, 1H); 7.97 (s, 1H); 8.06-8.14 (m, 1H); 8.49 (d, J=7.7 Hz, 1H); 8.77 (d, J=4.7 Hz, 1H).

### Intermediate 37. 4-Chloro-6-(2-furyl)-2-pyridin-2-ylpyrimidine

Obtained from Intermediate 36 (0.33 g) by the procedure described in Intermediate 10. 4-Chloro-6-(2-furyl)-2-(pyridin-2-yl)pyrimidine (0.36 g, 78%) was obtained as a brown solid.

MS (M⁺): 257.

### EXAMPLE 97

### 6-(2-Furyl)-2-pyridin-2-ylpyrimidin-4-amine

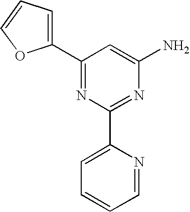

Obtained from Intermediate 37 (0.28 g) by the procedure described in Example 48. Purification by column chromatography with silica gel and methylene chloride/methanol (5%) as eluent gave 6-(2-furyl)-2-pyridin-2-ylpyrimidin-4-amine (0.16 mg, 62%) as an off-white solid.

δ (300 MHz, CDCl₃): 5.55 (bs, 2H); 6.51 (dd, J1=3.4 Hz, J2=1.7 Hz, 1H); 6.78 (s, 1H); 7.26 (d, J=3.4 Hz, 1H); 7.34 (dd, J1=8.1 Hz, J2=5.3 Hz, 1H); 7.52-7.51 (m, 1H); 7.80 (dt, J1=7.6 Hz, J2=1.7 Hz, 1H); 8.50 (d, J=8.1 Hz, 1H); 8.77-8.74 (m, 1H).

### EXAMPLE 98

### N-[6-(2-Furyl)-2-pyridin-2-ylpyrimidin yl]propanamide

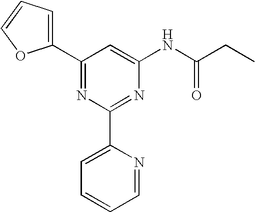

Obtained from the title compound of Example 97 (0.10 g) by the procedure described in Example 49. Purification by trituration with n-pentane gave N-[6-(2-furyl)-2-pyridin-2-ylpyrimidin-4-yl]propanamide (63 mg, 51%) as an off-white solid.

δ (300 MHz, CDCl₃): 1.24 (t, J=7-5 Hz, 3H); 2.45 (q, J=7.5 Hz, 2H); 6.56 (dd, J1=3.4, J2=1.8 Hz, 1H); 7.36 (d, J=3.4 Hz, 1H); 7.43-7.39 (m, 1H); 7.60-7.59 (m, 1H); 7.88 (dt, J1=7.6 Hz, J2=1.8 Hz, 1H); 8.48 (s, 2H); 8.60 (d, J=8.1 Hz, 1H); 8.82-8.81 (m, 1H).

### Intermediate 38. 3-Methylpyridine-2-carboxamidine (HCl)

Obtained from 3-methylpyridine-2-carbonitrile (5.15 g) by the procedure described in Intermediate 1. Purification by trituration with ethyl ether gave the title compound (3.13 g, 42%) as an off-white solid.

δ (300 MHz, DMSO-d₆): 2.41 (s, 3H), 7.56-7.67 (m, 5H); 8.40 (s, 1H); 8.56 (d, J=3.8 Hz, 1H).

### Intermediate 39. 6-Amino-2-(3-methylpyridin-2-yl)pyrimidin-4-ol

Obtained from Intermediate 38 (2.91 g) by the procedure described in Intermediate 11. Purification by column chromatography with silica gel and methylene chloride/methanol (from 2% to 5%) as eluent gave 6-amino-2-(3-methylpyridin-2-yl)pyrimidin-4-ol (0.58 g, 17%) as an off-white solid.

δ (300 MHz, DMSO-d₆): 2.52 (s, 3H); 5.05 (s, 1H); 6.57 (s, 2H); 7.47 (dd, J1=7.6 Hz, J2=4.7 Hz, 1H); 7.80 (d, J=7.6 Hz, 1H); 8.50 (d, J=4.7 Hz, 1H); 11.26 (bs, 1H).

### Intermediate 40. N-[6-Chloro-2-(3-methylpyridin-2-yl)pyrimidin-4-yl]propanamide

Obtained from Intermediate 39 (0.60 g) by the procedure described in Intermediate 12. Purification by column chromatography with silica gel and methylene chloride/methanol (5%) as eluent gave N-[6-chloro-2-(3-methylpyridin-2-yl)pyrimidin-4-yl]propanamide (0.14 g, 17%) as an off-white solid.

δ (300 MHz, DMSO-d₆): 1.10 (t, J=7.4 Hz, 3H); 2.40-2.50 (m, 5H); 7.60 (dd, J1=7.6 Hz, J2=4.7 Hz, 1H); 8.00 (d, J=7.6 Hz, 1H); 8.20 (s, 1H); 8.60 (d, J=4.7 Hz, 1H); 11.40 (bs, 1H).

### EXAMPLES 99 AND 100

### 2-(3-Methylpyridin-2-yl)-6-(1H-pyrazol-1-yl)pyrimidin-4-amine and N-[2-(3-methylpyridin-2-yl)-6-(1H-pyrazol-1-yl)pyrimidin-4-yl]propanamide

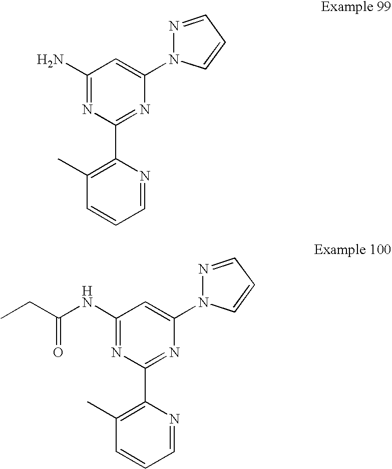

Obtained from Intermediate 40 (0.14 g) by the procedure described in Example 21 (reaction temperature: 110° C.). Purification by column chromatography with silica gel and methylene chloride/methanol (2%) as eluent 2-(3-methylpyridin-2-yl)-6-(1H-pyrazol-1-yl)pyrimidin-4-amine (12 mg, 8%) and N-[2-(3-methylpyridin-2-yl)-6-(1H-pyrazol-1-yl)pyrimidin-4-yl]propanamide (5 mg, 4%) as off-white solids.

### EXAMPLE 99

δ (300 MHz, DMSO-d₆): 2.33 (s, 3H); 6.54 (dd, J1=2.7 Hz, J2=1.7 Hz, 1H); 6.91 (s, 1H); 7.31 (bs, 2H); 7.37 (dd, J1=7.8 Hz, J2=4.7 Hz, 1H); 7.74-7.71 (m, 1H); 7.85 (dd, J1=1.7 Hz, J2=0.6 Hz, 1H); 8.38 (bs, 1H) 8.44 (dd, J1=4.7 Hz, J2=1.1 Hz, 1H); 8.50 (dd, J1=2.75 Hz, J2=0.6 Hz, 1H).

### EXAMPLE 100

δ (300 MHz, DMSO-d₆): 1.08 (t, J=7.6 Hz, 3H); 2.40 (s, 1H); 2.46 (q, J=7.6 Hz, 2H); 6.63 (dd, J1=2.8 Hz, J2=1.7 Hz, 1H); 7.81-7.78 (m, 1H); 7.96-7.94 (m, 1H); 8.51-8.49 (m, 1H); 8.62 (s, 1H); 8.64 (dd, J1=2.8 Hz, J2=0.6 Hz, 1H); 11.24 (bs, 1H).

### Intermediate 41. Pyridine-3-carboxamidine (HCl)

Obtained from pyridin-3-carbonitrile (10.0 g) by the procedure described in Intermediate 1. Purification by trituration with ethyl ether gave the title compound (11.64 g, 99%) as an off-white solid.

δ (200 MHz, DMSO-d₆): 7.66-7.70 (m, 1H); 8.23 (d, J=6.4 Hz, 1H); 8.80-8.90 (m, 5H); 9.00 (s, 1H).

### Intermediate 42. 2-(Pyridin-3-yl)pyrimidin-4,6-diol

Obtained from Intermediate 41 (11.64 g) by the procedure described in Intermediate 2. Purification by trituration with ethyl ether gave the title compound (13.68 g, 75%) as an off-white solid.

MS (M⁺): 189.

### Intermediate 43. 4,6-Dichloro-2-(pyridin-3-yl)pyrimidine

Obtained from Intermediate 42 (12.80 g) by the procedure described in Intermediate 3 (reaction time: 40 hours). Purification by trituration with ethyl ether gave 4,6-dichloro-2-(pyridin-3-yl)pyrimidine (6.50 g, 42%) as a solid, which was used in the next step without further characterisation.

### Intermediate 44. 6-Chloro-2-(pyridin-3-yl)pyrimidin-amine

Obtained from Intermediate 43 (2.00 g) by the procedure described in Intermediate 48 (reaction time: 21 hours). Purification by trituration with ethyl ether gave 6-chloro-2-(pyridin-3-yl)pyrimidin-4-amine (2.14 g, 78%) as a solid, which was used in the next step without further characterisation.

### EXAMPLE 101

### 6-(1H-Pyrazol-1-yl)-2-pyridin-3-ylpyrimidin-amine

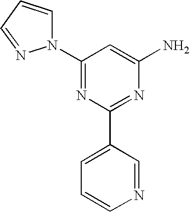

Obtained from Intermediate 44 (1.80 g) by the procedure described in Example 21. Purification by trituration with ethyl ether gave 6-(1H-pyrazol-1-yl)-2-pyridin-3-ylpyrimidin-4-amine (1.40 g, 67%) as an off-white solid.

δ (250 MHz, DMSO-d₆): 6.61-6.59 (m, 1H); 6.89 (s, 1H); 7.35 (bs, 2H); 7.57-7.51 (m, 1H); 7.87-7.86 (m, 1H); 8.71-8.66 (m, 2H); 8.86-8.83 (m, 1H); 9.55-9.53 (m, 1H).

### EXAMPLE 102

### N-[6-(1H-Pyrazol-1-yl)-2-pyridin-3-ylpyrimidin-yl]acetamide

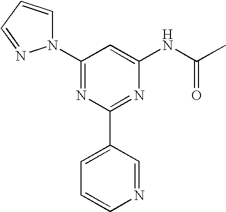

Obtained from the title compound of Example 101 (0.30 g) by the procedure described in Example 2. Purification by column chromatography with silica gel and methylene chloride/methanol (4%) as eluent gave N-[6-(1H-pyrazol-1-yl)-2-pyridin-3-ylpyrimidin-4-yl]acetamide (80 mg, 23%) as an off-white solid.

δ (250 MHz, CDCl₃): 2.33 (s, 3H); 6.53-6.51 (m, 1H); 7.46-7.40 (m, 1H); 7.83-7.82 (m, 1H); 8.56 (bs, 1H); 8.70-8.64 (m, 3H); 8.75-8.72 (m, 1H); 9.65-9.64 (m, 1H).

### EXAMPLE 103

### N-[6-(1H-Pyrazol-1-yl)-2-pyridin-3-ylpyrimidin-4-yl]propanamide

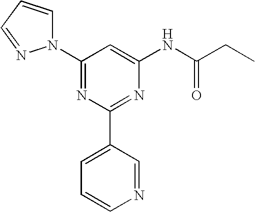

Obtained from the title compound of Example 101 (0.30 g) by the procedure described in Example 2. Purification by column chromatography with silica gel and methylene chloride/methanol (4%) as eluent gave N-[6-(1H-pyrazol-1-yl)-2-pyridin-3-ylpyrimidin-4-yl]propanamide (0.16 g, 41%) as an off-white solid.

δ (250 MHz, CDCl₃): 1.30 (t, J=7.6, 3H); 2.56 (q, J=7.6 Hz, 2H); 6.53-6.51 (m, 1H); 7.46-7.40 (m, 1H); 7.82-7.81 (m, 1H); 8.36 (bs, 1H); 8.75-8.64 (m, 4H); 9.64-9.63 (m, 1H).

### EXAMPLE 104

### 6-(3,5-Dimethyl-1H-pyrazol-1-yl)-2-pyridin-3-ylpyrimidin-4-amine

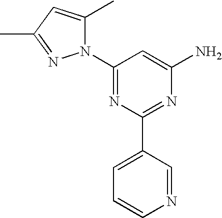

Obtained from Intermediate 44 (1.50 g) by the procedure described in Example 21. Purification by column chromatography with silica gel and methylene chloride/methanol (5%) as eluent gave 6-(3,5-dimethyl-1H-pyrazol-1-yl)-2-pyridin-3-ylpyrimidin-4-amine (1.25 g, 63%) as an off-white solid.

δ (250 MHz, DMSO-d₆): 2.20 (s, 3H); 2.76 (s, 3H); 6.15 (s, 1H); 6.86 (s, 1H); 7.18 (bs, 2H); 7.56-7.51 (m, 1H); 8.53-8.52 (m, 1H); 8.69-8.66 (m, 1H); 9.42-9.41 (m, 1H).

### EXAMPLE 105

### N-[6-(3,5-Dimethyl-1H-pyrazol-1-yl)-2-pyridin-3-ylpyrimidin-4-yl]-acetamide

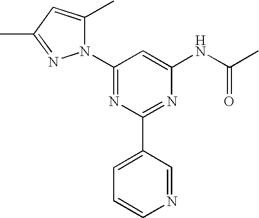

Obtained from the title compound of Example 104 (0.30 g) by the procedure described in Example 2. Purification by column chromatography with silica gel and methylene chloride/methanol (4%) as eluent, followed by a second column chromatography with silica gel and ethyl acetate/n-hexane/methanol (85:13:2) as eluent gave N-[6-(3,5-dimethyl-1H-pyrazol-1-yl)-2-pyridin-3-ylpyrimidin-4-yl]acetamide (92 mg, 26%) as an off-white solid.

δ (250 MHz, CDCl₃): 2.30 (s, 3H); 2.31 (s, 3H); 2.81 (s, 3H); 6.04 (s, 1H); 7.41 (dd, J1=7.9 Hz, J2=4.8 Hz, 1H); 8.46 (bs, 1H); 8.60-8.55 (m, 2H); 8.71 (dd, J1=4.8 Hz, J2=1.5 Hz, 1H); 9.58-9.56 (m, 1H).

### EXAMPLE 106

### N-[6-(3,5-Dimethyl-1H-pyrazol-1-yl)-2-pyridin-3-ylpyrimidin-4-yl]-propanamide

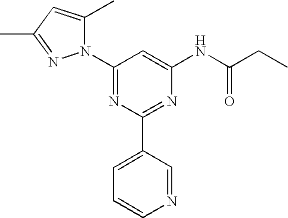

Obtained from the title compound of Example 104 (0.30 g) by the procedure described in Example 2. Purification by column chromatography with silica gel and methylene chloride/methanol (from 4% to 10%) as eluent gave N-[6-(3,5-dimethyl-1H-pyrazol-1-yl)-2-pyridin-3-ylpyrimidin-4-yl]propanamide (0.13 g, 34%) as an off-white solid.

δ (250 MHz, CDCl₃): 1.29 (t, J=7.6 Hz, 3H); 2.30 (s, 3H); 2.53 (q, J=7.6 Hz, 2H); 2.83 (s, 3H); 6.05 (s, 1H); 7.42 (ddd, J1=8.1 Hz, J2=4.8 Hz, 1H); 8.08 (bs, 1H); 8.60 (dt, J1=8.1 Hz, J2=2.0 Hz, 1H); 8.65 (s, 1H); 8.72 (dd, J1=4.8 Hz, J2=1.5 Hz, 1H); 9.59-9.57 (m, 1H).

### EXAMPLE 107

### N-[6-(3,5-Dimethyl-1H-pyrazol-1-yl)-2-pyridin-3-ylpyrimidin-4-yl]-3,3,3-trifluoropropanamide

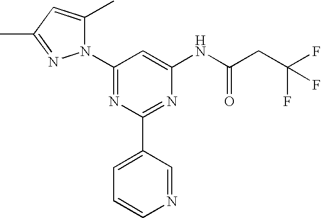

Obtained from the title compound of Example 104 (0.30 g) by the procedure described in Example 14. Purification by column chromatography with silica gel and methylene chloride/methanol (from 4% to 10%) as eluent gave N-[6-(3,5-dimethyl-1H-pyrazol-1-yl)-2-pyridin-3-ylpyrimidin-4-yl]-3,3,3-trifluoropropanamide (0.11 g, 28%) as an off-white solid.

δ (250 MHz, DMSO-d₆): 2.24 (s, 3H); 2.81 (s, 3H); 3.77 (q, J=10.9 Hz, 2H); 6.25 (s, 1H); 7.61 (dd, J1=7.9 Hz, J2=4.7 Hz, 1H); 8.47 (s, 1H); 8.60 (dd, J1=7.9 Hz, J2=1.4 Hz, 1H); 8.75 (dd, J1=4.7 Hz, J2=1.4 Hz, 1H); 9.46 (s, 1H); 11.41 (s, 1H).

### EXAMPLE 108

### 2-Pyridin-3-yl-6-(1H-1,2,4-triazol-1-yl)pyrimidin-4-amine

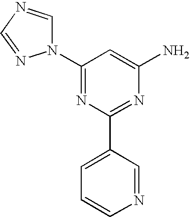

Obtained from Intermediate 44 (1.50 g) by the procedure described in Example 21. Purification by column chromatography with silica gel and methylene chloride/methanol (4%) as eluent gave 2-pyridin-3-yl-6-(1H-1,2,4-triazol-1-yl)pyrimidin-4-amine (0.26 g, 15%) as an off-white solid.

δ (250 MHz, DMSO-d₆): 6.83 (s, 1H); 7.57-7.52 (m, 3H); 8.34 (s, 1H); 8.74-8.69 (m, 2H); 9.58-9.57 (m, 1H); 9.67 (s, 1H).

### EXAMPLE 109

### 3,3,3-Trifluoro-N-[2-pyridin-3-yl-6-(1H-1,2,4-triazol-1-yl)pyrimidin-4-yl]propanamide

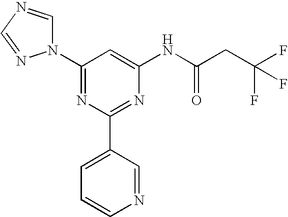

Obtained from the title compound of Example 108 (0.15 g) by the procedure described in Example 14. Purification by column chromatography with silica gel and methylene chloride/methanol (from 4% to 10%) as eluent gave 3,3,3-trifluoro-N-[2-pyridin-3-yl-6-(1H-1,2,4-triazol-1-yl)pyrimidinyl]propanamide (73 mg, 33%) as an off-white solid.

δ (250 MHz, DMSO-d₆): 3.83 (q, J=10.9 Hz, 2H); 7.95-7.90 (m, 1H); 8.45 (s, 1H); 8.46 (s, 1H); 8.97-8.93 (m, 1H); 9.10 (d, J=8.2 Hz, 1H); 9.74 (bs, 1H); 9.95 (s, 1H); 11.81 (s, 1H).

### Intermediate 45. 6-(2-Furyl)-2-pyridin-3-ylpyrimidin-4-ol

Obtained from Intermediate 35 (1.00 g) and Intermediate 41 (1.08 g) by the procedure described in Intermediate 36. Purification by trituration with n-pentane gave the title compound (0.27 g, 20%) as a brown solid.

δ (200 MHz, DMSO-d₆): 6.26 (s, 1H); 6.64 (d, J=1.7 Hz, 1H); 7.12 (d, J=3.4 Hz, 1H); 7.44-7.50 (m, 1H); 7.81 (s, 1H); 8.54 (s, 1H); 8.62 (d, J=4.7 Hz, 1H); 9.41 (s, 1H).

### Intermediate 46. 4-Chloro-6-(2-furyl)-2-pyridin-3-ylpyrimidine

Obtained from Intermediate 45 (0.69 g) by the procedure described in Intermediate 10. Purification by column chromatography with silica gel and methylene chloride/methanol (from pure methylene chloride to 15% methanol) gave the title compound (0.32 g, 43%) as a brown solid, which was used in the next step without further characterisation.

### EXAMPLE 110

### 6-(2-Furyl)-2-pyridin-3-ylpyrimidin-4-ylamine

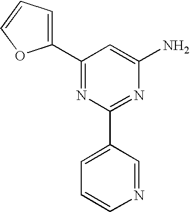

Obtained from Intermediate 46 (0.32 g) by the procedure described in Example 48. Purification by column chromatography with silica gel and methylene chloride gave 6-(2-furyl)-2-pyridin-3-ylpyrimidin-4-ylamine (80 mg, 27%) as an off-white solid.

δ (300 MHz, DMSO-d₆): 6.69 (dd, J1=3.6 Hz, J2=1.9 Hz, 1H); 6.70 (s, 1H); 7.15 (bs, 2H); 7.28 (dd, J1=3.3 Hz, J2=0.8 Hz, 1H); 7.51 (dd, J1=8.0 Hz, J2=4.7 Hz, 1H); 7.89 (dd, J1=1.9 Hz, J2=0.8 Hz, 1H); 8.61 (dt, J1=8.0 Hz, J2=1.9 Hz, 1H); 8.66 (bs, 1H); 9.47 (bs, 1H).

### EXAMPLE 111

### N-[6-(2-Furyl)-2-pyridin-3-ylpyrimidin-4-yl]propanamide

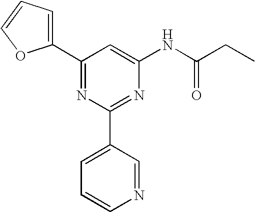

Obtained from the title compound of Example 110 (55 mg) by the procedure described in Example 49. Purification by trituration with n-pentane gave N-[6-(2-furyl)-2-pyridin-3-ylpyrimidin-4-yl]propanamide (28 mg, 41%) as an off-white solid.

δ (300 MHz, DMSO-d₆): 1.11 (t, J=7.6 Hz, 3H); 2.53 (q, J=7.6 Hz, 2H); 6.79-6.77 (m, 1H); 7.51 (d, J=3.6 Hz, 1H); 7.59 (dd, J1=8.5 Hz, J2=4.4 Hz, 1H); 8.03-8.02 (m, 1H); 8.37 (s, 1H); 8.75-8.68 (m, 2H); 9.57 (d, J=1.8 Hz, 1H); 11.01 (s, 1H).

### Intermediate 47. 6-Amino-2-pyridin-ylpyrimidin-4-ol

Obtained from pyridine-4-carboxamidine, hydrochloride (2.13 g) by the procedure described in Intermediate 9. Purification by trituration with ethyl ether gave the title compound (1.22 g, 48%) as an off-white solid.

δ (300 MHz, DMSO-d₆): 5.27 (s, 1H); 6.70 (s, 2H); 8.00 (d, J=6.1 Hz, 2H); 8.71 (d, J=6.1 Hz, 2H); 11.74 (bs, 1H).

### Intermediate 48. N-[6-Chloro-2-pyridin-4-ylpyrimidin-4-yl]propanamide

Obtained from Intermediate 47 (1.22 g) by the procedure described in Intermediate 12. Purification by column chromatography with silica gel and ethyl acetate/n-hexane (from 2:1 to 4:1) as eluent gave impure N-[6-chloro-2-pyridin-4-ylpyrimidin-4-yl]propanamide (0.90 g). Purification (0.49 g) by column chromatography with silica gel and chloroform/methanol (3%) as eluent gave the title compound (0.35 g, 38%) as an off-white solid.

δ (200 MHz, ClCD₃): 1.29 (t, J=7.5 Hz, 3H); 2.57 (q, J=7.5 Hz, 2H); 8.20 (d, J=6.1 Hz, 2H); 8.26 (s, 1H); 8.40 (bs, 1H); 8.80 (d, J=6.1 Hz, 2H).

### EXAMPLES 112 AND 113

### N-[6-(3,5-Dimethyl-1H-pyrazol-1-yl)-2-pyridin-4-ylpyrimidin-4-yl]propanamide and 6-(3,5-dimethyl-1H-pyrazol-1-yl)-2-pyridin-4-ylpyrimidin-4-amine

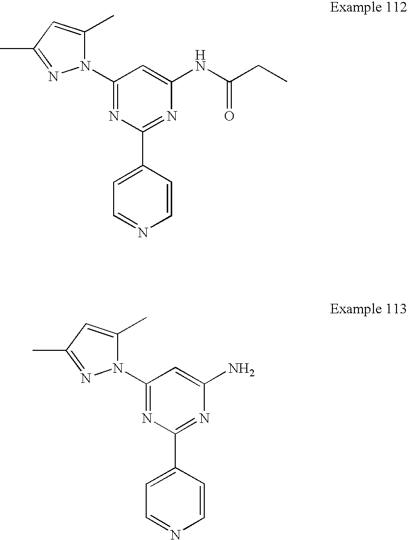

Obtained from Intermediate 48 (0.17 g) by the procedure described in Example 21 (reaction temperature: 85° C., reaction time: 20 hours). Purification by preparative HPLC-MS gave N-[6-(3,5-dimethyl-1H-pyrazol-1-yl)-2-pyridin-4-ylpyrimidin-4-yl]propanamide (19 mg, 9%) and 6-(3,5-dimethyl-1H-pyrazol-1-yl)-2-pyridin-4-ylpyrimidin-4-amine (5 mg, 3%) as off-white solids.

### EXAMPLE 112

δ (300 MHz, CDCl₃): 1.30 (t, J=7.4 Hz, 3H); 2.30 (s, 3H); 2.53 (q, J=7.4 Hz, 2H); 2.85 (s, 3H); 6.05 (s, 1H); 8.12 (bs, 1H); 8.17 (d, J=6.1 Hz, 2H); 8.70 (s, 1H); 8.77 (d, J=6.1 Hz, 2H).

### EXAMPLE 113

δ (300 MHz, CDCl₃): 2.33 (s, 3H); 2.84 (s, 3H); 5.02 (bs, 2H); 6.06 (s, 1H); 7.00 (s, 1H); 8.21-8.19 (m, 2H); 8.76 (bs, 2H).

### Intermediate 49. 6-(2-Furyl)-2-pyridin-4-ylpyrimidin-4-ol

Obtained from Intermediate 35 (1.00 g) and pyridine-4-carboxamidine, hydrochloride by the procedure described in Intermediate 36. Purification by trituration with n-pentane gave the title compound (0.38 g, 29%) as a brown solid.

δ (200 MHz, DMSO-d₆): 6.74 (dd, J1=3.4 Hz, J2=1.7 Hz, 1H); 7.37 (d, J=3.4 Hz, 1H); 7.96 (d, J=1.7 Hz, 1H); 8.16 (d, J=6.4 Hz, 2H); 8.79 (d, J=6.4 Hz, 2H).

### Intermediate 50. 4-Chloro-6-(2-furyl)-2-pyridin-4-ylpyrimidine

Obtained from Intermediate 49 (0.63 g) by the procedure described in Intermediate 15 (reaction time: 2 hours). 4-Chloro-6-(2-furyl)-2-pyridin-4-ylpyrimidine (0.51 g, 76%) was obtained as a brown solid.

δ (200 MHz, CDCl₃): 6.66-6.68 (m, 1H); 7.49 (d, J=3.4 Hz, 1H); 7.65 (d, J=1.7 Hz, 1H); 7.68 (s, 1H); 8.44 (d, J=4.9 Hz, 2H); 8.83 (d, J=4.9 Hz, 2H).

### EXAMPLE 114

### 6-(2-Furyl)-2-pyridin-4-ylpyrimidin-4-ylamine

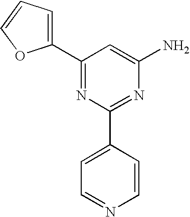

Obtained from Intermediate 50 (0.51 g) by the procedure described in Example 48. Purification by column chromatography with silica gel and methylene chloride/methanol (from pure methylene chloride to 2% methanol) as eluent gave 6-(2-furyl)-2-pyridin-4-ylpyrimidin-4-amine (0.24 g, 51%) as an off-white solid.

δ (300 MHz, CDCl₃): 4.99 (bs, 2H); 6.57-6.55 (m, 1H); 6.75 (s, 1H); 7.28 (d, J=3.6 Hz, 1H); 7.55-7.54 (m, 1H); 8.25 (d, J=6.1 Hz, 2H); 8.72 (d, J=6.1 Hz, 2H).

### EXAMPLE 115

### N-[6-(2-Furyl)-2-pyridin-ylpyrimidin-4-yl]propanamide

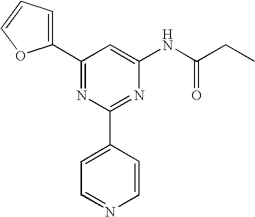

Obtained from the title compound of Example 114 (0.14 g) by the procedure described in Example 49. Purification by trituration with ethyl ether gave N-[6-(2-furyl)-2-pyridin-4-ylpyrimidin-4-yl]propanamide (0.13 g, 75%) as an off-white solid.

δ (300 MHz, DMSO-d₆): 1.10 (t, J=7.6 Hz, 3H); 2.53 (q, J=7.6 Hz, 2H); 6.78 (dd, J1=3.4 Hz, J2=1.8 Hz, 1H); 7.51 (d, J=3.4 Hz, 1H); 8.03 (d, J=2.4 Hz, 1H); 8.29 (d, J=6.1 Hz, 2H); 8.41 (s, 1H); 8.80 (d, J=6.1 Hz, 2H); 11.07 (s, 1H).

### Intermediate 51. 6-(2-Furyl)-2-(1,3-thiazol-2-yl)pyrimidin-4-ol

Obtained from thiazole-2-carboxamidine, hydrochloride (prepared by the procedure described in Intermediate 1) and Intermediate 35 (1.00 g) by the procedure described in Intermediate 36. Purification by column chromatography with silica gel and chloroform/methanol (5%) as eluent gave the title compound (0.40 g, 29%) as an off-white solid.

δ (300 MHz, CDCl₃): 6.60 (s, 1H); 6.74-6.72 (m, 1H); 7.27-7.24 (m, 1H); 7.97 (s, 1H); 8.15-8.12 (m, 2H).

### Intermediate 52. 4-Chloro-6-(2-furyl)-2-(1,3-thiazol-2-yl)pyrimidine

Obtained from Intermediate 51 (0.39 g) by the procedure described in Intermediate 10. Purification by column chromatography with silica gel and methylene chloride as eluent gave 4-chloro-6-(2-furyl)-2-(1,3-thiazol-2-yl)pyrimidine (0.20 g, 48%) as a pale yellow solid.

δ (300 MHz, CDCl₃): 6.64-6.62 (m, 1H); 7.53-7.51 (m, 1H); 7.59-7.57 (m, 1H); 7.64 (s, 1H); 7.67 (s, 1H); 8.09 (s, 1H).

### EXAMPLE 116

### 6-(2-Furyl)-2-(1,3-thiazol-2-yl)pyrimidin-4-amine

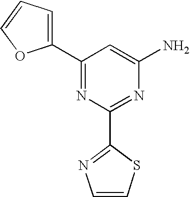

Obtained from Intermediate 52 (0.20 g) by the procedure described in Example 48. Purification by column chromatography with silica gel and methylene chloride/ethyl ether (7:3) as eluent gave 6-(2-furyl)-2-(1,3-thiazol-2-yl)pyrimidin-4-amine (95 mg, 51%) as an off-white solid.

δ (300 MHz, CDCl₃): 5.22 (bs, 2H); 6.56 (dd, J1=3.4 Hz, J2=1.8 Hz, 1H); 6.76 (s, 1H); 7.32 (dd, J1=3.6 Hz, J2=0.8 Hz, 1H); 7.48 (d, J=3.0 Hz, 1H); 7.55 (dd, J1=1.8 Hz, J2=0.8 Hz, 1H); 7.99 (d, J=3.0 Hz, 1H).

### EXAMPLE 117

### N-[6-(2-Furyl)-2-(1,3-thiazol-2-yl)pyrimidin-4-yl]propanamide

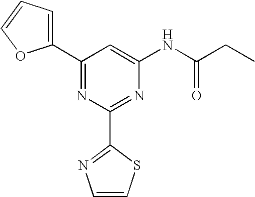

Obtained from the title compound of Example 116 (95 mg) by the procedure described in Example 49. Purification by column chromatography with silica gel and ethyl acetate/n-hexane (from 1:1 to pure ethyl acetate) as eluent gave N-[6-(2-furyl)-2-(1,3-thiazol-2-yl)pyrimidin-4-yl]propanamide (68 mg, 58%) as an off-white solid.

δ (300 MHz, CDCl₃): 1.27 (t, J=7.4 Hz, 3H); 2.48 (q, J=7.4 Hz, 2H); 6.59 (dd, J1=3.6 Hz, J2=1.7 Hz, 1H); 7.40 (dd, J1=3.6 Hz, J2=0.8 Hz, 1H); 7.53 (d, J=3.3 Hz, 1H); 7.63 (dd, J1=1.7 Hz, J2=0.8 Hz, 1H); 8.02 (d, J=3.3 Hz, 1H); 8.24 (bs, 1H); 8.47 (s, 1H).

### EXAMPLE 118

### 2-(4-Fluorophenyl)-N-[6-(2-furyl)-2-(1,3-thiazol-2-yl)pyrimidin-4-yl]acetamide

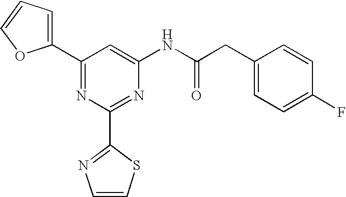

A solution of the title compound of Example 116 (98 mg, 0.4 mmol) and 4-fluorophenylacetyl chloride (164 μL, 1.20 mmol) in pyridine (6 mL) was heated at 120° C. overnight. The solvent was removed under reduced pressure. Methylene chloride was added (20 mL) and the solution was washed with water (2×10 mL), brine (10 mL), and dried (Na₂SO₄). The solvent was removed under reduced pressure. Purification by column chromatography with silica gel, eluting with ethyl acetate/n-hexane (from pure to 50% of n-hexane), gave 2-(4-fluorophenyl)-N-[6-(2-furyl)-2-(1,3-thiazol-2-yl)pyrimidinyl]acetamide (62 mg, 59%) as an off-white solid.

δ (300 MHz, CDCl₃): 3.74 (s, 2H); 6.58 (dd, J1=3.4 Hz, J2=1.8 Hz, 1H); 7.07 (t, J=8.6 Hz, 2H); 7.32-7.27 (m, 2H); 7.38 (dd, J1=3.4 Hz, J2=0.8 Hz, 1H); 7.52 (d, J=3.0 Hz, 1H); 7.61 (d, J=2.8 Hz, 1H); 8.00 (d, J=3.0 Hz, 1H); 8.22 (bs, 1H); 8.46 (s, 1H).

### EXAMPLE 119

### N-(Cyclopropylmethyl)-2-(2-furyl)-6-(1H-pyrazol-1-yl)pyrimidin-4-amine

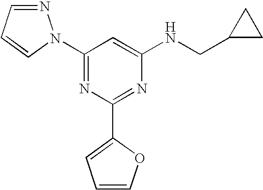

A solution of Intermediate 5 (0.50 g, 2.03 mmol) and cyclopropylmethylamine (0.43 g, 6.08 mmol) in pentanol (12.5 mL) was heated at 100° C. overnight. The solvent was removed under reduced pressure. Purification by column chromatography with silica gel, eluting with chloroform, gave N-(cyclopropylmethyl)-2-(2-furyl)-6-(1H-pyrazol-1-yl)pyrimidin-4-amine (0.55 g, 70%) as a solid.

m.p.: 100.9-101.7° C.

δ (300 MHz, CDCl₃): 0.27-0.32 (m, 2H); 0.57-0.63 (m, 2H); 1.06-1.18 (m, 1H); 3.24 (bs, 2H); 5.48 (bs, 1H); 6.47 (dd, J1=2.6 Hz, J2=1.6 Hz, 1H); 6.56 (dd, J1=3.3 Hz, J2=1.6 Hz, 1H); 6.78 (s, 1H); 7.29 (dd, J1=3.3 Hz, J2=0.8 Hz, 1H); 7.61 (dd, J1=1.9 Hz, J2=0.8 Hz, 1H); 7.76 (d, J=0.8 Hz, 1H); 8.66 (dd, J1=2.6 Hz, J2=0.8 Hz, 1H).

### EXAMPLE 120

### (2R)-2-{[2-(2-Furyl)-6-(1H-pyrazol-1-yl)pyrimidin-4-yl]amino}propan-1-ol

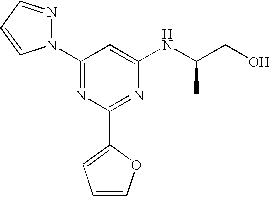

Obtained from Intermediate 5 (100 mg) and (R)-2-aminopropanol (189 μL, 2.43 mmol) by the procedure described in Example 119. Purification by column chromatography with silica gel and methylene chloride/methanol (from pure methylene chloride to 95:5) as eluent gave (2R)-2-{[2-(2-furyl)-6)-(1H-pyrazol-1-yl)pyrimidin-4-yl]amino}propan-1-ol (88 mg, 76%) as an off-white solid.

m.p.: 163.0-163.8° C.

δ (300 MHz, CDCl₃): 1.32 (d, J=6.7 Hz, 3H); 3.65-3.83 (m, 3H); 4.11 (bs, 1H); 5.27 (bs, 1H); 6.47 (dd, J1=2.5 Hz, J2=1.7 Hz, 1H); 6.55 (dd, J1=3.3 Hz, J2=1.7 Hz, 1H); 6.84 (s, 1H); 7.29 (dd, J1=3.3 Hz, J2=0.8 Hz, 1H); 7.61 (dd, J1=1.6 Hz, J2=0.8 Hz, 1H); 7.75 (dd, J1=1.6 Hz, J2=0.7 Hz, 1H); 8.64 (dd, J1=2.5 Hz, J2=0.7 Hz, 1H).

### EXAMPLE 121

### 3-{[2-(2-Furyl)-6-(1H-pyrazol-1-yl)pyrimidin-4-yl]amino}propan-1-ol

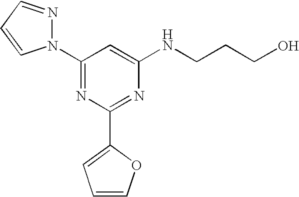

Obtained from Intermediate 5 (100 mg) and 3-amino-1-propanol (93 μL, 1.22 mmol) by the procedure described in Example 119. Purification by column chromatography with silica gel and methylene chloride/methanol (from pure methylene chloride to 95:5) as eluent gave 3-{[2-(2-furyl)-6-(1H-pyrazol-1-yl)pyrimidin-4-yl]amino}propan-1-ol (104 mg, 90%) as an off-white solid.

δ (300 MHz, CDCl₃): 1.80-1.86 (m, 2H); 3.70 (bs, 4H); 5.31 (bs, 1H); 6.47 (dd, J1=2.6 Hz, J2=1.6 Hz, 1H); 6.55 (dd, J1=3.5 Hz, J2=1.6 Hz, 1H); 6.82 (s, 1H); 7.29 (dd, J1=3.5 Hz, J2=0.8 Hz, 1H); 7.61 (dd, J1=1.6 Hz, J2=0.8 Hz, 1H); 7.75 (dd, J1=1.6 Hz, J2=0.8 Hz, 1H) 8.64 (dd, J1=2.6 Hz, J2=0.8 Hz, 1H).

### EXAMPLE 122

### N-[2-(2-Furyl)-6-(1H-pyrazol-1-yl)pyrimidin-4-yl]ethane-1,2-diamine

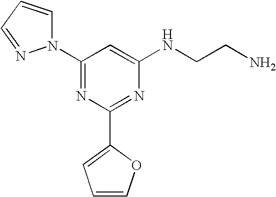

The precursor intermediate tert-butyl 2-{[2-(2-furyl)-6-(1H-pyrazol-1-yl)pyrimidin-4-yl]amino}ethylcarbamate was obtained from Intermediate 5 (145 mg) and N—BOC-ethylenediamine (279 μL, 1.76 mmol) by the procedure described in Example 119. Purification by column chromatography with silica gel and methylene chloride/methanol (from pure methylene chloride to 95:5) as eluent gave 351 mg, (80%) of the intermediate.

To a solution of the intermediate tert-butyl 2-{[2-(2-furyl)-6-(pyrazol-1-yl)pyrimidin-4-yl]amino}ethylcarbamate (0.28 g, 0.76 mmol) in chloroform (1.7 mL) was added trifluoroacetic acid (0.58 mL, 7.56 mmol). The mixture was stirred at room temperature for 3 hours. The solvent was remove under reduced pressure. To the residue was added water (25 mL), potassium carbonate until basic pH, and methylene chloride (2×20 mL). The organic layer was dried (Na₂SO₄) and the solvent removed under reduced pressure. Purification of the obtained residue by trituration with ethyl ether/isopropyl ether (1:1) gave N-[2-(2-furyl)-6-(1H-pyrazol-1-yl)pyrimidin-4-yl]ethane-1,2-diamine (77 mg, 38%) as an off white solid.

m.p.: 104.1-105.1° C.

δ (300 MHz, CDCl₃): 2.99-3.03 (t, J=5.9 Hz, 2H); 3.46-3.50 (m, 2H); 5.69 (bs, 1H); 6.47 (dd, J1=2.6 Hz, J2=1.6 Hz, 1H); 6.55 (dd, J1=3.4 Hz, J2=1.8 Hz, 1H); 6.83 (s, 1H); 7.29 (d, J=3.3 Hz, 1H) 7.60-7.61 (m, 1H); 7.75-7.76 (m, 1H); 8.65 (d, J=2.6 Hz, 1H).

### EXAMPLE 123

### 2-(2-Furyl)-N-[2-(4-methoxyphenyl)ethyl]-6-(1H-pyrazol-1-yl)-pyrimidin-amine

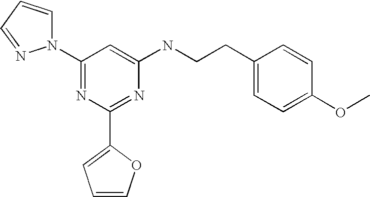

Obtained from Intermediate 5 (100 mg) and (4-methoxyphenyl)ethylamine (177 μL, 1.22 mmol) by the procedure described in Example 119. Purification by column chromatography with silica gel and n-hexane/ethyl acetate (from 10:1 to 2:1) as eluent gave 2-(2-furyl)-N-[2-(4-methoxyphenyl)ethyl]-6-(1H-pyrazol-1-yl)pyrimidin-4-amine (111 mg, 76%) as an oil.

δ (300 MHz, CDCl₃): 2.92 (t, J=7.0 Hz, 2H); 3.65 (bs, 2H); 3.80 (s, 3H); 5.28 (bs, 1H); 6.47 (dd, J1=2.5 Hz, J2=1.7 Hz, 1H); 6.55 (dd, J1=3.3 Hz, J2=1.7 Hz, 1H); 6.80 (s, 1H); 6.85-6.88 (m, 2H); 7.15-7.18 (m, 2H); 7.29 (dd, J=3.3 Hz, J2=0.7 Hz, 1H); 7.59-7.60 (m, 1H); 7.76 (d, J=1.1 Hz, 1H); 8.65 (dd, J1=2.6 Hz, J2=0.7 Hz, 1H).

### EXAMPLE 124

### N-[2-(3,4-Dimethoxyphenyl)ethyl]-2-(2-furyl)-6-(1H-pyrazol-1-yl)pyrimidin-4-amine

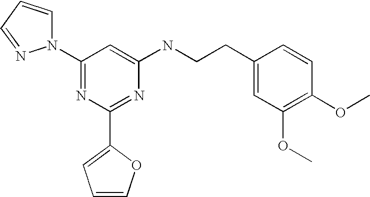

Obtained from Intermediate 5 (100 mg) and (3,4-dimethoxyphenyl)ethylamine (177 μL, 1.22 mmol) by the procedure described in Example 119. Purification by column chromatography with silica gel and methylene chloride/methanol (from pure methylene chloride to 99:1) as eluent gave N-[2-(3,4-dimethoxyphenyl)ethyl]-2-(2-furyl)-6-(1H-pyrazol-1-yl)pyrimidin-4-amine (77 mg, 49%) as an oil.

δ (300 MHz, CDCl₃): 2.92 (t, J=7.0 Hz, 2H); 3.67 (bs, 2H); 3.87 (s, 3H); 3.88 (s, 3H); 5.30 (bs, 1H); 6.47 (dd, J1=2.5 Hz, J2=1.7 Hz, 1H); 6.55 (dd, J1=3.3 Hz, J2=1.7 Hz, 1H); 6.75-6.76 (m, 1H); 6.80-6.82 (m, 3H); 7.29 (dd, J1=3.3 Hz, J2=0.9 Hz, 1H); 7.60 (dd, J1=1.7 Hz, J2=0.9 Hz, 1H); 7.75-7.77 (m, 1H); 8.65 (dd, J=2.6 Hz, J2=0.7 Hz, 1H).

### EXAMPLE 125

### 2-(2-Furyl)-6-(1H-pyrazol-1-yl)-N-[2-(pyridin-2-yl)ethyl]pyrimidin-4-amine

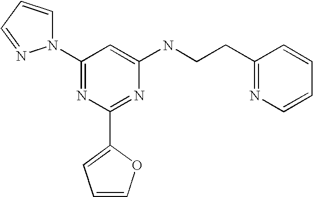

Obtained from Intermediate 5 (100 mg) and 2-(2-aminoethyl)pyridine (145 μL, 1.22 mmol) by the procedure described in Example 119. Purification by column chromatography with silica gel and methylene chloride/methanol (from pure methylene chloride to 8:2) as eluent gave 2-(2-furyl)-6-(1H-pyrazol-1-yl)-N-[2-(pyridin-2-yl)ethyl]pyrimidin-4-amine (86 mg, 64%) as an off-white solid.

m.p.: 110.2-110.9° C.

δ (300 MHz, CDCl₃): 3.14 (t, J=6.5 Hz, 2H); 3.88 (bs, 2H); 5.95 (bs, 1H); 6.47 (dd, J1=2.5 Hz, J2=1.7 Hz, 1H); 6.55 (dd, J1=3.3 Hz, J2=1.7 Hz, 1H); 6.84 (s, 1H); 7.14-7.20 (m, 2H); 7.29 (dd, J1=3.3 Hz, J2=0.8 Hz, 1H); 7.59-7.65 (m, 2H); 7.75-7.76 (m, 1H); 8.56-8.59 (m, 1H); 8.65 (dd, J1=2.5 Hz, J2=0.8 Hz, 1H).

### EXAMPLE 126

### 2-(2-Furyl)-6-(1H-pyrazol-1-yl)-N-[2-(pyridin-3-yl)ethyl]pyrimidin-4-amine

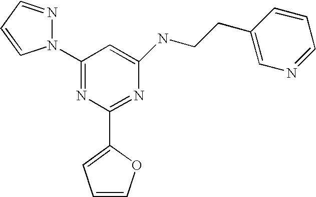

Obtained from Intermediate 5 (100 mg) and 3<2-aminoethyl)pyridine (149 mg, 1.22 mmol) by the procedure described in Example 119. Purification by column chromatography with silica gel and chloroform/methanol (from pure chloroform to 1% methanol) as eluent gave 2-(2-furyl)-6-(1H-pyrazol-1-yl)/[2-(pyridin-3-yl)ethyl]pyrimidin-4-amine (94 mg, 70%) as an off-white solid.

m.p.: 164.0-164.9° C.

δ (300 MHz, CDCl₃): 3.00 (t, J=6.9 Hz, 2H); 3.71-3.75 (m, 2H); 5.30 (s, 1H); 6.48 (s, 1H); 6.55-6.56 (m, 1H); 6.82 (s, 1H); 7.24-7.30 (m, 3H); 7.57-7.60 (m, 2H); 7.76 (s, 1H); 8.50-8.53 (m, 2H); 8.65-8.66 (m, 1H).

### EXAMPLE 127

### 2-(2-Furyl)-N-(3-phenylpropyl)-6-(1H-pyrazol-1-yl)pyrimidin-4-amine

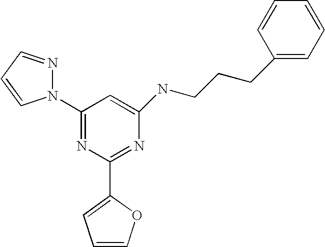

Obtained from Intermediate 5 (100 mg) and 3-phenylpropylamine (173 μL, 1.22 mmol) by the procedure described in Example 119. Purification by column chromatography with silica gel and n-hexane/ethyl acetate (from 10:1 to 2:1) as eluent gave 2-(2-furyl)-N-(3-phenylpropyl)-6-(1H-pyrazol-1-yl)pyrimidin-amine (124 mg, 89%) as an off-white solid.

δ (300 MHz, CDCl₃): 2.01 (q, J=7.5 Hz, 2H); 2.76 (t, J=7.5 Hz, 2H); 3.41 (bs, 2H); 5.34 (bs, 1H); 6.47 (dd, J1=2.5 Hz, J2=1.7 Hz, 1H); 6.56 (dd, J1=3.3 Hz, J2=1.7 Hz, 1H); 6.77 (s, 1H); 7.19-7.31 (m, 6H); 7.61 (s, 1H); 7.76 (s, 1H); 8.65-8.66 (m, 1H)

### EXAMPLE 128

### 2-(2-Furyl)-N-[3-(1H-imidazol-1-yl)propyl]-6-(1H-pyrazol-1-yl)pyrimidin-4-amine

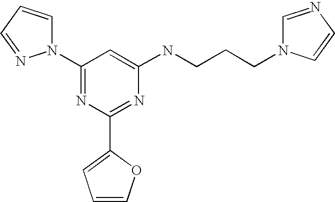

Obtained from Intermediate 5 (100 mg) and 3-(imidazol-1-yl)propylamine (145 μL, 1.22 mmol) by the procedure described in Example 119. Purification by column chromatography with silica gel and methylene chloride/methanol (from pure methylene chloride to 97:3) as eluent gave 2-(2-furyl)-N-[3-1H-imidazol-1-yl)propyl]-6-(1H-pyrazol-1-yl)pyrimidin-4-amine (122 mg, 90%) as an off-white solid.

δ (300 MHz, CDCl₃): 2.18 (q, J=6.7 Hz, 2H); 3.43-3.50 (m, 2H); 4.11 (t, J=6.7 Hz, 2H); 5.26 (bs, 1H); 6.49 (dd, J1=2.6 Hz, J2=1.7 Hz, 1H); 6.58 (dd, J1=3.3 Hz, J2=1.7 Hz, 1H); 6.79 (s, 1H); 6.97-6.98 (m, 1H); 7.10-7.11 (m, 1H); 7.30 (dd, J1=3.3 Hz, J2=0.8 Hz, 1H); 7.54 (bs, 1H); 7.63 (dd, J1=1.7 Hz, J2=0.8 Hz, 1H) 7.76-7.77 (dd, J1=1.7 Hz, J2=0.8 Hz, 1H); 8.66 (dd, J1=2.6 Hz, J2=0.8 Hz, 1H).

### Intermediate 53. 2-(2-Thienyl)pyrimidine-4,6-diol

Obtained from Intermediate 13 (1.35 g) by the procedure described in Intermediate 2. Purification by trituration with diisopropyl ether gave 2-(2-thienyl)pyrimidine-4,6-diol (0.44 g, 34%) as a pale yellow solid.

δ (300 MHz, DMSO-d₆): 5.15 (s, 1H); 7.07-7.19 (m; 1H); 7.72-7.78 (m, 1H); 8.00-8.02 (m, 1H).

### Intermediate 54. 4,6-Dichloro-2-(2-thienyl)pyrimidine

Obtained from Intermediate 53 (0.44 g) by the procedure described in Intermediate 3. Purification by trituration with diisopropyl ether gave 4,6-dichloro-2-(2-thienyl)pyrimidine (0.41 g, 78%) as a brown solid.

δ (300 MHz, CDCl₃): 7.12-7.20 (m, 2H); 7.54-7.60 (m, 1H); 8.05-8.08 (m, 1H).

### Intermediate 55. 4-Chloro-6-(1H-pyrazol-1-yl)-2-(2-thienyl)pyrimidine

Obtained from Intermediate 54 (0.69 g) by the procedure described in Intermediate 5. Purification by column chromatography with silica gel and n-hexane/ethyl acetate (from pure n-hexane to 3:1) as eluent gave 4-chloro-6-(1H-pyrazol-1-yl)-2-(2-thienyl)pyrimidine (0.49 g, 68%) as an off-white solid.

δ (300 MHz, CDCl₃): 6.56 (s, 1H); 7.16-7.20 (m, 1H); 7.54-7.58 (m, 1H); 7.75 (s, 1H); 7.84 (s, 1H); 8.08-8.11 (m, 1H); 8.68 (s 1H).

### EXAMPLE 129

### N-(Cyclopropylmethyl)-6-(1H-pyrazol-1-yl)-2-(2-thienyl)pyrimidin-4-amine

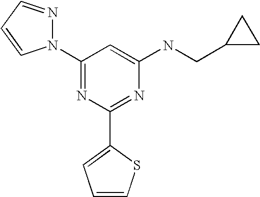

Obtained from Intermediate 55 (100 mg) and cyclopropylmethylamine (99 μL, 1.14 mmol) by the procedure described in Example 119. Purification by column chromatography with silica gel and n-hexane/ethyl acetate (from 95:5 to 90:10) as eluent gave N-cyclopropylmethyl)-6-(1H-pyrazol-1-yl)-2-(2-thienyl)pyrimidin-4-amine (98 mg, 87%) as an off-white solid.

m.p.: 134.6-135.3° C.

δ (300 MHz, CDCl₃): 0.31 (m, 2H); 0.60 (m, 2H); 1.08-1.18 (m, 1H); 3.27 (bs, 2H); 5.32 (bs, 1H); 6.47 (dd, J1=2.5 Hz, J2=1.7 Hz, 1H); 6.76 (s, 1H); 7.13 (dd, J1=4.9 Hz, J2=3.6 Hz, 1H); 7.44 (dd, J1=4.9 Hz, J2=1.2 Hz, 1H); 7.75 (bs, 1H); 7.97 (d, J1=3.6 Hz, J2=1.2 Hz, 1H); 8.67 (d, J=2.2 Hz, 1H).

### EXAMPLE 130

### (2R)-2-{[6-(1H-Pyrazol-1-yl)-2-(2-thienyl)pyrimidin-4-yl]amino}-propan-1-ol

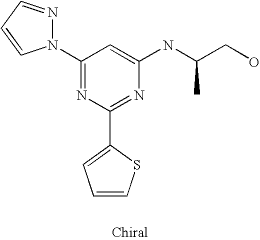

Obtained from Intermediate 55 (100 mg) and (R)-2-aminopropanol (177 μL, 2.28 mmol) by the procedure described in Example 119. Purification by column chromatography with silica gel and n-hexane/ethyl acetate (from pure n-hexane to 3:2) as eluent gave (2R)-2-{[6-(1H-pyrazol-1-yl)-2-(2-thienyl)pyrimidin-4-yl]amino}propan-1-ol (87 mg, 76%) as an off-white solid.

m.p.: 135.7-136.9° C.

δ (300 MHz, CDCl₃): 1.33 (d, J=6.9 Hz, 3H); 3.66-3.72 (m, 1H); 3.81-3.87 (m, 1H); 4.23 (bs, 1H); 5.15 (bs, 1H); 6.48-6.49 (m, 1H); 6.82 (s, 1H); 7.14 (dd, J1=5.1 Hz, J2=3.7 Hz, 1H); 7.46 (dd, J1=5.1 Hz, J2=1.2 Hz, 1H); 7.76 (m, 1H); 7.97 (dd, J1=3.7 Hz, J2=1.2 Hz, 1H); 8.66 (dd, J1=2.7 Hz, J2=1.9 Hz, 1H).

### EXAMPLE 131

### 3-{[6-(1H-Pyrazol-1-yl)-2-(2-thienyl)pyrimidin-4-yl]amino}propan-1-ol

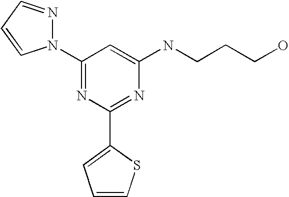

Obtained from Intermediate 55 (100 mg) and 3-amino-1-propanol (87 μL, 1.14 mmol) by the procedure described in Example 119. Purification by column chromatography with silica gel and n-hexane/ethyl acetate (from pure n-hexane to 3:2) as eluent gave 3-{[6-(1H-pyrazol-1-yl)-2-(2-thienyl)pyrimidin-4-yl]amino}propan-1-ol (94 mg, 82%) as an off-white solid.

m.p.: 129.9-130.8° C.

δ (300 MHz, CDCl₃): 1.87 (q, J=6.0 Hz, 2H); 3.73 (bs, 5H); 5.30 (s, 1H); 6.47 (dd, J1=2.6 Hz, J2=1.7 Hz, 1H); 6.79 (s, 1H); 7.13 (dd, J1=4.9 Hz, J2=3.6 Hz, 1H); 7.45 (dd, J1=4.9 Hz, J2=1.2 Hz, 1H); 7.75 (dd, J1=1.7 Hz, J2=0.8 Hz, 1H); 7.96 (dd, J1=3.6 Hz, J2=1.2 Hz, 1H); 8.65 (dd, J1=2.6 Hz, J2=0.8 Hz, 1H).

### EXAMPLE 132

### N-(2-Aminoethyl)-N-[6-(1H-pyrazol-1-yl)-2-(2-thienyl)pyrimidin-4-yl]amine

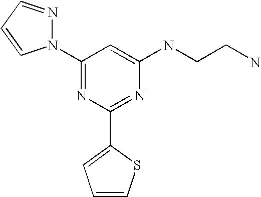

The intermediate tert-butyl 2-{[2-(2-thienyl)-6-(pyrazol-1-yl)pyrimidin-4-yl]amino}-ethylcarbamate was obtained from Intermediate 55 (100 mg) and N—BOC-ethylenediamine (180 μL, 1.14 mmol) by the synthetic procedure described in Example 122. Purification of the final product by column chromatography with silica gel and methylene chloride/methanol/NH₄OH (95:2.5:2.5) as eluent gave N-(2-aminoethyl)-N-[6-(1H-pyrazol-1-yl)-2-(2-thienyl)pyrimidin-4-yl]amine (86 mg, (47%) as a solid.

m.p.: 146.6-147.1° C.

δ (300 MHz, CDCl₃): 0.01 (t, J=5.8 Hz, 2H); 3.50 (bs, 2H); 5.59 (bs, 1H); 6.46-6.48 (m, 1H); 6.80 (s, 1H); 7.11-7.15 (m, 1H); 7.45 (dt, J1=4.9 Hz, J2=1.1 Hz, 1H); 7.75-7.76 (m, 1H); 7.97 (dd, J1=3.7 Hz, J2=1.1 Hz, 1H); 8.66 (d, J=2.7 Hz, 1H).

### EXAMPLE 133

### N-[2-(4-Methoxyphenyl)ethyl]-6-(1H-pyrazol-1-yl)-2-(2-thienyl)pyrimidin-4-amine

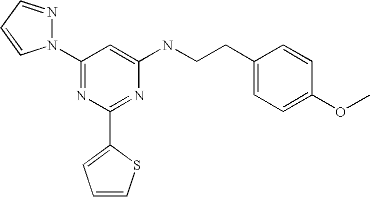

Obtained from Intermediate 55 (100 mg) and (4-methoxyphenyl)ethylamine (166 μL, 1.14 mmol) by the procedure described in Example 119. Purification by column chromatography with silica gel and n-hexane/ethyl acetate (from pure n-hexane to 4:1) as eluent gave N-[2-(4-methoxyphenyl)ethyl]-6-(pyrazol-1-yl)-2-(2-thienyl)pyrimidin-4-amine (121 mg, 84%) as an off-white solid.

m.p.: 99.6-100.4° C.

δ (300 MHz, CDCl₃): 2.92 (t, J=7.0 Hz, 2H); 3.67 (bs, 2H); 3.81 (m, 3H); 5.12 (bs, 1H); 6.47 (dd, J1=2.6 Hz, J2=1.7 Hz, 1H); 6.87 (dt, J1=4.4 Hz, J2=2.6 Hz, 2H); 7.11-7.26 (m, 3H); 7.45 (dd, J1=4.9 Hz, J2=1.4 Hz, 1H); 7.75 (d, J=0.8 Hz, 1H); 7.98 (dd, J1=3.6 Hz, J2=1.1 Hz, 1H); 8.66 (dd, J1=2.6 Hz, J2=0.8 Hz, 1H).

### EXAMPLE 134

### N-[2-(3,4-Dimethoxyphenyl)ethyl]-6-(1H-pyrazol-1-yl)-2-(2-thienyl)pyrimidin-4-amine

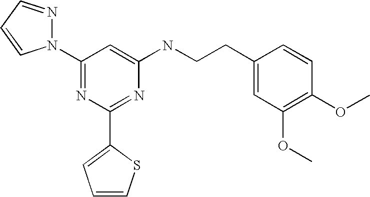

Obtained from Intermediate 55 (100 mg) and (3,4-dimethoxyphenyl)ethylamine (192 μL, 1.14 mmol) by the procedure described in Example 119. Purification by column chromatography with silica gel and n-hexane/ethyl acetate (from 85:15 to 70:30) as eluent gave N-[2-(3,4-dimethoxyphenyl)ethyl]-6-(1H-pyrazol-1-yl)-2-(2-thienyl)pyrimidin-4-amine (117 mg, 76%) as an oil.

m.p.: 116.2-117.3° C.

δ (300 MHz, CDCl₃): 2.93 (t, J=7.0 Hz, 2H); 3.70 (bs, 2H); 3.88 (s, 3H); 3.89 (m, 3H); 5.16 (bs, 1H); 6.47 (dd, J1=2.6 Hz, J2=1.7 Hz, 1H); 6.77-6.85 (m, 4H); 7.13 (dd, J1=4.9 Hz, J2=3.6 Hz, 1H); 7.45 (dd, J1=4.9 Hz, J2=1.4 Hz, 1H); 7.75 (d, J=0.8 Hz, 1H); 7.98 (dd, J1=3.6 Hz, J2=1.1 Hz, 1H); 8.66 (dd, J1=2.6 Hz, J2=0.8 Hz, 1H).

### EXAMPLE 135

### 6-(1H-Pyrazol-1-yl)-N-(2-pyridin-3-ylethyl)-2-(2-thienyl)pyrimidin-4-amine

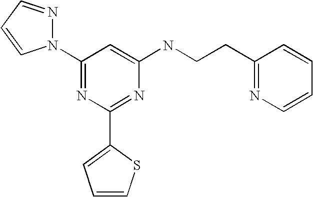

Obtained from Intermediate 55 (100 mg) and 2-(2-aminoethyl)pyridine (137 μL, 1.22 mmol) by the procedure described in Example 119. Purification by column chromatography with silica gel and n-hexane/ethyl acetate (from pure n-hexane to 2:3) as eluent gave 6 (1H-pyrazol-1-yl)-N-(2-pyridin-3-ylethyl)-2-(2-thienyl)pyrimidin-4-amine (58 mg, 44%) as an off-white solid.

m.p.: 132.9-133.6° C.

δ (300 MHz, CDCl₃): 3.16 (t, J=6.5 Hz, 2H); 3.90 (bs, 2H); 5.88 (t, J=5.2 Hz, 1H); 6.46 (dd, J1=2.6 Hz, J2=1.6 Hz, 1H); 6.79 (s, 1H); 7.11-7.21 (m, 3H); 7.44 (dd, J1=5.1 Hz, J2=1.2 Hz, 1H); 7.63 (dt, J1=7.7 Hz, J2=1.9 Hz, 1H); 7.74-7.75 (m, 1H); 7.98 (dd, J1=3.6 Hz, J2=0.7 Hz, 1H) 8.58 (m, 1H) 8.65 (dd, J1=2.6 Hz, J2=0.7 Hz, 1H).

### EXAMPLE 136

### 6-(1H-Pyrazol-1-yl)-N-(2-pyridin-2-ylethyl)-2-(2-thienyl)pyrimidin-4-amine

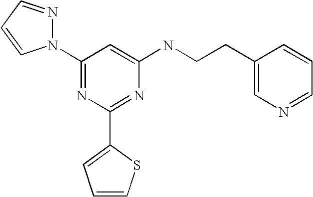

Obtained from Intermediate 55 (100 mg) and 3-(2-aminoethyl)pyridine (139 mg, 1.14 mmol) by the procedure described in Example 119. Purification by column chromatography with silica gel and n-hexane/ethyl acetate (from 1:1 to pure ethyl acetate) as eluent gave 6-(1H-pyrazol-1-yl)-N-(2-pyridin-2-ylethyl)-2-(2-thienyl)pyrimidin-4-amine (106 mg, 80%) as an off-white solid.

m.p.: 159.0-160.5° C.

δ (300 MHz, CDCl₃): 3.02 (t, J=7.14 Hz, 2H) 3.75 (m, 2H) 5.16 (s, 1H) 6.48 (m, J=2.75, 1.65 Hz, 1H) 6.78 (s, 1H) 7.14 (dd, J=5.08, 3.71 Hz, 1H) 7.25 (dd, J=4.53, 0.69 Hz, 1H) 7.28 (dd, J=4.81, 0.69 Hz, 1H) 7.47 (dd, J=4.94, 1.37 Hz, 1H) 7.59 (m, J=7.69, 1.65, 0.55 Hz, 1H) 7.75 (dd, J=1.51, 0.69 Hz, 1H) 7.99 (dd, J=3.71, 1.24 Hz, 1H) 8.51 (dd, J=4.67, 1.65 Hz, 1H) 8.55 (d, J=1.65 Hz, 1H) 8.67 (dd, J=2.75, 0.82 Hz, 1H).

### EXAMPLE 137

### N-(3-Phenylpropyl)-6-(1H-pyrazol-1-yl)-2-(2-thienyl)pyrimidin-4-amine

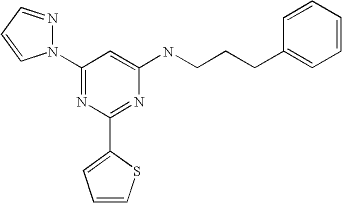

Obtained from Intermediate 55 (100 mg) and 3-phenylpropylamine (162 μL, 1.14 mmol) by the procedure described in Example 119. Purification by column chromatography with silica gel and n-hexane/ethyl acetate (from 95:5 to 90:10) as eluent gave N-(3-phenylpropyl)-6-(1H-pyrazol-1-yl)₂₋₂-thienyl)pyrimidin-4-amine (98 mg, 72%) as an off-white solid.

m.p.: 83.6-84.5° C.

δ (300 MHz, CDCl₃): 2.02 (q, J=7.4 Hz, 2H); 2.76 (t, J=7.4 Hz, 2H); 3.44 (bs, 2H); 5.17 (bs, 1H); 6.47 (dd, J1=2.7 Hz, J2=1.7 Hz, 1H); 6.74 (s, 1H); 7.22-7.31 (m, 6H); 7.45 (dd, J1=5.1 Hz, J2=1.2 Hz, 1H); 7.75 (dd, J1=1.7 Hz, J2=0.6 Hz, 1H); 7.95 (dd, J1=3.7 Hz, J2=1.2 Hz, 1H); 8.66 (dd, J1=2.7 Hz, J2=0.6 Hz, 1H).

### EXAMPLE 138

### N-[3-(1H-imidazol-1-yl)propyl]-6-(1H-pyrazol-1-yl)-2-(2-thienyl)pyrimidin-4-amine

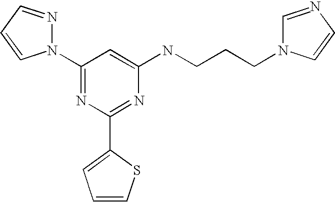

Obtained from Intermediate 5 (100 mg) and 3-(1H-imidazol-1-yl)propylamine (136 μL, 1.14 mmol) by the procedure described in Example 119. Purification by column chromatography with silica gel and methylene chloride/methanol (97:3) as eluent gave N-[3-(1H-imidazol-1-yl)propyl]-6-(1H-pyrazol-1-yl)₂₋₂-thienyl)pyrimidin-4-amine (130 mg, 98%) as an off-white solid.

δ (300 MHz, CDCl₃): 2.20 (q, J=7.0 Hz, 2H); 3.46-3.54 (m, 2H); 4.11 (t, J=7.0 Hz, 2H); 5.14 (bs, 1H); 6.48 (dd, J1=2.7 Hz, J2=1.7 Hz, 1H); 6.77 (s, 1H); 6.97 (t, J=1.2 Hz, 1H); 7.10 (t, J=1.2 Hz, 1H); 7.14 (dd, J1=5.1 Hz, J2=3.7 Hz, 1H); 7.47 (dd, J1=5.1 Hz, J2=1.2 Hz, 1H); 7.54 (s, 1H); 7.75 (dd, J1=1.5 Hz, J2=0.7 Hz, 1H) 7.97 (dd, J1=3.7 Hz, J2=1.2 Hz, 1H); 8.66 (dd, J1=2.7 Hz, J2=0.7 Hz, 1H).

### EXAMPLE 139

### Ethyl 6-(1H-pyrazol-1-yl)-2-(2-thienyl)pyrimidin-4-yl]carbamate

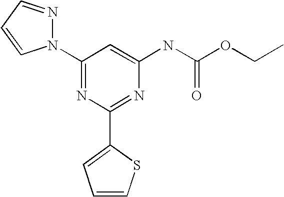

A solution of the title compound of Example 56 (0.37 g, 1.52 mmol), diethyl pyrocarbonate (246 μL, 1.67 mmol) and 4-dimethylaminopyridine (50 mg, 0.41 mmol) in tetrahydrofuran (4 mL) was heated at 45° C. overnight. The reaction was poured into water (40 mL) and extracted with ethyl acetate (2×25 mL). The organic layer was washed with water (2×25 mL), brine (25 mL), dried (Na₂SO₄), and the solvent was removed under reduced pressure. Purification by column chromatography with silica gel, eluting with n-hexane/ethyl acetate (9:1) as eluent gave ethyl 6-(1H-pyrazol-1-yl)-2-(2-thienyl)pyrimidin-4-ylcarbamate (34 mg, 7%) as an off-white solid.

δ (300 MHz, CDCl₃): 1.35 (t, J1=7.0 Hz, 3H); 4.31 (d, J=7.0 Hz, 2H); 6.50 (dd, J1=2.6 Hz, J2=1.2 Hz, 1H); 7.15 (dd, J1=5.1 Hz, J2=3.7 Hz, 1H); 7.49 (dd, J1=5.1 Hz, J2=1.6 Hz, 1H); 7.52 (bs, 1H); 7.80 (dd, J1=1.6 Hz, J2=0.7 Hz, 1H); 7.99 (dd, J1=3.7 Hz, J2=1.2 Hz, 1H); 8.33 (s, 1H); 8.66 (dd, J1=2.6 Hz, J2=0.7 Hz, 1H).

### EXAMPLE 140

### N-[2-(2-Furyl)-6-(1H-pyrazol-1-yl)pyrimidin-4-yl]-N′-[(1S*,2R)-2-phenylcyclopropyl]urea (*relative trans configuration)

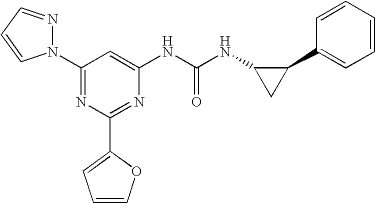

To a solution of the title compound of Example 1 (0.22 g, 0.97 mmol) in anhydrous tetrahydrofuran (14 mL), cooled at −78° C., was slowly added 2.5M solution of n-butyllithium in hexanes (0.78 mL). The mixture was stirred at −78° C. for 1 hour and then, a solution of (1S*,2R*)-2-phenylcyclopropylisocyanate (0.22 mg, 1.40 mmol) in anhydrous tetrahydrofuran (2 mL) was slowly added. The mixture was allowed to stand at room temperature for 2 hours. Water (15 mL) was added and the organic phase was diluted with ethyl acetate (20 mL). The organic layer was washed with brine (2×20 mL), dried (Na₂SO₄), and the solvent removed under reduced pressure. Purification by column chromatography with silica gel, eluting with methylene chloride/methanol (99:1) as eluent, followed by a preparative HPLC-MS purification gave N-[2-(2-furyl)-6-(1H-pyrazol-1-yl)pyrimidin-4-yl]-N′-[(1S*,2R*)-2-phenylcyclopropyl]urea (*relative trans configuration) (150 mg, 40%) as an off-white solid.

δ (400 MHz, DMSO-d₆): 1.18-1.32 (m, 1H); 2.05-2.12 (m, 1H): 2.81-2.88 (m, 1H): 6.64 (s, 1H); 6.76 (dd, J1=3.3 Hz, J2=1.8 Hz, 1H); 7.14-7.22 (m, 3H); 7.26-7.31 (m, 2H); 7.44 (d, J=3.1 Hz, 1H); 7.89 (s, 1H); 7.91 (s, 1H); 7.98 (s, 1H); 8.10 (bs, 1H); 8.75 (d, J=2.3 Hz, 1H); 9.96 (bs, 1H).

### EXAMPLE 141

### N-[2-(2-Furyl)-6-(1H-pyrazol-1-yl)pyrimidinyl]-N-propylurea

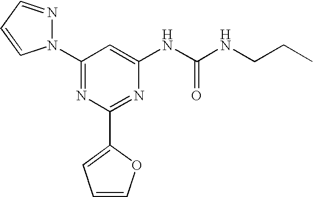

Obtained from the title compound of Example 1 (0.22 g) and propylisocyanate (0.12 g, 1.40 mmol) by the procedure described in Example 140. Purification by column chromatography with silica gel and methylene chloride/methanol (99:1) as eluent gave N-[2-(2-furyl)-6-(1H-pyrazol-1-yl)pyrimidin-4-yl]-N′-propylurea (68 mg, 20%) as an off-white solid.

δ (300 MHz, DMSO-d₆): 0.94 (t, J=7.4 Hz, 3H); 1.53 (h, J=7.4 Hz, 2H); 3.18 (q, J=7.4 Hz, 2H); 6.65 (dd, J1=2.7 Hz, J2=1.7 Hz, 1H); 6.75 (dd, J1=3.4 Hz, J2=1.7 Hz, 1H); 7.44 (dd, J1=3.4 Hz, J2=0.8 Hz, 1H); 7.86 (bs, 1H); 7.92 (dd, J1=1.7 Hz, J2=0.7 Hz, 1H); 7.96 (dd, J1=1.7 Hz, J2=0.8 Hz, 1H); 8.75 (dd, J1=2.7 Hz, J2=0.7 Hz, 1H); 9.97 (bs, 1H).

### EXAMPLE 142

### N-[2-(2-Furyl)-6-(1H-pyrazol-1-yl)pyrimidin-4-yl]-N′-isopropylurea

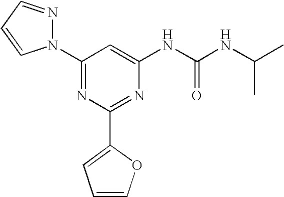

Obtained from the title compound of Example 1 (0.22 g) and isopropylisocyanate (0.12 g, 1.40 mmol) by the procedure described in Example 140. Purification by column chromatography with silica gel and methylene chloride/methanol (99:1) as eluent gave N-[2-(2-furyl)-6-(1H-pyrazol-1-yl)pyrimidin-4-yl]-N′-isopropylurea (147 mg, 48%) as an off-white solid.

δ (400 MHz, DMSO-d₆): 1.19 (d, J=6.7 Hz, 6H); 3.84 (h, J=6.7 Hz, 1H); 6.64 (s, 1H); 6.75 (s, 1H); 7.42 (d, J=3.1 Hz, 1H); 7.80 (s, 1H); 7.92 (s, 1H); 7.97 (s, 1H); 8.75 (d, J=2.3 Hz, 1H); 9.87 (s, 1H).

### EXAMPLE 143

### N-Cyclopentyl-N-[2-(2-furyl)-6-(1H-pyrazol-1-yl)pyrimidin-4-yl]urea

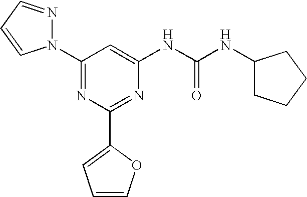

Obtained from the title compound of Example 1 (0.22 g) and cyclopentylisocyanate (0.16 g, 1.40 mmol) by the procedure described in Example 140. Purification by column chromatography with silica gel and methylene chloride/methanol (99:1) as eluent gave N-cyclopentyl-N′-[2-(2-furyl)-6-(1H-pyrazol-1-yl)pyrimidin-4-yl]urea (125 mg, 38%) as an off-white solid.

δ (300 MHz, DMSO-d₆): 1.42-1.54 (m, 2H); 1.55-1.66 (m, 2H); 1.67-1.75 (m, 2H); 1.84-1.96 (m, 2H); 3.99-4.09 (m, 1H); 6.65 (dd, J1=2.7 Hz, J2=1.6 Hz, 1H); 6.76 (dd, J1=3.4 Hz, J2=1.6 Hz, 1H); 7.43 (d, J=2.7 Hz, 1H); 7.76 (s, 1H); 7.92 (d, J=1.1 Hz, 1H); 7.97 (s, 1H); 8.10 (bs, 1H); 8.75 (d, J=2.7 Hz, 1H); 9.89 (s, 1H).

### EXAMPLE 144

### N-[2-(2-Furyl)-6-(1H-pyrazol-1-yl)pyrimidin-4-yl]-N′-(4-methoxyphenyl)urea

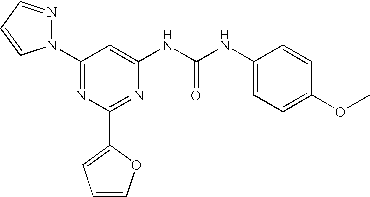

Obtained from the title compound of Example 1 (0.22 g) and 4-methoxybenzeneisocyanate (0.21 g, 1.40 mmol) by the procedure described in Example 140. Purification by column chromatography with silica gel and methylene chloride/methanol (98:2) as eluent gave N-[2-(2-furyl)-6-(1H-pyrazol-1-yl)pyrimidin-4-yl]-1-(4-methoxyphenyl)urea (82 mg, 22%) as an off-white solid.

δ (400 MHz, DMSO-d₆): 3.75 (s, 3H); 6.66 (dd, J1=2.7 Hz, J2-=1.8 Hz, 1H); 6.78 (dd, J1=3.3 Hz, J2=1.8 Hz, 1H); 6.93-7.0 (m, 2H); 7.46-7.52 (m, 3H); 7.89 (s, 1H); 7.94 (s, 1H); 8.03 (s, 1H); 8.78 (d, J=2.7 Hz, 1H); 10.13 (s, 1H); 10.17 (bs, 1H).

### EXAMPLE 145

### N-[2-(2-Furyl)-6-(1H-pyrazol-1-yl)pyrimidin-4-yl]-N′-(2-phenylethyl)urea

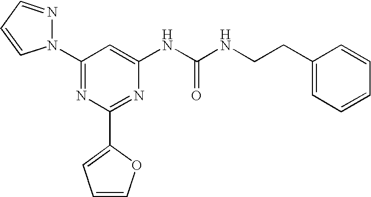

Obtained from the title compound of Example 1 (0.22 g) and phenethylisocyanate (0.21 g, 1.40 mmol) by the procedure described in Example 140. Purification by column chromatography with silica gel and methylene chloride/methanol (99:1) as eluent gave N-[2-(2-furyl)-6-(1H-pyrazol-1-yl)pyrimidinyl]-N′-(2-phenylethyl)urea (35 mg, 10%) as an off-white solid.

δ (400 MHz, DMSO-d₆): 2.83 (t, J=7.0 Hz, 2H); 3.47 (q, J=7.0 Hz, 2H); 6.64 (dd, J1=2.7 Hz, J2=1.6 Hz, 1H); 6.72 (dd, J1=3.3 Hz, J2=1.8 Hz, 1H); 7.18-7.24 (m, 1H); 7.26-7.34 (m, 5H); 7.64 (bs, 1H); 7.90-7.92 (m, 2H); 7.93-7.95 (m, 1H); 8.73 (d, J=2.7 Hz, 1H); 9.98 (bs, 1H).

### EXAMPLE 146

### N-Benzyl-N′-[2-(2-furyl)-6-(1H-pyrazol-1-yl)pyrimidin-4-yl]urea

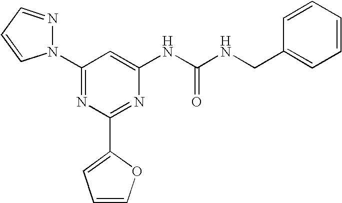

Obtained from the title compound of Example 1 (0.22 g) and benzylisocyanate (0.19 g, 1.40 mmol) by the procedure described in Example 140. Purification by column chromatography with silica gel and methylene chloride/methanol (from 99:1 to 85:5) as eluent, followed by a preparative HPLC-MS purification gave N-benzyl-N′-[2-(2-furyl)-6-(1H-pyrazol-1-yl)pyrimidin-4-yl]urea (10 mg, 3%) as an off-white solid.

MS (M⁺): 360.

### EXAMPLE 147

### N-[2-(2-Furyl)-6-(1H-pyrazol-1-yl)pyrimidin-4-yl]-3-methylbutanamide

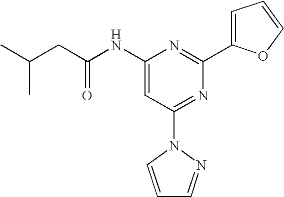

Obtained from the title compound of Example 1 (0.24 g) by the procedure described in Example 2. Purification by column chromatography with silica gel and ethyl acetate/n-hexane (1:1) as eluent gave N-[2-(2-furyl)-6-(1H-pyrazol-1-yl)pyrimidin-4-yl]-3-methylbutanamide (0.38 g, 85%) as an off-white solid.

δ (400 MHz, CDCl₃): 1.21 (s, 9H); 2.38 (s, 2H); 6.51-6.48 (m, 1H); 6.60-6.58 (m, 1H); 7.34 (d, J=3.6 Hz, 1H); 7.62 (s, 1H); 7.79 (s, 1H); 8.13 (bs, 1H); 8.58 (s, 1H); 8.62 (d, J=2.4 Hz, 1H).

### EXAMPLE 148

### N-[2-(2-Furyl)-6-(1H-pyrazol-1-yl)pyrimidin-4-yl]-3,3-dimethyl-butanamide

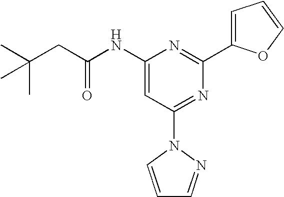

Obtained from the title compound of Example 1 (0.24 g) by the procedure described in Example 2. Purification by column chromatography with silica gel and ethyl acetate/n-hexane (1:1) as eluent gave N-[2-(2-furyl)-6-(1H-pyrazol-1-yl)pyrimidin-4-yl]-3,3-dimethylbutanamide (0.41 g, 91%) as an off-white solid.

δ (400 MHz, CDCl₃): 1.28 (d, J=7.0 Hz, 6H); 2.36 (d, J=7.0 Hz, 2H); 2.58 (h, J=7.0 Hz, 1H); 6.51-6.48 (m, 1H); 6.60-6.58 (m, 1H); 7.34 (d, J=3.6 Hz, 1H); 7.62 (s, 1H); 7.79 (s, 1H); 8.13 (bs, 1H); 8.58 (s, 1H); 8.62 (d, J=2.4 Hz, 1H).

### EXAMPLE 149

### N-[2-(2-Furyl)-6-(1H-pyrazol-1-yl)pyrimidin-4-yl]cyclopentanecarboxamide

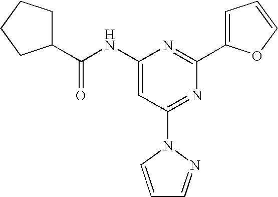

Obtained from the title compound of Example 1 (0.15 g) by the procedure described in Example 2. Purification by column chromatography with silica gel and methylene chloride as eluent followed by trituration with diethyl ether gave N-[2-(2-furyl)-6-(1H-pyrazol-1-yl)pyrimidin-4-yl]cyclopentanecarboxamide (0.12 g, 55%) as an off-white solid.

δ (250 MHz, CDCl₃): 1.98-1.60 (m, 8H); 2.76 (q, J=7.8 Hz, 1H); 6.49 (dd, J1=2.7 Hz, J2=1.5 Hz, 1H); 6.59 (dd, J1=3.3 Hz, J2=1.8 Hz, 1H); 7.34 (dd, J1=3.3 Hz, J2=0.9 Hz, 1H); 7.63 (m, 1H); 7.78 (m, 1H); 8.16 (bs, 1H); 8.61 (s, 1H); 8.62 (d, J=2.7 Hz, 1H).

### EXAMPLE 150

### 2-Chloro-N-[2-(2-furyl)-6-(1H-pyrazol-1-yl)pyrimidin-4-yl]-2-phenylacetamide

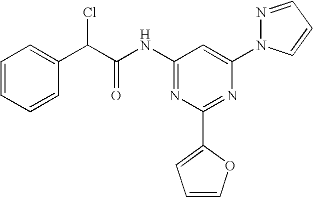

To a solution of the title compound of Example 1 (0.10 g, 0.44 mmol) and a catalytic amount of 4-dimethylaminopyridine in methylene chloride (2.3 mL) was added pyridine (71 μL, 0.88 mmol) and a solution of chloro(phenyl)acetyl chloride (139 μL, 0.88 mmol) in methylene chloride (1 mL). The mixture was stirred at room temperature for 3 hours and more pyridine (36 μL, 0.44 mmol) and chloro(phenyl)acetyl chloride (70 μL, 0.44 mmol) were added. The reaction was allowed to stand for 12 further hours at room temperature. The solution was diluted with methylene chloride (20 mL), washed with 1N citric acid (2×10 mL), saturated solution of sodium bicarbonate (2×10 mL), brine (10 mL) and dried (Na₂SO₄). The solvent was removed under reduced pressure. Purification by column chromatography with silica gel, eluting with ethyl acetate/n-hexane (3:7), gave 2-chloro-N-[2-(2-furyl)-6-(1H-pyrazol-1-yl)pyrimidin-4-yl]-2-phenylacetamide (0.108 g, 65%) as an off-white solid.

δ (400 MHz, CDCl₃): 5.53 (s, 1H); 6.49-6.50 (m, 1H); 6.61 (dd, J1=2.4 Hz, J2=1.6 Hz, 1H); 7.30-7.42 (m, 4H); 7.50-7.60 (m, 2H); 7.66 (s, 1H); 7.78 (s, 1H); 8.57 (s, 1H); 8.64 (d, J=2.8 Hz, 1H); 8.97 (s, 1H).

### EXAMPLE 151

### N-[2-(2-Furyl)-6-(1H-pyrazol-1-yl)pyrimidin-4-yl]-2-phenylacetamide

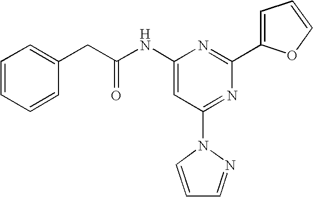

Obtained from the title compound of Example 1 (0.1 g) and phenylacetyl chloride (0.136 g, 0.88 mmol) by the procedure described in Example 150. Purification by column chromatography with silica gel and ethyl acetate/n-hexane (2:8) as eluent gave N-[2-(2-furyl)-6-(1H-pyrazol-1-yl)pyrimidin-4-yl]-2-phenylacetamide (58 mg, 38%) as an off-white solid.

δ (400 MHz, CDCl₃): 3.79 (s, 2H); 6.49 (dd, J1=2.8 Hz, J2=1.6 Hz, 1H); 6.57 (dd, J1=3.2 Hz, J2=1.6 Hz, 1H); 7.25-7.45 (m, 6H); 7.64 (s, 1H); 7.79 (s, 1H); 8.07 (bs, 1H); 8.59 (s, 1H); 8.62 (d, J=2.8 Hz, 1H).

### EXAMPLE 152

### 2-(4-Fluorophenyl)-N-[2-(2-furyl)-6-(1H-pyrazol-1-yl)pyrimidin-4-yl]acetamide

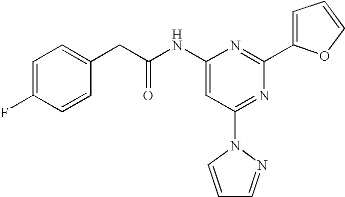

Obtained from the title compound of Example 1 (0.1 g) and (4-fluorophenyl)acetyl chloride (121 μL, 0.88 mmol) by the procedure described in Example 150. Purification by column chromatography with silica gel and ethyl acetate/n-hexane (3:7) as eluent gave 2-(4-fluorophenyl-N-[2-(2-furyl)-6-(1H-pyrazol-1-yl)pyrimidin-4-yl]acetamide (82 mg, 51%) as an off-white solid.

δ (400 MHz, CDCl₃): 3.78 (s, 2H); 6.51 (dd, J1=2.4 Hz, J2=1.6 Hz, 1H); 6.60 (dd, J1=2.8 Hz, J2=1.6 Hz, 1H); 7.10 (t, J=8.6 Hz, 2H); 7.25-7.35 (m, 3H); 7.64 (s, 1H); 7.81 (s, 1H); 8.08 (s, 1H); 8.61 (s, 1H); 8.64 (d, J=2.4 Hz, 1H).

### EXAMPLE 153

### N-[2-(2-Furyl)-6-(1H-pyrazol-1-yl)pyrimidin-4-yl]-2-(3-methoxyphenyl)acetamide

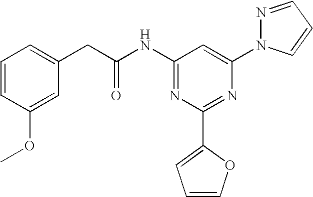

To a solution of the title compound of Example 1 (0.10 g, 0.44 mmol) and a catalytic amount of 4-dimethylaminopyridine in pyridine (2.3 mL) was added a solution of (3-methoxyphenyl)acetyl chloride (0.162 g, 0.88 mmol) in pyridine (1.3 mL). The mixture was stirred overnight at 80° C. The solution was diluted with ethyl acetate (30 mL), washed with 1N citric acid (3×20 mL), saturated solution of sodium bicarbonate (2×15 mL), brine (10 mL) and dried (Na₂SO₄). The solvent was removed under reduced pressure. Purification by column chromatography with silica gel, eluting with ethyl acetate/n-hexane (1:2) gave N-[2-(2-furyl)-6-(1H-pyrazol-1-yl)pyrimidin-4-yl]-2-(3-methoxyphenyl)acetamide (0.06 g, 36%) as an off-white solid.

δ (400 MHz, CDCl₃): 3.76 (s, 2H); 3.83 (s, 3H); 6.47-6.51 (m, 1H); 6.5-6.59 (m, 1H); 6.80-7.00 (m, 3H); 7.20-7.40 (m, 2H); 7.61 (s, 1H); 7.80 (s, 1H); 8.07 (s, 1H); 8.55-8.65 (m, 2H).

### EXAMPLE 154

### N-[2-(2-Furyl)-6-(1H-pyrazol-1-yl)pyrimidin-4-yl]-2-(2-methoxyphenyl)acetamide

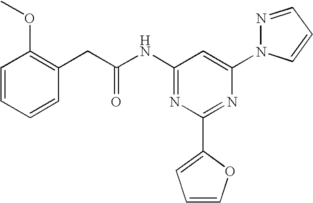

Obtained from the title compound of Example 1 (0.1 g) and (2-methoxyphenyl)acetyl chloride (0.162 g, 0.88 mmol) by the procedure described in Example 153. Purification by column chromatography with silica gel and ethyl acetate/n-hexane (1:2) as eluent gave N-[2-(2-furyl)-6-(1H-pyrazol-1-yl)pyrimidin-4-yl]-2-(2-methoxyphenyl)acetamide (78 mg, 47%) as an off-white solid.

δ (400 MHz, CDCl₃): 3.78 (s, 2H); 3.96 (s, 3H); 6.46-6.49 (m, 1H); 6.56-6.59 (m, 1H); 6.93-7.03 (m, 2H); 7.27-7.35 (m, 3H); 7.62 (s, 1H); 7.78 (s, 1H); 8.54 (bs, 1H); 8.56 (s, 1H); 8.62 (d, J=2.8 Hz, 1H).

### EXAMPLE 155

### 2-(3,4-Dichlorophenyl)-N-[2-(2-furyl)-6-(1H-pyrazol-1-yl)pyrimidin-4-yl]acetamide

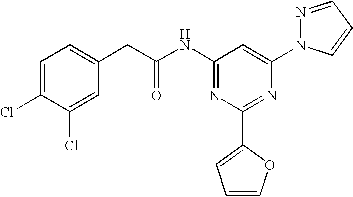

Obtained from the title compound of Example 1 (0.1 g) and (3,4-dichlorophenyl)acetyl chloride (0.197 g, 0.88 mmol) by the procedure described in Example 150. Purification by column chromatography with silica gel and ethyl acetate/n-hexane (3:7) as eluent gave 2-(3,4-dichlorophenyl)-N-[2-(2-furyl)-6-(1H-pyrazol-1-yl)pyrimidin-4-yl]acetamide (58 mg, 32%) as an off-white solid.

δ (400 MHz, CDCl₃): 3.72 (s, 2H); 6.49 (dd, J1=2.8 Hz, J2=2.0 Hz, 1H); 6.59 (dd, J1=3.6 Hz, J2=2.0 Hz, 1H); 7.16-7.20 (m, 1H); 7.32-7.35 (m, 1H); 7.42-7.47 (m, 2H); 7.62 (s, 1H); 7.79 (s, 1H); 8.13 (bs, 1H); 8.55 (s, 1H); 8.62 (d, J=2.8 Hz, 1H).

### EXAMPLE 156

### 2-(1,3-Dibenzodioxol-5-yl)-N-[2-(2-furyl)-6-(1H-pyrazol-1-yl)pyrimidin-4-yl]acetamide

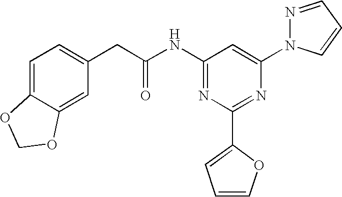

Obtained from the title compound of Example 1 (0.1 g) and benzo[1,3]dioxol-5-yl-acetyl chloride (0.175 g, 0.88 mmol) by the procedure described in Example 150. Purification by column chromatography with silica gel and ethyl acetate/n-hexane (1:2) as eluent gave 2-(1,3-benzodioxol-5-yl)-N-[2-(2-furyl)-6-(1H-pyrazol-1-yl)pyrimidinyl]acetamide (92 mg, 54%) as a yellow solid.

δ (400 MHz, CDCl₃): 3.70 (s, 2H); 5.99 (s, 2H); 6.49 (dd, J1=2.8 Hz, J2=1.6 Hz, 1H); 6.57 (dd, J1=3.6 Hz, J2=2.0 Hz, 1H); 6.75-6.84 (m, 3H); 7.33 (d, J=3.6 Hz, 1H); 7.61 (d, J=0.8 Hz, 1H); 7.79 (d, J=1.6 Hz, 1H); 8.04 (s, 1H); 8.59 (s, 1H); 8.62 (d, J=2.8 Hz, 1H).

### EXAMPLE 157

### 2-(3,4-Dihydroxyphenyl)-N-[2-(2-furyl)-6-(1H-pyrazol-1-yl)pyrimidin-4-yl]acetamide

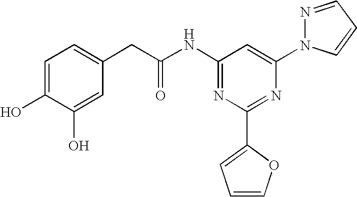

To a solution of the title compound of Example 11 (0.10 g, 0.247 mmol) in methylene chloride (2.5 mL) was added a 1M solution of boron tribromide in methylene chloride (2 mL, 2 mmol) at −40° C. under nitrogen. The solution was stirred at −40° C. for 30 min before warming to room temperature. The reaction was quenched by slow addition of ethanol (3.5 mL) at 0° C. and the reaction mixture was added to a solution of saturated solution of sodium bicarbonate (50 mL), diluted with ethyl acetate (30 mL) and the layers separated. The aqueous layer was extracted with ethyl acetate (2×30 mL), the organic layer was dried (Na₂SO₄), and the solvent was removed under reduced pressure. Purification by column chromatography with silica gel, eluting with methylene chloride/methanol (98:2) gave 2-(3,4-dihydroxyphenyl)-N-[2-(2-furyl)-6-(1H-pyrazol-1-yl)pyrimidin-4-yl]acetamide (39 mg, 42%) as an off-white solid.

δ (400 MHz, DMSO-d₆): 3.61 (s, 2H); 6.58-6.70 (m, 3H); 6.75 (dd, J1=3.2 Hz, J2=1.6 Hz, 1H); 6.77 (d, J=2.0 Hz, 1H); 7.48 (d, J=3.2 Hz, 1H); 7.92 (s, 1H); 7.97 (s, 1H); 8.42 (s, 1H); 8.74 (s, 1H); 8.77 (d, J=2.2 Hz, 1H); 8.86 (s, 1H); 11.29 (s, 1H).

### EXAMPLE 158

### 2-(2,5-Dimethoxyphenyl)-N-[2-(2-furyl)-6-(1H-pyrazol-1-yl)pyrimidin-4-yl]acetamide

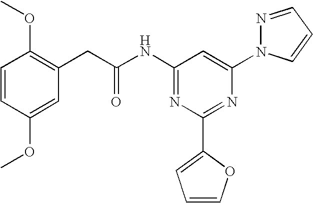

Obtained from the title compound of Example 1 (0.1 g) and (2,5-dimethoxyphenyl)acetyl chloride (0.19 g, 0.88 mmol) by the procedure described in Example 153. Purification by column chromatography with silica gel and ethyl acetate/n-hexane (2:8) as eluent gave 2-(2,5-dimethoxyphenyl)-N-[2-(2-furyl)-6-(1H-pyrazol-1-yl)pyrimidinyl]acetamide (165 mg, 93%) as an off-white solid.

δ (400 MHz, CDCl₃): 3.75 (s, 2H); 3.78 (s, 3H); 3.92 (s, 3H); 6.48 (dd, J1=2.8 Hz, J2=1.6 Hz, 1H); 6.57 (dd, J1=3.2 Hz, J2=1.6 Hz, 1H); 6.80-6.92 (m, 3H); 7.30 (d, J=3.6 Hz, 1H); 7.62 (s, 1H); 7.78 (s, 1H); 8.55 (s, 1H); 8.62 (d, J=2.8 Hz, 1H).

### Intermediate 56. (4-Chloro-3-methylphenyl)acetic acid

A suspension of 4-chloro-3-methylacetophenone (25 g, 0.148 mol) and sulfur (4.70 g, 0.146 mol) in morpholine (14.1 mL, 0.161 mol) was stirred at 140° C. overnight. The mixture was cooled and diluted with ethyl ether (50 mL). The resulting solid was filtered, washed with ethyl ether (2×25 mL) and solved in ethanol (540 mL). To this solution was added water (100 mL) and potassium hydroxide (84 g, 1.5 mol). The mixture was stirred at 90° C. overnight. The solvent was removed under reduced pressure and the reaction crude was diluted with water. The aqueous layer was washed with ethyl ether (2×50 mL), acidified with 1N hydrochloric acid and extracted with ethyl ether (3×50 mL). The organic layer was washed with 1N hydrochloric acid (2×25 mL), water (2×25 mL), brine (25 mL), dried (Na₂SO₄), and the solvent removed under reduced pressure. The resulting solid was filtered and washed with n-hexane (50 mL) to yield the title compound (11.15 g, 41%) as a yellow solid.

δ (300 MHz, CDCl₃): 2.34 (s, 3H); 3.60 (s, 2H); 7.02-7.11 (m, 1H); 7.16-7.22 (m, 1H); 7.27-7.34 (m, 1H).

### EXAMPLE 159

### 2-(4-Chloro-3-methylphenyl)-N-[2-(2-furyl)-6-(1H-pyrazol-1-yl)pyrimidin-4-yl]acetamide

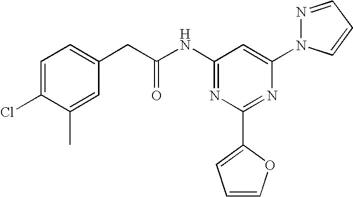

To a solution of (4-chloro-3-methylphenyl)acetic acid (0.162 g, 0.88 mmol) and a catalytic amount of DMF in methylene chloride (2 mL) was added oxalyl chloride (84 μL, 0.97 mmol), and the reaction mixture was stirred at room temperature for 3 hours. The solvent was removed under reduced pressure to give (4-chloro-3-methylphenyl)acetyl chloride that was used in the next step without further purification. 2-(4-Chloro-3-methylphenyl)-N-[2-(2-furyl)-6-(1H-pyrazol-1-yl)pyrimidin-4-yl]acetamide was obtained from the title compound of Example 1 (0.1 g) and (4-chloro-3-methylphenyl)acetyl chloride (0.179 g, 0.88 mmol) by the procedure described in Example 153. Purification by column chromatography with silica gel and ethyl acetate/n-hexane (1:9) as eluent gave the title compound (55 mg, 32%) as an off-white solid.

δ (400 MHz, CDCl₃): 2.39 (s, 3H); 3.71 (s, 2H); 6.49 (dd, J1=2.4 Hz, J2=1.6 Hz, 1H); 6.58 (dd, J1=3.2 Hz, J2=1.6 Hz, 1H); 7.08-7.13 (m, 1H); 7.19-7.26 (m, 1H); 7.32-7.37 (m, 2H); 7.62 (s, 1H); 7.79 (s, 1H); 8.06 (bs, 1H); 8.58 (s, 1H); 8.62 (d, J=2.8 Hz, 1H).

### EXAMPLE 160

### 2-(3,5-Dimethoxyphenyl)-N-[2-(2-furyl)-6-(1H-pyrazol-1-yl)pyrimidin-4-yl]acetamide

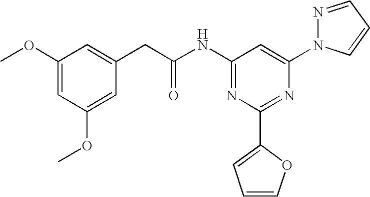

Obtained from the title compound of Example 1 (0.3 g) and (3,5-dimethoxyphenyl)acetyl chloride (0.567 g, 2.64 mmol) by the procedure described in Example 153. Purification by column chromatography with silica gel and ethyl acetate/n-hexane (2:8) as eluent gave 2-(3,5-dimethoxyphenyl)-N-[2-(2-furyl)-6-(1H-pyrazol-1-yl)pyrimidin-4-yl]acetamide (188 mg, 35%) as an off-white solid.

δ (400 MHz, CDCl₃): 3.71 (s, 2H); 3.79 (s, 6H); 6.41-6.44 (m, 1H); 6.45-6.48 (m, 2H); 6.49 (dd, J1=2.8 Hz, J2=2.0 Hz, 1H); 6.57 (dd, J1=3.6 Hz, J2=2.0 Hz, 1H); 7.32 (d, J=3.6 Hz, 1H); 7.61 (d, J=1.6 Hz, 1H); 7.80 (d, J=1.6 Hz, 1H); 8.08 (bs, 1H); 8.59 (s, 1H); 8.63 (d, J=2.8 Hz, 1H).

### EXAMPLE 161

### 2-[3-(Benzyloxy)-4-methoxyphenyl]-N-[2-(2-furyl)-6-(1H-pyrazol-1-yl)pyrimidin-4-yl]acetamide

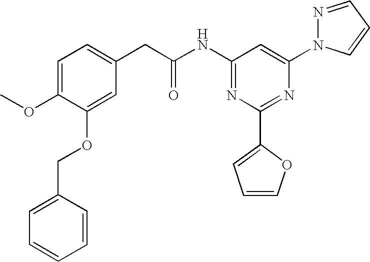

To solution of (3-benzyloxy-methoxyphenyl)acetic acid (0.299 g, 1.10 mmol) and a catalytic amount of DMF in methylene chloride (2.2 mL) was added oxalyl chloride (105 μL, 1.21 mmol) and the reaction mixture was stirred at room temperature for 3 hours. The solvent was removed under reduced pressure to give (3-benzyloxy-4-methoxyphenyl)acetyl chloride that was used in the next reaction without further purification. 2-[3-Benzyloxy)-4-methoxyphenyl]-N-[2-(2-furyl)-6-(H-pyrazol-1-yl)pyrimidin-4-yl]acetamide was obtained from the title compound of Example 1 (0.1 g) and (3-benzyloxy-4-methoxyphenyl)acetyl chloride (0.320 g, 1.10 mmol) by the procedure described in Example 153. Purification by column chromatography with silica gel and ethyl acetate/n-hexane (1:2) as eluent gave the title compound (86 mg, 41%) as a yellow solid.

δ (400 MHz, CDCl₃): 3.67 (s, 2H); 3.90 (s, 3H); 5.16 (s, 2H); 6.49-6.51 (m, 1H); 6.58 (dd, J1=3.2 Hz, J2=1.6 Hz, 1H); 6.82-6.94 (m, 3H); 7.20-7.26 (m, 1H); 7.30-7.36 (m, 3H); 7.42-7.45 (m, 2H); 7.62 (s, 1H); 7.81 (s, 1H); 7.99 (bs, 1H); 8.57 (s, 1H); 8.63 (d, J=2.8 Hz, 1H).

### Intermediate 57. Methyl [4-(benzyloxy)-3-methoxyphenyl]acetate

To a solution of (4-benzyloxy-3-methoxyphenyl)acetic acid (5.0 g, 18 mmol) in methanol (25 mL) was added concentrated sulphuric acid (1 mL). The reaction was heated at reflux overnight. The solvent was removed under reduced pressure and the crude was diluted with ethyl acetate (100 mL), washed with saturated solution of sodium bicarbonate (2×50 mL), brine (50 mL) and dried (Na₂SO₄). The solvent was removed under reduced pressure and methyl [4-(benzyloxy)-3-methoxyphenyl]acetate was obtained as an off-white solid.

MS (M⁺): 286.

### Intermediate 58. Methyl (4-hydroxy-3-methoxyphenyl)acetate

To a solution of Intermediate 57 (5.22 g, 18 mmol) in ethyl acetate (40 mL) was added palladium on charcoal (0.52 g, 1.83 mmol). The mixture was stirred under hydrogen and at room temperature for 7 hours. The palladium on charcoal was filtered over Celite® and the solvent removed under reduced pressure, to give methyl (4-hydroxy-3-methoxyphenyl)acetate (3.40 g, 64%) as an oil.

δ (250 MHz, CDCl₃): 3.57 (s, 2H); 3.71 (s, 3H); 3.86 (s, 2H); 6.79-6.89 (m, 3H)

### Intermediate 59. Methyl [4-(cyclobutyloxy)-3-methoxyphenyl]acetate

To a solution of Intermediate 58 (0.33 g, 1.7 mmol) in DMF (2 mL) was added cyclobutyl bromide (0.165 mL, 1.75 mmol) and cesium carbonate (0.56 g, 1.75 mmol). The reaction was heated at 60° C. overnight. The mixture was cooled, acidified with 10% hydrochloric acid (25 mL), and extracted with ethyl acetate (3×10 mL). The combined organic extracts were washed with brine (2×10 mL), dried (Na₂SO₄), and the solvent removed under reduced pressure. Purification by column chromatography with silica gel and ethyl acetate/n-hexane (1:4) as eluent gave the title compound (100 mg, 23%) as an off-white solid.

δ (250 MHz, CDCl₃): 1.59-1.72 (m, 1H); 1.77-1.89 (m, 1H); 2.20-2.28 (m, 2H); 2.39-2.51 (m, 2H); 3.55 (s, 2H); 3.69 (s, 3H); 3.86 (s, 3H); 4.62 (qt, J=7.0 Hz, 1H); 6.70 (d, J=8.0 Hz, 1H); 6.76 (dd, J1=8.0 Hz, J2=2.0 Hz, 1H); 6.79 (s, 1H).

### Intermediate 60. [4-(Cyclobutyloxy)-3-methoxyphenyl]acetic acid

A solution of Intermediate 59 (0.17 g, 0.67 mmol) and lithium hydroxide (72 mg, 1.72 mmol) in tetrahydrofuran (4 mL) and water (4 mL) was stirred at room temperature for 3 h. The organic solvent was removed under reduced pressure and the resulting aqueous solution was acidified with acetic acid until pH=5 and extracted with methylene chloride (3×50 mL). The organic layer was dried (MgSO₄) and the solvent removed under reduced pressure to give [4-(cyclobutyloxy)-3-methoxyphenyl]acetic acid as an oil (0.16 g, 99%).

δ (250 MHz, CDCl₃): 1.64-1.71 (m, 1H); 1.72-1.86 (m, 1H); 2.17-2.39 (m, 2H); 2.40-2.50 (m, 2H); 3.57 (s, 2H); 3.85 (s, 3H); 4.63 (qt, J=7.0 Hz, 1H); 6.68 (d, J=8.0 Hz, 1H); 6.75 (dd, J1=8.0 Hz, J2=2.0 Hz, 1H); 6.79 (s, 1H).

### EXAMPLE 162

### 2-[4-(Cyclobutyloxy)-3-methoxyphenyl]-N-[2-(2-furyl)-6-(1H-pyrazol-1-yl)pyrimidin-4-yl]acetamide

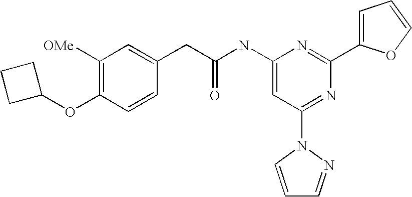

To a solution of Intermediate 60 (0.21 g, 0.89 mmol) in methylene chloride (2 mL) was added oxalyl chloride (0.11 g, 0.89 mmol) and a catalytic amount of DMF. The mixture was stirred at room temperature for 2 hours. This solution was added to a solution of the title compound of Example 1 (135 mg, 0.59 mmol) and pyridine (71 mg, 0.89 mmol) in methylene chloride (6 mL). The mixture was stirred at room temperature for 3 hours and diluted with methylene chloride (8 mL). The organic layer was washed with water (2×8 mL) and brine (8 mL), dried (Na₂SO₄), and the solvent removed under reduced pressure. Purification by column chromatography with silica gel, eluting with ethyl acetate/n-hexane (1:4), followed by a second column chromatography with silica gel and methylene chloride/acetonitrile (10%) as eluent gave 2-[4-(cyclobutyloxy)-3-methoxyphenyl]-N-[2-(2-furyl)-6-(1H-pyrazol-1-yl)pyrimidin-4-yl]acetamide (29 mg, 11%) as an off-white solid.

δ (250 MHz, CDCl₃): 3.71 (s, 2H); 3.88 (s, 3H); 4.67 (q, J=7.0 Hz, 1H); 6.49 (m, 1H); 6.57 (m, 1H); 6.74 (m, 1H); 6.83 (m, 2H); 7.35-7.26 (m, 1H); 7.62 (m, 1H); 7.81 (m, 1H); 8.08 (bs, 1H); 8.61 (m, 2H).

### Intermediate 61. 4-(Difluoromethoxy)-3-methoxybenzaldehyde

A solution of 4-hydroxy-3-methoxybenzaldehyde (2.0 g, 0.013 mol), sodium 2-chloro-2,2-difluoroacetate (4.8 g, 0.031 mol) and cesium carbonate (72 mg, 0.018 mol) in DMF (14 mL) and water (14 mL) was heated at 100° C. for 3.5 hours. The mixture was acidified with concentrated hydrochloric acid and extracted with ethyl acetate (2×25 mL). The organic layer was washed with water (2×25 mL), dried (MgSO₄) and the solvent removed under reduced pressure. Purification by column chromatography with silica gel and ethyl acetate/n-hexane (1:4) as eluent gave 4-(difluoromethoxy)-3-methoxybenzaldehyde (2.41 g, 91%) as an oil.

δ (250 MHz, CDCl₃): 3.76 (s, 3H); 6.49 (t, J_{F-H}=74.0 Hz, 1H); 7.11 (d, J=8.0 Hz, 1H); 7.27 (dd, J1=1.7 Hz, J2=8.0 Hz, 1H); 7.31 (d, J=1.7 Hz, 1H); 9.74 (s, 1H).

### Intermediate 62. [4-(Difluoromethoxy)-3-methoxyphenyl]methanol

To a solution of Intermediate 61 (2.7 g, 0.013 mol) in tetrahydrofuran (25 mL) and methanol (5 mL) was cooled at 0° C. and sodium borohydride (0.62 g, 0.016 mol) was added in small portions. The mixture was stirred at room temperature for 30 minutes, cooled at 0° C., and a solution of ammonium chloride (25 mL) was added. The crude was extracted with ethyl acetate (2×25 mL). The organic layer was washed with water (2×25 mL), brine (25 mL), dried (Na₂SO₄), and the solvent removed under reduced pressure. The title compound was obtained (2.7 g, 99%) as an oil.

δ (250 MHz, CDCl₃): 3.85 (s, 3H); 4.63 (s, 2H); 6.52 (t, J_{F-H}=75.0 Hz, 1H); 6.86 (dd, J1=2.0 Hz, J2=8.0 Hz, 1H); 6.98 (d, J=2.0 Hz, 1H); 7.10 (d, J=8.0 Hz, 1H).

### Intermediate 63. 4-(Chloromethyl)-1-(difluoromethoxy)-2-methoxybenzene

To a cooled solution of Intermediate 62 (1.46 g, 7.1 mmol) in methylene chloride (20 mL) was added pyridine (1.43 g, 18 mmol) and methane sulfonyl chloride (1.57 g, 13.7 mmol). The reaction was stirred at room temperature for 11 hours. The mixture was poured into a saturated solution of sodium bicarbonate (40 mL) and extracted with methylene chloride (2×20 mL). The combined organic extracts were washed with a saturated solution of sodium bicarbonate (2×15 mL), 1N hydrochloric acid (2×15 mL), brine (1×15 mL), dried (Na₂SO₄), and the solvent removed under reduced pressure. 4-(Chloromethyl)-1-(difluoromethoxy)-2-methoxybenzene was obtained (1.2 g, 60%) as an oil.

δ (250 MHz, CDCl₃): 3.60 (s, 3H); 4.56 (s, 2H); 6.55 (t, J_{F-H}=75.0 Hz, 1H); 6.94 (dd, J1=2.0 Hz, J2=8.0 Hz, 1H); 7.01 (d, J=2.0 Hz, 1H); 7.13 (d, J=8.0 Hz, 1H).

### Intermediate 64. [4-(Difluoromethoxy)-3-methoxyphenyl]acetonitrile

To a solution of Intermediate 63 (0.15 g, 0.69 mmol) in dimethylsulfoxide (1.6 mL) was added sodium cyanide (40 mg, 0.82 mmol). The mixture was stirred at room temperature for 7 hours. The reaction was poured into water (10 mL) and extracted with ethyl acetate (2×5 mL). The organic layer was washed with brine (2×5 mL), dried (Na₂SO₄), and the solvent removed under reduced pressure. The title compound was obtained (144 mg, 98%).

δ (250 MHz, CDCl₃): 3.71 (s, 2H); 3.84 (s, 3H); 6.53 (t, J_{F-H}=75.0 Hz, 1H); 6.83 (s, 1H); 6.88 (dd, J1=2.0 Hz, J2=8.0 Hz, 1H); 7.10 (d, J=8.0 Hz, 1H).

### Intermediate 65. [4-(Difluoromethoxy)-3-methoxyphenyl]acetic acid

A suspension of Intermediate 64 (0.60 g, 2.8 mmol) in 1N sodium hydroxide (20 mL) was heated at 110° C. for 3.5 hours. The resulting solution was acidified with 1N hydrochloric acid and extracted with ethyl acetate (2×25 mL). The combined extracts were washed with water (2×15 mL), dried (Na₂SO₄), and the solvent removed under reduced pressure. Purification by column chromatography with silica gel and methylene chloride/methanol (2%) as eluent gave [4-(difluoromethoxy)-3-methoxyphenyl]acetic acid (0.31 g, 48%).

δ (250 MHz, CDCl₃): 3.63 (s, 2H); 3.87 (s, 3H); 6.53 (t, J_{F-H}=75.0 Hz, 1H); 6.83 (s, 1H); 6.87 (dd, J1=2.0 Hz, J2=8.0 Hz, 1H); 7.11 (d, J=8.0 Hz, 1H).

### EXAMPLE 163

### N-[2-(2-Furyl)-6-(1H-pyrazol-1-yl)pyrimidin-4-yl]-2-(4 difluoromethoxy-3-methoxyphenyl)acetamide

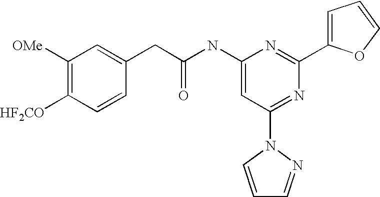

Obtained from the title compound of Example 1 (0.11 g) and Intermediate 65 (0.16 g, 0.70 mmol) by the procedure described in Example 162. Purification by column chromatography with silica gel and methylene chloride/acetonitrile (5%) as eluent gave N-[2-(2-furyl)-6-(1H-pyrazol-1-yl)pyrimidin-4-yl]-2-(4-difluoromethoxy-3-methoxyphenyl)acetamide (31 mg, 15%) as an off-white solid.

δ (250 MHz, CDCl₃): 3.75 (s, 2H); 3.89 (s, 3H); 6.49 (dd, J1=2.7 Hz, J2=1.5 Hz, 1H); 6.55 (t, J=75.2 Hz, 1H); 6.58 (dd, J1=3.3 Hz, J2=1.8 Hz, 1H); 6.95-6.86 (m, 2H); 7.16 (d, J=7.9 Hz, 1H); 7.33 (dd, J1=3.3 Hz, J2=0.6 Hz, 1H); 7.62 (dd, J1=1.5 Hz, J2=0.9 Hz, 1H); 7.79 (dd, J1=1.5 Hz, J2=0.6 Hz, 1H); 8.15 (bs, 1H); 8.58 (bs, 1H); 8.62 (dd, J1=2.7 Hz, J2=0.6 Hz, 1H).

### EXAMPLE 164

### N-[2-(2-Furyl)-6-(1H-pyrazol-1-yl)pyrimidin-4-yl]-2-(3,4,5-trimethoxyphenyl)acetamide

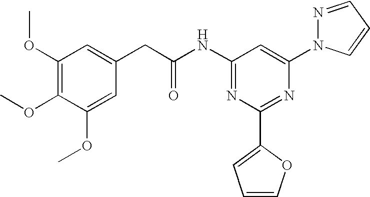

To a solution of (3,4,5-trimethoxyphenyl)acetic acid (0.199 g, 0.88 mmol) and a catalytic amount of DMF in methylene chloride (2 mL) was added oxalyl chloride (84 μL, 0.968 mmol) and the reaction mixture was stirred at room temperature for 3 hours. The solvent was removed under reduced pressure to give (3,4,5-trimethoxyphenyl)acetyl chloride that was used in the next reaction without further purification. N-[2-(2-furyl)-6-(1H-pyrazol-1-yl)pyrimidin-4-yl]-2-(3,4,5-trimethoxyphenyl)acetamide was obtained from the title compound of Example 1 (0.1 g) and (3,4,5-trimethoxyphenyl)acetyl chloride (0.215 g, 0.88 mmol) by the procedure described in Example 153. Purification by column chromatography with silica gel and ethyl acetate/n-hexane (2:8) as eluent gave the title compound (27 mg, 14%) as a red solid.

δ (400 MHz, CDCl₃): 3.72 (s, 2H); 3.86 (s, 3H); 3.88 (s, 6H); 6.50 (dd, J1=2.8 Hz, J2=1.6 Hz, 1H); 6.53 (s, 2H); 6.58 (dd, J1=3.6 Hz, J2=1.6 Hz, 1H); 7.33 (dd, J1=3.6 Hz, J2=0.8 Hz, 1H); 7.61 (dd, J1=2.0 Hz, J2=0.8 Hz, 1H); 7.80 (d, J=0.8 Hz, 1H); 8.11 (bs, 1H); 8.60 (s, 1H); 8.63 (dd, J1=2.0 Hz, J2=0.8 Hz, 1H).

### Intermediate 66. Methyl (3,4-dimethoxyphenyl)acetate

Obtained from (3,4-dimethoxyphenyl)acetic acid (5.0 g, 26 mmol) by the procedure described in Intermediate 57. Methyl (3,4-dimethoxyphenyl)acetate was obtained as an off-white solid (4.74 g, 88%).

δ (300 MHz, CDCl₃): 3.52 (s, 2H); 3.69 (s, 3H); 3.92 (s, 6H); 6.81 (s, 3H).

### Intermediate 67. Methyl 2-(3,4-dimethoxyphenyl)propanoate

A 1.6M solution of n-butyllithium in hexanes (4.83 mL, 7.74 mmol) was added to a stirred solution of diisopropylamine (1.18 mL, 8.40 mmol) in tetrahydrofuran (7 mL) at −78° C. under nitrogen. After 15 min, a solution of Intermediate 66 (1.0 g, 4.75 mmol) in tetrahydrofuran (14 mL) was slowly added at −78° C. and the solution was stirred at the same temperature for one hour. A solution of methyl iodide (0.59 mL, 9.5 mmol) in tetrahydrofuran (5 mL) was then added and the resulting mixture was stirred 30 min at −78° C. before warming to room temperature. The reaction mixture was poured into ice-water and extracted with ethyl acetate (3×50 mL). The organic layer was washed with brine (50 mL), dried (Na₂SO₄) and the solvent removed under reduced pressure to give methyl 2-(3,4-dimethoxyphenyl)propanoate as a brown oil (1.03 g, 97%).

δ (300 MHz, CDCl₃): 1.46 (d, J=7.0 Hz, 3H); 3.64 (s, 3H); 3.65 (q, J=7.0 Hz, 1H); 3.88 (s, 3H); 3.89 (s, 3H); 6.82 (s, 3H).

### Intermediate 68. 2-(3,4-Dimethoxyphenyl)propanoic acid

Obtained from Intermediate 67 (1 g, 4.45 mmol) by the procedure described in Intermediate 60. 2-(3,4-Dimethoxyphenyl)propanoic acid was obtained as an off-white solid (4.74 g, 88%).

δ (300 MHz, CDCl₃): 1.51 (d, J=7.0 Hz, 3H); 3.66 (q, J=7.0 Hz, 1H); 3.88 (s, 3H); 3.89 (s, 3H); 6.80-6.97 (m, 3H).

### EXAMPLE 165

### 2-(3,4-Dimethoxyphenyl)-N-[2-(2-furyl)-6-(1H-pyrazol-1-yl)pyrimidin-4-yl]propanamide

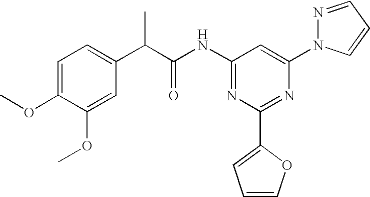

To a solution of 2-(3,4-dimethoxyphenyl)propanoic acid (0.185 g, 0.88 mmol) and a catalytic amount of DMF in methylene chloride (2 mL) was added oxalyl chloride (84 mL, 0.968 mmol) and the reaction mixture was stirred at room temperature for 3 hours. The solvent was removed under reduced pressure to give 2-(3,4-dimethoxyphenyl)propanoyl chloride that was used in the next reaction without further purification. 2-(3,4-Dimethoxyphenyl)-N-[2-(2-furyl)-6-(1H-pyrazol-1-yl)pyrimidin-4-yl]propanamide was obtained from the title compound of Example 1 (0.1 g) and 2-(3,4-dimethoxy phenyl)propanoyl chloride (0.201 g, 0.88 mmol) by the procedure described in Example 153. Purification by column chromatography with silica gel and ethyl acetate/n-hexane (2:8) as eluent gave 2-(3,4-dimethoxyphenyl)-N-[2-(2-furyl)-6-(1H-pyrazol-1-yl)pyrimidin-4-yl]propanamide (178 mg, 96%) as an off-white solid.

δ (400 MHz, CDCl₃): 1.58 (d, J=7.0 Hz, 3H); 3.68 (q, J=7.0 Hz, 1H); 3.88 (s, 3H); 3.89 (s, 3H); 6.49 (dd, J1=2.4 Hz, J2=1.6 Hz, 1H); 6.57 (dd, J1=3.2 Hz, J2=1.6 Hz, 1H); 6.82-6.91 (m, 3H); 7.31 (d, J=2.4 Hz, 1H); 7.60 (d, J=0.8 Hz, 1H); 7.79 (d, J=0.8 Hz, 1H); 8.00 (bs, 1H); 8.61 (d, J=2.4 Hz, 1H); 8.63 (s, 1H).

### EXAMPLE 166

### N-[2-(2-Furyl)-6-(1H-pyrazol-1-yl)pyrimidin-4-yl]benzamide

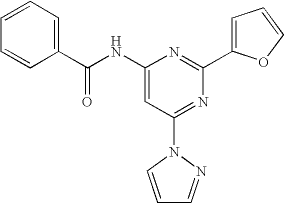

To a solution of the title compound of Example 1 (0.20 g, 0.88 mmol) in methylene chloride (10 mL) was added pyridine (89 mg, 1.06 mmol) and benzoyl chloride (0.15 g, 1.06 mmol). The mixture was stirred at room temperature for 18 hours and more pyridine (0.13 g, 1.6 mmol) and benzoyl chloride (0.22 g, 1.5 mmol) were added. The reaction was allowed to stand for 96 further hours at room temperature. The solution was diluted with methylene chloride (50 mL), washed with water (20 mL), with 1% sodium hydroxide (2×20 mL), and dried (Na₂SO₄). The solvent was removed under reduced pressure. Purification by column chromatography with silica gel, eluting with methylene chloride, followed by a second column chromatography with silica gel and ethyl acetate/n-hexane (1:4) as eluent gave N-[2-(2-furyl)-6-(1H-pyrazol-1-yl)pyrimidin-4-yl]benzamide (0.16 g, 49%) as an off-white solid.

δ (250 MHz, CDCl₃): 6.52 (dd, J1=2.7 Hz, J2=1.8 Hz, 1H); 6.61 (dd, J1=3.6 Hz, J2=1.8 Hz, 1H); 7.38 (dd, J1=3.3 Hz, J2=0.9 Hz, 1H); 7.63-7.5 (m, 3H); 7.66 (dd, J1=1.5 Hz, J2=0.9 Hz, 1H); 7.83 (dd, J1=1.5 Hz, J2=0.6 Hz, 1H); 7.98 (m, 2H); 8.67 (dd, J1=2.7 Hz, J2=0.6 Hz, 1H); 8.78 (s, 1H); 8.81 (bs, 1H).

### EXAMPLE 167

### N-[2-(2-Furyl)-6-(1H-pyrazol-1-yl)pyrimidin-4-yl]-3,4-dimethoxybenzamide

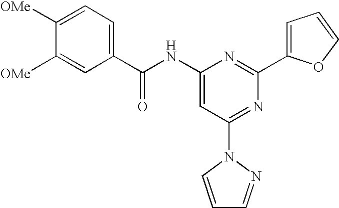

To a solution of the title compound of Example 1 (0.14 g, 0.62 mmol) in DMF (5 mL) was added sodium hydride (30 mg, 1.23 mmol). The mixture was stirred at room temperature for 1 hour. Then, a solution of 3,4-dimethoxybenzoyl chloride (0.49 g, 2.47 mmol) in DMF (4 mL) was added. The reaction was stirred at room temperature for 4 hours. The solvent was removed under reduced pressure. The crude was solved with methylene chloride (25 mL), washed with 10% sodium hydroxide (2×25 mL), water (2×25 mL), dried (Na₂SO₄), and the solvent removed under reduced pressure. Purification by column chromatography with silica gel and methylene chloride/methanol (0.5%) as eluent, followed by trituration with ethyl ether gave N-[2-(2-furyl)-6-(1H-pyrazol-1-yl)pyrimidin-4-yl]-3,4-dimethoxybenzamide (48 mg, 13%) as an off-white solid.

δ (250 MHz, CDCl₃): 3.97 (s, 3H); 3.99 (s, 3H); 6.51 (m, 1H); 6.61 (m, 1H); 6.95 (m, 1H); 7.39 (m, 1H); 7.55 (m, 2H); 7.66 (m, 1H); 7.82 (bs, 1H); 8.66 (m, 1H); 8.77 (m, 1H); 8.80 (bs, 1H).

### EXAMPLE 168

### 2,6-Difluoro-N-[2-(2-furyl)-6-(1H-pyrazol-1-yl)pyrimidin-4-yl]-benzamide

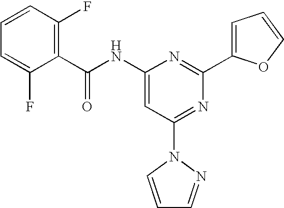

Obtained from the title compound of Example 1 (0.25 g) by the procedure described in Example 2. Purification by column chromatography with silica gel and ethyl acetate/n-hexane (1:1) as eluent gave 2,6-difluoro-N-[2-(2-furyl)-6-(1H-pyrazol-1-yl)pyrimidin-4-yl]-benzamide (0.38 g, 82%) as a white solid.

δ (400 MHz, CDCl₃): 6.51-6.48 (m, 1H); 6.60-6.58 (m, 1H); 7.34 (m, 2H); 7.62 (m, 2H); 8.13 (bs, 1H); 8.58 (s, 1H); 8.62 (d, J=2.4 Hz, 1H).

### EXAMPLE 169

### N-[2-(2-Furyl)-6-(1H-pyrazol-1-yl)pyrimidin-4-yl]-2-furamide

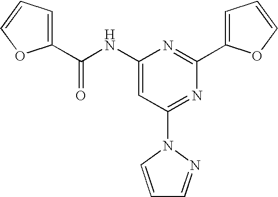

Obtained from the title compound of Example 1 (0.20 g) by the procedure described in Example 166. Purification by column chromatography with silica gel and methylene chloride/methanol (1%) as eluent gave N-[2-(2-furyl)-6-(1H-pyrazol-1-yl)pyrimidinyl]-2-furamide (210 mg, 65%) as an off-white solid.

δ (250 MHz, CDCl₃): 6.51 (dd, J1=2.7 Hz, J2=1.5 Hz, 1H); 6.61 (m, 2H); 7.36 (dd, J1=3.6 Hz, J2=0.9 Hz, 1H); 7.38 (dd, J1=3.6 Hz, J2=0.9 Hz, 1H); 7.57 (dd, J1=1.8 Hz, J2=0.9 Hz, 1H); 7.66 (dd, J1=1.8 Hz, J2=0.9 Hz, 1H); 7.82 (m, 1H); 8.66 (dd, J1-=2.7 Hz, J2=0.6 Hz, 1H); 8.70 (s, 1H); 8.96 (bs, 1H).

### EXAMPLE 170

### N-[2-(2-Furyl)-6-(1H-pyrazol-1-yl)pyrimidin-4-yl]thiophene-2-carboxamide

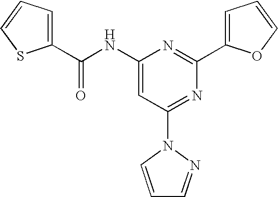

Obtained from the title compound of Example 1 (0.20 g) by the procedure described in Example 166. Purification by column chromatography with silica gel and methylene chloride/methanol (0.5%) as eluent gave N-[2-(2-furyl)-6-(1H-pyrazol-1-yl)pyrimidin-4-yl]thiophene-2-carboxamide (195 mg, 58%) as an off-white solid.

δ (250 MHz, CDCl₃): 6.51 (dd, J1=2.7 Hz, J2=1.5 Hz, 1H); 6.61 (dd, J1=3.3 Hz, J2=1.8 Hz, 1H); 7.18 (dd, J1=5.2 Hz, J2=3.9 Hz, 1H); 7.38 (dd, J1=3.3 Hz, J2=0.9 Hz, 1H); 7.67-7.64 (m, 2H); 7.76 (dd, J1=3.6 Hz, J2=0.9 Hz, 1H); 7.81 (dd, J1=1.5 Hz, J2=0.6 Hz, 1H); 8.65 (dd, J1=2.7 Hz, J2=0.6 Hz, 1H); 8.68 (bs, 1H); 8.71 (s, 1H).

### EXAMPLE 171

### N-[2-(2-Furyl)-6-(1H-pyrazol-1-yl)pyrimidin-4-yl]nicotinamide

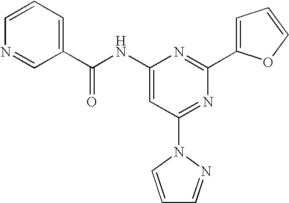

Obtained from the title compound of Example 1 (0.20 g) by the procedure described in Example 166. Purification by trituration with ethyl ether gave N-[2-(2-furyl)-6-(1H-pyrazol-1-yl)pyrimidin-4-yl]nicotinamide (43 mg, 13%) as an off-white solid.

δ (250 MHz, DMSO-d₆): 6.69 (m, 1H); 6.77 (m, 1H); 7.56 (m, 2H); 7.98 (m, 2H); 8.37 (m, 1H); 8.60 (bs, 1H); 8.80 (m, 2H); 9.15 (bs, 1H); 11.75 (s, 1H).

### EXAMPLE 172

### N-[2-(2-Furyl)-6-(1H-pyrazol-1-yl)pyrimidin-4-yl]isonicotinamide

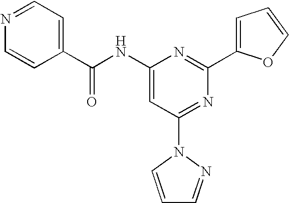

Obtained from the title compound of Example 1 (0.20 g) by the procedure described in Example 166. Purification by trituration with methylene chloride/diethyl ether gave N-[2-(2-furyl)-6-(1H-pyrazol-1-yl)pyrimidin-4-yl]isonicotinamide (43 mg, 36%) as an off-white solid.

δ (250 MHz, CDCl₃): 6.53 (dd, J1=2.7 Hz, J2=1.8 Hz, 1H); 6.61 (dd, J1=3.3 Hz, J2=1.8 Hz, 1H); 7.38 (dd, J1=3.3 Hz, J2=0.9 Hz, 1H); 7.65 (dd, J1=1.5 Hz, J2=0.6 Hz, 1H); 7.85-7.78 (m, 3H); 8.66 (m, 1H); 8.74 (s, 1H); 8.86 (m, 2H); 8.91 (bs, 1H).

### EXAMPLE 173

### N-[2-(2-Furyl)-6-(1H-pyrazol-1-yl)pyrimidin-4-yl]-1-naphthamide

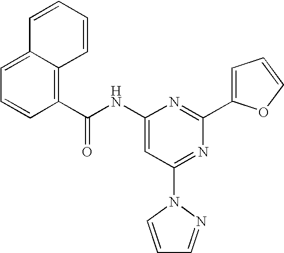

Obtained from the title compound of Example 1 (0.22 g) by the procedure described in Example 2. Purification by column chromatography with silica gel and ethyl acetate/n-hexane (1:1) as eluent gave N-[2-(2-furyl)-6-(1H-pyrazol-1-yl)pyrimidin-4-yl]-1-naphthamide (0.44 g, 87%) as a white solid.

δ (400 MHz, CDCl₃): 6.51-6.48 (m, 1H); 6.60-6.58 (m, 1H); 7.34 (m, 4H); 7.62 (m, 5H); 7.79 (s, 1H); 8.13 (bs, 1H); 8.58 (s, 1H); 8.62 (d, J=2.4 Hz, 1H).

### EXAMPLE 174

### N-[2-(2-Furyl)-6-(1H-pyrazol-1-yl)pyrimidin-4-yl]quinoline-2-carboxamide

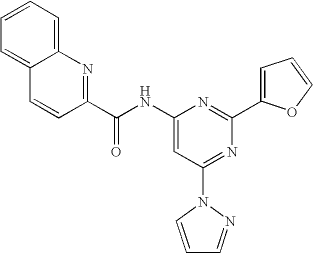

Obtained from the title compound of Example 1 (0.20 g) by the procedure described in Example 2. Purification by column chromatography with silica gel and ethyl acetate/n-hexane (1:1) as eluent gave N-[2-(2-furyl-6-(1H-pyrazol-1-yl)pyrimidin-4-yl]-quinoline-2-carboxamide (0.32 g, 67%) as a white solid.

δ (400 MHz, CDCl₃): 6.51-6.48 (m, 1H); 6.60-6.58 (m, 1H); 7.34 (m, 4H); 7.62 (m, 4H); 7.79 (s, 1H); 8.13 (bs, 1H); 8.58 (s, 1H); 8.62 (d, J=2.4 Hz, 1H).

### EXAMPLE 175

### (2E)-3-(3,4-Dimethoxyphenyl)-N-[6-(3,5-dimethyl-1H-pyrazol-1-yl)-2-(2-furyl)pyrimidin-4-yl]acrylamide

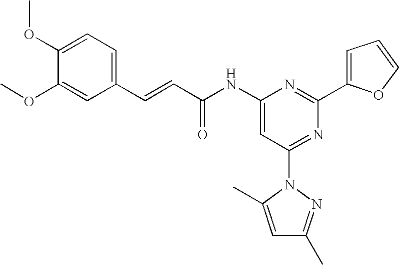

Obtained from the title compound of Example 21 (0.30 g), (2E)-(3,4-dimethoxyphenyl)acrylic acid (2.24 g, 10.8 mmol) and pyridine (6 mL, 74 mmol) by the procedure described in Example 20. Purification by column chromatography with silica gel and methylene chloride/methanol (0.3%) as eluent, followed by trituration with ethyl ether gave (2E)-3-(3,4-dimethoxyphenyl)-N-[6-(3,5-dimethyl-1H-pyrazol-1-yl)-2-(2-furyl)pyrimidin-4-yl]acrylamide (0.15 g, 27%) as an off-white solid.

δ (250 MHz, DMSO-d₆): 2.26 (s, 3H); 2.76 (s, 3H); 3.81 (s, 3H); 3.83 (s, 3H); 6.24 (s, 1H); 6.75 (s, 1H); 7.32-6.99 (m, 6H); 7.64 (d, J=15.5 Hz, 1H); 7.98 (s, 1H); 8.51 (s, 1H).

### EXAMPLE 176 AND EXAMPLE 177

### 2-(2-Furyl)-6-(2H-1,2,3-triazol-2-yl)pyrimidin-4-amine and 2-(2-furyl)-6-(1H-1,2,3-triazol-1-yl)pyrimidin-4-amine

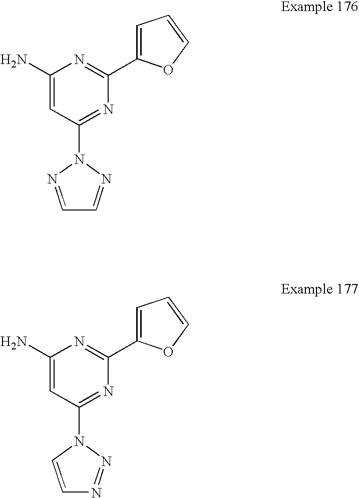

Obtained from Intermediate 4 (0.51 g) by the procedure described in Example 21. Purification by column chromatography with silica gel and methylene chloride/methanol (from 0.5% to 2%) as eluent gave 2-(2-furyl)-(2H-1,2,3-triazol-2-yl)pyrimidin-4-amine (0.16 g, 27%) and 2-(2-furyl)-6-(1H-1,2,3-triazol-1-yl)pyrimidin-4-amine (0.30 g, 51%) as off-white solids.

### EXAMPLE 176

δ (250 MHz, CDCl₃): 5.25 (bs, 2H); 6.55-6.57 (m, 1H); 6.99 (s, 1H); 7.25 (s, 1H); 7.60 (s, 1H); 7.92 (s, 1H).

### EXAMPLE 177

δ (250 MHz, CDCl₃): 6.67-6.68 (m, 1H); 6.70 (s, 1H); 7.32 (s, 1H); 7.52 (bs, 2H); 7.89 (s, 1H); 7.99 (s, 1H); 8.86 (s, 1H).

### EXAMPLE 178

### N-[2-(2-Furyl)-6-(2H-1,2,3-triazol-2-yl)pyrimidin-4-yl]propanamide

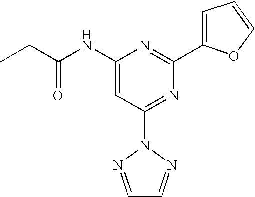

Obtained from the title compound of Example 176 (0.11 g) by the procedure described in Example 2. Purification by column chromatography with silica gel and methylene chloride/methanol (from 0.5% to 1%) as eluent gave N-[2-(2-furyl)-6-(2H-1,2,3-triazol-2-yl)pyrimidinyl]propanamide (95 mg, 70%) as an off-white solid.

δ (250 MHz, CDCl₃): 1.27 (t, J=7.6 Hz, 3H); 2.50 (c, J=7.6 Hz, 2H); 6.59 (dd, J1=3.3 Hz, J2=1.5 Hz, 1H); 7.46 (dd, J1=3.6 Hz, J2=0.9 Hz, 1H); 7.64 (dd, J1=1.8 Hz, J2=0.9 Hz, 1H); 7.96 (s, 2H); 8.26 (bs, 1H); 8.72 (s, 1H).

### EXAMPLE 179

### 2-(3,4-Dimethoxyphenyl)-N-[2-(2-furyl)-6-(2H-1,2,3-triazol-2-yl)pyrimidin-4-yl]acetamide

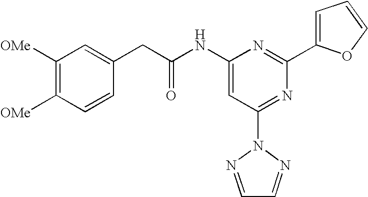

Obtained from the title compound of Example 176 (0.10 g) by the procedure described in Example 2. Purification by column chromatography with silica gel and ethyl acetate/n-hexane (1:1) as eluent, followed by trituration with ethyl ether/n-hexane gave 2-(3,4-dimethoxyphenyl)-N-[2-(2-furyl)-6-(2H-1,2,3-triazol-2-yl)-pyrimidin-yl]acetamide (74 mg, 42%) as an off-white solid.

δ (250 MHz, CDCl₃): 3.75 (s, 2H); 3.90 (s, 6H); 6.57 (dd, J1=3.6 Hz, J2=1.8 Hz, 1H); 6.84 (s, 1H); 6.89 (bs, 2H); 7.42 (dd, J1=3.6 Hz, J2=0.9 Hz, 1H); 7.62 (dd, J1=1.8 Hz, J2=0.9 Hz, 1H); 7.96 (s, 2H); 8.20 (bs, 1H); 8.72 (s, 1H).

### EXAMPLE 180

### N-[2-(2-Furyl)-6-(1H-1,2,3-triazol-1-yl)pyrimidin-yl]propanamide

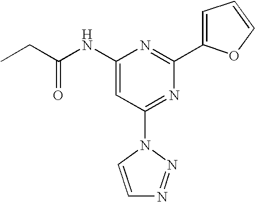

Obtained from the title compound of Example 177 (0.15 g) by the procedure described in Example 2. Purification by column chromatography with silica gel and methylene chloride/methanol (0.5%) as eluent, followed by trituration with ethyl ether gave N-[2-(2-furyl)-6-(1H-1,2,3-triazol-2-yl)pyrimidin-4-yl]propanamide (95 mg, 56%) as an off-white solid.

δ (250 MHz, DMSO-d₆): 1.09 (t, J=7.4 Hz, 3H); 6.76 (dd, J1=3.4 Hz, J2=1.7 Hz, 1H); 7.51 (d, J=3.7 Hz, 1H); 7.98 (bs, 1H); 8.07 (d, J=1.0 Hz, 1H); 8.64 (s, 1H); 9.02 (d, J=1.0 Hz, 1H); 11.36 (s, 1H).

### Intermediate 69. Ethyl 3-oxo-3-(1,3-thiazol-2-yl)propanoate

Obtained from 2-acetylthiazole (5.0 g) by the procedure described in Intermediate 35. The title compound was obtained (4.4 g, 56%) as an oil by distillation under reduced pressure.

δ (250 MHz, CDCl₃): 1.23 (t, 3H); 4.15 (m, 4H); 7.71 (d, J=5.3 Hz, 1H); 7.99 (d, J=5.3 Hz, 1H).

### Intermediate 70. 2-(2-Furyl)-6-(1,3-thiazol-2-yl)pyrimidin-4-ol

Obtained from Intermediate 69 (2.30 g) and Intermediate 1 (2.0 g) by the procedure described in Intermediate 36. Purification by trituration with ethyl ether gave 2-(2-furyl)-6-(1,3-thiazol-2-yl)pyrimidin-4-ol (1.3 g, 46%) as an off-white solid.

δ (250 MHz, CDCl₃): 6.76-6.78 (m, 1H); 6.87 (m, 1H); 7.62 (s, 1H); 8.05 (m, 1H); 8.09 (m, 2H); 12.92 (bs, 1H).

### Intermediate 71. 4-Chloro-2-(2-furyl)-6-(1,3-thiazol-2-yl)pyrimidine

Obtained from Intermediate 70 (2.80 g) by the procedure described in Intermediate 15. Purification by column chromatography with silica gel and methylene chloride/methanol (3%) as eluent gave 4-chloro-2-(2-furyl)-6-(1,3-thiazol-2-yl)pyrimidine (1.87 g, 62%) as an off-white solid.

MS (M⁺): 263.

### EXAMPLE 181

### 2-(2-Furyl)-6-(1,3-thiazol-2-yl)pyrimidin-amine

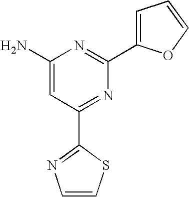

Obtained from Intermediate 71 (1.87 g) by the procedure described in Example 48. Purification by column chromatography with silica gel and methylene chloride/methanol (from 2% to 5%) as eluent gave 2-(2-furyl)-6-(1,3-thiazol-2-yl)pyrimidin-4-amine (1.30 g, 74%) as an off-white solid.

δ (250 MHz, DMSO-d₆): 6.66 (dd, J1=3.3 Hz, J2=1.6 Hz, 1H); 7.04 (s, 1H); 7.17 (dd, J1=3.3 Hz, J2=0.6 Hz, 1H); 7.30 (bs, 2H); 7.88-7.87 (m, 1H); 7.93 (d, J=3.0 Hz, 1H); 8.03 (d, J=3.0 Hz, 1H).

### EXAMPLE 182

### N-[2-(2-Furyl)-6-(1,3-thiazol-2-yl)pyrimidin-4-yl]propanamide

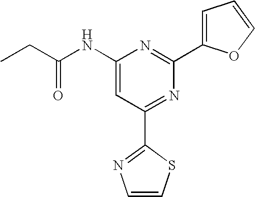

Obtained from the title compound of Example 181 (0.22 g) by the procedure described in Example 2. Purification by column chromatography with silica gel and methylene chloride/methanol (1%) as eluent gave N-[2-(2-furyl)-6-(1,3-thiazol-2-yl)pyrimidin-4-yl]propanamide (0.22 g, 90%) as an off-white solid.

δ (250 MHz, CDCl₃): 1.27 (t, J=7.6 Hz, 3H); 2.48 (c, J=7.6 Hz, 2H); 6.59 (dd, J1=3.6 Hz, J2=1.8 Hz, 1H); 7.40 (dd, J1=3.3 Hz, J2=0.9 Hz, 1H); 7.55 (d, J=3.3 Hz, 1H); 7.64 (m, 1H); 8.02 (d, J=3.3 Hz, 1H); 8.13 (bs, 1H); 8.78 (s, 1H).

### EXAMPLE 183

### 3-(3,4-Dimethoxyphenyl)-N-[2-(2-furyl)-6-(1,3-thiazol-2-yl)pyrimidin-4-yl]propanamide

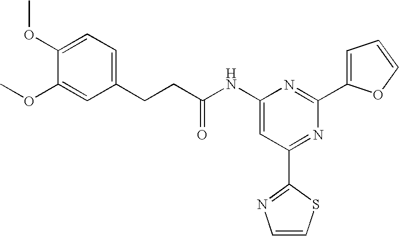

Obtained from the title compound of Example 181 (0.2 g) by the procedure described in Example 14. Purification by column chromatography with silica gel and methylene chloride/methanol (1%) as eluent gave 3-(3,4-dimethoxyphenyl)-N-[2-(2-furyl)-6-(1,3-thiazol-2-yl)pyrimidin-4-yl]propanamide (86 mg, 24%) as an off-white solid.

δ (250 MHz, CDCl₃): 2.72 (t, J=7.6 Hz, 2H); 3.02 (t, J=7.6 Hz, 2H); 3.85 (s, 3H); 3.86 (s, 3H); 6.59 (dd, J1=3.3 Hz, J2=1.8 Hz, 1H); 6.79-6.76 (m, 3H); 7.39 (d, J=3.3 Hz, 1H); 7.56 (d, J1=3.3 Hz, 1H); 7.63 (m, 1H); 8.03 (d, J=3.3 Hz, 1H); 8.05 (bs, 1H); 8.77 (s, 1H).

### EXAMPLE 184

### 2,6-Di-2-furylpyrimidin-4-amine

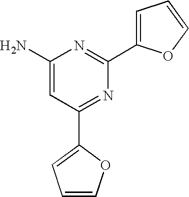

Obtained from Intermediate 4 (0.2 g) by the procedure described in Example 54. Purification by column chromatography with silica gel and ethyl acetate/n-hexane (7:3) as eluent gave 2,6-di-2-furylpyrimidin-4-amine (0.27 g, 89%) as an off-white solid.

δ (250 MHz, CDCl₃): 5.09 (bs, 2H); 6.62-6.46 (m, 3H); 7.28-7.17 (m, 2H); 7.57-7.47 (m, 2H).

### EXAMPLE 185

### N-(2,6-Di-2-furylpyrimidin-4-yl)-2-(3,4-dimethoxyphenyl)acetamide

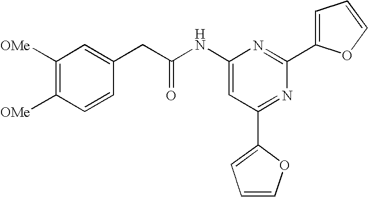

Obtained from the title compound of Example 184 (0.2 g) by the procedure described in Example 2. Purification by column chromatography with silica gel and ethyl acetate/n-hexane (7:3) as eluent gave N-(2,6-di-2-furylpyrimidin-4-yl)-2-(3,4-dimethoxyphenyl)acetamide (0.22 g, 60%) as an off-white solid.

δ (250 MHz, CDCl₃): 3.72 (s, 2H); 3.90 (2s,6H); 6.55 (dd, J1=3.3 Hz, J2=1.8 Hz, 1H); 6.57 (dd, J1=3.3 Hz, J2=1.8 Hz, 1H); 6.83 (m, 1H); 6.88 (m, 2H); 7.32 (m, 2H); 7.60 (m, 2H); 8.12 (bs, 1H); 8.34 (s, 1H).

### Intermediate 72. N-[6-Chloro-2-(2-furyl)pyrimidin-4-yl]acetamide

Obtained from Intermediate 4 (0.89 g) by the procedure described in Example 2. Purification by column chromatography with silica gel and methylene chloride/methanol (from 0.5% to 1%) as eluent gave N-[6-chloro-2-(2-furyl)pyrimidinyl]acetamide (1.03 g, 95%) as an off-white solid.

δ (250 MHz, CDCl₃): 2.21 (s, 3H); 6.56-6.57 (m, 1H); 7.29 (d, J=3.8 Hz, 1H); 7.60 (m, 1H); 8.03 (s, 1H); 8.29 (bs, 1H).

### EXAMPLE 186

### 6-(1,3-Benzothiazol-2-yl)-21(2-furyl)pyrimidin-4-amine

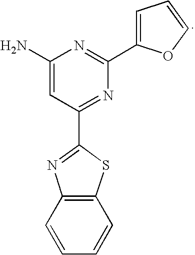

To a solution of Intermediate 72 (0.15 g, 0.63 mmol) in anhydrous DMF (2 mL) were added 2-tributylstannanylbenzothiazole (0.32 g, 0.76 mmol) and bis(triphenylphosphine) palladium (II) chloride (90 mg, 0.13 mmol). The mixture was stirred at 80° C. overnight and more tributylstannanylbenzothiazole (0.30 g, 0.70 mmol) and bis(triphenylphosphine) palladium (II) chloride (70 mg, 0.10 mmol) were added. The mixture was stirred at 80° C. for 6 hours. The crude reaction was filtered through Celite® and the solvent was removed under reduced pressure. The residue was solved in chloroform (25 mL) and the resulting solution was washed with saturated solution of sodium bicarbonate (2×25 mL), water (2×25 mL), brine (25 mL), dried (Na₂SO₄), and the solvent removed under reduced pressure. Purification by column chromatography with silica gel and ethyl acetate/n-hexane (from 3:7 to 2:3) as eluent gave N-[6-(1,3-benzothiazol-2-yl)-2-(2-furyl)pyrimidin-4-yl]acetamide (0.15 g, 71%). Hydrolisis of the resulting solid in methanol (5 mL) and 15% hydrochloric acid (10 mL) at 70° C. for 3 hours, followed by purification by column chromatography with silica gel and methylene chloride/methanol (2%) as eluent, gave the title compound (93 mg, 71%) as an off-white solid.

δ (250 MHz, DMSO-d₆): 6.69 (dd, J1=3.3 Hz, J2=1.7 Hz, 1H); 7.22 (m, 2H); 7.41 (bs, 2H); 7.56 (m, 2H); 7.90 (dd, J1=1.7 Hz, J2=1.0 Hz, 1H); 8.12 (dd, J1=7.7 Hz, J2=1.0 Hz, 1H); 8.20 (dd, J1=7.7 Hz, J2=1.0 Hz, 1H).

### Intermediate 73. 2-(5-Methyl-2-furyl)-6-(1,3-thiazol-2-yl)pyrimidin-4-ol

To a solution of potassium tertbutoxide (0.57 g, 6.03 mmol) in butanol (2 mL) were added Intermediate 69 (0.85 g, 4.26 mmol) and Intermediate 8 (0.75 g, 4.69 mmol). The mixture was heated at 135° C. for 3 hours. The crude reaction was poured into water (20 mL) and acidified with 10% hydrochloric acid (25 mL). The resulting solid was filtered, washed with water (2×25 mL) and dried. The title compound was obtained (0.64 g, 50%) as an off-white solid.

δ (250 MHz, CDCl₃): 2.45 (s, 3H); 6.38 (d, J=2.8 Hz, 1H); 6.77 (s, 1H); 7.44 (d, J=2.8 Hz, 1H); 7.98 (d, J=2.8 Hz, 1H); 8.03 (d, J=2.8 Hz, 1H).

### Intermediate 74. 4-Chloro-2-(5-methyl-2-furyl)-6-(1,3-thiazol-2-yl)pyrimidine

Obtained from Intermediate 73 (0.63 g) by the procedure described in Intermediate 15. Purification by column chromatography with silica gel and methylene chloride as eluent gave 4-chloro-2-(5-methyl-2-furyl)-6-(1,3-thiazol-2-yl)pyrimidine (0.44 g, 66%) as an off-white solid.

δ (250 MHz, CDCl₃): 2.41 (s, 3H); 6.15 (d, J=4.8 Hz, 1H); 7.31 (d, J=3.2 Hz, 1H); 7.53 (d, J=3.2 Hz, 1H); 7.81 (s, 1H); 8.03 (d, J=4.8 Hz, 1H).

### EXAMPLE 187

### 2-(5-Methyl-2-furyl)-6-(1,3-thiazol-2-yl)pyrimidin-4-amine

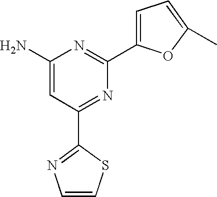

Obtained from Intermediate 74 (0.25 g) by the procedure described in Example 48. Purification by trituration with ethyl ether gave 2-(5-methyl-2-furyl)-6-(1,3-thiazol-2-yl)pyrimidin-4-amine (0.12 g, 53%) as an off-white solid.

δ (250 MHz, DMSO-d₆): 2.38 (s, 3H); 6.29 (dd, J1=3.0 Hz, J2=1.0 Hz, 1H); 6.99 (m, 1H); 7.08 (d, J=3.4 Hz, 1H); 7.28 (bs, 2H); 7.93 (dd, J1=3.0 Hz, J2=1.0 Hz, 1H); 8.03 (dd, J1=3.0 Hz, J2=1.0 Hz, 1H).

### Intermediate 75. 6-(1,3-Thiazol-2-yl)-2-(2-thienyl)pyrimidin-ol

Obtained from Intermediate 69 (1.00 g) and Intermediate 13 (0.98 g) by the procedure described in Intermediate 73. Purification by trituration iwtyh ethyl ether gave the title compound (0.44 g, 66%) as an off-white solid.

MS (M⁺): 261.

### Intermediate 76. 4-Chloro-6-(1,3-thiazol-2-yl)-2-(2-thienyl)pyrimidine

Obtained from Intermediate 75 (0.45 g) by the procedure described in Intermediate 15. Purification by column chromatography with silica gel and methylene chloride/methanol (5%) as eluent gave 4-chloro-6-(1,3-thiazol-2-yl)-2-(2-thienyl)pyrimidine (0.48 g, 99%) as an off-white solid.

MS (M⁺): 279.

### EXAMPLE 188

### 6-(1,3-Thiazol-2-yl)-2-(2-thienyl)pyrimidin-4-amine

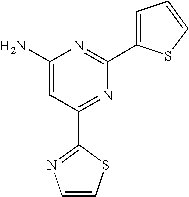

Obtained from Intermediate 76 (0.25 g) by the procedure described in Example 48. Purification by trituration with ethyl ether gave 6-(1,3-thiazol-2-yl)-2-(2-thienyl)pyrimidin-4-amine (94 mg, 40%) as an off-white solid.

δ (250 MHz, CDCl₃): 5.08 (bs, 2H); 7.10 (s, 1H), 7.14 (dd, J1=4.8 Hz, J2=3.6 Hz, 1H); 7.46 (dd, J1=4.8 Hz, J2=1.2 Hz, 1H); 7.52 (d, J=3.3 Hz, 1H); 7.96 (d, J=3.3 Hz, 1H); 8.01 (dd, J1=3.6 Hz, J2=1.2 Hz, 1H).

### EXAMPLE 189

### 2-(3,4-Dimethoxyphenyl)-N-[6-(2-furyl)-2-(1,3-thiazol-2-yl)pyrimidin-4-yl]acetamide

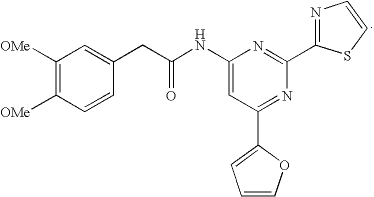

Obtained from the title compound of Example 116 (0.14 g) by the procedure described in Example 2. Purification by column chromatography with silica gel and ethyl acetate/n-hexane (from 1:1 to pure ethyl acetate) as eluent gave 2-(3,4-dimethoxyphenyl)-N-[6-(2-furyl)-2-(1,3-thiazol-2-yl)pyrimidin-4-yl]acetamide (0.17 g, 75%) as an off-white solid.

δ (250 MHz, CDCl₃): 3.73 (s, 2H); 3.89 (s, 3H); 3.90 (s, 3H); 6.59 (dd, J1=3.3 Hz, J2=1.8 Hz, 1H); 6.82 (m, 1H); 6.87 (m, 2H); 7.39 (dd, J1=3.3 Hz, J2=0.9 Hz, 1H); 7.52 (d, J=3.3 Hz, 1H); 7.63 (m, 1H); 8.00 (d, J=3.3 Hz, 1H); 8.26 (bs, 1H); 8.50 (s, 1H).

### Intermediate 77. 6-Amino-2-(1,3-thiazol-2-yl)pyrimidin-4-ol

To a solution of potassium tertbutoxide (2.5 g, 22.24 mmol) in butanol (12.7 mL), ethyl cyanoacetate (2.37 mL, 22.24 mmol) and thiazole-2-carboxamidine (HCl) (1.82 g, 11.12 mmol) were added. The mixture was heated at 135° C. for 3 hours. The solvent was evaporated and the residue was diluted with water (50 mL). The aqueous solution was neutralized by addition of acetic acid, washed with ethyl ether (2×25 mL) and purified using a Bond-Elut C18 column. The final product was eluted using methanol as eluent. Final purification by flash chromatography with silica gel and chloroform/methanol (9:1) as eluent gave 6-amino-2-(1,3-thiazol-2-yl)pyrimidin-4-ol (350 mg, 16%) as a yellow solid.

δ (300 MHz, DMSO-d₆): 5.21 (s, 1H), 6.72 (s, 2H), 8.00-8.10 (m, 2H), 11.60 (s, 1H).

### Intermediate 78. 6-Chloro-2-(1,3-thiazol-2-yl)pyrimidin-4-amine

A solution of Intermediate 77 (687 mg, 3.54 mmol) in phosphorus oxychloride (2.14 mL, 23.0 mmol) was heated at 100° C. for 2 hours. The solvent was removed under reduced pressure and the resulting residue was treated with ice-water. The solution was neutralized with solid sodium bicarbonate and extracted with chloroform (3×50 mL). The combined organic extracts were washed with water (2×50 mL), dried (MgSO₄), and the solvent removed under reduced pressure, to yield 6-chloro-2-(1,3-thiazol-2-yl)pyrimidin-4-amine (122.4 mg, 16%) as a brown solid.

δ (300 MHz, CDCl₃): 5.37 (bs, 2H); 6.47 (s, 1H); 7.54 (d, J=3.1 Hz, 1H); 8.01 (d, J=3.1 Hz, 1H).

### EXAMPLE 190

### 6-(1H-Pyrazol-1-yl)-2-(1,3-thiazol-2-yl)pyrimidin-4-amine

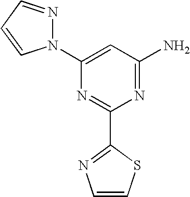

A suspension of Intermediate 78 (122.4 mg, 0.58 mmol), pyrazole (58.5 mg, 0.86 mmol) and potassium carbonate (118.9 mg, 0.86 mmol) in DMSO (4 mL) was stirred at 150° C. during 6 hours. After this reaction time, more pyrazole (58.5 mg, 0.86 mmol) and potassium carbonate (118.9 mg, 0.86 mmol) were added and the mixture was stirred at 150° C. overnight. The reaction mixture was diluted with water (25 mL) and extracted with chloroform (2×25 mL). The solvent was removed under reduced pressure. Purification by column chromatography with silica gel and methylene chloride/methanol (9:1) as eluent, followed by a trituration with ethyl ether gave of 6-(1H-pyrazol-1-yl)-2-(1,3-thiazol-2-yl)pyrimidin-amine (50 mg, 35%) as an off-white solid.

δ (300 MHz, CDCl₃): 5.30 (bs, 2H); 6.50 (dd, J1=2.7 Hz, J2=1.6 Hz, 1H); 7.04 (s, 1H); 7.54 (d, J=3.0 Hz, 1H); 7.78 (s, 1H); 8.04 (d, J=3.3 Hz, 1H); 8.69 (d, J=1.9 Hz, 1H).

### EXAMPLE 191

### 2-(3,4-Dimethoxyphenyl)-N-[6-(1H-pyrazol-1-yl)-2-(1,3-thiazol-2-yl)pyrimidin-4-yl]acetamide

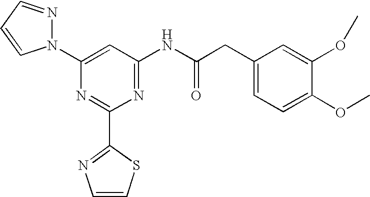

Obtained from the title compound of Example 190 (50 mg) and 3,4-dimethoxyphenylacetyl chloride (106 μL, 0.615 mmol) by the procedure described in Example 150. Purification by column chromatography with silica gel using chloroform/methanol (75:1) as eluent gave 2-(3,4-dimethoxyphenyl)-N-[6-(1H-pyrazol-1-yl)-2-(1,3-thiazol-2-yl)pyrimidin-4-yl]acetamide (35 mg, 40%) as a solid foam.

δ (300 MHz, CDCl₃): 3.74 (s, 2H); 3.89 (s, 6H); 6.53 (dd, J1=2.7 Hz, J2=1.4 Hz, 1H); 6.83 (s, 1H); 6.85-6.89 (m, 2H); 7.56 (d, J=3.0 Hz, 1H); 7.83 (m, 1H); 8.03 (d, J=3.3 Hz, 1H), 8.26 (s, 1H); 8.66 (m, 1H); 8.77 (s, 1H).

### EXAMPLE 192

### 2-(2-Furyl)-N-methyl-6-(1,3-thiazol-2-yl)pyrimidin-4-amine

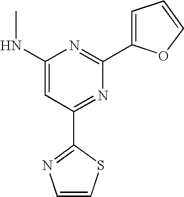

A suspension of Intermediate 71 (0.2 g, 0.76 mmol) in a solution of 33% methylamine in ethanol (5 mL) was heated at 40° C. in a pressure reactor for 2 hours. The solvent was partially removed under reduced pressure. The resulting solid was filtered, washed with ethyl ether (25 mL), and dried. 2-(2-Furyl)-N-methyl-6-(1,3-thiazol-2-yl)pyrimidin-4-amine was obtained (90 mg, 46%) as an off-white solid.

δ (250 MHz, CDCl₃): 3.06 (d, J=5.2 Hz, 3H); 5.40 (bs, 1H); 6.55 (dd, J1=3.3 Hz, J2=1.8 Hz, 1H); 7.05 (s, 1H); 7.34 (dd, J1=3.3 Hz, J2=0.9 Hz, 1H); 7.52 (d, J=3.3 Hz, 1H); 7.61 (dd, J1=1.8 Hz, J2=0.9 Hz, 1H); 7.96 (d, J=3.3 Hz, 1H).

### EXAMPLE 193

### N-(Cyclopropylmethyl)-2-(2-furyl)-6-(1,3-thiazol-2-yl)pyrimidin-4-amine

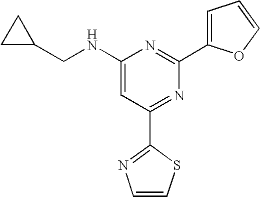

To a solution of Intermediate 71 (0.2 g, 0.76 mmol) in anhydrous DMF (5 mL) was added aminomethylcyclopropane (60 mg, 0.84 mmol) and cesium carbonate (0.27 g, 0.84 mmol). The mixture was heated at 80° C. for 8 hours. The solution was poured into water (25 mL) and extracted with ethyl acetate (2×15 mL). The organic layer was washed with water (2×10 mL) and brine (10 mL), dried (Na₂SO₄), and the solvent removed under reduced pressure. The resulting solid was purified by column chromatography with silica gel and ethyl acetate/n-hexane (from 10% to 20%) as eluent to give N-(cyclopropylmethyl)-2-(2-furyl)-6-(1,3-thiazol-2-yl)pyrimidin-4-amine (0.11 g, 81%) as an off-white solid.

δ (250 MHz, CDCl₃): 0.32-0.26 (m, 2H); 0.63-0.55 (m, 2H); 1.25-1.05 (m, 1H); 3.26 (bs, 2H); 5.48 (bs, 1H); 6.56-6.54 (m, 1H); 7.02 (s, 1H); 7.33 (d, J=3.3 Hz, 1H); 7.51 (d, J=3.0 Hz, 1H); 7.51 (d, J=3.0 Hz, 1H); 7.95 (d, J=3.3 Hz, 1H).

### EXAMPLE 194

### N-[2-(3,4-Dimethoxyphenyl)ethyl]-2-(2-furyl)-6-(1,3-thiazol-2-yl)pyrimidin-4-amine

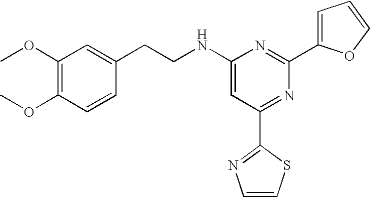

Obtained from Intermediate 71 (0.20 g) by the procedure described in Example 193. Purification by column chromatography with silica gel and ethyl acetate/n-hexane (from 20% to 35%) as eluent gave N-[2-(3,4-dimethoxyphenyl)ethyl]-2-(2-furyl)-6-(1,3-thiazol-2-yl)-pyrimidin-4-amine (0.13 g, 66%) as an off-white solid.

δ (250 MHz, CDCl₃): 2.92 (t, J=6.9 Hz, 2H); 3.69 (bs, 2H); 3.86 (s, 3H); 3.87 (s, 3H); 5.24 (bs, 1H); 6.56-6.54 (m, 1H); 6.84-6.75 (m, 3H); 7.04 (bs, 1H); 7.33 (d, J=3.3 Hz, 1H); 7.51 (d, J=3.0 Hz, 1H); 7.61 (bs, 1H); 7.95 (d, J=3.3 Hz, 1H).

### EXAMPLE 195

### N-(Cyclopropylmethyl)-6-(2-furyl)-2-(1,3-thiazol-2-yl)pyrimidin-4-amine

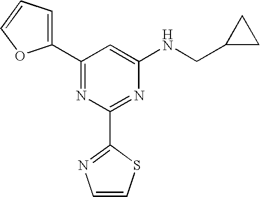

Obtained from Intermediate 52 (100 mg) and cyclopropylmethylamine (81 mg, 98 μl) by the procedure described in Example 119. Purification by column chromatography using a 10 g silica Bond-Elut cartridge eluting with n-hexane/ethyl acetate (from 1:0 to 3:7), followed by a second column chromatography using a 10 g silica Bond-Elut cartridge and n-hexane/ethyl acetate (from 4:1 to 3:2) as eluent gave N-cyclopropylmethyl)-6-(2-furyl)-2-(1,3-thiazol-2-yl)pyrimidin-4-amine (85 mg, 75%) as an off-white solid.

m.p.: 135.9-136.4° C.

δ (400 MHz, CDCl₃): 0.33 (m, 2H); 0.60 (m, 2H); 1.14 (m, 1H); 3.27 (bs, 2H); 5.48 (bs, 1H); 6.56-6.58 (m, 1H); 6.67 (s, 1H); 7.33 (d, J=3.1 Hz, 1H); 7.47 (m, 1H); 7.56 (s, 1H); 8.00 (m, 1H).

### EXAMPLE 196

### N-[2-(3,4-Dimethoxyphenyl)ethyl]-6-(2-furyl)-2-(1,3-thiazol-2-yl)pyrimidin-4-amine

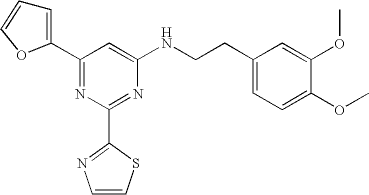

Obtained from Intermediate 52 (100 mg) and 2-(3,4-dimethoxyphenyl)ethylamine (207 mg, 192 μl) by the procedure described in Example 119. Purification by column chromatography using a 10 g silica Bond-Elut cartridge eluting with chloroform/methanol (99:1), followed by a second column chromatography using a 10 g silica Bond-Elut cartridge and n-hexane/ethyl acetate (from 1:0 to 3:2) as eluent gave N-[2-(3,4-dimethoxyphenyl)ethyl]-6-(2-furyl)-2-(1,3-thiazol-2-yl)pyrimidin-4-amine (110 mg, 71%) as solid foam.

m.p.: 83.0-83.8° C.

δ (400 MHz, CDCl₃): 2.94 (t, J=6.8 Hz, 2H); 3.70 (bs, 2H); 3.87 (s, 3H); 3.88 (s, 3H); 5.30 (bs, 1H); 6.55-6.58 (m, 1H); 6.64 (s, 1H); 6.76-6.85 (m, 3H); 7.33 (d, J=3.2 Hz, 1H); 7.47 (d, J=2.7 Hz, 1H); 7.55 (s, 1H); 7.99 (d, J=3.1 Hz; 1H).

### EXAMPLE 197

### 6-(2-Furyl)-N-(2-pyridin-3-ylethyl)-2-(1,3-thiazol-2-yl)pyrimidin-4-amine

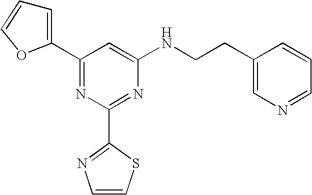

Obtained from Intermediate 52 (100 mg) and 2-pyridin-3-yl-ethylamine (139 mg, 1.14 mmol) by the procedure described in Example 119. Purification by column chromatography using a 10 g silica Bond-Elut cartridge eluting with chloroform/methanol (from pure chloroform to 98:2), followed by a second column chromatography using a 10 g silica Bond-Elut cartridge and methylene chloride/ethyl ether/methanol (from 1:1:0 to 1:1:0.1) as eluent gave 6-(2-furyl)-N-(2-pyridin-3-ylethyl)-2-(1,3-thiazol-2-yl)pyrimidin-4-amine (103 mg, 77%) an oil.

δ (400 MHz, CDCl₃): 3.02 (t, J=7.2 Hz, 2H); 3.75 (bs, 2H); 5.30 (bs, 1H); 6.58 (dd, J1=3.5 Hz, J2=2.0 Hz, 1H); 6.66 (s, 1H); 7.25-7.28 (m, 1H); 7.35 (d, J=3.5 Hz, 1H); 7.48 (d, J=3.1 Hz, 1H); 7.56 (s, 1H); 7.60 (m, 1H); 8.01 (d, J=13.1 Hz, 1H); 8.51 (m, 1H); 8.56 (d, J=2.4 Hz, 1H).

### EXAMPLE 198

### 6-(2-Furyl)-N-[(1S*,2R*)-2-phenylcyclopropyl]-2-(1,3-thiazol-2-yl)pyrimidin-4-amine (*relative trans configuration)

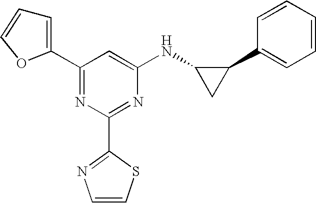

Obtained from Intermediate 52 (100 mg) and trans-2-phenylcyclopropylamine (187 mg, 1.40 mmol) by the procedure described in Example 119. Purification by column chromatography using a 10 g silica Bond-Elut cartridge eluting with n-hexane/ethyl acetate (from 1:0 to 6:4) gave 6-(2-furyl)-N-[(1S*,2R*)-2-phenylcyclopropyl]-2-(1,3-thiazol-2-yl)pyrimidin-4-amine (*relative trans configuration) (115 mg, 84%) as a solid foam.

m.p.: 90.0-91.9° C.

δ (400 MHz, CDCl₃): 1.32-1.37 (m, 1H); 1.46-1.51 (m, 1H); 2.15-2.20 (m, 1H); 2.81 (bs; 1H); 5.82 (bs, 1H); 6.56 (m, 1H); 6.93 (s, 1H); 7.19-7.38 (m, 6H); 7.47 (m, 1H); 7.54 (s, 1H); 7.99 (m, 1H).

### EXAMPLE 199

### Ethyl [2-(2-furyl)-6-(1H-pyrazol-1-yl)pyrimidin-4-yl]carbamate

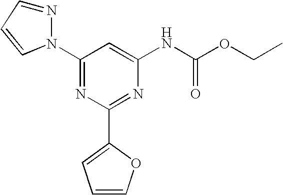

Obtained from the title compound of Example 1 (0.79 g) by the procedure described in Example 139. Purification by column chromatography with silica gel and chloroform as eluent, followed by a preparative HPLC/MS purification gave ethyl [2-(2-furyl)₆₋₁H-pyrazol-1-yl)pyrimidin-4-yl]carbamate (45 mg, 2%) as an off-white solid.

m.p.: 121.3-121.8° C.

δ (400 MHz, CDCl₃): 1.34 (t, J=7.2 Hz, 3H); 4.30 (q, J=7.2 Hz, 2H); 6.49 (m, 1H); 6.58 (m, 1H); 7.34 (d, J=3.1 Hz, 1H); 7.62 (s, 1H); 7.64 (s, 1H), 7.79 (s, 1H), 8.37 (s, 1H), 8.65 (d, J=2.7 Hz, 1H).

### Intermediate 79. Cyclopentylmethyl 4-nitrophenyl carbonate

To a solution of cyclopentylmethanol (0.54 mL, 4.99 mmol) and pyridine (0.60 mL, 7.43 mmol) in ethanol free chloroform (5 mL), cooled at 0-5° C. and under nitrogen, was added a solution of 4-nitrophenylchloroformate (1.26 g, 6.24 mmol) in ethanol free chloroform (5 mL). The reaction mixture was stirred at 0-5° C. for 1 hour and diluted with chloroform (150 mL). The organic layer was washed with water (50 mL), 2N hydrochloric acid (40 mL), water (2×50 mL), 4% sodium bicarbonate (2×40 mL) and water (50 mL), dried (MgSO₄), and the solvent removed under reduced pressure. The resulting oil was stirred in a mixture of ethyl ether/isopropyl ether at 0-5° C. for 1 hour to give a solid that was filtered, washed with isopropyl ether, and discarded. The mother liquors of filtration were combined, diluted with an equal volume of n-hexane, stirred at room temperature for 15 minutes. The resulting solid was filtered and the mother liquors were concentrated under reduced pressure to give cyclopentylmethyl 4-nitrophenyl carbonate (1.04 g, 78%) as an oil.

δ (300 MHz, CDCl₃): 1.23-1.40 (m, 2H); 1.56-1.74 (m, 4H); 1.77-1.89 (m, 2H); 2.32 (h, J=9.0 Hz, 1H); 4.19 (d, J=9.0 Hz, 1H); 7.39 (d, J=9.0 Hz, 2H); 8.29 (d, J=9.0 Hz, 2H).

### EXAMPLE 200

### Cyclopentylmethyl [2-(2-furyl)-6-(1H-pyrazol-1-yl)pyrimidin-4-yl]-carbamate

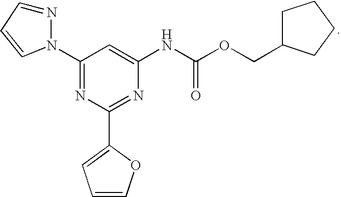

To a solution of the title compound of Example 1 (0.34 g, 1.51 mmol) in anhydrous tetrahydrofuran (15 mL), cooled at −78° C. and under nitrogen, was added a solution of 1.6M solution of n-butyllithium in hexanes (1.18 ml, 1.89 mmol). After 30 min at −78° C. a solution of Intermediate 79 (0.5 g, 1.89 mmol) in anhydrous tetrahydrofuran (8 mL) was added. The reaction mixture was maintained 30 minutes at −78° C., allowed to warm to room temperature and stirred at this temperature for 68 hours. The reaction mixture was poured into water (150 mL) and extracted with ethyl acetate (2×50 mL). The organic layer was washed with water (50 mL) and brine (50 mL), dried (MgSO₄), and the solvent removed under reduced pressure. Purification by column chromatography with silica gel and n-hexane/ethyl acetate (4:1) as eluent gave cyclopentylmethyl [2-(2-furyl)-6-(1H-pyrazol-1-yl)pyrimidin-4-yl]carbamate (180 mg, 34%) of as a solid foam.

m.p.: 60.0-61.3° C.

δ (400 MHz, CDCl₃): 1.24-1.34 (m, 2H); 1.54-1.68 (m, 4H); 1.74-1.82 (m, 2H); 2.22-2.32 (m, 1H); 4.13 (m, 2H); 6.50 (m, 1H); 6.59 (m, 1H); 7.34 (d, J=3.1 Hz, 1H); 7.63 (m, 1H); 7.64 (s, 1H); 7.79 (s, 1H); 8.36 (s, 1H); 8.65 (d, J=2.7 Hz, 1H).

### EXAMPLE 201

### Benzyl [2-(2-furyl)-6-(1H-pyrazol-1-yl)pyrimidin-yl]carbamate

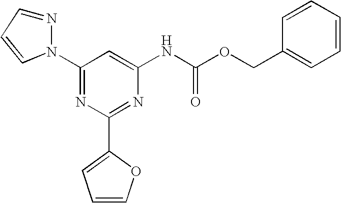

Obtained from the title compound of Example 1 (0.79 g) and benzyl chloroformate (0.2 mL, 1.40 mmol) by the procedure described in Example 200. Purification by column chromatography using silica gel and n-hexane/ethyl acetate (3:1) as eluent, followed by a HPLC/MS purification, gave benzyl [2-(2-furyl)-6-(1H-pyrazol-1-yl)pyrimidin-4-yl]carbamate (38 mg, 10%) as an off-white solid.

δ (400 MHz, CDCl₃): 5.27 (s, 2H); 6.49 (m, 1H); 6.57 (dd, J1=3.5 Hz, J2=1.6 Hz, 1H); 7.30-7.44 (m, 6H); 7.62 (s, 1H); 7.73 (s, 1H); 7.80 (s, 1H); 8.38 (s, 1H); 8.64 (d, J=2.7 Hz, 1H).

### Intermediate 80. 3,4-Dimethoxybenzyl 4-nitrophenyl carbonate

Obtained from 3,4-dimethoxybenzylalcohol (0.50 g, 2.97 mmol) by the procedure described in Intermediate 79. Purification by trituration with a mixture of ethyl ether/isopropyl ether (1:2), followed by a second trituration with ethyl ether gave the title compound (0.71 g, 71%).

δ (300 MHz, CDCl₃): 3.91 (s, 3H); 3.92 (s, 3H); 5.24 (s, 2H); 6.89 (d, J=8.4 Hz, 1H); 6.99 (dd, J1=8.4 Hz, J2=2.1 Hz, 1H); 7.04 (d, J=2.1 Hz, 1H); 7.38 (d, J=9.3 Hz, 2H); 9.28 (d, J=9.3 Hz, 2H).

### EXAMPLE 202

### 3,4-Dimethoxybenzyl [2-(2-furyl)-6-(1H-pyrazol-1-yl)pyrimidin-4-yl]carbamate

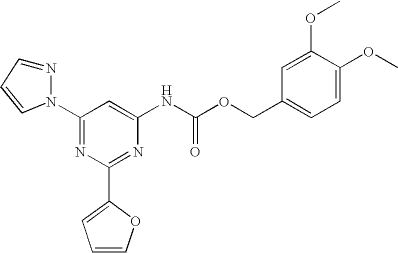

Obtained from the title compound of Example 1 (0.20 g) and Intermediate 80 (0.37 g, 1.10 mmol) by the procedure described in Example 200. Purification by column chromatography using silica gel and n-hexane/ethyl acetate (from 2:1 to 0:1) as eluent gave 3,4-dimethoxybenzyl [2-(2-furyl)-6-(1H-pyrazol-1-yl)pyrimidin-4-yl]carbamate (90 mg, 24%) as an off-white solid.

m.p.: 147.8-148.5° C.

δ (400 MHz, CDCl₃): 3.88 (s, 3H); 3.91 (s, 3H); 5.21 (s, 2H); 6.50 (m, 1H); 6.58 (m, 1H); 6.85-7.01 (m, 3H); 7.33 (m, 1H); 7.62 (s, 1H); 7.68 (s, 1H); 7.80 (s, 1H); 8.38 (s, 1H); 8.65 (m, 1H).

### Intermediate 81. 4-Nitrophenyl pyridin-3-ylmethyl carbonate

Obtained from pyridin-3-ylmethanol (0.51 g, 4.63 mmol) by the procedure described in Intermediate 79. Purification by trituration with a mixture of ethyl ether/diisopropyl ether (1:2), followed by a second trituration with ethyl ether gave the title compound (0.57 g, 45%).

δ (300 MHz, CDCl₃): 5.35 (s, 2H); 7.36 (s, 1H); 7.39 (d, J=9.0 Hz, 2H); 7.75-7.83 (m, 1H); 8.29 (d, J=9.0 Hz, 2H); 8.64-8.65 (m, 1H); 8.71 (s, 1H).

### EXAMPLE 203

### Pyridin-3-ylmethyl [2-(2-furyl)-6-(1H-pyrazol-1-yl)pyrimidin-4-yl]-carbamate

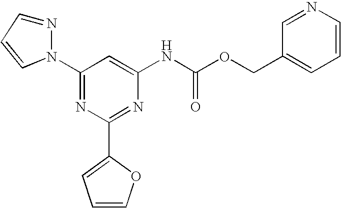

Obtained from the title compound of Example 1 (0.375 g) and Intermediate 81 (0.57 g, 2.06 mmol) by the procedure described in Example 200. Purification by trituration with ethyl ether gave pyridin-3-ylmethyl [2-(2-furyl)-6-(1H-pyrazol-1-yl)pyrimidinyl]carbamate (0.26 g, 43%) as an off-white solid.

m.p.: 253.6-255.8° C.

δ (400 MHz, DMSO-d₆): 5.29 (s, 2H); 6.66 (s, 1H); 6.74 (s, 1H); 7.45 (m, 2H); 7.88-7.96 (m, 3H); 8.23 (s, 1H); 8.57 (s, 1H); 8.69 (s, 1H); 8.77 (s, 1H); 11.17 (s, 1H).

### Intermediate 82. 4-Methoxyphenyl 4-nitrophenyl carbonate

Obtained from 4-methoxyphenol (0.50 g, 4.03 mmol) by the procedure described in Intermediate 79. Purification by trituration with isopropyl ether, followed by a second trituration with ethyl ether gave 4-methoxyphenyl 4-nitrophenyl carbonate (0.79 g, 68%).

δ (300 MHz, CDCl₃): 3.84 (s, 3H); 6.94 (d, J=9.0 Hz, 2H); 7.20 (d, J=9.0 Hz, 2H); 7.51 (d, J=9.0 Hz, 2H); 8.35 (d, J=9.0 Hz, 2H).

### EXAMPLE 204

### 4-Methoxyphenyl [2-(2-furyl)-6-(1H-pyrazol-1-yl)pyrimidin-4-yl]-carbamate

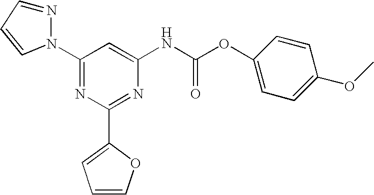

Obtained from the title compound of Example 1 (0.49 g) and Intermediate 82 (0.78 g, 2.70 mmol)) by the procedure described in Example 200. Purification by column chromatography with silica gel and n-hexane/ethyl acetate (from 5:2 to 2:1) as eluent, followed by a trituration with a mixture of diethyl ether/diisopropyl ether (1:1) gave 4-methoxyphenyl [2-(2-furyl)-6-(1H-pyrazol-1-yl)pyrimidin-4-yl]carbamate (0.11 g, 13%) as an off-white solid.

m.p.: 171.9-173.0° C.

δ (400 MHz, CDCl₃): 3.82 (s, 3H); 6.49 (m, 1H); 6.61 (m, 1H); 6.92 (m, 2H); 7.13 (m, 2H); 7.37 (d, J=3.1 Hz, 1H); 7.65 (m, 1H); 7.77 (s, 1H); 7.94 (s, 1H); 8.40 (s, 1H); 8.65 (d, J=2.7 Hz, 1H).

### Intermediate 83. 3,4-Dimethoxyphenyl 4-nitrophenyl carbonate

Obtained from 3,4-dimethoxyphenol (0.50 g, 3.24 mmol) by the procedure described in Intermediate 79. Purification by trituration with isopropyl ether gave 3,4-dimethoxyphenyl 4-nitrophenyl carbonate (0.96 g, 92%).

δ (300 MHz, CDCl₃): 3.90 (s, 6H); 6.82 (s, 1H); 6.85-6.91 (m, 2H); 7.51 (d, J=9.0 Hz, 2H); 8.32 (d, J=9.0 Hz, 2H).

### EXAMPLE 205

### 3,4-Dimethoxyphenyl [2-(2-furyl)-6-(1H-pyrazol-1-yl)pyrimidin-4-yl]carbamate

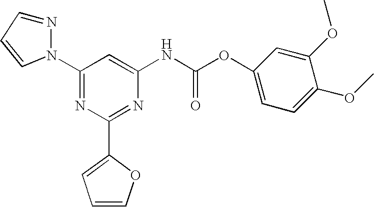

Obtained from the title compound of Example 1 (0.54 g) and Intermediate 83 (0.95 g, 2.98 mmol)) by the procedure described in Example 200. Purification by column chromatography with silica gel and n-hexane/ethyl acetate (from 2:1 to 1:2) as eluent, followed by preparative HPLC/MS purification gave 3,4-dimethoxyphenyl [2-(2-furyl-6-(1H-pyrazol-1-yl)pyrimidin-yl]carbamate (46 mg, 5%) as an off-white solid.

δ (300 MHz, CDCl₃): 3.89 (s, 3H); 3.90 (s, 3H); 6.49 (dd, J1=2.7 Hz; J2=1.6 Hz, 1H); 6.61 (dd, J1=3.4 Hz, J2=1.8 Hz, 1H); 6.72-6.90 (m, 3H); 7.38 (d, J=3.3 Hz; 1H); 7.66 (m, 1H); 7.78 (m, 1H); 8.05 (s, 1H); 8.40 (s, 1H); 8.65 (d, J=2.7 Hz, 1H).

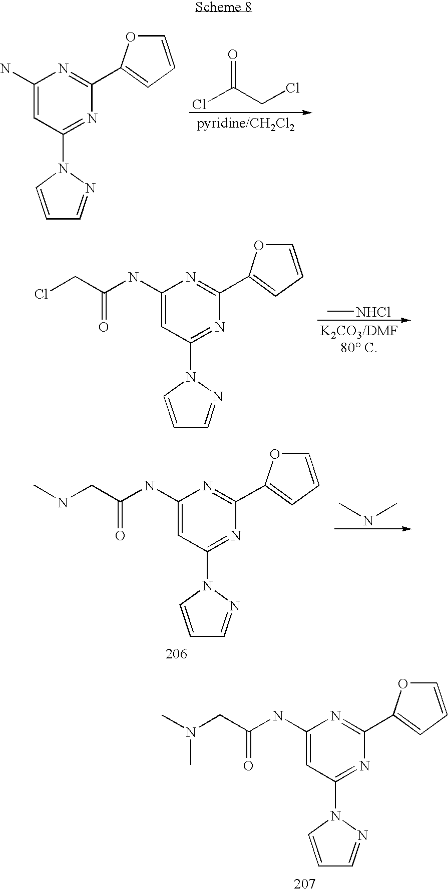

### EXAMPLE 206

### (N-(2-Furan-2-yl-6-pyrazol-1-yl-pyrimidinyl)-2-methylamino acetamide

To 5 mL dichloromethane were added 0.3 g (1.3 mmol) of the compound of Example 1 (i.e., Compound 1), 0.22 g chloroacetyl chloride (0.20 mmol, 1.5 eq) and 0.16 g pyridine. The reaction mixture was stirred at r/t for 2 hours. The reaction was quenched with 5 mL saturated sodium bicarbonate and extracted; the aqueous solution was washed with an additional 5 mL dichloromethane. The organic layers combined and dried under sodium sulfate, concentrated to a yellow solid (0.4 g, 100% crude yield).

To 1 mL DMF were added 100 mg the product obtained above, 50 mg methylamine HCl salt, and 100 mg potassium carbonate. The reaction mixture was heated at 80 C for 6 hours. Purified by Prep LC-MS, clean separation. The fractions were concentrated, redissolved in dichloromethane and extracted with diluted ammonia solution to remove the TFA. The organic layer was dried under sodium sulfate, concentrated to a slight yellow solid (25 mg, 25.4% yield). LCMS (APCI) m/z 299.0 (MH⁺). δ (300 MHz, CDCl₃): 2.52 (s, 3H), 3.43 (s, 2H), 6.48-6.50 (m, 1H), 6.57-5.59 (m, 1H); 7.36 (d, J=3.3 Hz, 1H), 7.64-7.65 (m, 1H), 7.80 (s, 1H), 8.62-8.67 (m, 2H), 9.97 (s, 1H).

### EXAMPLE 207

### 2-Dimethylamino-N-(2-furan-2-yl-6-pyrazol-1-yl-pyrimidin-4-yl)-acetamide

To 5 mL dichloromethane were added 0.3 g (1.3 mmol) of the compound of Example 1 (i.e., Compound 1), 6.22 g chloroacetyl chloride (0.20 mmol, 1.5 eq) and 0.16 g pyridine. The reaction mixture was stirred at r/t for 2 hours. The reaction was quenched with 5 mL saturated sodium bicarbonate and extracted; the aqueous solution was washed with an additional 5 mL dichloromethane. The organic layers combined and dried under sodium sulfate, concentrated to a yellow solid (0.4, 100% crude yield).

To 1 mL DMF were added 100 mg the product obtained above, 1.0 mL dimethylamine in THF (2.0 M). The reaction mixture was stirred at r/t for 2 hours. Purified by Prep LC-MS, clean separation. The fractions were concentrated, redissolved in dichloromethane and extracted with diluted ammonia solution to remove the TFA. The organic layer was dried under sodium sulfate, concentrated to a slight yellow solid (50 mg, 48.6% yield). LCMS (APCI) m/z 313.0 (MH⁺). δ (300 MHz, CDCl₃): 2.48 (s, 6H), 3.32 (s, 2H), 6.46-6.48 (m, 1H), 6.54-6.56 (m, 1H), 7.32 (s, 1H), 7.61 (s, 1H), 7.77 (s, 1H), 8.55-8.60 (m, 2H), 10.0 (s, 1H).

### EXAMPLE 208

### 2-Methylamino-N-[2-(5-methyl-furan-2-yl)-6-thiazol-2-yl-pyrimidin-4-yl]-acetamide

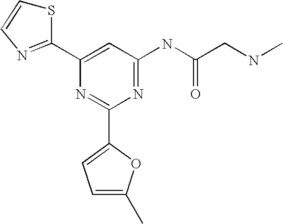

The compound was obtained by starting with the product of Example 187 (i.e., Compound 187) and following the procedure of Example 206. The mixture was purified by RPHPLC-MS to give 19.5 mg product as the TFA salt. LCMS (APCI) m/z 330.0 (MH⁺).

### EXAMPLE 209

### 2-Dimethylamino-N-[2-(5-methyl-furan-2-yl)-6-thiazol-2-yl-pyrimidin-4-yl]-acetamide

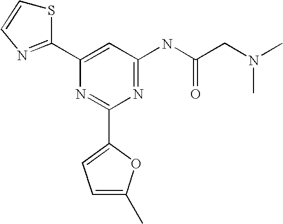

The compound was obtained by starting with the product of Example 187 (i.e., Compound 187) and following the procedure of Example 207. The mixture was purified by RPHPLC-MS to give 28.0 mg product as the TFA salt. LCMS (APCI) m/z 330.0 (MH⁺). δ (300 MHz, CDCl₃): 2.45 (s, 3H), 2.99 (s, 3H), 3.40 (m, 3H), 4.05 (s, 2H), 6.21 (d, J=3.3 Hz, 1H), 7.30 (d, J=3.3 Hz, 1H), 7.32 (s, 1H), 7.60 (d, J=2.4 Hz, 1H), 8.02 (d, J=2.4 Hz, 1H).

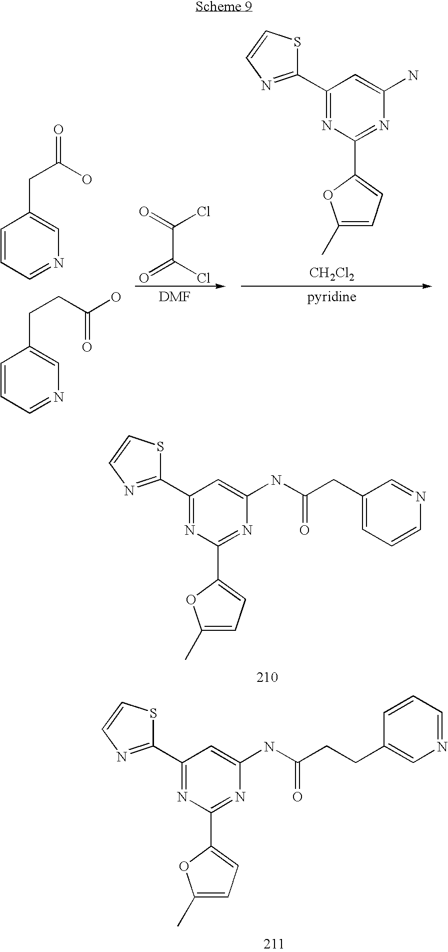

### EXAMPLE 210

### N-[2-(5-Methylfuran-2-yl)-6-thiazol-2-yl-pyrimidin-4-yl]-2-pyridin-3-yl-propionamide

To 3 mL THF were added 200 mg (1.5 mmol, 1 eq) 3-pyridylacetic acid, 200 mg (1.1 eq) oxalyl chloride, followed by a drop of DMF. The reaction mixture was stirred at r/t for 1 hour. Solvents was removed by nitrogen flow and the residue was resuspended in 3 mL dichloromethane followed by the aniline and 0.2 mL pyridine. The reaction mixture was stirred at r/t for 6 hours. The reaction mixture was extracted with 3 mL saturated sodium bicarbonate, dried under sodium sulfate and concentrated, purified by prep TLC plates using 95% chloroform, 4.9% methanol and 0.1% ammonia. Obtained a white solid. LCMS (APCI) m/z 377.9 (MH⁺). δ (300 MHz, CDCl₃): 2.45 (s, 3H), 3.78 (s, 2H), 6.18-6.20 (m, 1H), 7.30-7.34 (m, 2H), 7.53 (d, J=3.3 Hz, 1H), 7.71 (d, J=8.1 Hz, 1H), 7.99 (d, J=3.3 Hz, 1H), 8.32 (s, 1H), 8.58 (s, 2H), 8.69 (s, 1H).

### EXAMPLE 211

### N-[2-(5-Methylfuran-2-yl)-6-thiazol-2-yl-pyrimidin-4-yl]-3-pyridin-3-yl-propionamide

Compound 211 was prepared as shown in Scheme 9 according to the procedure described in Example 210. LCMS (APCI) m/z 392.0 (MH⁺). δ (300 MHz, CDCl₃): 2.45 (s, 3H), 2.76 (t, J=7.2 Hz, 2H), 3.08 (t, J=7.2 Hz, 2H), 6.18-6.20 (m, 1H), 7.19-7.23 (m, 1H), 7.31 (d, J=3.3 Hz, 1H), 7.54 (d, J=3.3 Hz, 1H), 7.57-7.60 (m, 1H), 8.02 (d, J=3.3 Hz, 1H), 8.19 (s, 1H), 8.49 (d, J=3.3 Hz, 1H), 8.52 (s, 1H), 8.70 (s, 1H).

1. A compound of formula (I) 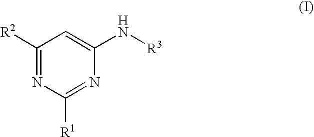
wherein
R¹ and R² independently represent a monocyclic or polycyclic heteroaryl group optionally substituted by one or more substituents selected from the group consisting of halogen atoms, straight or branched, optionally substituted lower alkyl, cycloalkyl, hydroxy, straight or branched, optionally substituted lower alkoxy, —SH, straight or branched optionally substituted lower alkylthio, cyano, —NR′R″, —CO₂R′, wherein R′ and R″ each independently represents a hydrogen atom or a straight or branched, optionally substituted lower alkyl group or R′ and R″ together with the nitrogen atom to which they are attached form a cyclic group;
R³ represents a group selected from —COR⁴, —CON(R⁴)R⁵, —COOR⁴ and —R⁶;
each of R⁴ and R⁶ independently represents a group selected from:
(a) hydrogen;
(b) a straight or branched lower alkyl group which is optionally substituted by one or more halogen atoms or by one or more cycloalkyl, hydroxy, lower alkoxy, lower alkylthio, amino, mono- or dialkylamino, alkoxyalkyl, hydroxycarbonyl, alkoxycarbonyl and nitrile groups; and
(c) a group of formula: 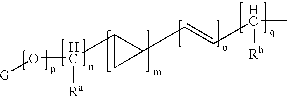
wherein:
m, o and p are independently 0 or 1;
n and q are independently selected from integers from 0 to 6;
R^{a} and R^{b} are independently a hydrogen atom or a lower alkyl group;
G is a group selected from cycloalkyl, aryl and heteroaryl groups which are optionally substituted by one or more halogen atoms or by one or more lower alkyl, cycloalkyl, lower haloalkyl, hydroxy, lower alkoxy, lower alkylthio, amino, mono- or dialkylamino, hydroxyalkyl, alkoxyalkyl, hydroxycarbonyl, alkoxycarbonyl and nitrile groups;
R⁵ represents hydrogen atom or a lower alkyl, cycloalkyl or benzyl group; or
R⁴ and R⁵ together with the nitrogen atom to which they are attached form a saturated or unsaturated ring which is optionally substituted by one or more lower alkyl, cycloalkyl or benzyl groups;
or pharmaceutically acceptable salts thereof;
with the proviso that the compound is not one of 2,6-dipyridin-4-ylpyrimidin-4-amine, 4-(3-methoxyanilino)-2,6-di(2-pyridinyl)pyrimidine, 4-(2,5-dimethoxyanilino)-2,6-di(2-pyridinyl)pyrimidine, 4-(5-methoxy-2-methylanilino)-2,6-di(2-pyridinyl)pyrimidine, 4-(2-methoxy-5-methylanilino)-2,6-di(2-pyridinyl)pyrimidine, 4-(2-chloro-5-methoxyanilino)-2,6-di(2-pyridinyl)pyrimidine, and 4-(2,5-dimethylanilino)-2,6-di(2-pyridinyl)pyrimidine.

2. A compound according to claim 1
with the proviso that when p, m and o are simultaneously zero then G is not an optionally substituted aryl group.

3. A compound according to claim 1 wherein R¹ represents a monocyclic heteroaryl group selected from the group consisting of furyl, thienyl, thiazolyl, oxazolyl, pyrazinyl, pyrazolyl, pyridazinyl, imidazolyl, triazolyl, pyrimidinyl and pyridyl groups which groups are optionally substituted by one or more substituents selected from the group consisting of halogen atoms, straight or branched, optionally substituted lower alkoxy and straight or branched, optionally substituted lower alkyl.

4. A compound according to claim 3 wherein R¹ represents a monocyclic heteroaryl group selected from the group consisting of furyl, thienyl, pyrazolyl, triazolyl, thiazolyl and pyridyl groups which groups are optionally substituted by one or more substituents selected from the group consisting of halogen atoms, straight or branched, optionally substituted lower alkoxy and straight or branched, optionally substituted lower alkyl.

5. A compound according to claim 4 wherein R¹ represents a monocyclic heteroaryl group selected from the group consisting of furyl, thienyl and pyrazolyl groups which groups are optionally substituted by one or more substituents selected from the group consisting of halogen atoms and straight or branched, optionally substituted lower alkyl.

6. A compound according to claim 5 wherein R¹ represents an unsubstituted furyl group.

7. A compound according to claim 4 wherein R² represents a monocyclic heteroaryl group selected from the group consisting of pyrazolyl, furyl, thiazolyl, oxazolyl, pyridyl, pyrimidinyl, pyrazinyl, pyridazinyl, thienyl, imidazolyl and triazolyl groups which groups are optionally substituted by one or more substituents selected from the group consisting of halogen atoms, straight or branched, optionally substituted lower alkoxy and straight or branched, optionally substituted lower alkyl.

8. A compound according to claim 7 wherein R² represents a monocyclic heteroaryl group selected from the group consisting of pyrazolyl, furyl, thiazolyl, pyridyl, thienyl and triazolyl groups which groups are optionally substituted by one or more substituents selected from the group consisting of halogen atoms, straight or branched, optionally substituted lower alkoxy and straight or branched, optionally substituted lower alkyl.

9-13. (canceled)

14. A compound according to claim 1 wherein the compound is:
2-(2-Furyl)-6-(1H-pyrazol-1-yl)pyrimidin-4-amine;
N-[2-(2-Furyl)-6-(1H-pyrazol-1-yl)pyrimidin-4-yl]acetamide;
N-[2-(2-Furyl)-6-(1H-pyrazol-1-yl)pyrimidin-4-yl]propanamide;
N-[2-(2-Furyl)-6-(1H-pyrazol-1-yl)pyrimidin-4-yl]-2-methylpropanamide;
N-[2-(2-Furyl)-6-(1H-pyrazol-1-yl)pyrimidin-4-yl]-2,2-dimethyl-propanamide;
N-[2-(2-Furyl)-6-(1H-pyrazol-1-yl)pyrimidin-4-yl]cyclopropane-carboxamide;
N-[2-(2-Furyl)-6-(1H-pyrazol-1-yl)pyrimidin-4-yl]cyclobutane-carboxamide;
N-[2-(2-Furyl)-6-(1H-pyrazol-1-yl)pyrimidin-4-yl]cyclohexane-carboxamide;
3-Cyclopentyl-N-[2-(2-furyl)-6-(1H-pyrazol-1-yl)pyrimidin-4-yl]propanamide;
N-[2-(2-Furyl)-6-(1H-pyrazol-1-yl)pyrimidin-4-yl]-2-(4-methoxyphenyl)acetamide;
2-(3,4-Dimethoxyphenyl)-N-[2-(2-furyl)-6-(1H-pyrazol-1-yl)pyrimidin-4-yl]acetamide;
N-[2-(2-Furyl)-6-(1H-pyrazol-1-yl)pyrimidin-4-yl]-3-phenyl-propanamide;
(2S)—N-[2-(2-Furyl)-6-(1H-pyrazol-1-yl)pyrimidin-4-yl]-2-phenylcyclopropanecarboxamide;
3,3,3-Trifluoro-N-[2-(2-furyl)-6-(1H-pyrazol-1-yl)pyrimidin-4-yl]propanamide;
3-(3,4-Dimethoxyphenyl)-N-[2-(2-furyl)-6-(1H-pyrazol-1-yl)pyrimidin-4-yl]propanamide;
N-[2-(2-Furyl)-6-(1H-pyrazol-1-yl)pyrimidin-4-yl]-2-methyl-3-phenylpropanamide;
N-[2-(2-Furyl)-6-(1H-pyrazol-1-yl)pyrimidin-4-yl]-3-phenoxy-propanamide;
N-[2-(2-Furyl)-6-(1H-pyrazol-1-yl)pyrimidin-4-yl]-3-pyridin-3-ylpropanamide;
N-[2-(2-Furyl)-6-(1H-pyrazol-1-yl)pyrimidin-4-yl]-2-pyridin-3-yl-acetamide;
(2E)-3-(3,4-Dimethoxyphenyl)-N-[2-(2-furyl)-6-(1H-pyrazol-1-yl)pyrimidin-4-yl]acrylamide;
6-(3,5-Dimethyl-1H-pyrazol-1-yl)-2-(2-furyl)pyrimidin-4-amine;
N-[6-(3,5-Dimethyl-1H-pyrazol-1-yl)-2-(2-furyl)pyrimidin-4-yl]acetamide;
N-[2-(3,5-Dimethyl-1H-pyrazol-1-yl)-6-(2-furyl)pyrimidin-4-yl]propanamide;
N-[6-(3,5-Dimethyl-1H-pyrazol-1-yl)-2-(2-furyl)pyrimidin-4-yl]-2-methylpropanamide;
N-[6-(3,5-Dimethyl-1H-pyrazol-1-yl)-2-(2-furyl)pyrimidin-4-yl]-2,2-dimethylpropanamide;
N-[6-(3,5-Dimethyl-1H-pyrazol-1-yl)-2-(2-furyl)pyrimidin-4-yl]-cyclopropanecarboxamide;
3-Cyclopentyl-N-[6-(3,5-dimethyl-1H-pyrazol-1-yl)-2-(2-furyl)pyrimidin-4-yl]propanamide;
N-[6-(3,5-dimethyl-1H-pyrazol-1-yl)-2-(2-furyl)pyrimidin-4-yl]-2-(4-methoxyphenyl)acetamide;
2-(3,4-Dimethoxyphenyl)-N-[6-(3,5-dimethyl-1H-pyrazol-1-yl)-2-(2-furyl)pyrimidin-4-yl]acetamide;
N-[6-(3,5-Dimethyl-1H-pyrazol-1-yl)-2-(2-furyl)pyrimidin-4-yl]-3-phenylpropanamide;
N-[6-(3,5-Dimethyl-1H-pyrazol-1-yl)-2-(2-furyl)pyrimidin-4-yl]-3,3,3-trifluoropropanamide;
3-(3,4-Dimethoxyphenyl)-N-[6-(3,5-dimethyl-1H-pyrazol-1-yl)-2-(2-furyl)pyrimidin-4-yl]propanamide;
N-[6-(3,5-Dimethyl-1H-pyrazol-1-yl)-2-(2-furyl)pyrimidin-4-yl]-3-phenoxypropanamide;
N-[6-(3,5-Dimethyl-1H-pyrazol-1-yl)-2-(2-furyl)pyrimidin-4-yl]-2-pyridin-3-ylacetamide;
N-[6-(3,5-Dimethyl-1H-pyrazol-1-yl)-2-(2-furyl)pyrimidin-4-yl]-3-pyridin-3-ylpropanamide;
2-(2-Furyl)-6-(4-methyl-1H-pyrazol-1-yl)pyrimidin-4-amine;
N-[2-(2-Furyl)-6-(4-methyl-1H-pyrazol-1-yl)pyrimidin-4-yl]-propanamide;
2-(2-Furyl)-6-(3-methyl-1H-pyrazol-1-yl)pyrimidin-4-amine;
N-[2-(2-Furyl)-6-(3-methyl-1H-pyrazol-1-yl)pyrimidin-4-yl]propanamide;
2-(2-Furyl)-6-[3-(trifluoromethyl)-1H-pyrazol-1-yl]pyrimidin-4-amine;
N-{2-(2-Furyl)-6-[3-(trifluoromethyl)-1H-pyrazol-1-yl]pyrimidin-4-yl}-propanamide;
2-(2-Furyl)-6-[5-methyl-3-(trifluoromethyl)-1H-pyrazol-1-yl]pyrimidin-4-amine;
N-{2-(2-furyl)-6-[5-methyl-3-(trifluoromethyl)-1H-pyrazol-1-yl]-pyrimidin-4-yl}propanamide;
2-(2-Furyl)-6-(1H-1,2,4-triazol-1-yl)pyrimidin-4-amine;
N-[2-(2-furyl)-6-(1H-1,2,4-triazol-1-yl)pyrimidin-4-yl]acetamide;
N-[2-(2-Furyl)-6-(1H-1,2,4-triazol-1-yl)pyrimidin-4-yl]propanamide;
3,3,3-Trifluoro-N-[2-(2-furyl)-6-(1H-1,2,4-triazol-1-yl)pyrimidin-4-yl]-propanamide;
2-(5-Bromo-2-furyl)-6-(1H-pyrazol-1-yl)pyrimidin-4-amine;
N-[2-(5-bromo-2-furyl)-6-(1H-pyrazol-1-yl)pyrimidin-4-yl]propanamide;
2-(5-Chloro-2-furyl)-6-(1H-pyrazol-1-yl)pyrimidin-4-amine;
N-[2-(5-Chloro-2-furyl)-6-(1H-pyrazol-1-yl)pyrimidin-4-yl]propanamide;
2-(5-Methyl-2-furyl)-6-(1H-pyrazol-1-yl)pyrimidin-4-amine;
N-[2-(5-methyl-2-furyl)-6-(1H-pyrazol-1-yl)pyrimidin-4-yl]propanamide;
N-[2-(2-Furyl)-6-pyridin-3-ylpyrimidin-4-yl]propanamide;
2-(2-Furyl)-6-pyridin-3-ylpyrimidin-4-amine;
6-(1H-Pyrazol-1-yl)-2-(2-thienyl)pyrimidin-4-amine;
N-[6-(1H-Pyrazol-1-yl)-2-(2-thienyl)pyrimidin-4-yl]acetamide;
N-[6-(1H-Pyrazol-1-yl)-2-(2-thienyl)pyrimidin-4-yl]propanamide;
3-Cyclopentyl-N-[6-(1H-pyrazol-1-yl)-2-(2-thienyl)pyrimidin-4-yl]propanamide;
3-Phenyl-N-[6-(1H-pyrazol-1-yl)-2-(2-thienyl)pyrimidin-4-yl]-propanamide;
3,3,3-Trifluoro-N-[6-(1H-pyrazol-1-yl)-2-(2-thienyl)pyrimidin-4-yl]-propanamide;
3-(3,4-Dimethoxyphenyl)-N-[6-(1H-pyrazol-1-yl)-2-(2-thienyl)pyrimidin-4-yl]propanamide;
3-Phenoxy-N-[6-(1H-pyrazol-1-yl)-2-(2-thienyl)pyrimidin-4-yl]-propanamide;
N-[6-(1H-Pyrazol-1-yl)-2-(2-thienyl)pyrimidin-4-yl]-2-pyridin-3-yl-acetamide;
N-[6-(1H-Pyrazol-1-yl)-2-(2-thienyl)pyrimidin-4-yl]-3-pyridin-3-ylpropanamide;
(2E)-3-(3,4-Dimethoxyphenyl)-N-[6-(1H-pyrazol-1-yl)-2-(2-thienyl)-pyrimidin-4-yl]acrylamide;
6-(3,5-Dimethyl-1H-pyrazol-1-yl)-2-(2-thienyl)pyrimidin-4-amine;
N-[6-(3,5-Dimethyl-1H-pyrazol-1-yl)-2-(2-thienyl)pyrimidin-4-yl]-acetamide;
N-[6-(3,5-Dimethyl-1H-pyrazol-1-yl)-2-(2-thienyl)pyrimidin-4-yl]-propanamide;
N-[6-(3,5-Dimethyl-1H-pyrazol-1-yl)-2-(2-thienyl)pyrimidin-4-yl]-3,3,3-trifluoropropanamide;
2-(2-Thienyl)-6-(1H-1,2,4-triazol-1-yl)pyrimidin-4-amine;
N-[2-(2-Thienyl)-6-(1H-1,2,4-triazol-1-yl)pyrimidin-4-yl]acetamide;
N-[2-(2-Thienyl)-6-(1H-1,2,4-triazol-1-yl)pyrimidin-4-yl]propanamide;
3,3,3-Trifluoro-N-[2-(2-thienyl)-6-(1H-1,2,4-triazol-1-yl)pyrimidin-4-yl]propanamide;
N-[2-(3-Methyl-2-thienyl)-6-(1H-pyrazol-1-yl)pyrimidin-4-yl]-propanamide;
6-(2-Furyl)-2-(1H-pyrazol-1-yl)pyrimidin-4-amine;
N-[6-(2-Furyl)-2-(1H-pyrazol-1-yl)pyrimidin-4-yl]acetamide;
N-[6-(2-Furyl)-2-(1H-pyrazol-1-yl)pyrimidin-4-yl]propanamide;
3,3,3-Trifluoro-N-[6-(2-furyl)-2-(1H-pyrazol-1-yl)pyrimidin-4-yl]-propanamide;
2-(3,5-Dimethyl-1H-pyrazol-1-yl)-6-(2-furyl)pyrimidin-4-amine;
N-[2-(3,5-Dimethyl-1H-pyrazol-1-yl)-6-(2-furyl)pyrimidin-4-yl]-propanamide;
N-[2-(3,5-Dimethyl-1H-pyrazol-1-yl)-6-(2-furyl)pyrimidin-4-yl]-2-(4-methoxyphenyl)acetamide;
6-(2-Furyl)-2-(1H-1,2,4-triazol-1-yl)pyrimidin-4-amine;
N-[6-(2-Furyl)-2-(1H-1,2,4-triazol-1-yl)pyrimidin-4-yl]propanamide;
2-(1H-Pyrazol-1-yl)-6-pyridin-2-ylpyrimidin-4-amine;
N-[2-(1H-Pyrazol-1-yl)-6-pyridin-2-ylpyrimidin-4-yl]propanamide;
2-(3,5-Dimethyl-1H-pyrazol-1-yl)-6-pyridin-2-ylpyrimidin-4-amine;
N-[2-(3,5-Dimethyl-1H-pyrazol-1-yl)-6-pyridin-2-ylpyrimidin-4-yl]propanamide;
N-[2-(3,5-Dimethyl-1H-pyrazol-1-yl)-6-pyridin-2-ylpyrimidin-4-yl]-2-(4-methoxyphenyl)acetamide;
2-(1H-Pyrazol-1-yl)-6-pyridin-3-ylpyrimidin-4-amine;
N-[2-(1H-Pyrazol-1-yl)-6-pyridin-3-ylpyrimidin-4-yl]propanamide;
2-(3,5-Dimethyl-1H-pyrazol-1-yl)-6-pyridin-3-ylpyrimidin-4-amine;
N-[2-(3,5-Dimethyl-1H-pyrazol-1-yl)-6-pyridin-3-ylpyrimidin-4-yl]-propanamide;
N-[2-(3,5-Dimethyl-1H-pyrazol-1-yl)-6-pyridin-3-ylpyrimidin-4-yl]-2-(4-methoxyphenyl)acetamide;
2-(1H-Pyrazol-1-yl)-6-pyridin-4-ylpyrimidin-4-amine;
N-[2-(1H-Pyrazol-1-yl)-6-pyridin-4-ylpyrimidin-4-amine;
6-(2-Furyl)-2-pyridin-2-ylpyrimidin-4-amine;
N-[6-(2-Furyl)-2-pyridin-2-ylpyrimidin-4-yl]propanamide;
2-(3-Methylpyridin-2-yl)-6-(1H-pyrazol-1-yl)pyrimidin-4-amine;
N-[2-(3-methylpyridin-2-yl)-6-(1H-pyrazol-1-yl)pyrimidin-4-yl]propanamide;
6-(1H-Pyrazol-1-yl)-2-pyridin-3-ylpyrimidin-4-amine;
N-[6-(1H-Pyrazol-1-yl)-2-pyridin-3-ylpyrimidin-4-yl]acetamide;
N-[6-(1H-Pyrazol-1-yl)-2-pyridin-3-ylpyrimidin-4-yl]propanamide;
6-(3,5-Dimethyl-1H-pyrazol-1-yl)-2-pyridin-3-ylpyrimidin-4-amine;
N-[6-(3,5-Dimethyl-1H-pyrazol-1-yl)-2-pyridin-3-ylpyrimidin-4-yl]-acetamide;
N-[6-(3,5-Dimethyl-1H-pyrazol-1-yl)-2-pyridin-3-ylpyrimidin-4-yl]-propanamide;
N-[6-(3,5-Dimethyl-1H-pyrazol-1-yl)-2-pyridin-3-ylpyrimidin-4-yl]-3,3,3-trifluoropropanamide;
2-Pyridin-3-yl-6-(1H-1,2,4-triazol-1-yl)pyrimidin-4-amine;
3,3,3-Trifluoro-N-[2-pyridin-3-yl-6-(1H-1,2,4-triazol-1-yl)pyrimidin-4-yl]propanamide;
6-(2-Furyl)-2-pyridin-3-ylpyrimidin-4-ylamine;
N-[6-(2-Furyl)-2-pyridin-3-ylpyrimidin-4-yl]propanamide;
N-[6-(3,5-Dimethyl-1H-pyrazol-1-yl)-2-pyridin-4-ylpyrimidin-4-yl]propanamide;
6-(3,5-dimethyl-1H-pyrazol-1-yl)-2-pyridin-4-ylpyrimidin-4-amine;
6-(2-Furyl)-2-pyridin-4-ylpyrimidin-4-ylamine;
N-[6-(2-Furyl)-2-pyridin-4-ylpyrimidin-4-yl]propanamide;
6-(2-Furyl)-2-(1,3-thiazol-2-yl)pyrimidin-4-amine;
N-[6-(2-Furyl)-2-(1,3-thiazol-2-yl)pyrimidin-4-yl]propanamide;
2-(4-Fluorophenyl)-N-[6-(2-furyl)-2-(1,3-thiazol-2-yl)pyrimidin-4-yl]acetamide;
N-(Cyclopropylmethyl)-2-(2-furyl)-6-(1H-pyrazol-1-yl)pyrimidin-4-amine;
(2R)-2-{[2-(2-Furyl)-6-(1H-pyrazol-1-yl)pyrimidin-4-yl]amino}propan-1-ol;
3-{[2-(2-Furyl)-6-(1H-pyrazol-1-yl)pyrimidin-4-yl]amino}propan-1-ol;
N-[2-(2-Furyl)-6-(1H-pyrazol-1-yl)pyrimidin-4-yl]ethane-1,2-diamine;
2-(2-Furyl)-N-[2-(4-methoxyphenyl)ethyl]-6-(1H-pyrazol-1-yl)-pyrimidin-4-amine;
N-[2-(3,4-Dimethoxyphenyl)ethyl]-2-(2-furyl)-6-(1H-pyrazol-1-yl)-pyrimidin-4-amine;
2-(2-Furyl)-6-(1H-pyrazol-1-yl)-N-[2-(pyridin-2-yl)ethyl]pyrimidin-4-amine;
2-(2-Furyl)-6-(1H-pyrazol-1-yl)-N-[2-(pyridin-3-yl)ethyl]pyrimidin-4-amine;
2-(2-Furyl)-N-(3-phenylpropyl)-6-(1H-pyrazol-1-yl)pyrimidin-4-amine;
2-(2-Furyl)-N-[3-(1H-imidazol-1-yl)propyl]-6-(1H-pyrazol-1-yl)-pyrimidin-4-amine;
N-(Cyclopropylmethyl)-6-(1H-pyrazol-1-yl)-2-(2-thienyl)pyrimidin-4-amine;
(2R)-2-{[6-(1H-Pyrazol-1-yl)-2-(2-thienyl)pyrimidin-4-yl]amino}-propan-1-ol;
3-{[6-(1H-Pyrazol-1-yl)-2-(2-thienyl)pyrimidin-4-yl]amino}propan-1-ol;
N-(2-Aminoethyl)-N-[6-(1H-pyrazol-1-yl)-2-(2-thienyl)pyrimidin-4-yl]amine;
N-[2-(4-Methoxyphenyl)ethyl]-6-(1H-pyrazol-1-yl)-2-(2-thienyl)-pyrimidin-4-amine;
N-[2-(3,4-Dimethoxyphenyl)ethyl]-6-(1H-pyrazol-1-yl)-2-(2-thienyl)-pyrimidin-4-amine;
6-(1H-Pyrazol-1-yl)-N-(2-pyridin-3-ylethyl)-2-(2-thienyl)pyrimidin-4-amine;
6-(1H-Pyrazol-1-yl)-N-(2-pyridin-2-ylethyl)-2-(2-thienyl)pyrimidin-4-amine;
N-(3-Phenylpropyl)-6-(1H-pyrazol-1-yl)-2-(2-thienyl)pyrimidin-4-amine;
N-[3-(1H-imidazol-1-yl)propyl]-6-(1H-pyrazol-1-yl)-2-(2-thienyl)-pyrimidin-4-amine;
Ethyl 6-(1H-pyrazol-1-yl)-2-(2-thienyl)pyrimidin-4-yl]carbamate;
N-[2-(2-Furyl)-6-(1H-pyrazol-1-yl)pyrimidin-4-yl]-N′-[(1S*,2R*)-2-phenylcyclopropyl]urea (*relative trans configuration);
N-[2-(2-Furyl)-6-(1H-pyrazol-1-yl)pyrimidin-4-yl]-N′-propylurea;
N-[2-(2-Furyl)-6-(1H-pyrazol-1-yl)pyrimidin-4-yl]-N′-isopropylurea;
N-Cyclopentyl-N′-[2-(2-furyl)-6-(1H-pyrazol-1-yl)pyrimidin-4-yl]urea;
N-[2-(2-Furyl)-6-(1H-pyrazol-1-yl)pyrimidin-4-yl]-N′-(4-methoxy-phenyl)urea;
N-[2-(2-Furyl)-6-(1H-pyrazol-1-yl)pyrimidin-4-yl]-N′-(2-phenylethyl)-urea;
N-Benzyl-N′-[2-(2-furyl)-6-(1H-pyrazol-1-yl)pyrimidin-4-yl]urea;
N-[2-(2-Furyl)-6-(1H-pyrazol-1-yl)pyrimidin-4-yl]-3-methylbutanamide;
N-[2-(2-Furyl)-6-(1H-pyrazol-1-yl)pyrimidin-4-yl]-3,3-dimethyl-butanamide;
N-[2-(2-Furyl)-6-(1H-pyrazol-1-yl)pyrimidin-4-yl]cyclopentane-carboxamide;
2-Chloro-N-[2-(2-furyl)-6-(1H-pyrazol-1-yl)pyrimidin-4-yl]-2-phenyl-acetamide;
N-[2-(2-Furyl)-6-(1H-pyrazol-1-yl)pyrimidin-4-yl]-2-phenylacetamide;
2-(4-Fluorophenyl)-N-[2-(2-furyl)-6-(1H-pyrazol-1-yl)pyrimidin-4-yl]acetamide;
N-[2-(2-Furyl)-6-(1H-pyrazol-1-yl)pyrimidin-4-yl]-2-(3-methoxyphenyl)acetamide;
N-[2-(2-Furyl)-6-(1H-pyrazol-1-yl)pyrimidin-4-yl]-2-(2-methoxyphenyl)acetamide;
2-(3,4-Dichlorophenyl)-N-[2-(2-furyl)-6-(1H-pyrazol-1-yl)pyrimidin-4-yl]acetamide;
2-(1,3-Dibenzodioxol-5-yl)-N-[2-(2-furyl)-6-(1H-pyrazol-1-yl)pyrimidin-4-yl]acetamide;
2-(3,4-Dihydroxyphenyl)-N-[2-(2-furyl)-6-(1H-pyrazol-1-yl)pyrimidin-4-yl]acetamide;
2-(2,5-Dimethoxyphenyl)-N-[2-(2-furyl)-6-(1H-pyrazol-1-yl)pyrimidin-4-yl]acetamide;
2-(4-Chloro-3-methylphenyl)-N-[2-(2-furyl)-6-(1H-pyrazol-1-yl)-pyrimidin-4-yl]acetamide;
2-(3,5-Dimethoxyphenyl)-N-[2-(2-furyl)-6-(1H-pyrazol-1-yl)pyrimidin-4-yl]acetamide;
2-[3-(Benzyloxy)-4-methoxyphenyl]-N-[2-(2-furyl)-6-(1H-pyrazol-1-yl)pyrimidin-4-yl]acetamide;
2-[4-(Cyclobutyloxy)-3-methoxyphenyl]-N-[2-(2-furyl)-6-(1H-pyrazol-1-yl)pyrimidin-4-yl]acetamide;
N-[2-(2-Furyl)-6-(1H-pyrazol-1-yl)pyrimidin-4-yl]-2-(4-difluoromethoxy-3-methoxyphenyl)acetamide;
N-[2-(2-Furyl)-6-(1H-pyrazol-1-yl)pyrimidin-4-yl]-2-(3,4,5-trimethoxyphenyl)acetamide;
2-(3,4-Dimethoxyphenyl)-N-[2-(2-furyl)-6-(1H-pyrazol-1-yl)pyrimidin-4-yl]propanamide;
N-[2-(2-Furyl)-6-(1H-pyrazol-1-yl)pyrimidin-4-yl]benzamide;
N-[2-(2-Furyl)-6-(1H-pyrazol-1-yl)pyrimidin-4-yl]-3,4-dimethoxy-benzamide;
2,6-Difluoro-N-[2-(2-furyl)-6-(1H-pyrazol-1-yl)pyrimidin-4-yl]-benzamide;
N-[2-(2-Furyl)-6-(1H-pyrazol-1-yl)pyrimidin-4-yl]-2-furamide;
N-[2-(2-Furyl)-6-(1H-pyrazol-1-yl)pyrimidin-4-yl]thiophene-2-carboxamide;
N-[2-(2-Furyl)-6-(1H-pyrazol-1-yl)pyrimidin-4-yl]nicotinamide;
N-[2-(2-Furyl)-6-(1H-pyrazol-1-yl)pyrimidin-4-yl]isonicotinamide;
N-[2-(2-Furyl)-6-(1H-pyrazol-1-yl)pyrimidin-4-yl]-1-naphthamide;
N-[2-(2-Furyl)-6-(1H-pyrazol-1-yl)pyrimidin-4-yl]quinoline-2-carboxamide;
(2E)-3-(3,4-Dimethoxyphenyl)-N-[6-(3,5-dimethyl-1H-pyrazol-1-yl)-2-(2-furyl)pyrimidin-4-yl]acrylamide;
2-(2-Furyl)-6-(2H-1,2,3-triazol-2-yl)pyrimidin-4-amine;
2-(2-furyl)-6-(1H-1,2,3-triazol-1-yl)pyrimidin-4-amine;
N-[2-(2-Furyl)-6-(2H-1,2,3-triazol-2-yl)pyrimidin-4-yl]propanamide;
2-(3,4-Dimethoxyphenyl)-N-[2-(2-furyl)-6-(2H-1,2,3-triazol-2-yl)-pyrimidin-4-yl]acetamide;
N-[2-(2-Furyl)-6-(1H-1,2,3-triazol-1-yl)pyrimidin-4-yl]propanamide;
2-(2-Furyl)-6-(1,3-thiazol-2-yl)pyrimidin-4-amine;
N-[2-(2-Furyl)-6-(1,3-thiazol-2-yl)pyrimidin-4-yl]propanamide;
3-(3,4-Dimethoxyphenyl)-N-[2-(2-furyl)-6-(1,3-thiazol-2-yl)pyrimidin-4-yl]propanamide;
2,6-Di-2-furylpyrimidin-4-amine;
N-(2,6-Di-2-furylpyrimidin-4-yl)-2-(3,4-dimethoxyphenyl)acetamide;
6-(1,3-Benzothiazol-2-yl)-2-(2-furyl)pyrimidin-4-amine;
2-(5-Methyl-2-furyl)-6-(1,3-thiazol-2-yl)pyrimidin-4-amine;
6-(1,3-Thiazol-2-yl)-2-(2-thienyl)pyrimidin-4-amine;
2-(3,4-Dimethoxyphenyl)-N-[6-(2-furyl)-2-(1,3-thiazol-2-yl)pyrimidin-4-yl]acetamide;
6-(1H-Pyrazol-1-yl)-2-(1,3-thiazol-2-yl)pyrimidin-4-amine;
2-(3,4-Dimethoxyphenyl)-N-[6-(1H-pyrazol-1-yl)-2-(1,3-thiazol-2-yl)pyrimidin-4-yl]acetamide;
2-(2-Furyl)-N-methyl-6-(1,3-thiazol-2-yl)pyrimidin-4-amine;
N-(Cyclopropylmethyl)-2-(2-furyl)-6-(1,3-thiazol-2-yl)pyrimidin-4-amine;
N-[2-(3,4-Dimethoxyphenyl)ethyl]-2-(2-furyl)-6-(1,3-thiazol-2-yl)-pyrimidin-4-amine;
N-(Cyclopropylmethyl)-6-(2-furyl)-2-(1,3-thiazol-2-yl)pyrimidin-4-amine;
N-[2-(3,4-Dimethoxyphenyl)ethyl]-6-(2-furyl)-2-(1,3-thiazol-2-yl)-pyrimidin-4-amine;
6-(2-Furyl)-N-(2-pyridin-3-ylethyl)-2-(1,3-thiazol-2-yl)pyrimidin-4-amine;
6-(2-Furyl)-N-[(1S*,2R*)-2-phenylcyclopropyl]-2-(1,3-thiazol-2-yl)-pyrimidin-4-amine (*relative trans configuration);
Ethyl [2-(2-furyl)-6-(1H-pyrazol-1-yl)pyrimidin-4-yl]carbamate;
Cyclopentylmethyl [2-(2-furyl)-6-(1H-pyrazol-1-yl)pyrimidin-4-yl]-carbamate;
Benzyl [2-(2-furyl)-6-(1H-pyrazol-1-yl)pyrimidin-4-yl]carbamate; 3,4-Dimethoxybenzyl [2-(2-furyl)-6-(1H-pyrazol-1-yl)pyrimidin-4-yl]carbamate;
Pyridin-3-ylmethyl [2-(2-furyl)-6-(1H-pyrazol-1-yl)pyrimidin-4-yl]-carbamate;
4-Methoxyphenyl [2-(2-furyl)-6-(1H-pyrazol-1-yl)pyrimidin-4-yl]-carbamate; or
3,4-Dimethoxyphenyl [2-(2-furyl)-6-(1H-pyrazol-1-yl)pyrimidin-4-yl]carbamate.

15. (canceled)

16. A method for treating ischemia, supraventricular arrhythmias, acute renal failure, myocardial reperfusion injury, allergic reactions including but not limited to rhinitis, urticaria, scleroderm arthritis, inflammatory bowel diseases, asthma, diabetes mellitus, obesity, Parkinson disease, Huntington's disease, dystonias, restless leg syndrome, or dyskinesias, comprising administering to a subject in need thereof a pharmaceutically effective amount of a compound according to claim 1.

17. (canceled)

18. A method according to claim 16 wherein the condition is ischemia, supraventricular arrhythmias, Parkinson disease, Huntington's disease, dystonias, restless leg syndrome, or dyskinesias.

19. A pharmaceutical composition comprising a compound according to claim 1 and a pharmaceutically acceptable diluent or carrier.

20-27. (canceled)

28. A pharmaceutical composition comprising a compound according to claim 1; and another compound selected from (a) L-DOPA, (b) a dopamine antagonist, (c) an inhibitor of dopamine decarboxylase (d) a catechol-O-methyltransferase inhibitor and (e) an inhibitor of monoamine oxidase.

29-34. (canceled)
